(19) United States
(12) Patent Application Publication
(10) Pub. No.: US 2014/0065620 A1
(43) Pub. Date: Mar. 6, 2014

(54) NUCLEIC ACIDS FOR DETECTING BREAST CANCER

(71) Applicant: MAYO FOUNDATION FOR MEDICAL EDUCATION AND RESEARCH, Rochester, MN (US)

(72) Inventors: Edith A. Perez, Rochester, MN (US); E. Aubrey Thompson, JR., Jacksonville, FL (US); Yan Asmann, Rochester, MN (US)

(73) Assignee: MAYO FOUNDATION FOR MEDICAL EDUCATION AND RESEARCH, Rochester, MN (US)

(21) Appl. No.: 13/725,414

(22) Filed: Dec. 21, 2012

Related U.S. Application Data
(60) Provisional application No. 61/581,627, filed on Dec. 29, 2011.

(51) Int. Cl. C12Q 1/68

(52) U.S. Cl. CPC C12Q 1/6886
USPC 435/6.12; 536/23.4

(57) ABSTRACT

This document provides methods and materials involved in detecting breast cancer. For example, nucleic acids for detecting gene rearrangements (e.g., translocations) associated with breast cancer as well as methods and materials for detecting breast cancer are provided.

### CROSS-REFERENCE TO RELATED APPLICATIONS

This application claims the benefit of U.S. Provisional Application Ser. No. 61/581,627, filed Dec. 29, 2011. The disclosure of the prior application is considered part of (and is incorporated by reference in) the disclosure of this application.

### BACKGROUND

1. Technical Field

This document relates to methods and materials involved in detecting breast cancer. For example, this document provides nucleic acids for detecting gene rearrangements (e.g., translocations) associated with breast cancer as well as methods and materials for detecting breast cancer.

2. Background Information

Gene fusion events resulting from inversions, interstitial deletion, or translocations represent one of the most common types of genomic rearrangement. So far, the majority of fusion genes have been identified in leukemias, lymphomas, and sarcomas. Recently, the discovery of TMPRSS2-ERG fusions in prostate cancer and EML4-ALK fusion in non-small cell lung tumors suggests that gene fusion events may as well occur with a relatively high frequency in solid tumors, leading to the generation of fusion proteins with unique oncogenic properties. The BCR-ABL1 fusion gene can be used as a diagnostic marker for chronic myelogenous leukemia (CML), and is a drug target of Imatinib (Gleevec) in cells that harbor the BCR-ABL1 fusion gene. The prostate cancer specific TMPRSS2-ERG fusion events place growth regulatory genes under the influence of an androgen-regulated promoter, giving rise to an oncogene that has the potential to amplify normal androgen-dependent growth.

### SUMMARY

This document provides methods and materials involved in detecting breast cancer. For example, this document provides nucleic acids for detecting gene rearrangements (e.g., translocations) associated with breast cancer as well as methods and materials for detecting breast cancer. As described herein, a patient sample (e.g., a breast tissue sample) can be assessed for the presence or absence of one or more of the gene rearrangements set forth in Table 3, 4, 5, 6, 8, or 10. In some cases, the presence of one or more gene rearrangements set forth in Table 3, 4, 5, 6, 8, or 10 can indicate that the patient has breast cancer. Detecting a gene rearrangement set forth in Table 3, 4, 5, 6, 8, or 10 can allow clinicians and patients to diagnose breast cancer in an efficient and effective manner.

In general, one aspect of this document features a primer pair comprising, or consisting essentially of, first and second primers, wherein an amplification reaction comprising the first and second primers has the ability to amplify a nucleic acid having a fusion partner A sequence and a fusion partner B sequence, wherein the fusion partner A sequence is present in a first human gene set forth in Table 3, 4, 5, 6, 8, or 10 and the fusion partner B sequence is present in a second human gene set forth in Table 3, 4, 5, 6, 8, or 10 as being a fusion partner with the first human gene. The fusion partner A sequence can be at least 10 nucleotides. The fusion partner A sequence can be at least 50 nucleotides. The fusion partner A sequence can be at least 100 nucleotides. The fusion partner B sequence can be at least 10 nucleotides. The fusion partner B sequence can be at least 50 nucleotides. The fusion partner B sequence can be at least 100 nucleotides. The first primer can be between 13 and 100 nucleotides in length. The first primer can be between 15 and 50 nucleotides in length. The second primer can be between 13 and 100 nucleotides in length. The second primer can be between 15 and 50 nucleotides in length. The fusion partner A sequence can be present in a human LIMA1 nucleic acid, and the fusion partner B sequence can be present in a human USP22 nucleic acid. The fusion partner A sequence can be present in a human LIMA1 nucleic acid, and the fusion partner B sequence can be present in a human USP22 nucleic acid. The fusion partner A sequence can be present in a human ACACA nucleic acid, and the fusion partner B sequence can be present in a human STAC2 nucleic acid. The fusion partner A sequence can be present in a human FAM102A nucleic acid, and the fusion partner B sequence can be present in a human CIZ1 nucleic acid. The fusion partner A sequence can be present in a human GLB1 nucleic acid, and the fusion partner B sequence can be present in a human CMTM7 nucleic acid. The fusion partner A sequence can be present in a human MED1 nucleic acid, and the fusion partner B sequence can be present in a human STXBP4 nucleic acid. The fusion partner A sequence can be present in a human PIP4K2B nucleic acid, and the fusion partner B sequence can be present in a human RAD51C nucleic acid. The fusion partner A sequence can be present in a human RAB22A nucleic acid, and the fusion partner B sequence can be present in a human MYO9B nucleic acid. The fusion partner A sequence can be present in a human RPS6KB 1 nucleic acid, and the fusion partner B sequence can be present in a human SNF8 nucleic acid. The fusion partner A sequence can be present in a human STARD3 nucleic acid, and the fusion partner B sequence can be present in a human DOK5 nucleic acid. The fusion partner A sequence can be present in a human TRPC4AP nucleic acid, and the fusion partner B sequence can be present in a human MRPL45 nucleic acid. The fusion partner A sequence can be present in a human ZMYND8 nucleic acid, and the fusion partner B sequence can be present in a human CEP250 nucleic acid. The fusion partner A sequence can be present in a human CTAGE5 nucleic acid, and the fusion partner B sequence can be present in a human SIP1 nucleic acid. The fusion partner A sequence can be present in a human MLL5 nucleic acid, and the fusion partner B sequence can be present in a human LHFPL3 nucleic acid. The fusion partner A sequence can be present in a human SEC22B nucleic acid, and the fusion partner B sequence can be present in a human NOTCH2 nucleic acid. The fusion partner A sequence can be present in a human EIF3K nucleic acid, and the fusion partner B sequence can be present in a human CYP39A1 nucleic acid. The fusion partner A sequence can be present in a human RAB7A nucleic acid, and the fusion partner B sequence can be present in a human LRCH3 nucleic acid. The fusion partner A sequence can be present in a human RNF187 nucleic acid, and the fusion partner B sequence can be present in a human OBSCN nucleic acid. The fusion partner A sequence can be present in a human SLC37A1 nucleic acid, and the fusion partner B sequence can be present in a human ABCG1 nucleic acid. The fusion partner A sequence can be present in a human EXOC7 nucleic acid, and the fusion partner B sequence can be present in a human CYTH1 nucleic acid. The fusion partner A sequence can be present in a human BRE nucleic acid, and the fusion partner B sequence can be present in a human DPYSL5 nucleic acid. The fusion partner A sequence can be present in a human CD151 nucleic acid, and the fusion partner B sequence can be present in a human DRD4 nucleic acid. The fusion partner A sequence can be present in a human LDLRAD3 nucleic acid, and the fusion partner B sequence can be present in a human TCP11L1 nucleic acid. The fusion partner A sequence can be present in a human RFT1 nucleic acid, and the fusion partner B sequence can be present in a human UQCRC2 nucleic acid. The fusion partner A sequence can be present in a human GSDMC nucleic acid, and the fusion partner B sequence can be present in a human PVT1 nucleic acid. The fusion partner A sequence can be present in a human INTS1 nucleic acid, and the fusion partner B sequence can be present in a human PRKAR1B nucleic acid. The fusion partner A sequence can be present in a human POLDIP2 nucleic acid, and the fusion partner B sequence can be present in a human BRIP1 nucleic acid. The fusion partner A sequence can be present in a human MYH9 nucleic acid, and the fusion partner B sequence can be present in a human EIF3D nucleic acid. The fusion partner A sequence can be present in a human BRIP1 nucleic acid, and the fusion partner B sequence can be present in a human TMEM49 nucleic acid. The fusion partner A sequence can be present in a human SUPT4H1 nucleic acid, and the fusion partner B sequence can be present in a human CCDC46 nucleic acid. The fusion partner A sequence can be present in a human TMEM104 nucleic acid, and the fusion partner B sequence can be present in a human CDK12 nucleic acid. The fusion partner A sequence can be present in a human RIMS2 nucleic acid, and the fusion partner B sequence can be present in a human ATP6V1C1 nucleic acid. The fusion partner A sequence can be present in a human TIAL1 nucleic acid, and the fusion partner B sequence can be present in a human C10orf119 nucleic acid. The fusion partner A sequence can be present in a human MECP2 nucleic acid, and the fusion partner B sequence can be present in a human TMLHE nucleic acid. The fusion partner A sequence can be present in a human ARID1A nucleic acid, and the fusion partner B sequence can be present in a human MAST2 nucleic acid. The fusion partner A sequence can be present in a human UBR5 nucleic acid, and the fusion partner B sequence can be present in a human SLC25A32 nucleic acid. The fusion partner A sequence can be present in a human KLHDC2 nucleic acid, and the fusion partner B sequence can be present in a human SNTB1 nucleic acid. The fusion partner A sequence can be present in a human ARID1A nucleic acid, and the fusion partner B sequence can be present in a human WDTC1 nucleic acid. The fusion partner A sequence can be present in a human HDGF nucleic acid, and the fusion partner B sequence can be present in a human S100A10 nucleic acid. The fusion partner A sequence can be present in a human PPP1R12B nucleic acid, and the fusion partner B sequence can be present in a human SNX27 nucleic acid. The fusion partner A sequence can be present in a human SRGAP2 nucleic acid, and the fusion partner B sequence can be present in a human PRPF3 nucleic acid. The fusion partner A sequence can be present in a human WIPF2 nucleic acid, and the fusion partner B sequence can be present in a human ERBB2 nucleic acid.

In another aspect, this document features an isolated nucleic acid comprising, or consisting essentially of, a fusion partner A sequence and a fusion partner B sequence, wherein the fusion partner A sequence is present in a first human gene set forth in Table 3, 4, 5, 6, 8, or 10 and the fusion partner B sequence is present in a second human gene set forth in Table 3, 4, 5, 6, 8, or 10 as being a fusion partner with the first human gene. The fusion partner A sequence can be at least 10 nucleotides. The fusion partner A sequence can be at least 50 nucleotides. The fusion partner A sequence can be at least 100 nucleotides. The fusion partner B sequence can be at least 10 nucleotides. The fusion partner B sequence can be at least 50 nucleotides. The fusion partner B sequence can be at least 100 nucleotides. The fusion partner A sequence can be present in a human LIMA1 nucleic acid, and the fusion partner B sequence can be present in a human USP22 nucleic acid. The fusion partner A sequence can be present in a human LIMA1 nucleic acid, and the fusion partner B sequence can be present in a human USP22 nucleic acid. The fusion partner A sequence can be present in a human ACACA nucleic acid, and the fusion partner B sequence can be present in a human STAC2 nucleic acid. The fusion partner A sequence can be present in a human FAM102A nucleic acid, and the fusion partner B sequence can be present in a human CIZ1 nucleic acid. The fusion partner A sequence can be present in a human GLB1 nucleic acid, and the fusion partner B sequence can be present in a human CMTM7 nucleic acid. The fusion partner A sequence can be present in a human MED1 nucleic acid, and the fusion partner B sequence can be present in a human STXBP4 nucleic acid. The fusion partner A sequence can be present in a human PIP4K2B nucleic acid, and the fusion partner B sequence can be present in a human RAD51C nucleic acid. The fusion partner A sequence can be present in a human RAB22A nucleic acid, and the fusion partner B sequence can be present in a human MYO9B nucleic acid. The fusion partner A sequence can be present in a human RPS6KB1 nucleic acid, and the fusion partner B sequence can be present in a human SNF8 nucleic acid. The fusion partner A sequence can be present in a human STARD3 nucleic acid, and the fusion partner B sequence can be present in a human DOK5 nucleic acid. The fusion partner A sequence can be present in a human TRPC4AP nucleic acid, and the fusion partner B sequence can be present in a human MRPL45 nucleic acid. The fusion partner A sequence can be present in a human ZMYND8 nucleic acid, and the fusion partner B sequence can be present in a human CEP250 nucleic acid. The fusion partner A sequence can be present in a human CTAGE5 nucleic acid, and the fusion partner B sequence can be present in a human SIP1 nucleic acid. The fusion partner A sequence can be present in a human MLL5 nucleic acid, and the fusion partner B sequence can be present in a human LHFPL3 nucleic acid. The fusion partner A sequence can be present in a human SEC22B nucleic acid, and the fusion partner B sequence can be present in a human NOTCH2 nucleic acid. The fusion partner A sequence can be present in a human EIF3K nucleic acid, and the fusion partner B sequence can be present in a human CYP39A1 nucleic acid. The fusion partner A sequence can be present in a human RAB7A nucleic acid, and the fusion partner B sequence can be present in a human LRCH3 nucleic acid. The fusion partner A sequence can be present in a human RNF187 nucleic acid, and the fusion partner B sequence can be present in a human OBSCN nucleic acid. The fusion partner A sequence can be present in a human SLC37A1 nucleic acid, and the fusion partner B sequence can be present in a human ABCG1 nucleic acid. The fusion partner A sequence can be present in a human EXOC7 nucleic acid, and the fusion partner B sequence can be present in a human CYTH1 nucleic acid. The fusion partner A sequence can be present in a human BRE nucleic acid, and the fusion partner B sequence can be present in a human DPYSL5 nucleic acid. The fusion partner A sequence can be present in a human CD151 nucleic acid, and the fusion partner B sequence can be present in a human DRD4 nucleic acid. The fusion partner A sequence can be present in a human LDLRAD3 nucleic acid, and the fusion partner B sequence can be present in a human TCP11L1 nucleic acid. The fusion partner A sequence can be present in a human RFT1 nucleic acid, and the fusion partner B sequence can be present in a human UQCRC2 nucleic acid. The fusion partner A sequence can be present in a human GSDMC nucleic acid, and the fusion partner B sequence can be present in a human PVT1 nucleic acid. The fusion partner A sequence can be present in a human INTS1 nucleic acid, and the fusion partner B sequence can be present in a human PRKAR1B nucleic acid. The fusion partner A sequence can be present in a human POLDIP2 nucleic acid, and the fusion partner B sequence can be present in a human BRIP1 nucleic acid. The fusion partner A sequence can be present in a human MYH9 nucleic acid, and the fusion partner B sequence can be present in a human EIF3D nucleic acid. The fusion partner A sequence can be present in a human BRIP1 nucleic acid, and the fusion partner B sequence can be present in a human TMEM49 nucleic acid. The fusion partner A sequence can be present in a human SUPT4H1 nucleic acid, and the fusion partner B sequence can be present in a human CCDC46 nucleic acid. The fusion partner A sequence can be present in a human TMEM104 nucleic acid, and the fusion partner B sequence can be present in a human CDK12 nucleic acid. The fusion partner A sequence can be present in a human RIMS2 nucleic acid, and the fusion partner B sequence can be present in a human ATP6V1C1 nucleic acid. The fusion partner A sequence can be present in a human TIAL1 nucleic acid, and the fusion partner B sequence can be present in a human C10orf119 nucleic acid. The fusion partner A sequence can be present in a human MECP2 nucleic acid, and the fusion partner B sequence can be present in a human TMLHE nucleic acid. The fusion partner A sequence can be present in a human ARID1A nucleic acid, and the fusion partner B sequence can be present in a human MAST2 nucleic acid. The fusion partner A sequence can be present in a human UBR5 nucleic acid, and the fusion partner B sequence can be present in a human SLC25A32 nucleic acid. The fusion partner A sequence can be present in a human KLHDC2 nucleic acid, and the fusion partner B sequence can be present in a human SNTB1 nucleic acid. The fusion partner A sequence can be present in a human ARID1A nucleic acid, and the fusion partner B sequence can be present in a human WDTC1 nucleic acid. The fusion partner A sequence can be present in a human HDGF nucleic acid, and the fusion partner B sequence can be present in a human S100A10 nucleic acid. The fusion partner A sequence can be present in a human PPP1R12B nucleic acid, and the fusion partner B sequence can be present in a human SNX27 nucleic acid. The fusion partner A sequence can be present in a human SRGAP2 nucleic acid, and the fusion partner B sequence can be present in a human PRPF3 nucleic acid. The fusion partner A sequence can be present in a human WIPF2 nucleic acid, and the fusion partner B sequence can be present in a human ERBB2 nucleic acid.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a flow chart of the work flow of the fusion detection algorithm implemented in SnowShoes-FTD.

FIG. 2 contains photographs of PCR validation of candidate fusion products. The PCR primers were designed using the template sequences generated by SnowShoes-FTD. The double stranded cDNA libraries were constructed using total RNAs from each of the cell lines. The primer sequences and the expected PCR product sizes for each of the fusion candidates were detailed in Table 5. (a) The PCR products from 50 fusion candidates with unique isoforms. The fusion candidates were grouped by the cell lines in which the fusion candidates were discovered. (b) The PCR products from 5 fusion candidates with two fusion isoforms each. Note that there are multiple PCR bands in the lanes for CDK12-TMEM104, and the lowest bands were those from the fusion product.

FIG. 3 contains schematics of in-frame fusion transcripts and their predicted protein sequences. (a) Starting from the fusion junction spanning reads that aligned to both fusion partner genes, the two junction boundary exons from fusion partner genes A and B were identified. (b) Obtaining the IDs and sequences of all exons belonging to the two fusion partner genes A and B based on the curated refFlat file. In this example, Gene A has 7 exons with the 3^{rd} exon as the fusion boundary exon, and gene B has 10 exons with the 6^{th} exon as the fusion boundary exon. (c) Obtaining all known transcripts for the two fusion partner genes. Gene A has two known transcripts (A1 and A2) both of which contain the fusion boundary exon. Gene B has 4 known transcripts (B 1→B4) and three of which (B1, B3, and B4) contain the fusion boundary exon. (d) Generating the list of exhaustive fusion transcripts using the known transcripts containing the fusion boundary exons. There are 6 possible fusion transcripts: A 1-B1, A 1-B3, A 1-B4, A2-B1, A2-B3, and A2-B4. Note that because the differences between the transcripts B1 and B4 are “fused out”, the fusion transcript of A1-B1 is identical to that of A1-B4. Similarly, A2-B1 is identical to A2-B4. The fusion transcripts that cause frame shift in gene B are defined as “out of frame”, and the ones that did not cause any frame shift are defined as “in frame” fusions. Each of the in frame fusions are translated into amino acid sequences of the fusion proteins.

FIG. 4 contains a detailed description of ARID1A_MAST2 (a) and WIPF2_ERBB2 (b) fusion transcripts. Using the process described in FIG. 3, SnowShoes-FTD uses the RNA sequence of all known transcripts of the fusion partners to predict the sequence of all potential in frame and out of frame fusion transcripts. Abundance of individual exons for each of the fusion partners, normalized to total exon abundance, was extracted from the mRNA-Seq data.

FIG. 5 is a photograph of RT-PCR results performed using the PCR primers provided by Maher et al. (Proc. Natl. Acad. Sci. USA, 106(30):12353-8 (2009)) for five indicated fusion transcripts. The PCR validated four of the fusion products (lanes 2-5). However, the fusion product was not observed for ARGAP19_DRG1 (lane 6). The first lane is the 50-pb ladder.

FIG. 6. Multiple fusion transcripts are expressed in breast tumors of different subtypes. Subtype specific fusion transcripts are identified with oval symbols. All fusion transcripts are given according to orientation 5 fusion partner->3′ fusion partner. Transcripts are further identified according to sentinel status in each tumor subtype (S), redundancy in each subtype (R), and fusion transcript isoforms detection in each subtype (I). Fusion products are identified as follows: 3′UTR=fusion that changes 3′UTR of 5′ fusion partner; 5′UTR=fusion in 5′UTR of 5′ fusion partner; CIF=coding in frame fusion to produce a chimaeric protein; CTT=C-terminal truncation of 5′ fusion partner resulting from frame shift.

FIG. 7. Chromosomal distribution of fusion transcripts and fusion partner genes is non-random. Connection between the chromosomal loci of fusion transcripts in shown in Panel A for all sentinel fusions as well as for tumor subtype specific fusion transcripts. The chromosomal ‘heat map’ (Panel B) shows the top four (red) and bottom four (green) chromosomes, identified by the genomic coordinates of fusion partner genes.

FIG. 8. Chromosomal mapping of fusion partner genes reveals tumor sup type specific clusters. Chromosomal mapping was carried out using PheGen (NCBI) to assign chromosomal coordinates of all fusion gene partners. Clusters that are uniquely associated with HER2+ tumors are designated by an arrow with a single asterisk (Chr1q21.22-21.3), whereas an arrow with two asterisks designates a large ER+ cluster at chr11q13.1-q13.3, and an arrow with three asterisks identifies TN clusters at chr8q24.3, chr12q13.13, and chr17q25.1-25.3.

FIG. 9 is a listing of predicted chimeric protein products of fusion transcripts. Amino acids pertaining to 5′ fusion partners are highlighted with a single underline. Amino acids pertaining to 3′ fusion partners fused in frame are highlighted with a double underline. Amino acids that are inserted at fusions junctions are highlighted with a wavy underline.

FIG. 10 is a listing of the predicted amino acid sequence of the ARID1A->MAST2 fusion protein (SEQ ID NO:1530). This chimeric protein arises from a fusion transcript in which exon 1 of ARID1A (with start codon) is spliced in frame to exon 2 of MAST2. Underlined amino acids are derived from exon 1 of ARID1A, whereas the other amino acids are derived from MAST2.

FIG. 11 is a photograph demonstrating shRNA knockdown of the ARID1A->MAST2 fusion transcript.

FIG. 12 is a graph demonstrating that knockdown of the ARID1A->MAST2 fusion transcript by shRNA inhibits growth of MDA-MB-468 cultures.

### DETAILED DESCRIPTION

This document provides methods and materials involved in assessing gene rearrangements (e.g., translocations). For example, this document provides methods and materials for determining whether or not a sample (e.g., breast tissue sample) from a mammal (e.g., a human) contains a gene rearrangement set forth in Table 3, 4, 5, 6, 8, or 10. In some cases, the methods and materials provided herein can be used to detect the presence of a gene rearrangement set forth in Table 3, 4, 5, 6, 8, or 10 within a breast tissue sample, thereby indicating that the breast tissue is likely to be cancerous. Detecting a gene rearrangement set forth in Table 3, 4, 5, 6, 8, or 10 can be used to diagnose breast cancer in a mammal, typically when known clinical symptoms of or known risk factors for breast cancer also are present.

The term “nucleic acid” as used herein can be RNA or DNA, including cDNA, genomic DNA, and synthetic (e.g. chemically synthesized) DNA. The nucleic acid can be double-stranded or single-stranded. Where single-stranded, the nucleic acid can be the sense strand or the antisense strand. In addition, nucleic acid can be circular or linear.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism or cell from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

In one embodiment, this document provides a primer pair having the ability to amplify a nucleic acid that includes (a) a first nucleic acid sequence from one gene listed in a gene rearrangement set forth in Table 3, 4, 5, 6, 8, or 10 (e.g., a fusion partner A sequence) and (b) a second nucleic acid sequence from another gene that is listed in Table 3, 4, 5, 6, 8, or 10 as being in combination with that one gene (e.g., a fusion partner B sequence). For example, this document provides primer pairs that have the ability to amplify a nucleic acid that includes a LIMA1 nucleic acid sequence (e.g., a fusion partner A sequence) and a USP22 nucleic acid sequence (e.g., a fusion partner B sequence). The primers of the primer pair can be any appropriate length including, without limitation, lengths ranging from about 10 nucleotides to about 100 nucleotides (e.g., from about 15 nucleotides to about 100 nucleotides, from about 20 nucleotides to about 100 nucleotides, from about 15 nucleotides to about 75 nucleotides, from about 15 nucleotides to about 50 nucleotides, from about 15 nucleotides to about 25 nucleotides, from about 13 nucleotides to about 50 nucleotides, or from about 17 nucleotides to about 50 nucleotides).

The primers can be designed to amplify any appropriate length of the fusion partner A sequence and the fusion partner B sequence. For example, the fusion partner A sequence of an amplified nucleic acid can be about 5 to about 2500 nucleotides in length (e.g., about 10 to about 2500 nucleotides in length, about 15 to about 2500 nucleotides in length, about 20 to about 2500 nucleotides in length, about 25 to about 2500 nucleotides in length, about 20 to about 1000 nucleotides in length, about 20 to about 500 nucleotides in length, or about 50 to about 100 nucleotides in length), and the fusion partner B sequence of that amplified nucleic acid can be about 5 to about 2500 nucleotides in length (e.g., about 10 to about 2500 nucleotides in length, about 15 to about 2500 nucleotides in length, about 20 to about 2500 nucleotides in length, about 25 to about 2500 nucleotides in length, about 20 to about 1000 nucleotides in length, about 20 to about 500 nucleotides in length, or about 50 to about 100 nucleotides in length). In some cases, the combined length of the fusion partner A and fusion partner B sequences that are amplified can be between about 50 and about 5000 nucleotides (e.g., between about 75 and about 5000 nucleotides, between about 100 and about 5000 nucleotides, between about 250 and about 5000 nucleotides, between about 500 and about 5000 nucleotides, between about 50 and about 2500 nucleotides, between about 500 and about 2500 nucleotides, or between about 50 and about 1000 nucleotides). In some cases, the primer pairs provided herein have the ability to amplify a junction region of a gene rearrangement that involves a two gene fusion set forth in Table 3, 4, 5, 6, 8, or 10. For example, a primer pair provided herein can amplify a junction region between a RAB7A nucleic acid sequence and a LRCH3 nucleic acid sequence.

Examples of particular primer pairs for amplifying a gene rearrangement provided herein include, without limitation, those primer pairs set forth in Table 5.

This document also provides isolated nucleic acid molecules having (a) a first nucleic acid sequence from one gene listed in a gene rearrangement set forth in Table 3, 4, 5, 6, 8, or 10 (e.g., a fusion partner A sequence) and (b) a second nucleic acid sequence from another gene that is listed in Table 3, 4, 5, 6, 8, or 10 as being in combination with that one gene (e.g., a fusion partner B sequence). For example, this document provides isolated nucleic acid molecules that include a LIMA1 nucleic acid sequence (e.g., a fusion partner A sequence) and a USP22 nucleic acid sequence (e.g., a fusion partner B sequence). Other examples of isolated nucleic acid molecules provided herein include, without limitation, those having a sequence set forth in the “Fusion Transcript Coding Sequence” column of Table 6 as well as those having a sequence that encodes an amino acid sequence set forth in the “Fusion Protein Sequence” column of Table 6. The isolated nucleic acid molecules provided herein can be any appropriate length including, without limitation, lengths ranging from about 50 and about 5000 nucleotides (e.g., between about 75 and about 5000 nucleotides, between about 100 and about 5000 nucleotides, between about 250 and about 5000 nucleotides, between about 500 and about 5000 nucleotides, between about 50 and about 2500 nucleotides, between about 500 and about 2500 nucleotides, or between about 50 and about 1000 nucleotides).

As described herein, the primer pairs and isolated nucleic acid molecules provided herein can be used to determine whether or not a patient has breast cancer. For example, a patient sample (e.g., a breast tissue sample) can be assessed for the presence or absence of one or more of the gene rearrangements set forth in Table 3, 4, 5, 6, 8, or 10 using a primer pair provided herein or an isolated nucleic acid that was amplified using an amplification reaction. In some cases, the presence of one or more gene rearrangements set forth in Table 3, 4, 5, 6, 8, or 10 can indicate that the patient has breast cancer.

This document also provides methods for detecting the presence of breast cancer. Such methods can include detecting the presence of one or more gene rearrangements set forth in Table 3, 4, 5, 6, 8, or 10. Any appropriate method can be used to detect a gene rearrangement set forth in Table 3, 4, 5, 6, 8, or 10. For example, the nucleic acid amplification techniques described herein can be used to detect a gene rearrangement set forth in Table 3, 4, 5, 6, 8, or 10.

The invention will be further described in the following examples, which do not limit the scope of the invention described in the claims.

### EXAMPLES

### Example 1

### Identification and Characterization of Fusion Transcripts in Breast Cancer and Normal Cell Lines

### Breast Cell Lines

Twenty-two breast cancer cell lines and one non-tumorigenic breast epithelial cell line (MCF10A) were obtained from the American Type Culture Collection (ATCC) (Table 1). All cell lines were thawed and expanded to allow for isolation of total RNA from low passage cells, which should exhibit minimal deviation from the ATCC type reference cells. Eight primary human mammary epithelial cell (HMEC) cultures were established from biopsies of Mayo Clinic patients undergoing evaluation of suspected breast lesions (Table 1). All of the biopsy samples from which the cell lines were derived were assessed as benign.

### RNA Preparation and Sequencing

Total RNA extraction was performed using Exiqon's miRCURY RNA Isolation Kit. One microgram of total RNA was used for the sequencing library preparation, which was modified from conventional Illumina mRNA-Seq protocols to facilitate paired end RNA sequence analysis (Sun et al., PLoS ONE, 6(2):e17490 (2011)). The cDNA fragments were amplified by PCR and sequenced at both ends for 50 bases (50-base pair-end sequencing) using the Illumina Genome Analyzer IIx. Sequencing was carried out at the Illumina assay development facility at Hayward, Calif. and at the Mayo Clinic Advanced Genomic Technology Center at Rochester, Minn. The FASTQ read files for each sample were used for further analysis.

### Construction of Exhaustive One-Directional Exon Junction Database

The exon-exon boundary database was generated using the exon and gene definition files downloaded from UCSC Table Browser (table: refFlat; track: RefSeq Genes; group: Genes and Gene Prediction Tracks) in reference to human genome build 36 (hg18). Among 35,983 total transcripts in the refFlat file, 765 transcripts with alternative haplotypes and 1,482 transcripts with multiple/redundant genomic locations were removed. Based on the exon boundaries of all transcripts defined in the curated refFlat file, all possible one-directional combinations of exon-exon boundary sequences for the sequencing length of 50 bases were generated to ensure that no reads will map to more than one junction using a developed algorithm. The curated refFlat file and its future updated versions in reference to both Genome Build 36 and 37, as well as the FASTA files of exon-exon boundary sequences for different sequencing lengths (50-, 75-, and 100-base) can be downloaded from the following website: http://mayoresearch.mayo.edu/mayo/research/biostat/stand-alone-packages.cfm.

### Analytic Workflow for Fusion Detection

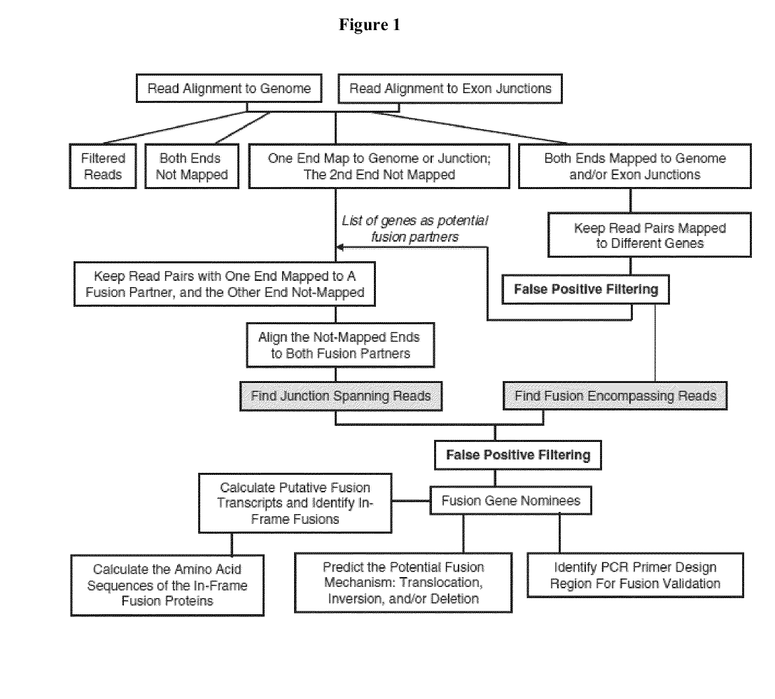

With reference to FIG. 1, the SnowShoes-FTD tool consisted of (i) read alignments to both reference genome and exon junction database; (ii) annotation of aligned read pairs to identify potential fusion candidates; (iii) filtering of false positive candidates; (iv) generation of a continuous sequence region spanning fusion junction points for PCR primer design for experimental validation; (v) prediction of fusion mechanism; and (vi) prediction of the in-frame vs. out of frame fusion products and generation of the predicted protein sequences of the in-frame fusion products based on known transcripts of the two partner genes. In addition, the tool filtered out reads mapped with poor quality as described above.

### Read Alignment and Filtering for Fusion Detection

The two ends of RNA-Seq reads were aligned to both the Human Reference Genome Build 36 (hg18) and exon junctions using BWA (Li and Durbin, Bioinformatics, 25(14):1754-60 (2009)) with a seed length of 32 allowing 4% of maximum edit distance. The BWA aligned reads were stored in the Sequence Alignment/Map (SAM) format (Li et al., Bioinformatics, 25(16):2078-9 (2009)). The pairs of SAM files from the alignment of two ends of the same sample were sorted according to read IDs using SAMtools (Li et al., Bioinformatics, 25(16):2078-9 (2009)). The reads with neither end mapped to genome or exon junctions are not informative and were filtered out. If the Phred-scaled Mapping Quality Score (MAPQ) of either end was less than 20, the end pair was considered low quality and was excluded from further analysis. Note that this also filtered out read pairs with either or both ends mapped to multiple locations since BWA assigns a MAPQ of zero to such reads.

### Annotation of Aligned Reads

After filtering, the reads remaining in the SAM files were categorized into 5 groups: (1) reads with both ends mapped to genome locations; (2) reads with both ends mapped to exon junctions; (3) reads with one end mapped to the genome and the other mapped to exons; (4) reads with one end mapped to the genome and the other end not mapped; and (5) reads with one end mapped to exon junctions and the other not mapped. All mapped ends were annotated using the genes and exons defined in the curated refFlat file. For a read to be annotated as being mapped to a gene, it was required that either the start or the end of the read be mapped within the boundaries of an exon of that gene. If a read aligned to both genome and an exon junction, the annotation from the exon junction alignment took precedence.

### False Positive Filtering

There were two steps of filtering to minimize the false fusion rate that could plague nomination of fusion gene candidates. The first filtering step was performed on the reads pairs that were annotated to two different genes, also known as fusion encompassing reads. This began with the filtering of fusion candidates with significant sequence similarities between the two fusion partners.

In addition, a gene distance filter was implemented to exclude fusions formed by two genes that were within M kb of each other on the reference genome, in order to eliminate chimeric transcripts that might arise from overlapping genes or transcriptional read through of adjacent genes. Furthermore, the fusion candidates with less than N fusion encompassing reads were filtered out. The second filtering step focused on the fusion candidates with supporting evidences of both fusion encompassing read pairs and fusion junction spanning reads. The mapping orientations of the end pairs were compared to the orientations of the two fusion partner genes on the genome, and the fusion candidates with inconsistent mapping orientations between end pairs were filtered out. Also, the algorithm required at least X unique fusion junction spanning reads and no more than Y fusion junction points per fusion candidate. These thresholds (M, N, X, and Y) were user defined.

### Prediction of the Fusion Mechanism

If a fusion product was formed by two partner genes from two different chromosomes, a translocation was listed as the mechanism of fusion. The translocation event can be accompanied by inversion of the two partner genes that have the opposite strand orientations. When the two partner genes were located on the same chromosome, the mechanism of the fusion could be translocation alone, inversion alone, and inversion and translocation concurrently. These three scenarios were determined based on the strand orientations and the relative chromosomal positions of the two partners. However, when an intra-chromosomal fusion arose without altering the relative orders of the two partners with the same strand orientation, the fusion can be the consequence of a translocation or an interstitial deletion.

### Prediction of the Fusion Protein Product

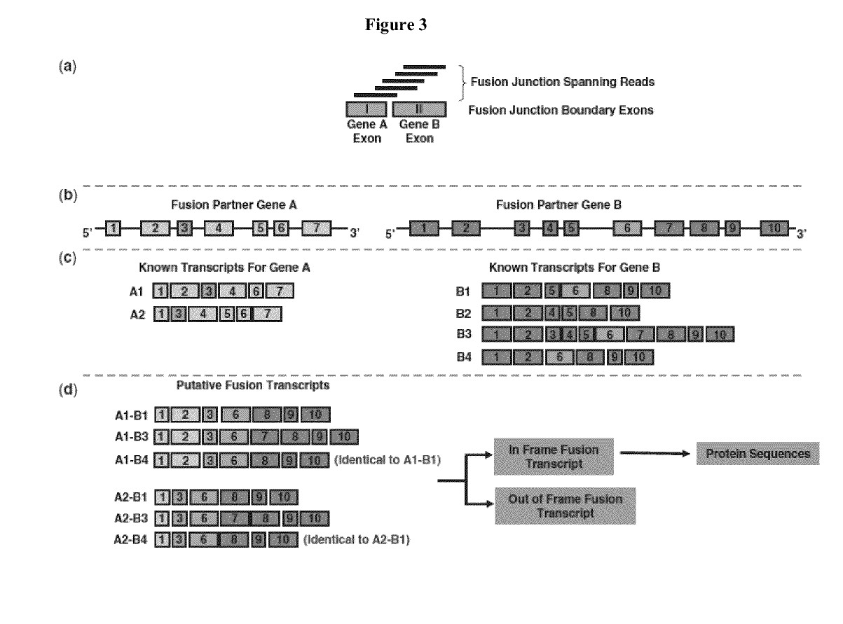

Prediction of the fusion protein sequences was carried out using all of the known transcripts of the two fusion partner genes as defined in the refFlat file. As shown in FIG. 3, the two exons from each of the two fusion partner genes that aligned to the fusion spanning reads (fusion boundary exons) were first identified. Next, among all know transcripts of the two fusion partner genes, the transcripts containing the boundary exons were identified, and a list of putative fusion transcripts was generated. Each of the putative fusion transcripts was then translated into predicted amino acid sequence, and each of the putative fusion proteins was characterized as whether it's in frame. In addition, the fusion products were categorized as: (1) coding region to coding region fusion which results in in-frame fusion product, a frame-shift for the 3′ gene, or an in-frame fusion with a single amino acid mutation at the fusion junction point. The single amino acid mutation was listed in the SnowShoes-FTD output; (2) 5′ UTR to coding region fusion in which the promoter of the 5′ gene fused in front of a coding region of the 3′ gene; (3) 5′ UTR to 3′ UTR fusion in which coding regions from both partner genes were fused out; (4) 3′ UTR to 3′ UTR fusion in which the 5′ gene was intact but the coding region of the 3′ gene was fused out; (5) 5′ UTR to 5′ UTR fusion in which the promoter of the 5′ gene potentially drives the expression of 3′ gene as the consequence of the fusion; (6) 3′ UTR to 5′ UTR or coding region fusion in which the stop codon of the 5′ gene terminates the translation of any coding regions of the 3′ gene; (7) coding region to 5′ UTR fusion in which the sequence between the coding region of the 5′ gene and the start codon of the 3′ gene may result in an insertion of single or multiple amino acids that are listed in the output file; (8) the coding region to 3′ UTR fusion which may result in the shortening of the 5′ gene with or without the addition of foreign amino acids.

### Nucleotide Sequences Spanning Fusion Junction Points for PCR Primer Design

The chromosomal orientations of the two fusion partners, the mapping orientations of the two ends from fusion encompassing read pairs, as well as the sequence and orientation of the fusion junction spanning read(s) were used to report a template region for PCR primer design in order to quickly validate the fusion candidates with RT-PCR. From 5′ to 3′, the template region consisted of the exon region from partner A from the start of the exon to the fusion junction point, a “∥” sign that signified the fusion junction point, and the exon region from partner B from the start of the fusion junction point to the end of the exon. Since the orientation of the primer template region did not necessarily define directionality (5′ to 3′) of the fusion transcript, it was necessary to use double stranded cDNAs as the template for PCR validation.

### PCR and Sanger Sequencing Validations of Fusion Candidates

Double stranded cDNA were synthesized using the total RNAs from each of the 31 cell lines. To minimize potential artifacts that might arise during library construction, different cDNA libraries were constructed and used for sequencing and for PCR validation. PCR primers were designed using the template regions recommended by SnowShoes-FTD. The 5′ and 3′ primers were complementary to the template regions that represent the two fusion partners, respectively. The fusion transcript was considered validated if a PCR product of the predicted size was detected. The PCR bands from randomly selected fusion transcripts were sequenced using Sanger sequencing to further confirm the nucleotide sequence of the predicted fusion junctions.

### Quantification of Gene and Exon Expression Levels

The gene expression levels were calculated as the sum of the individual exon read counts and exon junction read counts. The expression levels of genes and exons were normalized using the total aligned reads from the sample and the length of the exon or gene (Reads per kilo-bases per million, RPKM).

### Results

### Flexibility of the Choice of Sequence Alignment Tools

There are several sequencing platforms and multiple sequence alignment algorithms designed for Next Generation sequencing of transcriptome. The SnowShoes-FTD worked with raw or post-alignment files of different platforms. When FASTQ files obtained from Illumina Genome Analyzer or HiSeq sequencers were provided as input, SnowShoes-FTD was designed such that the user can choose BWA or Bowtie (Langmead et al., Genome Biol, 10(3):R25 (2009)) for alignment. SnowShoes-FTD also was designed to accept post-alignment files (BAM) for both genome and exon junction alignments from different sequencing platforms including Life Technologies' SOLiD sequencer. Since the exon junction database generated by SnowShoes-FTD was preferred over other publically available junction databases, the user needed to align the reads to the exon junctions provided by SnowShoes-FTD if BAM files were provided as input files. The results reported herein were obtained using FASTQ as input files and BWA as the aligner.

### User-Defined Parameters for SnowShoes-FTD

The following parameters were user-defined for detection of fusion transcripts using SnowShoes-FTD: (i) the minimum number of fusion encompassing reads (default value: 10); (ii) the minimum number of unique fusion junction spanning reads (must be ≧1 with a default set to 2); (iii) the minimum distance between the two fusion partner genes if both are located on the same chromosome (default value: 100 kb); (iv) the maximum number of fusion isoforms allowed between two fusion partners (default value: 2); and (v) whether the fusion transcripts feature junction points at exon boundaries (default=Yes). The default values of the parameters were chosen to minimize false positive rate. For example, the minimum number of unique fusion junction spanning reads was set to 2 by default to avoid the false detection of fusion junction spanning reads arising from the PCR artifacts which may give multiple junction spanning reads that are identical in alignment positions. In addition, the limit of the maximum fusion isoforms between two partner genes was based on the hypothesis that if there are too many fusion isoforms between two partners, the fusion event would appear to be existing by random fusion events without obvious biological significances.

### List of Reference Files Available

A list of reference files was available for download in preparation for the fusion transcript detection using SnowShoes-FTD: (1) the one-directional exhaustive exon-exon junction database generated for read-lengths 50-, 75-, and 100-bases. This was provided in the FASTA format; and (2) the curated gene and exon definition files (refFlat files) from both genome builds 36 and 37. The gene and exon definition files are updated periodically. All reference files can be obtained from the SnowShoes website: http://mayoresearch.mayo.edu/mayo/research/biostat/stand-alone-packages.cfm.

### Detection of Fusion Transcripts in 31 Breast Cell Lines

The SnowShoes-FTD tool was applied to the 50-base pair-end RNA-Seq data from 22 breast cancer cell lines, one established non-tumorigenic breast cell line (MCF10A), and 8 primary HMEC cultures (Table 1). The fusion transcript candidates of these 31 breast cell lines were nominated using the default parameter values based on genome build 36 (hg18). As shown in Table 2, read pairs sequenced per sample totaled to 18-33 millions, among which 45-58% had both ends mapped to the genome, 3-5% had both ends mapped to exon junctions, 11-18% had one end mapped to the genome and the other mapped to exon junctions, 5-15% had one end mapped to the genome and the other not mapped, 1-2% had one end mapped to exon junctions and the 2nd end not mapped. In addition, there were 2-9% of the read pairs with neither ends mapped to the genome or exon junctions. 11-20% of the reads were filtered out due to low mapping quality and/or redundant mapping.

TABLE 1
Sample information of the 31 breast cell lines.

| | | | Flow | |
|---|---|---|---|---|
| Sample | | | Cell | Run |
| Number | Sample ID | Sample Description | Lane # | Number |
| 1 | BT-474 | Cancer Cell Line | 1 | Run #1 |
| 2 | MCF10A | Non-Tumorigenic | 2 | |
| 3 | BT-20 | Cancer Cell Line | 3 | |
| 4 | MCF7 | Cancer Cell Line | 4 | |
| 5 | MDA-MB-468 | Cancer Cell Line | 6 | |
| 6 | T47D | Cancer Cell Line | 7 | |
| 7 | ZR-75-1 | Cancer Cell Line | 8 | |
| 8 | HCC1937 | Cancer Cell Line | 1 | Run #2 |
| 9 | HCC1954 | Cancer Cell Line | 2 | |
| 10 | HCC2218 | Cancer Cell Line | 3 | |
| 11 | HCC1599 | Cancer Cell Line | 4 | |
| 12 | HCC1395 | Cancer Cell Line | 5 | |
| 13 | BT549 | Cancer Cell Line | 6 | |
| 14 | Hs578T | Cancer Cell Line | 7 | |
| 15 | MDA-MB-175V-II | Cancer Cell Line | 8 | |
| 16 | MDA-MB-361 | Cancer Cell Line | 1 | Run #3 |
| 17 | MDA-MB-436 | Cancer Cell Line | 2 | |
| 18 | MDA-MB-453 | Cancer Cell Line | 3 | |
| 19 | SK-BR-3 | Cancer Cell Line | 4 | |
| 20 | UACC812 | Cancer Cell Line | 5 | |
| 21 | HCC1187 | Cancer Cell Line | 6 | |
| 22 | HCC1428 | Cancer Cell Line | 7 | |
| 23 | HCC1806 | Cancer Cell Line | 8 | |
| 24 | DHF 168 | Normal HMEC* | 1 | Run #4 |
| 25 | BSO19B | Normal HMEC | 2 | |
| 26 | BSO28 | Normal HMEC | 3 | |
| 27 | BSO29 | Normal HMEC | 4 | |
| 28 | BSO30 | Normal HMEC | 5 | |
| 29 | BSO32N | Normal HMEC | 6 | |
| 30 | BSO36 | Normal HMEC | 7 | |
| 31 | BSO37 | Normal HMEC | 8 | |

HMEC: Human Mammalian Epithelial Cells Primarily cultured from benign breast biopsy samples.

TABLE 2

| Row | Cell Line: | BT-474 | MCF10A | BT-20 | MCF7 | MDA-MB-468 | T47D |
|---|---|---|---|---|---|---|---|
| A | Total Read Pairs | 33,108,579 | 29,942,274 | 33,004,454 | 29,777,246 | 32,629,020 | 27,834,336 |
| B | Both ends mapped to | 967,599 | 1,185,024 | 1,465,055 | 1,287,318 | 1,325,523 | 1,233,551 |
| | exon junctions | | | | | | |
| C | Both ends mapped to | 15,472,214 | 16,126,686 | 17,293,975 | 15,491,395 | 15,622,395 | 14,106,192 |
| | genome | | | | | | |
| D | End 1 map to genome; | 1,921,698 | 2,306,577 | 2,490,157 | 2,275,646 | 1,780,577 | 1,991,704 |
| | End 2 map to junction | | | | | | |
| E | End 1 map to junctions; | 1,956,072 | 2,344,307 | 2,532,582 | 2,301,062 | 1,796,695 | 2,022,605 |
| | End 2 map to genome | | | | | | |
| F | End 1 map to genome, | 3,165,404 | 1,534,609 | 1,937,424 | 1,434,814 | 2,527,943 | 1,531,133 |
| | End 2 not mapped | | | | | | |
| G | End 1 not mapped, End 2 | 2,082,290 | 918,848 | 1,029,301 | 1,005,043 | 1,321,144 | 956,473 |
| | map to genome | | | | | | |
| H | End 1 map to exon | 451,657 | 266,065 | 351,723 | 262,611 | 446,131 | 283,188 |
| | junction, End 2 not | | | | | | |
| | mapped | | | | | | |
| I | End 1 not mapped, End 2 | 288,340 | 130,259 | 153,356 | 157,406 | 209,534 | 144,249 |
| | map to exon junction | | | | | | |
| J | Both Ends Not Mapped | 1,413,251 | 987,653 | 1,083,161 | 861,804 | 3,150,078 | 873,260 |
| K | Filtered (MapQ, | 5,390,054 | 4,142,246 | 4,667,720 | 4,700,147 | 4,449,000 | 4,691,981 |
| | Mappability) | | | | | | |
| L | Total Read Pairs | 33,108,579 | 29,942,274 | 33,004,454 | 29,777,246 | 32,629,020 | 27,834,336 |
| M | Both Ends Mapped to | 967,599 | 1,185,024 | 1,465,055 | 1,287,318 | 1,325,523 | 1,233,551 |
| | Exon Junctions | | | | | | |
| N | Both Ends Mapped to | 15,472,214 | 16,126,686 | 17,293,975 | 15,491,395 | 15,622,395 | 14,106,192 |
| | Genome | | | | | | |
| O | One End Mapped to | 3,877,770 | 4,650,884 | 5,022,739 | 4,576,708 | 3,577,272 | 4,014,309 |
| | Genome, One End | | | | | | |
| | Mapped to Exon Junction | | | | | | |
| P | One End Mapped to | 5,247,694 | 2,453,457 | 2,966,725 | 2,439,857 | 3,849,087 | 2,487,606 |
| | Genome, One End Not | | | | | | |
| | Mapped | | | | | | |
| Q | One End Mapped of Exon | 739,997 | 396,324 | 505,079 | 420,017 | 655,665 | 427,437 |
| | Junction, One End Not | | | | | | |
| | Mapped | | | | | | |
| R | Both Ends Not Mapped | 1,413,251 | 987,653 | 1,083,161 | 861,804 | 3,150,078 | 873,260 |
| S | Filtered Read Pairs | 5,390,054 | 4,142,246 | 4,667,720 | 4,700,147 | 4,449,000 | 4,691,981 |
| T | Total Read Pairs | 33,108,579 | 29,942,274 | 33,004,454 | 29,777,246 | 32,629,020 | 27,834,336 |
| U | Both Ends Mapped to | 2.9225% | 3.9577% | 4.4390% | 4.3232% | 4.0624% | 4.4318% |
| | Exon Junctions | | | | | | |
| V | Both Ends Mapped to | 46.7317% | 53.8593% | 52.3989% | 52.0243% | 47.8788% | 50.6791% |
| | Genome | | | | | | |
| W | One End Mapped to | 11.7123% | 15.5328% | 15.2184% | 15.3698% | 10.9635% | 14.4221% |
| | Genome, One End | | | | | | |
| | Mapped to Exon Junction | | | | | | |
| X | One End Mapped to | 15.8500% | 8.1940% | 8.9889% | 8.1937% | 11.7965% | 8.9372% |
| | Genome, One End Not | | | | | | |
| | Mapped | | | | | | |
| Y | One End Mapped of Exon | 2.2351% | 1.3236% | 1.5303% | 1.4105% | 2.0095% | 1.5356% |
| | Junction, One End Not | | | | | | |
| | Mapped | | | | | | |
| Z | Both Ends Not Mapped | 4.2685% | 3.2985% | 3.2819% | 2.8942% | 9.6542% | 3.1373% |
| AA | Filtered Read Pairs | 16.2799% | 13.8341% | 14.1427% | 15.7844% | 13.6351% | 16.8568% |

| Row | ZR-75-1 | HCC1954 | HCC2218 | HCC1599 | HCC1395 | BT549 | Hs578T | MDA-MB-175V-II | MDA-MB-361 |
|---|---|---|---|---|---|---|---|---|---|
| A | 28,279,001 | 21,368,082 | 21,646,565 | 20,839,210 | 20,885,816 | 20,564,387 | 21,163,489 | 19,975,881 | 18,982,847 |
| B | 906,388 | 1,057,290 | 1,057,372 | 1,060,908 | 1,028,507 | 992,377 | 1,139,460 | 790,780 | 760,538 |
| C | 12,865,780 | 11,457,498 | 10,468,135 | 11,075,098 | 10,889,876 | 11,217,404 | 11,469,811 | 10,843,542 | 11,152,244 |
| D | 1,553,829 | 1,654,404 | 1,293,163 | 1,699,329 | 1,719,327 | 1,698,529 | 1,902,759 | 1,364,672 | 1,217,291 |
| E | 1,569,922 | 1,683,656 | 1,315,053 | 1,723,060 | 1,745,710 | 1,720,513 | 1,940,243 | 1,377,012 | 1,240,587 |
| F | 2,756,407 | 839,016 | 771,655 | 761,735 | 794,179 | 754,211 | 1,002,891 | 763,913 | 787,834 |
| G | 1,636,306 | 605,286 | 562,351 | 524,216 | 549,597 | 488,195 | 625,354 | 502,625 | 402,695 |
| H | 426,338 | 144,308 | 134,419 | 128,164 | 137,034 | 127,247 | 210,186 | 109,477 | 124,795 |
| I | 244,443 | 86,449 | 84,679 | 69,464 | 73,921 | 60,734 | 97,234 | 54,231 | 48,261 |
| J | 1,942,352 | 1,104,408 | 1,505,030 | 367,688 | 547,157 | 250,766 | 429,522 | 545,897 | 380,510 |
| K | 4,377,236 | 2,735,767 | 4,454,708 | 3,429,548 | 3,400,508 | 3,254,411 | 2,346,029 | 3,623,732 | 2,868,092 |
| L | 28,279,001 | 21,368,082 | 21,646,565 | 20,839,210 | 20,885,816 | 20,564,387 | 21,163,489 | 19,975,881 | 18,982,847 |
| M | 906,388 | 1,057,290 | 1,057,372 | 1,060,908 | 1,028,507 | 992,377 | 1,139,460 | 790,780 | 760,538 |
| N | 12,865,780 | 11,457,498 | 10,468,135 | 11,075,098 | 10,889,876 | 11,217,404 | 11,469,811 | 10,843,542 | 11,152,244 |
| O | 3,123,751 | 3,338,060 | 2,608,216 | 3,422,389 | 3,465,037 | 3,419,042 | 3,843,002 | 2,741,684 | 2,457,878 |
| P | 4,392,713 | 1,444,302 | 1,334,006 | 1,285,951 | 1,343,776 | 1,242,406 | 1,628,245 | 1,266,538 | 1,190,529 |
| Q | 670,781 | 230,757 | 219,098 | 197,628 | 210,955 | 187,981 | 307,420 | 163,708 | 173,056 |
| R | 1,942,352 | 1,104,408 | 1,505,030 | 367,688 | 547,157 | 250,766 | 429,522 | 545,897 | 380,510 |
| S | 4,377,236 | 2,735,767 | 4,454,708 | 3,429,548 | 3,400,508 | 3,254,411 | 2,346,029 | 3,623,732 | 2,868,092 |
| T | 28,279,001 | 21,368,082 | 21,646,565 | 20,839,210 | 20,885,816 | 20,564,387 | 21,163,489 | 19,975,881 | 18,982,847 |
| U | 3.2052% | 4.9480% | 4.8847% | 5.0909% | 4.9244% | 4.8257% | 5.3841% | 3.9587% | 4.0064% |
| V | 45.4959% | 53.6197% | 48.3593% | 53.1455% | 52.1401% | 54.5477% | 54.1962% | 54.2832% | 58.7491% |
| W | 11.0462% | 15.6217% | 12.0491% | 16.4228% | 16.5904% | 16.6260% | 18.1586% | 13.7250% | 12.9479% |
| X | 15.5335% | 6.7592% | 6.1627% | 6.1708% | 6.4339% | 6.0415% | 7.6937% | 6.3403% | 6.2716% |
| Y | 2.3720% | 1.0799% | 1.0122% | 0.9483% | 1.0100% | 0.9141% | 1.4526% | 0.8195% | 0.9116% |
| Z | 6.8685% | 5.1685% | 6.9527% | 1.7644% | 2.6198% | 1.2194% | 2.0295% | 2.7328% | 2.0045% |
| AA | 15.4788% | 12.8031% | 20.5793% | 16.4572% | 16.2814% | 15.8255% | 11.0853% | 18.1405% | 15.1089% |

| | MDA-MB- | MDA-MB- | | | | | | | | |
|---|---|---|---|---|---|---|---|---|---|---|
| Row | 436 | 453 | SK-BR-3 | UACC812 | HCC1187 | HCC1428 | HCC1806 | HCC1937 | BN1 | BN2 |
| A | 19,326,929 | 18,821,975 | 18,958,559 | 19,338,997 | 19,807,859 | 19,126,250 | 18,714,788 | 18,104,523 | 21,550,821 | 21,353,151 |
| B | 1,013,331 | 853,132 | 879,624 | 872,827 | 982,195 | 905,990 | 969,604 | 860,993 | 1,060,260 | 1,094,922 |
| C | 10,245,609 | 10,758,747 | 9,956,488 | 10,852,009 | 10,622,768 | 10,149,823 | 9,707,659 | 10,205,243 | 11,809,197 | 11,606,028 |
| D | 1,668,058 | 1,425,541 | 1,516,716 | 1,480,106 | 1,449,627 | 1,434,064 | 1,436,302 | 1,496,280 | 1,906,149 | 1,657,758 |
| E | 1,687,689 | 1,436,359 | 1,531,754 | 1,500,490 | 1,467,590 | 1,446,630 | 1,451,970 | 1,507,298 | 1,918,891 | 1,680,564 |
| F | 703,619 | 627,395 | 700,675 | 722,458 | 903,348 | 845,077 | 900,149 | 534,762 | 654,737 | 750,144 |
| G | 445,262 | 393,266 | 430,142 | 434,050 | 512,627 | 486,248 | 496,522 | 397,224 | 443,296 | 552,817 |
| H | 121,839 | 90,442 | 114,555 | 111,533 | 162,509 | 148,712 | 168,810 | 75,098 | 98,768 | 98,206 |
| I | 59,423 | 44,966 | 55,628 | 52,103 | 74,421 | 69,319 | 74,394 | 41,912 | 51,730 | 57,704 |
| J | 403,083 | 225,327 | 428,803 | 275,411 | 524,031 | 645,819 | 546,545 | 338,321 | 470,350 | 495,573 |
| K | 2,979,016 | 2,966,800 | 3,344,174 | 3,038,010 | 3,108,743 | 2,994,568 | 2,962,833 | 2,647,392 | 3,137,443 | 3,359,435 |
| | MDA-MB- | MDA-MB- | | | | | | | MDA-MB- | MDA-MB- |
| Row | 436 | 453 | SK-BR-3 | UACC812 | HCC1187 | HCC1428 | HCC1806 | HCC1937 | 436 | 453 |
| L | 19,326,929 | 18,821,975 | 18,958,559 | 19,338,997 | 19,807,859 | 19,126,250 | 18,714,788 | 18,104,523 | 21,550,821 | 21,353,151 |
| M | 1,013,331 | 853,132 | 879,624 | 872,827 | 982,195 | 905,990 | 969,604 | 860,993 | 1,060,260 | 1,094,922 |
| N | 10,245,609 | 10,758,747 | 9,956,488 | 10,852,009 | 10,622,768 | 10,149,823 | 9,707,659 | 10,205,243 | 11,809,197 | 11,606,028 |
| O | 3,355,747 | 2,861,900 | 3,048,470 | 2,980,596 | 2,917,217 | 2,880,694 | 2,888,272 | 3,003,578 | 3,825,040 | 3,338,322 |
| P | 1,148,881 | 1,020,661 | 1,130,817 | 1,156,508 | 1,415,975 | 1,331,325 | 1,396,671 | 931,986 | 1,098,033 | 1,302,961 |
| Q | 181,262 | 135,408 | 170,183 | 163,636 | 236,930 | 218,031 | 243,204 | 117,010 | 150,498 | 155,910 |
| R | 403,083 | 225,327 | 428,803 | 275,411 | 524,031 | 645,819 | 546,545 | 338,321 | 470,350 | 495,573 |
| S | 2,979,016 | 2,966,800 | 3,344,174 | 3,038,010 | 3,108,743 | 2,994,568 | 2,962,833 | 2,647,392 | 3,137,443 | 3,359,435 |
| T | 19,326,929 | 18,821,975 | 18,958,559 | 19,338,997 | 19,807,859 | 19,126,250 | 18,714,788 | 18,104,523 | 21,550,821 | 21,353,151 |
| U | 5.2431% | 4.5326% | 4.6397% | 4.5133% | 4.9586% | 4.7369% | 5.1810% | 4.7557% | 4.9198% | 5.1277% |
| V | 53.0121% | 57.1606% | 52.5171% | 56.1146% | 53.6291% | 53.0675% | 51.8716% | 56.3685% | 54.7970% | 54.3528% |
| W | 17.3631% | 15.2051% | 16.0797% | 15.4124% | 14.7276% | 15.0615% | 15.4331% | 16.5902% | 17.7489% | 15.6339% |
| X | 5.9445% | 5.4227% | 5.9647% | 5.9802% | 7.1486% | 6.9607% | 7.4629% | 5.1478% | 5.0951% | 6.1020% |
| Y | 0.9379% | 0.7194% | 0.8977% | 0.8461% | 1.1961% | 1.1400% | 1.2995% | 0.6463% | 0.6983% | 0.7301% |
| Z | 2.0856% | 1.1971% | 2.2618% | 1.4241% | 2.6456% | 3.3766% | 2.9204% | 1.8687% | 2.1825% | 2.3208% |
| AA | 15.4138% | 15.7624% | 17.6394% | 15.7092% | 15.6945% | 15.6568% | 15.8315% | 14.6228% | 14.5583% | 15.7327% |

| Row | BN3 | BN4 | BN5 | BN6 | BN7 | BN8 | Min | Max |
|---|---|---|---|---|---|---|---|---|
| A | 20,924,924 | 22,510,790 | 21,057,269 | 24,033,748 | 21,682,601 | 20,257,198 | 18,104,523 | 33,108,579 |
| B | 1,045,586 | 1,149,385 | 958,317 | 1,146,878 | 1,083,300 | 945,339 | 760,538 | 1,465,055 |
| C | 11,204,204 | 12,296,033 | 11,542,896 | 13,355,714 | 12,005,466 | 11,203,857 | 9,707,659 | 17,293,975 |
| D | 1,861,254 | 2,049,317 | 1,723,515 | 2,076,865 | 1,673,894 | 1,721,057 | 1,217,291 | 2,490,157 |
| E | 1,868,123 | 2,062,445 | 1,736,873 | 2,089,358 | 1,689,254 | 1,732,145 | 1,240,587 | 2,532,582 |
| F | 657,416 | 645,611 | 639,013 | 762,606 | 741,286 | 646,232 | 534,762 | 3,165,404 |
| G | 445,708 | 425,788 | 417,370 | 495,086 | 515,542 | 425,145 | 393,266 | 2,082,290 |
| H | 99,404 | 97,012 | 91,801 | 113,551 | 111,449 | 93,334 | 75,098 | 451,657 |
| I | 51,038 | 44,827 | 45,871 | 54,262 | 65,365 | 47,633 | 41,912 | 288,340 |
| J | 432,782 | 425,987 | 685,134 | 428,998 | 494,662 | 512,827 | 225,327 | 3,150,078 |
| K | 3,259,409 | 3,314,385 | 3,216,479 | 3,510,430 | 3,302,383 | 2,929,629 | 2,346,029 | 5,390,054 |
| Row | SK-BR-3 | UACC812 | HCC1187 | HCC1428 | HCC1806 | HCC1937 | Min | Max |
| L | 20,924,924 | 22,510,790 | 21,057,269 | 24,033,748 | 21,682,601 | 20,257,198 | 18,104,523 | 33,108,579 |
| M | 1,045,586 | 1,149,385 | 958,317 | 1,146,878 | 1,083,300 | 945,339 | 760,538 | 1,465,055 |
| N | 11,204,204 | 12,296,033 | 11,542,896 | 13,355,714 | 12,005,466 | 11,203,857 | 9,707,659 | 17,293,975 |
| O | 3,729,377 | 4,111,762 | 3,460,388 | 4,166,223 | 3,363,148 | 3,453,202 | 2,457,878 | 5,022,739 |
| P | 1,103,124 | 1,071,399 | 1,056,383 | 1,257,692 | 1,256,828 | 1,071,377 | 931,986 | 5,247,694 |
| Q | 150,442 | 141,839 | 137,672 | 167,813 | 176,814 | 140,967 | 117,010 | 739,997 |
| R | 432,782 | 425,987 | 685,134 | 428,998 | 494,662 | 512,827 | 225,327 | 3,150,078 |
| S | 3,259,409 | 3,314,385 | 3,216,479 | 3,510,430 | 3,302,383 | 2,929,629 | 2,346,029 | 5,390,054 |
| T | 20,924,924 | 22,510,790 | 21,057,269 | 24,033,748 | 21,682,601 | 20,257,198 | 18,104,523 | 33,108,579 |
| U | 4.9968% | 5.1059% | 4.5510% | 4.7719% | 4.9962% | 4.6667% | 2.9225% | 5.3841% |
| V | 53.5448% | 54.6228% | 54.8167% | 55.5707% | 55.3691% | 55.3080% | 45.4959% | 58.7491% |
| W | 17.8227% | 18.2657% | 16.4332% | 17.3349% | 15.5108% | 17.0468% | 10.9635% | 18.2657% |
| X | 5.2718% | 4.7595% | 5.0167% | 5.2330% | 5.7965% | 5.2889% | 4.7595% | 15.8500% |
| Y | 0.7190% | 0.6301% | 0.6538% | 0.6982% | 0.8155% | 0.6959% | 0.6301% | 2.3720% |
| Z | 2.0683% | 1.8924% | 3.2537% | 1.7850% | 2.2814% | 2.5316% | 1.1971% | 9.6542% |
| AA | 15.5767% | 14.7235% | 15.2749% | 14.6063% | 15.2306% | 14.4622% | 11.0853% | 20.5793% |

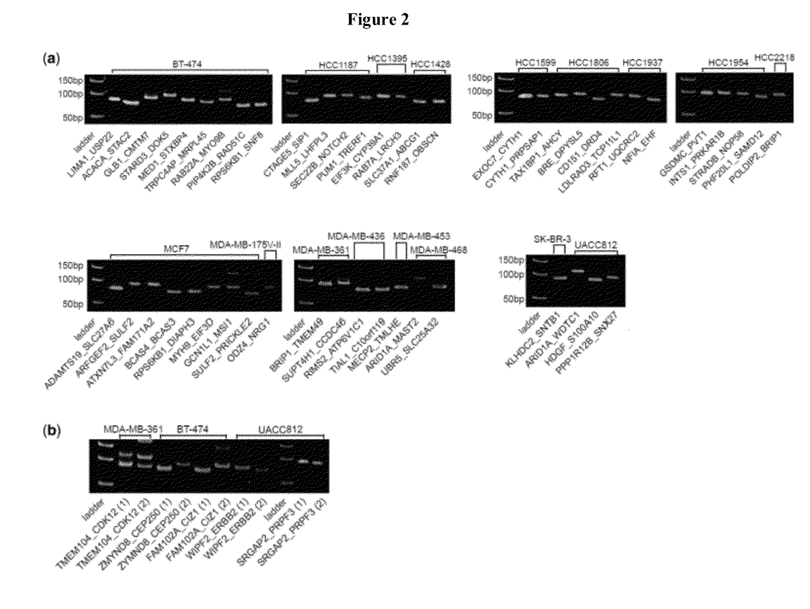

55 fusion transcript candidates were nominated (Tables 3 and 4). Fifty of these had unique isoforms while the rest had 2 isoforms. As shown in FIG. 2A, all 50 fusion transcripts with a single fusion isoform were validated as evidenced by generation of PCR products of the predicted sizes. Several fusion transcripts were randomly selected for further validation using Sanger sequencing of the PCR bands. All PCR products were confirmed by Sanger sequencing with the observation that the predicted DNA sequence conformed to the actual DNA sequence of the PCR product. All isoforms were similarly validated for the 5 fusion candidates with two isoforms (FIG. 2B). The sequences of the primers used in PCR validations are set forth in Table 5, which includes the primers for the alternative isoforms of the 5 fusion candidates with 2 isoforms each.

TABLE 3
List of fusion transcripts identified.

| | | | | | Total | Between | # of |
|---|---|---|---|---|---|---|---|
| | | | In | | Read | Exon | Fusion |
| FUSION Transcript | Mechanism | Type | Frame | Strand | Pairs | Boundaries | Isoforms |
| LIMA1->USP22 | T | inter-chr | YES | − | 16 | YES | 1 |
| ACACA->STAC2 | T | intra-chr | YES | − | 72 | YES | 1 |
| FAM102A->CIZ1 | T | intra-chr | | − | 31 | YES | 2 |
| GLB1->CMTM7 | I | intra-chr | YES | − | 13 | YES | 1 |
| MED1->STXBP4 | I AND T | intra-chr | YES | − | 54 | YES | 1 |
| PIP4K2B->RAD51C | I AND T | intra-chr | | − | 15 | YES | 1 |
| RAB22A->MYO9B | T | inter-chr | | + | 16 | YES | 1 |
| RPS6KB1->SNF8 | I AND T | intra-chr | YES | + | 162 | YES | 1 |
| STARD3->DOK5 | T | inter-chr | | + | 21 | YES | 1 |
| TRPC4AP->MRPL45 | I AND T | inter-chr | YES | − | 27 | YES | 1 |
| ZMYND8->CEP250 | I | intra-chr | | − | 189 | YES | 2 |
| CTAGE5->SIP1 | T | intra-chr | | + | 64 | YES | 1 |
| MLL5->LHFPL3 | T | intra-chr | | + | 23 | YES | 1 |
| PUM1->TRERF1 | T | inter-chr | | − | 58 | YES | 1 |
| SEC22B->NOTCH2 | I AND T | intra-chr | | + | 22 | YES | 1 |
| EIF3K->CYP39A1 | I AND T | inter-chr | YES | + | 91 | YES | 1 |
| RAB7A->LRCH3 | DOR T | intra-chr | | + | 14 | YES | 1 |
| RNF187->OBSCN | T | intra-chr | | + | 11 | YES | 1 |
| SLC37A1->ABCG1 | T | intra-chr | YES | + | 20 | YES | 1 |
| CYTH1->PRPSAP1 | DOR T | intra-chr | YES | − | 33 | YES | 1 |
| EXOC7->CYTH1 | T | intra-chr | YES | − | 20 | YES | 1 |
| BRE->DPYSL5 | T | intra-chr | YES | + | 13 | YES | 1 |
| CD151->DRD4 | T | intra-chr | | + | 11 | YES | 1 |
| LDLRAD3->TCP11L1 | T | intra-chr | | + | 25 | YES | 1 |
| RFT1->UQCRC2 | I AND T | inter-chr | YES | − | 102 | YES | 1 |
| TAX1BP1->AHCY | I AND T | inter-chr | YES | + | 54 | YES | 1 |
| NFIA->EHF | T | inter-chr | YES | + | 18 | YES | 1 |
| GSDMC->PVT1 | I | intra-chr | | − | 23 | YES | 1 |
| INTS1->PRKAR1B | DOR T | intra-chr | YES | − | 24 | YES | 1 |
| PHF20L1->SAMD12 | I AND T | intra-chr | YES | + | 106 | YES | 1 |
| STRADB->NOP58 | DOR T | intra-chr | YES | + | 10 | YES | 1 |
| POLDIP2->BRIP1 | T | intra-chr | | − | 13 | YES | 1 |
| ADAMTS19->SLC27A6 | T | intra-chr | | + | 30 | YES | 1 |
| ARFGEF2->SULF2 | I AND T | intra-chr | YES | + | 421 | YES | 1 |
| ATXN7L3->FAM171A2 | T | intra-chr | | − | 10 | YES | 1 |
| BCAS4->BCAS3 | T | inter-chr | | + | 1697 | YES | 1 |
| GCN1L1->MSI1 | T | intra-chr | YES | − | 25 | YES | 1 |
| MYH9->EIF3D | T | intra-chr | YES | − | 16 | YES | 1 |
| RPS6KB1->DIAPH3 | I AND T | inter-chr | | + | 25 | YES | 1 |
| SULF2->PRICKLE2 | T | inter-chr | | − | 26 | YES | 1 |
| ODZ4->NRG1 | I AND T | inter-chr | YES | − | 12 | YES | 1 |
| BRIP1->TMEM49 | I | intra-chr | | − | 28 | YES | 1 |
| SUPT4H1->CCDC46 | T | intra-chr | | − | 17 | YES | 1 |
| TMEM104->CDK12 | T | intra-chr | YES | + | 10 | YES | 2 |
| RIMS2->ATP6V1C1 | T | intra-chr | YES | + | 11 | YES | 1 |
| TIAL1->C10orf119 | T | intra-chr | | − | 12 | YES | 1 |
| MECP2->TMLHE | T | intra-chr | | − | 29 | YES | 1 |
| ARID1A->MAST2 | DOR T | intra-chr | YES | + | 18 | YES | 1 |
| UBR5->SLC25A32 | T | intra-chr | | − | 28 | YES | 1 |
| KLHDC2->SNTB1 | I AND T | inter-chr | YES | + | 25 | YES | 1 |
| ARID1A->WDTC1 | DOR T | intra-chr | YES | + | 23 | YES | 1 |
| HDGF->S100A10 | DOR T | intra-chr | YES | − | 154 | YES | 1 |
| PPP1R12B->SNX27 | T | intra-chr | YES | + | 45 | YES | 1 |
| SRGAP2->PRPF3 | T | intra-chr | YES | + | 22 | YES | 2 |
| WIPF2->ERBB2 | T | intra-chr | YES | + | 66 | YES | 2 |

The fusion transcripts are named as the 5′ gene -> 3′ gene. For example, LIMA1-> USP22 is a fusion transcript formed between two partner genes, LIMA1 and USP22, in which LIMA1 is the 5′ gene and USP22 is the 3′ gene.
In the fusion mechanism column, T stands for translocation; I stands for inversion; and D stands for interstitial deletion.
Intra-chr: intra-chromosomal fusion;
Inter-chr: inter-chromosomal fusion.

TABLE 4

| Row | FUSION GENE | Potential Fusion Mechanism | Type |
|---|---|---|---|
| 1 | ACACA->STAC2 | Translocation | intra-chromosomal |
| 2 | ADAMTS19->SLC27A6 | Translocation | intra-chromosomal |
| 3 | ARFGEF2->SULF2 | Inversion AND Translocation | intra-chromosomal |
| 4 | ARID1A->MAST2 | Interstitial_Deletion OR Translocation | intra-chromosomal |
| 5 | ARID1A->WDTC1 | Interstitial_Deletion OR Translocation | intra-chromosomal |
| 6 | ATXN7L3->FAM171A2 | Translocation | intra-chromosomal |
| 7 | BCAS4->BCAS3 | Translocation | inter-chromosomal |
| 8 | BRE->DPYSL5 | Translocation | intra-chromosomal |
| 9 | BRIP1->TMEM49 | Inversion Alone | intra-chromosomal |
| 10 | CD151->DRD4 | Translocation | intra-chromosomal |
| 11 | CTAGE5->SIP1 | Translocation | intra-chromosomal |
| 12 | CYTH1->PRPSAP1 | Interstitial_Deletion OR Translocation | intra-chromosomal |
| 13 | EIF3K->CYP39A1 | Inversion AND Translocation | inter-chromosomal |
| 14 | EXOC7->CYTH1 | Translocation | intra-chromosomal |
| 15 | FAM102A->CIZ1 | Translocation | intra-chromosomal |
| 16 | FAM102A->CIZ1 | Translocation | intra-chromosomal |
| 17 | GCN1L1->MSI1 | Translocation | intra-chromosomal |
| 18 | GLB1->CMTM7 | Inversion Alone | intra-chromosomal |
| 19 | GSDMC->PVT1 | Inversion Alone | intra-chromosomal |
| 20 | HDGF->S100A10 | Interstitial_Deletion OR Translocation | intra-chromosomal |
| 21 | INTS1->PRKAR1B | Interstitial_Deletion OR Translocation | intra-chromosomal |
| 22 | KLHDC2->SNTB1 | Inversion AND Translocation | inter-chromosomal |
| 23 | LDLRAD3->TCP11L1 | Translocation | intra-chromosomal |
| 24 | LIMA1->USP22 | Translocation | inter-chromosomal |
| 25 | MECP2->TMLHE | Translocation | intra-chromosomal |
| 26 | MED1->STXBP4 | Inversion AND Translocation | intra-chromosomal |
| 27 | MLL5->LHFPL3 | Translocation | intra-chromosomal |
| 28 | MYH9->EIF3D | Translocation | intra-chromosomal |
| 29 | NFIA->EHF | Translocation | inter-chromosomal |
| 30 | ODZ4->NRG1 | Inversion AND Translocation | inter-chromosomal |
| 31 | PHF20L1->SAMD12 | Inversion AND Translocation | intra-chromosomal |
| 32 | PIP4K2B->RAD51C | Inversion AND Translocation | intra-chromosomal |
| 33 | POLDIP2->BRIP1 | Translocation | intra-chromosomal |
| 34 | PPP1R12B->SNX27 | Translocation | intra-chromosomal |
| 35 | PRPF3->SRGAP2 | Interstitial_Deletion OR Translocation | intra-chromosomal |
| 36 | PUM1->TRERF1 | Translocation | inter-chromosomal |
| 37 | RAB22A->MYO9B | Translocation | inter-chromosomal |
| 38 | RAB7A->LRCH3 | Interstitial_Deletion OR Translocation | intra-chromosomal |
| 39 | RFT1->UQCRC2 | Inversion AND Translocation | inter-chromosomal |
| 40 | RIMS2->ATP6V1C1 | Translocation | intra-chromosomal |
| 41 | RNF187->OBSCN | Translocation | intra-chromosomal |
| 42 | RPS6KB1->DIAPH3 | Inversion AND Translocation | inter-chromosomal |
| 43 | RPS6KB1->SNF8 | Inversion AND Translocation | intra-chromosomal |
| 44 | SEC22B->NOTCH2 | Inversion AND Translocation | intra-chromosomal |
| 45 | SLC37A1->ABCG1 | Translocation | intra-chromosomal |
| 46 | SRGAP2->PRPF3 | Translocation | intra-chromosomal |
| 47 | STARD3->DOK5 | Translocation | inter-chromosomal |
| 48 | STRADB->NOP58 | Interstitial_Deletion OR Translocation | intra-chromosomal |
| 49 | SULF2->PRICKLE2 | Translocation | inter-chromosomal |
| 50 | SUPT4H1->CCDC46 | Translocation | intra-chromosomal |
| 51 | TAX1BP1->AHCY | Inversion AND Translocation | inter-chromosomal |
| 52 | TIAL1->C10orf119 | Translocation | intra-chromosomal |
| 53 | TMEM104->CDK12 | Translocation | intra-chromosomal |
| 54 | TMEM104->CDK12 | Translocation | intra-chromosomal |
| 55 | TRPC4AP->MRPL45 | Inversion AND Translocation | inter-chromosomal |
| 56 | UBR5->SLC25A32 | Translocation | intra-chromosomal |
| 57 | WIPF2->ERBB2 | Translocation | intra-chromosomal |
| 58 | WIPF2->ERBB2 | Translocation | intra-chromosomal |
| 59 | ZMYND8->CEP250 | Inversion Alone | intra-chromosomal |
| 60 | ZMYND8->CEP250 | Inversion Alone | intra-chromosomal |

| Row | Inversion | Exon Mapping Information | Fusion Strand |
|---|---|---|---|
| 1 | NO | E2:chr17:STAC2:NM_198993:34627645:34627952:−: | REVERSE Strand |
| | | 285_307\|\|E53:chr17:ACACA:NM_198839:32553565:32553662: | |
| | | −:1_27 | |
| 2 | NO | E1:chr5:ADAMTS19:NM_133638:128824001:128824074:+: | FORWARD Strand |
| | | 45_73\|\|E9:chr5:SLC27A6:NM_014031:128391936:128392034: | |
| | | +:1_19 | |
| 3 | YES | E3:chr20:SULF2:NM_198596:45798853:45799093:−: | FORWARD_Strand |
| | | 211_240\|\|E1:chr20:ARFGEF2:NM_006420:46971681:46971954: | |
| | | +:273_254 | |
| 4 | NO | E2:chr1:MAST2:NM_015112:46062691:46062839:+:21_1\|\|E1: | FORWARD_Strand |
| | | chr1:ARID1A:NM_006015:26895108:26896618:+:1510_1482 | |
| 5 | NO | E1:chr1:ARID1A:NM_006015:26895108:26896618:+:1487_1510\|\| | FORWARD Strand |
| | | E4:chr1:WDTC1:NM_015023:27481316:27481363:+: | |
| | | 1_26 | |
| 6 | NO | E1:chr17:ATXN7L3:NM_001098833:39630913:39631055:−: | REVERSE_Strand |
| | | 26_1\|\|E4:chr17:FAM171A2:NM_198475:39789323:39789482: | |
| | | −:159_136 | |
| 7 | NO | E1:chr20:BCAS4:NM_017843:48844873:48845117:+:221_244\|\| | FORWARD Strand |
| | | E24:chr17:BCAS3:NM_001099432:56800469:56800637: | |
| | | +:1_23 | |
| 8 | NO | E2:chr2:DPYSL5:NM_020134:26974867:26975132:+:19_1\|\| | FORWARD_Strand |
| | | E8:chr2:BRE:NM_199192:28205641:28205751:+:110_80 | |
| 9 | YES | E3:chr17:BRIP1:NM_032043:57291938:57292050:−: | REVERSE_Strand |
| | | 25_1\|\|E10:chr17:TMEM49:NM_030938:55249854:55249916: | |
| | | +:1_25 | |
| 10 | NO | E4:chr11:CD151:NM_139030:826768:826843:+:52_75\|\|E4:chr11: | FORWARD Strand |
| | | DRD4:NM_000797:630400:630703:+:1_26 | |
| 11 | NO | E9:chr14:SIP1:NM_001009182:38675394:38675928:+:24_1\|\| | FORWARD_Strand |
| | | E20:chr14:CTAGE5:NM_203354:38865818:38865977:+:159_134 | |
| 12 | NO | E1:chr17:CYTH1:NM_004762:74289878:74289971:−: | REVERSE_Strand |
| | | 27_1\|\|E3:chr17:PRPSAP1:NM_002766:71852346:71852413: | |
| | | −:67_44 | |
| 13 | | E3:chr6:CYP39A1:NM_016593:46715189:46715364:−: | FORWARD_Strand |
| | | 152_175\|\|E6:chr19:EIF3K:NM_013234:43815080:43815158: | |
| | | +:78_53 | |
| 14 | NO | E3:chr17:CYTH1:NM_004762:74217326:74217409:−: | REVERSE Strand |
| | | 58_83\|\|E6:chr17:EXOC7:NM_001145297:71605471:71605694: | |
| | | −:1_25 | |
| 15 | NO | E4:chr9:CIZ1:NM_012127:129989962:129990034:−: | REVERSE Strand |
| | | 55_72\|\|E1:chr9:FAM102A:NM_001035254:129782091:129782633: | |
| | | −:1_32 | |
| 16 | NO | E1:chr9:FAM102A:NM_001035254:129782091:129782633:−: | REVERSE_Strand |
| | | 26_1\|\|E5:chr9:CIZ1:NM_012127:129987646:129987876:−: | |
| | | 230_207 | |
| 17 | NO | E2:chr12:GCN1L1:NM_006836:119112483:119112586:−: | REVERSE_Strand |
| | | 22_1\|\|E12:chr12:MSI1:NM_002442:119269631:119269700: | |
| | | −:69_42 | |
| 18 | YES | E3:chr3:CMTM7:NM_138410:32458335:32458509:+:28_1\|\| | REVERSE_Strand |
| | | E15:chr3:GLB1:NM_001079811:33030551:33030806:−:1_22 | |
| 19 | YES | E9:chr8:PVT1:NR_003367:129182407:129182681:+:24_1\|\| | REVERSE_Strand |
| | | E5:chr8:GSDMC:NM_031415:130844053:130844159:−: | |
| | | 1_25 | |
| 20 | NO | E1:chr1:HDGF:NM_004494:154987758:154988167:−: | REVERSE_Strand |
| | | 25_1\|\|E2:chr1:S100A10:NM_002966:150225198:150225351: | |
| | | −:153_129 | |
| 21 | NO | E14:chr7:INTS1:NM_001080453:1500977:1501055:−: | REVERSE_Strand |
| | | 25_1\|\|E2:chr7:PRKAR1B:NM_002735:717491:717690:−: | |
| | | 199_175 | |
| 22 | YES | E12:chr14:KLHDC2:NM_014315:49319009:49319062:+:27_53\|\| | FORWARD_Strand |
| | | E5:chr8:SNTB1:NM_021021:121630182:121630379:−: | |
| | | 197_175 | |
| 23 | NO | E2:chr11:LDLRAD3:NM_174902:36014228:36014375:+:122_147\|\| | FORWARD Strand |
| | | E4:chr11:TCP11L1:NM_001145541:33035236:33035357: | |
| | | +:1_24 | |
| 24 | NO | E4:chr12:LIMA1:NM_016357:48902070:48902535:−: | REVERSE_Strand |
| | | 25_1\|\|E2:chr17:USP22:NM_015276:20872446:20872579:−: | |
| | | 133_109 | |
| 25 | NO | E5:chrX:TMLHE:NM_018196:154407310:154407487:−: | REVERSE Strand |
| | | 153_177\|\|E2:chrX:MECP2:NM_004992:153010835:153010959: | |
| | | −:1_25 | |
| 26 | YES | E17:chr17:STXBP4:NM_178509:50573669:50573727:+:24_1\|\| | REVERSE_Strand |
| | | E1:chr17:MED1:NM_004774:34860816:34861053:−:1_26 | |
| 27 | NO | E13:chr7:MLL5:NM_182931:104509370:104509480:+:87_110\|\| | FORWARD Strand |
| | | E3:chr7:LHFPL3:NM_199000:104333869:104336239:+: | |
| | | 1_26 | |
| 28 | NO | E2:chr22:EIF3D:NM_003753:35251991:35252124:−: | REVERSE Strand |
| | | 112_133\|\|E1:chr22:MYH9:NM_002473:35113797:35114009: | |
| | | −:1_28 | |
| 29 | NO | E2:chr1:NFIA:NM_001145511:61326408:61326940:+:506_532\|\| | FORWARD Strand |
| | | E5:chr11:EHF:NM_012153:34629664:34629733:+:1_24 | |
| 30 | YES | E4:chr8:NRG1:NM_013960:32572887:32573065:+:32_1\|\|E12: | REVERSE_Strand |
| | | chr11:ODZ4:NM_001098816:78242796:78243007:−:1_19 | |
| 31 | YES | E5:chr8:SAMD12:NM_001101676:119270875:119279152:−: | FORWARD_Strand |
| | | 8254_8277\|\|E9:chr8:PHF20L1:NM_032205:133886041:133886167: | |
| | | +:126_100 | |
| 32 | YES | E7:chr17:PIP4K2B:NM_003559:34187465:34187579:−: | REVERSE_Strand |
| | | 27_1\|\|E6:chr17:RAD51C:NM_058216:54156399:54156460: | |
| | | +:1_20 | |
| 33 | NO | E17:chr17:BRIP1:NM_032043:57148093:57148206:−: | REVERSE Strand |
| | | 90_113\|\|E2:chr17:POLDIP2:NM_015584:23706947:23707029: | |
| | | −:1_27 | |
| 34 | NO | E1:chr1:PPP1R12B:NM_002481:200584452:200584893:+:417_441\|\| | FORWARD Strand |
| | | E8:chr1:SNX27:NM_030918:149922455:149922545: | |
| | | +:1_25 | |
| 35 | NO | E8:chr1:PRPF3:NM_004698:148577259:148577426:+:140_167\|\| | FORWARD Strand |
| | | E4:chr1:SRGAP2:NM_001170637:204632668:204632884: | |
| | | +:1_22 | |
| 36 | NO | E4:chr1:PUM1:NM_001020658:31186584:31186706:−: | REVERSE_Strand |
| | | 29_1\|\|E5:chr6:TRERF1:NM_033502:42343869:42345564:−: | |
| | | 1695_1675 | |
| 37 | NO | E2:chr20:RAB22A:NM_020673:56319504:56319584:+:59_80\|\| | FORWARD Strand |
| | | E3:chr19:MYO9B:NM_004145:17117206:17117301:+:1_28 | |
| 38 | NO | E1:chr3:RAB7A:NM_004637:129927668:129927892:+:204_224\|\| | FORWARD Strand |
| | | E16:chr3:LRCH3:NM_032773:199076690:199076739: | |
| | | +:1_30 | |
| 39 | YES | E10:chr3:RFT1:NM_052859:53113008:53113153:−: | REVERSE_Strand |
| | | 23_1\|\|E9:chr16:UQCRC2:NM_003366:21890346:21890442: | |
| | | +:1_27 | |
| 40 | NO | E1:chr8:RIMS2:NM_001100117:104582151:104582466:+:288_315\|\| | FORWARD Strand |
| | | E9:chr8:ATP6V1C1:NM_001695:104144358:104144451: | |
| | | +:1_22 | |
| 41 | NO | E2:chr1:RNF187:NM_001010858:226743283:226743376:+: | FORWARD Strand |
| | | 70_93\|\|E79:chr1:OBSCN:NM_052843:226605164:226605267: | |
| | | +:1_26 | |
| 42 | YES | E28:chr13:DIAPH3:NM_001042517:59137723:59138981:−: | FORWARD_Strand |
| | | 1235_1258\|\|E6:chr17:RPS6KB1:NM_003161:55362259:55362317: | |
| | | +:58_33 | |
| 43 | YES | E1:chr17:RPS6KB1:NM_003161:55325224:55325468:+:220_244\|\| | FORWARD_Strand |
| | | E2:chr17:SNF8:NM_007241:44376285:44376336:−: | |
| | | 51_27 | |
| 44 | YES | E27:chr1:NOTCH2:NM_024408:120266781:120266924:−: | FORWARD_Strand |
| | | 119_143\|\|E1:chr1:SEC22B:NM_004892:143807763:143807978: | |
| | | +:215_191 | |
| 45 | NO | E12:chr21:SLC37A1:NM_018964:42852136:42852268:+:111_132\|\| | FORWARD Strand |
| | | E5:chr21:ABCG1:NM_207174:42570073:42570124: | |
| | | +:1_28 | |
| 46 | NO | E3:chr1:SRGAP2:NM_001170637:204623991:204624054:+: | FORWARD Strand |
| | | 45_63\|\|E15:chr1:PRPF3:NM_004698:148588256:148588318: | |
| | | +:1_31 | |
| 47 | NO | E1:chr17:STARD3:NM_001165937:35046858:35047010:+:128_152\|\| | FORWARD Strand |
| | | E7:chr20:DOK5:NM_018431:52693403:52693524: | |
| | | +:1_25 | |
| 48 | NO | E5:chr2:STRADB:NM_018571:202045922:202046044:+:99_122\|\| | FORWARD Strand |
| | | E11:chr2:NOP58:NM_015934:202870346:202870481: | |
| | | +:1_26 | |
| 49 | NO | E1:chr20:SULF2:NM_001161841:45848198:45848767:−: | REVERSE_Strand |
| | | 25_1\|\|E8:chr3:PRICKLE2:NM_198859:64054566:64060641: | |
| | | −:6075_6051 | |
| 50 | NO | E4:chr17:CCDC46:NM_001037325:61115708:61115798:−: | REVERSE Strand |
| | | 67_90\|\|E4:chr17:SUPT4H1:NM_003168:53779547:53779601: | |
| | | −:1_26 | |
| 51 | YES | E1:chr7:TAX1BP1:NM_001079864:27746262:27746413:+:126_151\|\| | FORWARD_Strand |
| | | E2:chr20:AHCY:NM_001161766:32346861:32347052: | |
| | | −:191_168 | |
| 52 | NO | E3:chr10:TIAL1:NM_003252:121337653:121337750:−: | REVERSE_Strand |
| | | 26_1\|\|E2:chr10:C10orf119:NM_024834:121609300:121609386: | |
| | | −:86_63 | |
| 53 | NO | E14:chr17:CDK12:NM_016507:34940382:34944326:+:22_1\|\| | FORWARD_Strand |
| | | E5:chr17:TMEM104:NM_017728:70297933:70298030:+:97_70 | |
| 54 | NO | E5:chr17:TMEM104:NM_017728:70297933:70298030:+:72_97\|\| | FORWARD Strand |
| | | E2:chr17:CDK12:NM_015083:34940409:34944326:+:1_24 | |
| 55 | YES | E3:chr20:TRPC4AP:NM_199368:33129509:33129638:−: | REVERSE_Strand |
| | | 26_1\|\|E7:chr17:MRPL45:NM_032351:33731535:33731709: | |
| | | +:1_21 | |
| 56 | NO | E1:chr8:UBR5:NM_015902:103493576:103493671:−: | REVERSE_Strand |
| | | 26_1\|\|E2:chr8:SLC25A32:NM_030780:104489037:104489188: | |
| | | −:151_128 | |
| 57 | NO | E1:chr17:WIPF2:NM_133264:35629099:35629270:+:148_171\|\| | FORWARD Strand |
| | | E4:chr17:ERBB2:NM_001005862:35104766:35104960: | |
| | | +:1_26 | |
| 58 | NO | E1:chr17:WIPF2:NM_133264:35629099:35629270:+:145_171\|\| | FORWARD Strand |
| | | E5:chr17:ERBB2:NM_001005862:35116768:35116920: | |
| | | +:1_23 | |
| 59 | YES | E20:chr20:ZMYND8:NM_183047:45286376:45286616:−: | REVERSE_Strand |
| | | 23_1\|\|E21:chr20:CEP250:NM_007186:33541876:33542044: | |
| | | +:1_27 | |
| 60 | YES | E20:chr20:ZMYND8:NM_183047:45286376:45286616:−: | REVERSE_Strand |
| | | 27_1\|\|E22:chr20:CEP250:NM_007186:33542451:33542586: | |
| | | +:1_20 | |

| | Total | | Alignment | Orientation of | |
|---|---|---|---|---|---|
| | Read | Alignment Orientation | Orientations | Two Fusion | Fusion between |
| Row | Pairs | Consistency of Two Ends | of Two Ends | Partners | exon boundaries |
| 1 | 72 | YES | f_r | r_r | YES |
| 2 | 30 | YES | f_r | f_f | YES |
| 3 | 421 | YES | f_f | f_r | YES |
| 4 | 18 | YES | f_r | f_f | YES |
| 5 | 23 | YES | f_r | f_f | YES |
| 6 | 10 | YES | f_r | r_r | YES |
| 7 | 1697 | NO\|f_f = \|r_r = 3\|f_r = 1566 | f_r\|r_r | f_f | YES |
| 8 | 13 | YES | f_r | f_f | YES |
| 9 | 28 | YES | r_r | f_r | YES |
| 10 | 11 | YES | f_r | f_f | YES |
| 11 | 64 | YES | f_r | f_f | YES |
| 12 | 33 | YES | f_r | r_r | YES |
| 13 | 91 | YES | f_f | f_r | YES |
| 14 | 20 | YES | f_r | r_r | YES |
| 15 | 31 | YES | f_r | r_r | YES |
| 16 | 31 | YES | f_r | r_r | YES |
| 17 | 25 | YES | f_r | r_r | YES |
| 18 | 13 | YES | r_r | f_r | YES |
| 19 | 23 | YES | r_r | f_r | YES |
| 20 | 154 | YES | f_r | r_r | YES |
| 21 | 24 | YES | f_r | r_r | YES |
| 22 | 25 | YES | f_f | f_r | YES |
| 23 | 25 | YES | f_r | f_f | YES |
| 24 | 16 | YES | f_r | r_r | YES |
| 25 | 29 | YES | f_r | r_r | YES |
| 26 | 54 | YES | r_r | f_r | YES |
| 27 | 23 | YES | f_r | f_f | YES |
| 28 | 16 | YES | f_r | r_r | YES |
| 29 | 18 | YES | f_r | f_f | YES |
| 30 | 12 | YES | r_r | f_r | YES |
| 31 | 106 | YES | f_f | f_r | YES |
| 32 | 15 | YES | r_r | f_r | YES |
| 33 | 13 | YES | f_r | r_r | YES |
| 34 | 45 | YES | f_r | f_f | YES |
| 35 | 22 | YES | f_r | f_f | YES |
| 36 | 58 | YES | f_r | r_r | YES |
| 37 | 16 | YES | f_r | f_f | YES |
| 38 | 14 | YES | f_r | f_f | YES |
| 39 | 102 | YES | r_r | f_r | YES |
| 40 | 11 | YES | f_r | f_f | YES |
| 41 | 11 | YES | f_r | f_f | YES |
| 42 | 25 | YES | f_f | f_r | YES |
| 43 | 162 | YES | f_f | f_r | YES |
| 44 | 22 | YES | f_f | f_r | YES |
| 45 | 20 | YES | f_r | f_f | YES |
| 46 | 22 | YES | f_r | f_f | YES |
| 47 | 21 | YES | f_r | f_f | YES |
| 48 | 10 | YES | f_r | f_f | YES |
| 49 | 26 | YES | f_r | r_r | YES |
| 50 | 17 | YES | f_r | r_r | YES |
| 51 | 54 | YES | f_f | f_r | YES |
| 52 | 12 | YES | f_r | r_r | YES |
| 53 | 10 | YES | f_r | f_f | YES |
| 54 | 10 | YES | f_r | f_f | YES |
| 55 | 27 | YES | r_r | f_r | YES |
| 56 | 28 | YES | f_r | r_r | YES |
| 57 | 66 | YES | f_r | f_f | YES |
| 58 | 66 | YES | f_r | f_f | YES |
| 59 | 189 | YES | r_r | f_r | YES |
| 60 | 189 | YES | r_r | f_r | YES |

| | Number | | Recommended Sequence for |
|---|---|---|---|
| | of Fusion | | Primer Design |
| Row | Isoforms | Description | (SEQ ID NO:) |
| 1 | 1 | Breast Cancer Cell Line | 1 |
| 2 | 1 | Breast Cancer Cell Line | 2 |
| 3 | 1 | Breast Cancer Cell Line | 3 |
| 4 | 1 | Breast Cancer Cell Line | 4 |
| 5 | 1 | Breast Cancer Cell Line | 5 |
| 6 | 1 | Breast Cancer Cell Line | 6 |
| 7 | 1 | Breast Cancer Cell Line | 7 |
| 8 | 1 | Breast Cancer Cell Line | 8 |
| 9 | 1 | Breast Cancer Cell Line | 9 |
| 10 | 1 | Breast Cancer Cell Line | 10 |
| 11 | 1 | Breast Cancer Cell Line | 11 |
| 12 | 1 | Breast Cancer Cell Line | 12 |
| 13 | 1 | Breast Cancer Cell Line | 13 |
| 14 | 1 | Breast Cancer Cell Line | 14 |
| 15 | 2 | Breast Cancer Cell Line | 15 |
| 16 | 2 | Breast Cancer Cell Line | 16 |
| 17 | 1 | Breast Cancer Cell Line | 17 |
| 18 | 1 | Breast Cancer Cell Line | 18 |
| 19 | 1 | Breast Cancer Cell Line | 19 |
| 20 | 1 | Breast Cancer Cell Line | 20 |
| 21 | 1 | Breast Cancer Cell Line | 21 |
| 22 | 1 | Breast Cancer Cell Line | 22 |
| 23 | 1 | Breast Cancer Cell Line | 23 |
| 24 | 1 | Breast Cancer Cell Line | 24 |
| 25 | 1 | Breast Cancer Cell Line | 25 |
| 26 | 1 | Breast Cancer Cell Line | 26 |
| 27 | 1 | Breast Cancer Cell Line | 27 |
| 28 | 1 | Breast Cancer Cell Line | 28 |
| 29 | 1 | Breast Cancer Cell Line | 29 |
| 30 | 1 | Breast Cancer Cell Line | 30 |
| 31 | 1 | Breast Cancer Cell Line | 31 |
| 32 | 1 | Breast Cancer Cell Line | 32 |
| 33 | 1 | Breast Cancer Cell Line | 33 |
| 34 | 1 | Breast Cancer Cell Line | 34 |
| 35 | 2 | Breast Cancer Cell Line | 35 |
| 36 | 1 | Breast Cancer Cell Line | 36 |
| 37 | 1 | Breast Cancer Cell Line | 37 |
| 38 | 1 | Breast Cancer Cell Line | 38 |
| 39 | 1 | Breast Cancer Cell Line | 39 |
| 40 | 1 | Breast Cancer Cell Line | 40 |
| 41 | 1 | Breast Cancer Cell Line | 41 |
| 42 | 1 | Breast Cancer Cell Line | 42 |
| 43 | 1 | Breast Cancer Cell Line | 43 |
| 44 | 1 | Breast Cancer Cell Line | 44 |
| 45 | 1 | Breast Cancer Cell Line | 45 |
| 46 | 2 | Breast Cancer Cell Line | 46 |
| 47 | 1 | Breast Cancer Cell Line | 47 |
| 48 | 1 | Breast Cancer Cell Line | 48 |
| 49 | 1 | Breast Cancer Cell Line | 49 |
| 50 | 1 | Breast Cancer Cell Line | 50 |
| 51 | 1 | Breast Cancer Cell Line | 51 |
| 52 | 1 | Breast Cancer Cell Line | 52 |
| 53 | 2 | Breast Cancer Cell Line | 53 |
| 54 | 2 | Breast Cancer Cell Line | 54 |
| 55 | 1 | Breast Cancer Cell Line | 55 |
| 56 | 1 | Breast Cancer Cell Line | 56 |
| 57 | 2 | Breast Cancer Cell Line | 57 |
| 58 | 2 | Breast Cancer Cell Line | 58 |
| 59 | 2 | Breast Cancer Cell Line | 59 |
| 60 | 2 | Breast Cancer Cell Line | 60 |

TABLE 5

| | Primer 1 | Primer 2 | | |
|---|---|---|---|---|
| | (SEQ | (SEQ | Product | |
| Fusion Gene | ID NO:) | ID NO:) | Size | Cell Line |
| LIMA1->USP22 | 333 | 393 | 86 | BT-20 |
| ACACA->STAC2 | 334 | 394 | 80 | BT-474 |
| ZMYND8->CEP250 | 335 | 395 | 83 | BT-474 |
| isoform 1 | | | | |
| ZMYND8->CEP250 | 336 | 396 | 96 | BT-474 |
| isoform 2 | | | | |
| FAM102A->CIZ1 | 337 | 397 | 84 | BT-474 |
| isoform 1 | | | | |
| FAM102A->CIZ1 | 338 | 398 | 99 | BT-474 |
| isoform 2 | | | | |
| GLB1->CMTM7 | 339 | 399 | 98 | BT-474 |
| STARD3->DOK5 | 340 | 400 | 111 | BT-474 |
| MED1->STXBP4 | 341 | 401 | 94 | BT-474 |
| TRPC4AP->MRPL45 | 342 | 402 | 89 | BT-474 |
| RAB22A->MYO9B | 343 | 403 | 98 | BT-474 |
| PIP4K2B->RAD51C | 344 | 404 | 81 | BT-474 |
| RPS6KB1->SNF8 | 345 | 405 | 82 | BT-474 |
| CTAGE5->SIP1 | 346 | 406 | 80 | HCC1187 |
| MLL5->LHFPL3 | 347 | 407 | 91 | HCC1187 |
| SEC22B->NOTCH2 | 348 | 408 | 97 | HCC1187 |
| PUM1->TRERF1 | 349 | 409 | 90 | HCC1187 |
| EIF3K->CYP39A1 | 350 | 410 | 96 | HCC1395 |
| RAB7A->LRCH3 | 351 | 411 | 100 | HCC1395 |
| SLC37A1->ABCG1 | 352 | 412 | 88 | HCC1428 |
| RNF187->OBSCN | 353 | 413 | 92 | HCC1428 |
| EXOC7->CYTH1 | 354 | 414 | 83 | HCC1599 |
| CYTH1->PRPSAP1 | 355 | 415 | 84 | HCC1599 |
| TAX1BP1->AHCY | 356 | 416 | 91 | HCC1806 |
| BRE->DPYSL5 | 357 | 417 | 97 | HCC1806 |
| CD151->DRD4 | 358 | 418 | 84 | HCC1806 |
| LDLRAD3->TCP11L1 | 359 | 419 | 100 | HCC1806 |
| RFT1->UQCRC2 | 360 | 420 | 99 | HCC1806 |
| NFIA->EHF | 361 | 421 | 92 | HCC1937 |
| GSDMC->PVT1 | 362 | 422 | 95 | HCC1954 |
| INTS1->PRKAR1B | 363 | 423 | 100 | HCC1954 |
| STRADB->NOP58 | 364 | 424 | 98 | HCC1954 |
| PHF20L1->SAMD12 | 365 | 425 | 92 | HCC1954 |
| POLDIP2->BRIP1 | 366 | 426 | 99 | HCC2218 |
| ADAMTS19->SLC27A6 | 367 | 427 | 81 | MCF7 |
| ARFGEF2->SULF2 | 368 | 428 | 98 | MCF7 |
| ATXN7L3->FAM171A2 | 369 | 429 | 100 | MCF7 |
| BCAS4->BCAS3 | 370 | 430 | 82 | MCF7 |
| RPS6KB1->DIAPH3 | 371 | 431 | 83 | MCF7 |
| MYH9->EIF3D | 372 | 432 | 97 | MCF7 |
| GCN1L1->MSI1 | 373 | 433 | 98 | MCF7 |
| SULF2->PRICKLE2 | 374 | 434 | 81 | MCF7 |
| ODZ4->NRG1 | 375 | 435 | 98 | MDA-MB- |
| | | | | 175V-II |
| BRIP1->TMEM49 | 376 | 436 | 91 | MDA-MB- |
| | | | | 361 |
| SUPT4H1->CCDC46 | 377 | 437 | 96 | MDA-MB- |
| | | | | 361 |
| TMEM104->CDK12 | 378 | 438 | 90 | MDA-MB- |
| isoform 1 | | | | 361 |
| TMEM104->CDK12 | 379 | 439 | 87 | MDA-MB- |
| isoform 2 | | | | 361 |
| RIMS2->ATP6V1C1 | 380 | 440 | 80 | MDA-MB- |
| | | | | 436 |
| TIAL1->C10orf119 | 381 | 441 | 80 | MDA-MB- |
| | | | | 436 |
| MECP2->TMLHE | 382 | 442 | 88 | MDA-MB- |
| | | | | 453 |
| ARID1A->MAST2 | 383 | 443 | 120 | MDA-MB- |
| | | | | 468 |
| UBR5->SLC25A32 | 384 | 444 | 95 | MDA-MB- |
| | | | | 468 |
| KLHDC2->SNTB1 | 385 | 445 | 90 | SK-BR-3 |
| ARID1A->WDTC1 | 386 | 446 | 114 | UACC812 |
| WIPF2->ERBB2 | 387 | 447 | 98 | UACC812 |
| isoform 1 | | | | |
| WIPF2->ERBB2 | 388 | 448 | 91 | UACC812 |
| isoform 2 | | | | |
| HDGF->S100A10 | 389 | 449 | 88 | UACC812 |
| PPP1R12B->SNX27 | 390 | 450 | 98 | UACC812 |
| SRGAP2->PRPF3 | 391 | 451 | 92 | UACC812 |
| isoform 1 | | | | |
| SRGAP2->PRPF3 | 392 | 452 | 90 | UACC812 |
| isoform 2 | | | | |

Among the 55 fusion candidates, 30 were in-frame (Tables 3 and 6). A fusion product was defined as “in frame” when there was no frame shift in the 3′ gene, regardless whether there is single amino acid mutation or single/multiple amino acid insertion at the fusion junction point. The fusion junction point mutations were also listed in Table 6. In addition, the list of fusion transcripts as the result of exhaustive combinations of all transcripts from two partner genes may contain identical fusion products if the differences between the transcripts from the same partner are “fused out.” For example, as shown in FIG. 3D, the fusion transcript of A1-B4 was identical to that of A1-B1, and the fusion transcript of A2-B4 was identical to that of A2-B1. These identical fusion proteins were flagged in the SnowShoes output file (Table 6).

TABLE 6

| # | FUSION | NOTE | Transcripts | In frame | Junction Point Mutations | Boundary Exon 5′ Gene |
|---|---|---|---|---|---|---|
| 1 | ACACA->STAC2 | | NM_198834->NM_198993 | YES | | E49: chr17: 32553565-32553662 |
| 2 | ACACA->STAC2 | | NM_198836->NM_198993 | YES | | E49: chr17: 32553565-32553662 |
| 3 | ACACA->STAC2 | | NM_198837->NM_198993 | YES | | E47: chr17: 32553565-32553662 |
| 4 | ACACA->STAC2 | | NM_198838->NM_198993 | YES | | E48: chr17: 32553565-32553662 |
| 5 | ACACA->STAC2 | | NM_198839->NM_198993 | YES | | E53: chr17: 32553565-32553662 |
| 6 | ADAMTS19->SLC27A6 | | NM_133638->NM_001017372 | | | E1: chr5: 128824001-128824074 |
| 7 | ADAMTS19->SLC27A6 | | NM_133638->NM_014031 | | | E1: chr5: 128824001-128824074 |
| 8 | ARFGEF2->SULF2 | | NM_006420->NM_001161841 | YES | GGT->ACC(G->T) | E1: chr20: 46971681-46971954 |
| 9 | ARFGEF2->SULF2 | | NM_006420->NM_018837 | YES | GGT->ACC(G->T) | E1: chr20: 46971681-46971954 |
| 10 | ARFGEF2->SULF2 | | NM_006420->NM_198596 | YES | GGT->ACC(G->T) | E1: chr20: 46971681-46971954 |
| 11 | ARID1A->MAST2 | | NM_006015->NM_015112 | YES | | E1: chr1: 26895108-26896618 |
| 12 | ARID1A->MAST2 | | NM_139135->NM_015112 | YES | | E1: chr1: 26895108-26896618 |
| 13 | ARID1A->WDTC1 | | NM_006015->NM_015023 | YES | | E1: chr1: 26895108-26896618 |
| 14 | ARID1A->WDTC1 | | NM_139135->NM_015023 | YES | | E1: chr1: 26895108-26896618 |
| 15 | ATXN7L3->FAM171A2 | | NM_001098833->NM_198475 | | | E1: chr17: 39630913-39631055 |
| 16 | ATXN7L3->FAM171A2 | | NM_020218->NM_198475 | | | E1: chr17: 39630913-39631055 |
| 17 | BCAS4->BCAS3 | | NM_001010974->NM_001099432 | | | E1: chr20: 48844873-48845117 |
| 18 | BCAS4->BCAS3 | | NM_001010974->NM_017679 | | | E1: chr20: 48844873-48845117 |
| 19 | BCAS4->BCAS3 | | NM_017843->NM_001099432 | | | E1: chr20: 48844873-48845117 |
| 20 | BCAS4->BCAS3 | | NM_017843->NM_017679 | | | E1: chr20: 48844873-48845117 |
| 21 | BCAS4->BCAS3 | | NM_198799->NM_001099432 | | | E1: chr20: 48844873-48845117 |
| 22 | BCAS4->BCAS3 | | NM_198799->NM_017679 | | | E1: chr20: 48844873-48845117 |
| 23 | BRE->DPYSL5 | | NM_004899->NM_020134 | YES | INSERTION: CAGAAC(QN) | E7: chr2: 28205641-28205751 |
| 24 | BRE->DPYSL5 | | NM_199191->NM_020134 | YES | INSERTION: CAGAAC(QN) | E7: chr2: 28205641-28205751 |
| 25 | BRE->DPYSL5 | | NM_199192->NM_020134 | YES | INSERTION: CAGAAC(QN) | E7: chr2: 28205641-28205751 |
| 26 | BRE->DPYSL5 | | NM_199193->NM_020134 | YES | INSERTION: CAGAAC(QN) | E8: chr2: 28205641-28205751 |
| 27 | BRE->DPYSL5 | | NM_199194->NM_020134 | YES | INSERTION: CAGAAC(QN) | E8: chr2: 28205641-28205751 |
| 28 | BRIP1->TMEM49 | | NM_032043->NM_030938 | | | E3: chr17: 57291938-57292050 |
| 29 | CD151->DRD4 | | NM_001039490->NM_000797 | | | E4: chr11: 826768-826843 |
| 30 | CD151->DRD4 | | NM_004357->NM_000797 | | | E5: chr11: 826768-826843 |
| 31 | CD151->DRD4 | | NM_139029->NM_000797 | | | E5: chr11: 826768-826843 |
| 32 | CD151->DRD4 | | NM_139030->NM_000797 | | | E4: chr11: 826768-826843 |
| 33 | CTAGE5->SIP1 | | NM_005930->NM_001009182 | | | E20: chr14: 38865818-38865977 |
| 34 | CTAGE5->SIP1 | | NM_005930->NM_001009183 | | | E20: chr14: 38865818-38865977 |
| 35 | CTAGE5->SIP1 | | NM_005930->NM_003616 | | | E20: chr14: 38865818-38865977 |
| 36 | CTAGE5->SIP1 | | NM_203354->NM_001009182 | | | E20: chr14: 38865818-38865977 |
| 37 | CTAGE5->SIP1 | | NM_203354->NM_001009183 | | | E20: chr14: 38865818-38865977 |
| 38 | CTAGE5->SIP1 | | NM_203354->NM_003616 | | | E20: chr14: 38865818-38865977 |
| 39 | CTAGE5->SIP1 | | NM_203355->NM_001009182 | | | E19: chr14: 38865818-38865977 |
| 40 | CTAGE5->SIP1 | | NM_203355->NM_001009183 | | | E19: chr14: 38865818-38865977 |
| 41 | CTAGE5->SIP1 | | NM_203355->NM_003616 | | | E19: chr14: 38865818-38865977 |
| 42 | CTAGE5->SIP1 | | NM_203356->NM_001009182 | | | E20: chr14: 38865818-38865977 |
| 43 | CTAGE5->SIP1 | | NM_203356->NM_00100918 | | | E20: chr14: 38865818-38865977 |
| 44 | CTAGE5->SIP1 | | NM_203356->NM_003616 | | | E20: chr14: 38865818-38865977 |
| 45 | CYTH1->PRPSAP1 | | NM_004762->NM_002766 | YES | GAA->TTC(E->F) | E1: chr17: 74289878-74289971 |
| 46 | CYTH1->PRPSAP1 | | NM_017456->NM_002766 | YES | GAA->TTC(E->F) | E1: chr17: 74289878-74289971 |
| 47 | EIF3K->CYP39A1 | | NM_013234->NM_016593 | YES | GCA->TGC(A->C) | E6: chr19: 43815080-43815158 |
| 48 | EXOC7->CYTH1 | | NM_001013839->NM_004762 | YES | GTT->AAC(V->N) | E5: chr17: 71605471-71605694 |
| 49 | EXOC7->CYTH1 | | NM_001013839->NM_017456 | YES | GTT->AAC(V->N) | E5: chr17: 71605471-71605694 |
| 50 | EXOC7->CYTH1 | | NM_001145297->NM_004762 | YES | GTT->AAC(V->N) | E5: chr17: 71605471-71605694 |
| 51 | EXOC7->CYTH1 | | NM_001145297->NM_017456 | YES | GTT->AAC(V->N) | E5: chr17: 71605471-71605694 |
| 52 | EXOC7->CYTH1 | | NM_001145298->NM_004762 | YES | GTT->AAC(V->N) | E5: chr17: 71605471-71605694 |
| 53 | EXOC7->CYTH1 | | NM_001145298->NM_017456 | YES | GTT->AAC(V->N) | E5: chr17: 71605471-71605694 |
| 54 | EXOC7->CYTH1 | | NM_001145299->NM_004762 | YES | GTT->AAC(V->N) | E5: chr17: 71605471-71605694 |
| 55 | EXOC7->CYTH1 | | NM_001145299->NM_017456 | YES | GTT->AAC(V->N) | E5: chr17: 71605471-71605694 |
| 56 | EXOC7->CYTH1 | | NM_015219->NM_004762 | YES | GTT->AAC(V->N) | E5: chr17: 71605471-71605694 |
| 57 | EXOC7->CYTH1 | | NM_015219->NM_017456 | YES | GTT->AAC(V->N) | E5: chr17: 71605471-71605694 |
| 58 | EXOC7->CYTH1 | | NR_028133->NM_004762 | YES | | E4: chr17: 71605471-71605694 |
| 59 | EXOC7->CYTH1 | | NR_028133->NM_017456 | YES | | E4: chr17: 71605471-71605694 |
| 60 | FAM102A->CIZ1 | | NM_001035254-> | | | E1: chr9: 129782091-129782633 |
| | | | NM_001131015 | | | |
| 61 | FAM102A->CIZ1 | | NM_001035254-> | | | E1: chr9: 129782091-129782633 |
| | | | NM_001131015 | | | |
| 62 | FAM102A->CIZ1 | | NM_001035254-> | | | E1: chr9: 129782091-129782633 |
| | | | NM_001131016 | | | |
| 63 | FAM102A->CIZ1 | | NM_001035254-> | | | E1: chr9: 129782091-129782633 |
| | | | NM_001131016 | | | |
| 64 | FAM102A->CIZ1 | | NM_001035254-> | | | E1: chr9: 129782091-129782633 |
| | | | NM_001131017 | | | |
| 65 | FAM102A->CIZ1 | | NM_001035254-> | | | E1: chr9: 129782091-129782633 |
| | | | NM_001131017 | | | |
| 66 | FAM102A->CIZ1 | | NM_001035254-> | | | E1: chr9: 129782091-129782633 |
| | | | NM_001131018 | | | |
| 67 | FAM102A->CIZ1 | | NM_001035254-> | | | E1: chr9: 129782091-129782633 |
| | | | NM_001131018 | | | |
| 68 | FAM102A->CIZ1 | | NM_001035254->NM_012127 | | | E1: chr9: 129782091-129782633 |
| 69 | FAM102A->CIZ1 | | NM_001035254->NM_012127 | | | E1: chr9: 129782091-129782633 |
| 70 | GCN1L1->MSI1 | | NM_006836->NM_002442 | YES | GCC->GGC(A->G) | E2: chr12: 119112483-119112586 |
| 71 | GLB1->CMTM7 | | NM_000404->NM_138410 | YES | | E15: chr3: 33030551-33030806 |
| 72 | GLB1->CMTM7 | | NM_000404->NM_181472 | YES | | E15: chr3: 33030551-33030806 |
| 73 | GLB1->CMTM7 | | NM_001079811->NM_138410 | YES | | E15: chr3: 33030551-33030806 |
| 74 | GLB1->CMTM7 | | NM_001079811->NM_181472 | YES | | E15: chr3: 33030551-33030806 |
| 75 | GLB1->CMTM7 | | NM_001135602->NM_138410 | YES | | E12: chr3: 33030551-33030806 |
| 76 | GLB1->CMTM7 | | NM_001135602->NM_181472 | YES | | E12: chr3: 33030551-33030806 |
| 77 | GSDMC->PVT1 | | NM_031415->NR_003367 | | | E5: chr8: 130844053-130844159 |
| 78 | HDGF-> | | NM_004494->NM_002966 | YES | | E1: chr1: 154987758-154988167 |
| | S100A10 | | | | | |
| 79 | INTS1-> | | NM_001080453-> | YES | | E14: chr7: 1500977-1501055 |
| | PRKAR1B | | NM_001164758 | | | |
| 80 | INTS1->PRKAR1B | | NM_001080453-> | YES | | E14: chr7: 1500977-1501055 |
| | | | NM_001164759 | | | |
| 81 | INTS1->PRKAR1B | | NM_001080453-> | YES | | E14: chr7: 1500977-1501055 |
| | | | NM_001164760 | | | |
| 82 | INTS1->PRKAR1B | | NM_001080453-> | YES | | E14: chr7: 1500977-1501055 |
| | | | NM_001164761 | | | |
| 83 | INTS1->PRKAR1B | | NM_001080453-> | YES | | E14: chr7: 1500977-1501055 |
| | | | NM_001164762 | | | |
| 84 | INTS1->PRKAR1B | | NM_001080453->NM_002735 | YES | | E14: chr7: 1500977-1501055 |
| 85 | KLHDC2->SNTB1 | | NM_014315->NM_021021 | YES | CGG->CCT(R->P) | E12: chr14: 49319009-49319062 |
| 86 | LDLRAD3->TCP11L1 | | NM_174902->NM_001145541 | | | E2: chr11: 36014228-36014375 |
| 87 | LDLRAD3->TCP11L1 | | NM_174902->NM_018393 | | | E2: chr11: 36014228-36014375 |
| 88 | LIMA1->USP22 | | NM_001113546->NM_015276 | YES | | E4: chr12: 48902070-48902535 |
| 89 | LIMA1->USP22 | | NM_016357->NM_015276 | YES | | E4: chr12: 48902070-48902535 |
| 90 | MECP2->TMLHE | | NM_004992->NM_001184797 | | | E2: chrX: 153010835-153010959 |
| 91 | MECP2->TMLHE | | NM_004992->NM_018196 | | | E2: chrX: 153010835-153010959 |
| 92 | MED1->STXBP4 | | NM_004774->NM_178509 | YES | TGT->GGT(C->G) | E1: chr17: 34860816-34861053 |
| 93 | MLL5->LHFPL3 | | NM_018682->NM_199000 | | | E12: chr7: 104509370-104509480 |
| 94 | MLL5->LHFPL3 | | NM_182931->NM_199000 | | | E13: chr7: 104509370-104509480 |
| 95 | MYH9->EIF3D | | NM_002473->NM_003753 | YES | | E1: chr22: 35113797-35114009 |
| 96 | NFIA->EHF | | NM_001134673->NM_012153 | YES | | E2: chr1: 61326408-61326940 |
| 97 | NFIA->EHF | | NM_001145511->NM_012153 | YES | | E2: chr1: 61326408-61326940 |
| 98 | NFIA->EHF | | NM_001145512->NM_012153 | YES | | E3: chr1: 61326408-61326940 |
| 99 | NFIA->EHF | | NM_005595->NM_012153 | YES | | E2: chr1: 61326408-61326940 |
| 100 | ODZ4->NRG1 | | NM_001098816-> | YES | | E12: chr11: 78242796-78243007 |
| | | | NM_001160002 | | | |
| 101 | ODZ4->NRG1 | | NM_001098816-> | YES | | E12: chr11: 78242796-78243007 |
| | | | NM_001160004 | | | |
| 102 | ODZ4->NRG1 | | NM_001098816-> | YES | | E12: chr11: 78242796-78243007 |
| | | | NM_001160005 | | | |
| 103 | ODZ4->NRG1 | | NM_001098816-> | YES | | E12: chr11: 78242796-78243007 |
| | | | NM_001160007 | | | |
| 104 | ODZ4->NRG1 | | NM_001098816-> | YES | | E12: chr11: 78242796-78243007 |
| | | | NM_001160008 | | | |
| 105 | ODZ4->NRG1 | | NM_001098816->NM_004495 | YES | | E12: chr11: 78242796-78243007 |
| 106 | ODZ4->NRG1 | | NM_001098816->NM_013956 | YES | | E12: chr11: 78242796-78243007 |
| 107 | ODZ4->NRG1 | | NM_001098816->NM_013957 | YES | | E12: chr11: 78242796-78243007 |
| 108 | ODZ4->NRG1 | | NM_001098816->NM_013958 | YES | | E12: chr11: 78242796-78243007 |
| 109 | ODZ4->NRG1 | | NM_001098816->NM_013960 | YES | | E12: chr11: 78242796-78243007 |
| 110 | ODZ4->NRG1 | | NM_001098816->NM_013962 | YES | | E12: chr11: 78242796-78243007 |
| 111 | ODZ4->NRG1 | | NM_001098816->NM_013964 | YES | | E12: chr11: 78242796-78243007 |
| 112 | PHF20L1->SAMD12 | | NM_016018->NM_001101676 | YES | GCA->TGC(A->C) | E8: chr8: 133886041-133886167 |
| 113 | PHF20L1->SAMD12 | | NM_032205->NM_001101676 | YES | GCA->TGC(A->C) | E8: chr8: 133886041-133886167 |
| 114 | PHF20L1->SAMD12 | | NM_198513->NM_001101676 | YES | GCA->TGC(A->C) | E7: chr8: 133886041-133886167 |
| 115 | PIP4K2B->RAD51C | | NM_003559->NM_058216 | | | E7: chr17: 34187465-34187579 |
| 116 | POLDIP2->BRIP1 | | NM_015584->NM_032043 | | | E2: chr17: 23706947-23707029 |
| 117 | PPP1R12B->SNX27 | | NM_001167857->NM_030918 | YES | | E1: chr1: 200584452-200584893 |
| 118 | PPP1R12B->SNX27 | | NM_001167858->NM_030918 | YES | | E1: chr1: 200584452-200584893 |
| 119 | PPP1R12B->SNX27 | | NM_002481->NM_030918 | YES | | E1: chr1: 200584452-200584893 |
| 120 | PRPF3->SRGAP2 | | NM_004698->NM_001042758 | | | E8: chr1: 148577259-148577426 |
| 121 | PRPF3->SRGAP2 | | NM_004698->NM_001170637 | | | E8: chr1: 148577259-148577426 |
| 122 | PRPF3->SRGAP2 | | NM_004698->NM_015326 | | | E8: chr1: 148577259-148577426 |
| 123 | PUM1->TRERF1 | | NM_001020658->NM_033502 | | | E20: chr1: 31186584-31186706 |
| 124 | PUM1->TRERF1 | | NM_014676->NM_033502 | | | E20: chr1: 31186584-31186706 |
| 125 | RAB22A->MYO9B | | NM_020673->NM_001130065 | | | E2: chr20: 56319504-56319584 |
| 126 | RAB22A->MYO9B | | NM_020673->NM_004145 | | | E2: chr20: 56319504-56319584 |
| 127 | RAB7A->LRCH3 | | NM_004637->NM_032773 | | | E1: chr3: 129927668-129927892 |
| 128 | RFT1->UQCRC2 | | NM_052859->NM_003366 | YES | | E10: chr3: 53113008-53113153 |
| 129 | RIMS2->ATP6V1C1 | | NM_001100117->NM_001695 | YES | | E1: chr8: 104582151-104582466 |
| 130 | RNF187-> | | NM_001010858-> | | | E2: chr1: 226743283-226743376 |
| | OBSCN | | NM_001098623 | | | |
| 131 | RNF187-> | | NM_001010858->NM_052843 | | | E2: chr1: 226743283-226743376 |
| | OBSCN | | | | | |
| 132 | RPS6KB1-> | | NM_003161->NM_001042517 | | | E6: chr17: 55362259-55362317 |
| | DIAPH3 | | | | | |
| 133 | RPS6KB1->SNF8 | | NM_003161->NM_007241 | YES | | E1: chr17: 55325224-55325468 |
| 134 | SEC22B-> | | NM_004892->NM_024408 | | | E1: chr1: 143807763-143807978 |
| | NOTCH2 | | | | | |
| 135 | SLC37A1-> | | NM_018964->NM_004915 | YES | | E12: chr21: 42852136-42852268 |
| | ABCG1 | | | | | |
| 136 | SLC37A1-> | | NM_018964->NM_016818 | YES | | E12: chr21: 42852136-42852268 |
| | ABCG1 | | | | | |
| 137 | SLC37A1-> | | NM_018964->NM_207174 | YES | | E12: chr21: 42852136-42852268 |
| | ABCG1 | | | | | |
| 138 | SLC37A1-> | | NM_018964->NM_207627 | YES | | E12: chr21: 42852136-42852268 |
| | ABCG1 | | | | | |
| 139 | SLC37A1-> | | NM_018964->NM_207628 | YES | | E12: chr21: 42852136-42852268 |
| | ABCG1 | | | | | |
| 140 | SLC37A1-> | | NM_018964->NM_207629 | YES | | E12: chr21: 42852136-42852268 |
| | ABCG1 | | | | | |
| 141 | SRGAP2-> | | NM_001042758->NM_004698 | YES | | E2: chr1: 204623991-204624054 |
| | PRPF3 | | | | | |
| 142 | SRGAP2-> | | NM_001170637->NM_004698 | YES | | E2: chr1: 204623991-204624054 |
| | PRPF3 | | | | | |
| 143 | SRGAP2-> | | NM_015326->NM_004698 | YES | | E2: chr1: 204623991-204624054 |
| | PRPF3 | | | | | |
| 144 | STARD3->DOK5 | 5′UTR of STARD3 fused into the | NM_001165937->NM_018431 | | | E1: chr17: 35046858-35047010 |
| | | coding region of DOK5 | | | | |
| 145 | STARD3->DOK5 | 5′UTR of STARD3 | NM_001165938-> | | | E1: chr17: 35046858-35047010 |
| | | fused into the coding | NM_018431 | | | |
| | | region of DOK5 | | | | |
| 146 | STARD3->DOK5 | 5′UTR of STARD3 | NM_006804->NM_018431 | | | E1: chr17: 35046858-35047010 |
| | | fused into the coding | | | | |
| | | region of DOK5 | | | | |
| 147 | STRADB->NOP58 | | NM_018571->NM_015934 | YES | | E5: chr2: 202045922-202046044 |
| 148 | SULF2->PRICKLE2 | 5′UTR of SULF2 fused | NM_001161841-> | | | E1: chr20: 45848198-45848767 |
| | | into the coding region of | NM_198859 | | | |
| | | PRICKLE2 | | | | |
| 149 | SULF2->PRICKLE2 | | NM_018837->NM_198859 | | | E21: chr20: 45848198-45848767 |
| 150 | SULF2->PRICKLE2 | 5′UTR of SULF2 fused | NM_198596->NM_198859 | | | E1: chr20: 45848198-45848767 |
| | | into the coding region of | | | | |
| | | PRICKLE2 | | | | |
| 151 | SUPT4H1->CCDC46 | | NM_003168-> | | | E4: chr17: 53779547-53779601 |
| | | | NM_001037325 | | | |
| 152 | SUPT4H1->CCDC46 | | NM_003168->NM_145036 | | | E4: chr17: 53779547-53779601 |
| 153 | TAX1BP1->AHCY | 5′UTR of TAX1BP1 | NM_001079864-> | | | E1: chr7: 27746262-27746413 |
| | | fused into the coding | NM_000687 | | | |
| | | region of AHCY | | | | |
| 154 | TAX1BP1->AHCY | 5′UTR of TAX1BP1 | NM_001079864-> | YES | | E1: chr7: 27746262-27746413 |
| | | fused into the 5′ UTR of | NM_001161766 | | | |
| | | AHCY | | | | |
| 155 | TAX1BP1->AHCY | 5′UTR of TAX1BP1 | NM_006024->NM_000687 | | | E1: chr7: 27746262-27746413 |
| | | fused into the coding | | | | |
| | | region of AHCY | | | | |
| 156 | TAX1BP1->AHCY | 5′UTR of TAX1BP1 | NM_006024-> | YES | | E1: chr7: 27746262-27746413 |
| | | fused into the 5′ UTR of | NM_001161766 | | | |
| | | AHCY | | | | |
| 157 | TIAL1->C10orf119 | | NM_001033925-> | | | E2: chr10: 121337653-121337750 |
| | | | NM_024834 | | | |
| 158 | TIAL1->C10orf119 | | NM_003252->NM_024834 | | | E2: chr10: 121337653-121337750 |
| 159 | TMEM104->CDK12 | | NM_017728->NM_015083 | | | E5: chr17: 70297933-70298030 |
| 160 | TMEM104->CDK12 | | NM_017728->NM_015083 | YES | GAG->CAG(E->Q) | E5: chr17: 70297933-70298030 |
| 161 | TMEM104->CDK12 | | NM_017728->NM_016507 | YES | GCA->CCA(A->P) | E5: chr17: 70297933-70298030 |
| 162 | TMEM104->CDK12 | | NM_017728->NM_016507 | | | E5: chr17: 70297933-70298030 |
| 163 | TRPC4AP->MRPL45 | | NM_015638->NM_032351 | YES | | E2: chr20: 33129509-33129638 |
| 164 | TRPC4AP->MRPL45 | | NM_199368->NM_032351 | YES | | E2: chr20: 33129509-33129638 |
| 165 | UBR5->SLC25A32 | | NM_015902->NM_030780 | | | E1: chr8: 103493576-103493671 |
| 166 | WIPF2->ERBB2 | | NM_133264->NM_001005862 | YES | | E1: chr17: 35629099-35629270 |
| 167 | WIPF2->ERBB2 | | NM_133264->NM_001005862 | YES | | E1: chr17: 35629099-35629270 |
| 168 | WIPF2->ERBB2 | | NM_133264->NM_004448 | | | E1: chr17: 35629099-35629270 |
| 169 | ZMYND8->CEP250 | | NM_012408->NM_007186 | | | E19: chr20: 45286376-45286616 |
| 170 | ZMYND8->CEP250 | | NM_012408->NM_007186 | | | E19: chr20: 45286376-45286616 |
| 170 | ZMYND8->CEP250 | | NM_183047->NM_007186 | | | E19: chr20: 45286376-45286616 |
| 172 | ZMYND8->CEP250 | | NM_183047->NM_007186 | | | E19: chr20: 45286376-45286616 |
| 173 | ZMYND8->CEP250 | | NM_183048->NM_007186 | | | E19: chr20: 45286376-45286616 |
| 174 | ZMYND8->CEP250 | | NM_183048->NM_007186 | | | E19: chr20: 45286376-45286616 |

| | | Fusion Transcript | |
|---|---|---|---|
| | | Coding Sequence | |
| # | Boundary Exon 3′ Gene | (SEQ ID:) | Fusion Protein Sequence (SEQ ID NO: or GenBank Accession No.) |
| 1 | E2: chr17: 34627645-34627952 | 61 | 235 |
| 2 | E2: chr17: 34627645-34627952 | 62 | 236 |
| 3 | E2: chr17: 34627645-34627952 | 63 | 237 |
| 4 | E2: chr17: 34627645-34627952 | 64 | 238 |
| 5 | E2: chr17: 34627645-34627952 | 65 | Identical Fusion Product as in NM_198836->NM_198993 |
| 6 | E8: chr5: 128391936-128392034 | 66 | 239 |
| 7 | E9: chr5: 128391936-128392034 | 67 | Identical Fusion Product as in NM_133638->NM_001017372 |
| 8 | E3: chr20: 45798853-45799093 | 68 | 240 |
| 9 | E3: chr20: 45798853-45799093 | 60 | Identical Fusion Product as in NM_006420->NM_001161841 |
| 10 | E3: chr20: 45798853-45799093 | 70 | 241 |
| 11 | E2: chr1: 46062691-46062839 | 71 | 242 |
| 12 | E2: chr1: 46062691-46062839 | 72 | Identical Fusion Product as in NM_006015->NM_015112 |
| 13 | E4: chr1: 27481316-27481363 | 73 | 243 |
| 14 | E4: chr1: 27481316-27481363 | 74 | Identical Fusion Product as in NM_006015->NM_015023 |
| 15 | E4: chr17: 39789323-39789482 | 75 | 244 |
| 16 | E4: chr17: 39789323-39789482 | 76 | Identical Fusion Product as in NM_001098833->NM_198475 |
| 17 | E24: chr17: 56800469-56800637 | 77 | 245 |
| 18 | E23: chr17: 56800469-56800637 | 78 | Identical Fusion Product as in NM_001010974->NM_001099432 |
| 19 | E24: chr17: 56800469-56800637 | 79 | Identical Fusion Product as in NM_001010974->NM_001099432 |
| 20 | E23: chr17: 56800469-56800637 | 80 | Identical Fusion Product as in NM_001010974->NM_001099432 |
| 21 | E24: chr17: 56800469-56800637 | 81 | Identical Fusion Product as in NM_001010974->NM_001099432 |
| 22 | E23: chr17: 56800469-56800637 | 82 | Identical Fusion Product as in NM_001010974->NM_001099432 |
| 23 | E2: chr2: 26974867-26975132 | 83 | 246 |
| 24 | E2: chr2: 26974867-26975132 | 84 | Identical Fusion Product as in NM_004899->NM_020134 |
| 25 | E2: chr2: 26974867-26975132 | 85 | Identical Fusion Product as in NM_004899->NM_020134 |
| 26 | E2: chr2: 26974867-26975132 | 86 | Identical Fusion Product as in NM_004899->NM_020134 |
| 27 | E2: chr2: 26974867-26975132 | 87 | Identical Fusion Product as in NM_004899->NM_020134 |
| 28 | E10: chr17: 55249854-55249916 | 88 | 247 |
| 29 | E4: chr11: 630400-630703 | 89 | 248 |
| 30 | E4: chr11: 630400-630703 | 90 | Identical Fusion Product as in NM_001039490->NM_000797 |
| 31 | E4: chr11: 630400-630703 | 91 | Identical Fusion Product as in NM_001039490->NM_000797 |
| 32 | E4: chr11: 630400-630703 | 92 | Identical Fusion Product as in NM_001039490->NM_000797 |
| 33 | E9: chr14: 38675394-38675928 | 93 | 249 |
| 34 | E9: chr14: 38675394-38675928 | 94 | Identical Fusion Product as in NM_005930->NM_001009182 |
| 35 | E10: chr14: 38675394-38675928 | 95 | Identical Fusion Product as in NM_005930->NM_001009182 |
| 36 | E9: chr14: 38675394-38675928 | 96 | 250 |
| 37 | E9: chr14: 38675394-38675928 | 97 | Identical Fusion Product as in NM_203354->NM_001009182 |
| 38 | E10: chr14: 38675394-38675928 | 98 | Identical Fusion Product as in NM_203354->NM_001009182 |
| 39 | E9: chr14: 38675394-38675928 | 99 | 251 |
| 40 | E9: chr14: 38675394-38675928 | 100 | Identical Fusion Product as in NM_203355->NM_001009182 |
| 41 | E10: chr14: 38675394-38675928 | 101 | Identical Fusion Product as in NM_203355->NM_001009182 |
| 42 | E9: chr14: 38675394-38675928 | 102 | 252 |
| 43 | E9: chr14: 38675394-38675928 | 103 | Identical Fusion Product as in NM_203356->NM_001009182 |
| 44 | E10: chr14: 38675394-38675928 | 104 | Identical Fusion Product as in NM_203356->NM_001009182 |
| 45 | E3: chr17: 71852346-71852413 | 105 | 253 |
| 46 | E3: chr17: 71852346-71852413 | 106 | Identical Fusion Product as in NM_004762->NM_002766 |
| 47 | E3: chr6: 46715189-46715364 | 107 | 254 |
| 48 | E2: chr17: 74217326-74217409 | 108 | 255 |
| 49 | E2: chr17: 74217326-74217409 | 109 | 256 |
| 50 | E2: chr17: 74217326-74217409 | 110 | Identical Fusion Product as in NM_001013839->NM_004762 |
| 51 | E2: chr17: 74217326-74217409 | 111 | Identical Fusion Product as in NM_001013839->NM_017456 |
| 52 | E2: chr17: 74217326-74217409 | 112 | Identical Fusion Product as in NM_001013839->NM_004762 |
| 53 | E2: chr17: 74217326-74217409 | 113 | Identical Fusion Product as in NM_001013839->NM_017456 |
| 54 | E2: chr17: 74217326-74217409 | 114 | Identical Fusion Product as in NM_001013839->NM_004762 |
| 55 | E2: chr17: 74217326-74217409 | 115 | Identical Fusion Product as in NM_001013839->NM_017456 |
| 56 | E2: chr17: 74217326-74217409 | 116 | Identical Fusion Product as in NM_001013839->NM_004762 |
| 57 | E2: chr17: 74217326-74217409 | 117 | Identical Fusion Product as in NM_001013839->NM_017456 |
| 58 | E2: chr17: 74217326-74217409 | 118 | the entire EXOC7 protein |
| 59 | E2: chr17: 74217326-74217409 | 119 | the entire EXOC7 protein |
| 60 | E4: chr9: 129989962-129990034 | 120 | 257 |
| 61 | E5: chr9: 129987646-129987876 | 121 | 258 |
| 62 | E4: chr9: 129989962-129990034 | 122 | 259 |
| 63 | E5: chr9: 129987646-129987876 | 123 | 260 |
| 64 | E4: chr9: 129989962-129990034 | 124 | 261 |
| 65 | E5: chr9: 129987646-129987876 | 125 | 262 |
| 66 | E17: chr9: 129989962-129990034 | 126 | 263 |
| 67 | E4: chr9: 129987646-129987876 | 127 | Identical Fusion Product as in NM_001035254->NM_001131015 |
| 68 | E4: chr9: 129989962-129990034 | 128 | Identical Fusion Product as in NM_001035254->NM_001131016 |
| 69 | E5: chr9: 129987646-129987876 | 129 | Identical Fusion Product as in NM_001035254->NM_001131016 |
| 70 | E12: chr12: 119269631-119269700 | 130 | 264 |
| 71 | E2: chr3: 32458335-32458509 | 131 | 265 |
| 72 | E2: chr3: 32458335-32458509 | 132 | 266 |
| 73 | E2: chr3: 32458335-32458509 | 133 | 267 |
| 74 | E2: chr3: 32458335-32458509 | 134 | 268 |
| 75 | E2: chr3: 32458335-32458509 | 135 | 269 |
| 76 | E2: chr3: 32458335-32458509 | 136 | 270 |
| 77 | E9: chr8: 129182407-129182681 | 137 | 271 |
| 78 | E2: chr1: 150225198-150225351 | 138 | 272 |
| 79 | E2: chr7: 717491-717690 | 139 | 273 |
| 80 | 140 | | Identical Fusion Product as in NM_001080453->NM_001164758 |
| 81 | 141 | | Identical Fusion Product as in NM_001080453->NM_001164758 |
| 82 | 142 | | Identical Fusion Product as in NM_001080453->NM_001164758 |
| 83 | 143 | | Identical Fusion Product as in NM_001080453->NM_001164758 |
| 84 | 144 | | Identical Fusion Product as in NM_001080453->NM_001164758 |
| 85 | 145 | 274 | |
| 86 | 146 | 275 | |
| 87 | 147 | | Identical Fusion Product as in NM_174902->NM_001145541 |
| 88 | 148 | 276 | |
| 89 | 149 | | Identical Fusion Product as in NM_001113546->NM_015276 |
| 90 | 150 | 277 | |
| 91 | 151 | 278 | |
| 92 | 152 | 279 | |
| 93 | 153 | 280 | |
| 94 | 154 | | Identical Fusion Product as in NM_018682->NM_199000 |
| 95 | 155 | 281 | |
| 96 | 156 | 282 | |
| 97 | 157 | 283 | |
| 98 | 158 | 284 | |
| 99 | 159 | | Identical Fusion Product as in NM_001134673->NM_012153 |
| 100 | 160 | 285 | |
| 101 | 161 | 286 | |
| 102 | 162 | 287 | |
| 103 | 163 | 288 | |
| 104 | 164 | 289 | |
| 105 | E2: chr8: 32572887-32573065 | 165 | 290 |
| 106 | E2: chr8: 32572887-32573065 | 166 | 291 |
| 107 | E2: chr8: 32572887-32573065 | 167 | 292 |
| 108 | E2: chr8: 32572887-32573065 | 168 | 293 |
| 109 | E2: chr8: 32572887-32573065 | 169 | 294 |
| 110 | E2: chr8: 32572887-32573065 | 170 | 295 |
| 111 | E2: chr8: 32572887-32573065 | 171 | 296 |
| 112 | E5: chr8: 119270875-119279152 | 172 | 297 |
| 113 | E5: chr8: 119270875-119279152 | 173 | Identical Fusion Product as in NM_016018->NM_001101676 |
| 114 | E5: chr8: 119270875-119279152 | 174 | 298 |
| 115 | E7: chr17: 54156399-54156460 | 175 | 299 |
| 116 | E17: chr17: 57148093-57148206 | 176 | 300 |
| 117 | E8: chr1: 149922455-149922545 | 177 | 301 |
| 118 | E8: chr1: 149922455-149922545 | 178 | Identical Fusion Product as in NM_001167857->NM_030918 |
| 119 | E8: chr1: 149922455-149922545 | 179 | Identical Fusion Product as in NM_001167857->NM_030918 |
| 120 | E3: chr1: 204632668-204632884 | 180 | 302 |
| 121 | E3: chr1: 204632668-204632884 | 181 | Identical Fusion Product as in NM_004698->NM_001042758 |
| 122 | E3: chr1: 204632668-204632884 | 182 | Identical Fusion Product as in NM_004698->NM_001042758 |
| 123 | E5: chr6: 42343869-42345564 | 183 | 303 |
| 124 | E5: chr6: 42343869-42345564 | 184 | Identical Fusion Product as in NM_001020658->NM_033502 |
| 125 | E3: chr19: 17117206-17117301 | 185 | 304 |
| 126 | E3: chr19: 17117206-17117301 | 186 | Identical Fusion Product as in NM_020673->NM_001130065 |
| 127 | E16: chr3: 199076690-199076739 | 187 | 305 |
| 128 | E9: chr16: 21890346-21890442 | 188 | 306 |
| 129 | E9: chr8: 104144358-104144451 | 189 | 307 |
| 130 | E77: chr1: 226605164-226605267 | 190 | 308 |
| 131 | E77: chr1: 226605164-226605267 | 191 | Identical Fusion Product as in NM_001010858-> |
| | | | NM_001098623 |
| 132 | E28: chr13: 59137723-59138981 | 192 | 309 |
| 133 | E2: chr17: 44376285-44376336 | 193 | 310 |
| 134 | E27: chr1: 120266781-120266924 | 194 | 311 |
| 135 | E5: chr21: 42570073-42570124 | 195 | 312 |
| 136 | E5: chr21: 42570073-42570124 | 196 | 313 |
| 137 | E5: chr21: 42570073-42570124 | 197 | Identical Fusion Product as in NM_018964->NM_016818 |
| 138 | E6: chr21: 42570073-42570124 | 198 | Identical Fusion Product as in NM_018964->NM_016818 |
| 139 | E7: chr21: 42570073-42570124 | 199 | Identical Fusion Product as in NM_018964->NM_016818 |
| 140 | E5: chr21: 42570073-42570124 | 200 | Identical Fusion Product as in NM_018964->NM_016818 |
| 141 | E15: chr1: 148588256-148588318 | 201 | 314 |
| 142 | E15: chr1: 148588256-148588318 | 202 | Identical Fusion Product as in NM_001042758->NM_004698 |
| 143 | E15: chr1: 148588256-148588318 | 203 | Identical Fusion Product as in NM_001042758->NM_004698 |
| 144 | E7: chr20: 52693403-52693524 | 204 | 315 |
| 145 | E7: chr20: 52693403-52693524 | 205 | Identical Fusion Product as in NM_001165937->NM_018431 |
| 146 | E7: chr20: 52693403-52693524 | 206 | Identical Fusion Product as in NM_001165937->NM_018431 |
| 147 | E11: chr2: 202870346-202870481 | 207 | 316 |
| 148 | E8: chr3: 64054566-64060641 | 208 | 317 |
| 149 | E8: chr3: 64054566-64060641 | 209 | 318 |
| 150 | E8: chr3: 64054566-64060641 | 210 | Identical Fusion Product as in NM_001161841->NM_198859 |
| 151 | E4: chr17: 61115708-61115798 | 211 | 319 |
| 152 | E24: chr17: 61115708-61115798 | 212 | Identical Fusion Product as in NM_003168->NM_001037325 |
| 153 | E2: chr20: 32346861-32347052 | 213 | 320 |
| 154 | E2: chr20: 32346861-32347052 | 214 | 321 |
| 155 | E2: chr20: 32346861-32347052 | 215 | Identical Fusion Product as in NM_001079864->NM_000687 |
| 156 | E2: chr20: 32346861-32347052 | 216 | Identical Fusion Product as in NM_001079864->NM_001161766 |
| 157 | E2: chr10: 121609300-121609386 | 217 | 322 |
| 158 | E2: chr10: 121609300-121609386 | 218 | Identical Fusion Product as in NM_001033925->NM_024834 |
| 159 | E1: chr17: 34940382-34944326 | 219 | 323 |
| 160 | E14: chr17: 34940409-34944326 | 220 | 324 |
| 161 | E14: chr17: 34940382-34944326 | 221 | 325 |
| 162 | E1: chr17: 34940409-34944326 | 222 | Identical Fusion Product as in NM_017728->NM_015083 |
| 163 | E7: chr17: 33731535-33731709 | 223 | 326 |
| 164 | E7: chr17: 33731535-33731709 | 224 | Identical Fusion Product as in NM_015638->NM_032351 |
| 165 | E2: chr8: 104489037-104489188 | 225 | 327 |
| 166 | E4: chr17: 35104766-35104960 | 226 | 328 |
| 167 | E5: chr17: 35116768-35116920 | 227 | Identical Fusion Product as in NM_133264->NM_001005862 |
| 168 | E2: chr17: 35116768-35116920 | 228 | 329 |
| 169 | E21: chr20: 33541876-33542044 | 229 | 330 |
| 170 | E22: chr20: 33542451-33542586 | 230 | Identical Fusion Product as in NM_012408->NM_007186 |
| 171 | E21: chr20: 33541876-33542044 | 231 | Identical Fusion Product as in NM_012408->NM_007186 |
| 172 | E22: chr20: 33542451-33542586 | 232 | Identical Fusion Product as in NM_012408->NM_007186 |
| 173 | E21: chr20: 33541876-33542044 | 233 | 331 |
| 174 | E22: chr20: 33542451-33542586 | 234 | 332 |

### Fusion Genes Identified in MCF7 Cancer Cell Line

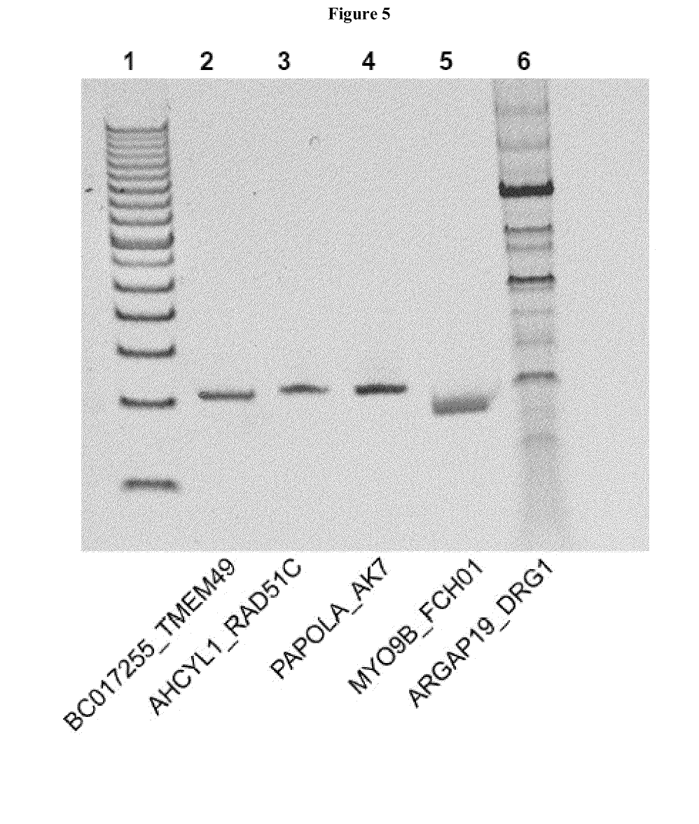

Fusion gene products in the MCF7 cell line had been previously described using a paired end sequencing protocol. The list of fusion transcripts identified in MCF7 cancer cell line using SnowShoes-FTD as described herein was compared to the list of transcripts described elsewhere (Maher et al., Proc. Natl. Acad. Sci. USA, 106(30):12353-8 (2009)). The SnowShoes-FTD identified and validated 5 novel fusion transcripts that were not reported by Maher et al.: ADAMTS19-SLC27A6, ATXN7L3-FAM171A2, GCN1L1-MSI1, MYH9-EIF3D, and RPS6KB1-DIAPH3. In addition, there were 5 fusion genes identified by Maher et al. that were not detected by SnowShoes-FTD: ARHGAP19-DRG1, BC017255-TMEM49, PAPOLA-AK7, AHCYL1-RAD51C, and FCHOL-MYO9B. It was found that (i) BC017255 was no longer in the RefSeq RNA database and (ii) the distance between PAPOLA-AK7 is 65 Kb which is smaller than the default setting of 100 Kb. In addition, no fusion junction spanning reads were observed to support this fusion. Therefore, this fusion transcript would only have been detected with a different distance threshold and by reducing the default for fusion spanning reads to 0. (iii) There are no junction spanning reads in the data set for AHCYL1-RAD51C although 10 fusion encompassing reads supporting the existence of this fusion transcript were found. (iv) There was only one fusion junction spanning read for FCHOL-MYO9B, and the default setting for SnowShoes-FTD was “at least two unique junction spanning reads.” On the other hand, no evidence was found in support of an ARHGAP19-DRG1 fusion, as the alignment file (SAM file) did not contain any read pairs that mapped to both of these genes. When RT-PCR was performed using the PCR primers provided by Maher et al. (FIG. 5), the results also supported the existence of the fusion products BC017255-TMEM49, PAPOLA-AK7, AHCYL1-RAD51C, and FCHOL-MYO9B, while no PCR product was observed for the ARHGAP19-DRG1 fusion. Thus, 4 out of 5 “known” fusion transcripts that were not identified by SnowShoes-FTD were explained by differences in the RefSeq database used for the analyses or by the choice of parameter settings for the various filtering steps. The ARHGAP19-DRG1 fusion transcript reported by Maher et al. did not appear to be expressed in the MCF7 cells that were obtained from ATCC and used in this study.

Edgren et al. (Genome Biol., 12(1):R6 (2011)) reported on detection of fusion transcripts in four breast cancer cell lines, including MCF7 in which three fusion transcripts were validated. The work described herein detected eight fusion transcripts in MCF, including two of the three reported by Edgren et al. (BCAS4_BCAS3 and ARFGEF2_SULF2).

### Pathway Analysis of Genes Involved in Fusion Transcripts in Breast Cancer Cell Lines

There were a total of 105 fusion partner genes from the 55 fusion candidates, among which 58 genes formed in-frame fusion transcripts of 30 chimeric RNAs. Pathway and regulatory network analyses of these 58 genes were performed using MetaCore (GeneGo Inc., San Diego, Calif.). There were two pathways that are enriched among these 58 genes: the non-genomic action of androgen receptor and ligand-independent activation of ESR1 and ESR2. Three GeneGo process networks were significantly enriched: androgen receptor signaling cross-talk, ESR1-nuclear pathway, and FGF/ERBB signaling. This observation suggests that fusion transcripts may have functional significance in signal transduction in breast cancer cells.

Structural Analysis of Fusion Transcripts Suggests a Preponderance of ‘Promoter Swap’ Mutations, One of which May Represent a Novel Mechanism for ERBB2 Overexpression

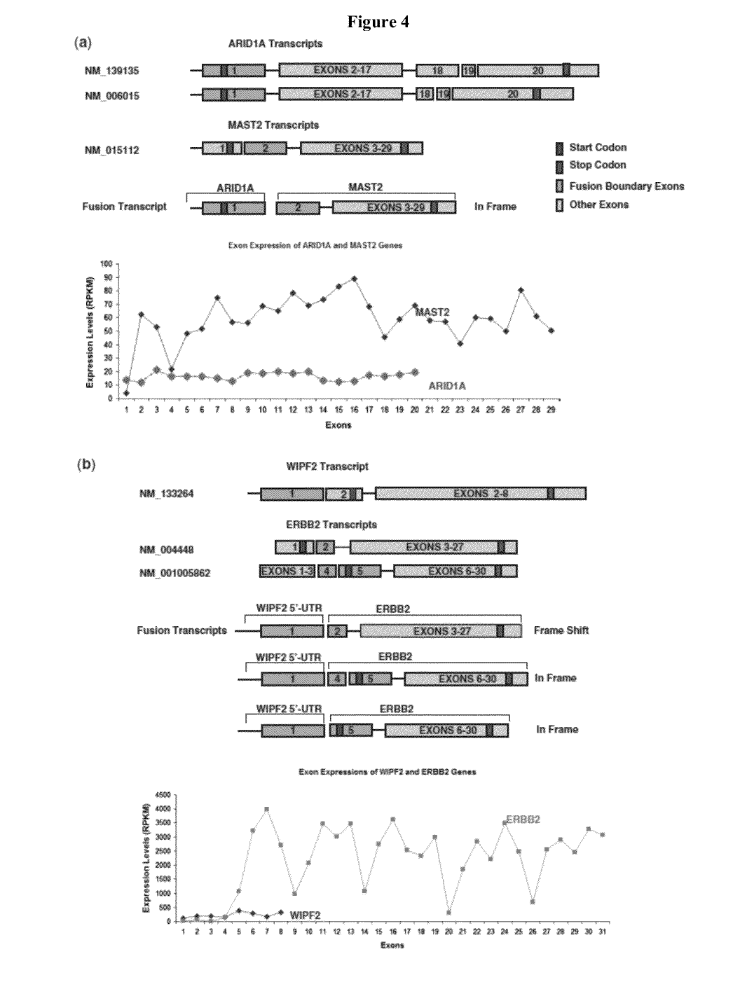

The analytical power of the SnowShoes-FTD pipeline lies in part in the very low false detection rate and in very large part in the downstream features that predict the structure of the hypothetical fusion transcripts and the amino acid sequence of the resultant translation products. Such analyses indicated that the nature of the fusion transcripts that were detected in breast cancer cells is strikingly non-random, as evidenced by the fact that 23 of the 60 confirmed chimeric transcripts result from fusion of exon 1 of the 5′/upstream partners to the 3′/downstream partners. The most probable cause of such chimeric RNAs is a genomic rearrangement that results in juxtaposition of a promoter that potentially alters the level of expression and/or the regulation of the downstream partner in response to changes in the cellular environment. In addition, all of the fusion transcripts that were reported and validated herein map precisely to exon/exon junctions between the upstream and downstream fusion partners, suggesting that such transcripts are processed. There were only five additional fusion transcripts in which the fusion junction points were in the middle of exons (detected with different parameter settings for SnowShoes-FTD). About half of the fusion events were in frame and therefore predicted to encode fusion proteins. The preponderance of such events in these samples suggests that some of the fusion transcripts may convey a growth advantage, such that transcript enrichment results from selection. For example, MDA-MB-468 cells express an ARID 1A_MAST2 fusion transcript (FIG. 4A) that might result from translocation without inversion of the ARID1A promoter (1p36.11) to the more centromeric MAST2 locus (1p34.1). Alternatively, this fusion transcript might result from interstitial deletion of those portions of chromosome 1 that intervene between exon 1 of ARID1A (coordinates 26896618) and exon 2 of MAST2 (coordinates 46062691). Juxtaposition of the ARID1A promoter would place control of MAST2, which is downstream of the RB1 pathway, as evidenced by the preponderance of E2F sites in the ARID1A promoter and by the observation that ARID1A is regulated in a cell cycle-dependent manner (Nagl et al., Embo J., 26(3):752-63 (2007)). Using SnowShoes-FTD, it was predicted that in-frame fusion between ARID1A exon 1 and MAST2 exon 3 will give rise to a chimeric transcript with a predicted open reading frame of 2118 amino acids. The N-terminal 378 amino acids of this hypothetical fusion protein were derived from ARID1A and appeared to contain no known or predicted functional domain. Conversely, the C-terminal 1740 amino acids were derived from MAST2 and contained the protein kinase, AGC kinase, and PDZ domains of the parental protein. It was likely that this fusion protein has serine/threonine kinase activity. Whether loss of the N-terminal 58 amino acids from MAST2, insertion of the 378 amino acid N-terminus of ARID1A, or aberrant expression of MAST2 driven from the ARID1A promoter conveyed novel oncogenic potential remains to be determined.

The level of exon expression of the fusion transcript was examined. As shown in FIG. 4A, exon 1 expression of MAST2 was significantly lower than the other exons (exon 2-29), which might be due to the fact the exon 1 was fused out. However, there were no obvious expression differences between the exons of the ARID1A gene. The most provocative chimeric transcript that was detected involves fusion of the WIPF2 and ERBB2 RNAs. Two isoforms of the fusion were predicted and validated. These chimeric transcripts were expressed in UACC812 cells, which were derived from a HER2+ tumor (Meltzer et al., Br. J. Cancer, 63(5):727-35 (1991)). The WIPF2 locus (also known as WIRE) is located at chr17q21.2 and is transcribed towards the telomere. ERBB2 is located at chr17q11.2, centromeric to WIPF2. Like WIPF2, ERBB2 is transcribed towards the telomere. It was therefore probable that this fusion transcript arose as a result of translocation without inversion of the WIPF2 promoter to give rise to two in-frame transcripts in which the 5′ untranslated region of WIPF2 is fused to one of several 5′ untranslated exons of ERBB2 (FIG. 4B). The genomic structure of this hypothetical translocation remains to be verified, but the net result of such an event would be to place ERBB2 expression under control of a promoter that appears, from analysis of potential transcription factor binding sites in the WIPF2 5′ flanking region, to be susceptible to regulation by NFκB, NOTCH, and MYC signaling. This hypothetical promoter swap may account, at least in part, for the observation that ERBB2 transcripts account for about 12,632 tags per million total tags, as determined from the mRNA-Seq data, which translates to about 1.3% of the total polyA+ mRNA pool in UACC812 cells. The observation that there was a dramatic increase in ERBB2 exon expression at the fusion junction (FIG. 4B) is consistent with this hypothesis.

SnowShoes-FTD Predicted Two WIPF2 ERBB2 Fusion Junctions which were Verified in UACC812 Cells

WIPF2 chromosomal coordinates 35629270 fused to ERBB2 coordinates 35104766 or 35116768. The latter coordinates fell within the coding sequence of one of the RefSeq variants of ERBB2 mRNA (exon 2 of NM_{—}004448) and would introduce a frame shift mutation in that variant (FIG. 4B). However, two of the three predicted fusion sequences (comprised of exon 1 of WIPF2 NM_{—}133264 fused to exon 4 or 5 of ERBB2 NM_{—}001005862) would produce transcripts that encode full length ERBB2 protein (FIG. 4B). The sequence of full length transcripts from mRNA-Seq data was not determined at this time. It might be necessary to clone and sequence longer cDNA fragments that correspond to the first few hundred nucleotides of the fusion transcript in order to determine which of the hypothetical transcripts are expressed. When the exon expression levels of ERBB2 were examined, exons 1-4 were found to be substantially less abundant than downstream exons, suggesting that the transcript with the first 4 exons of ERBB2 fused out might be the more plausible fusion product. These results may indicate that a novel mechanism accounts for ERBB2 overexpression in HER2+ breast cancer.

### Example 2

### Detection of Redundant Fusion Transcripts in Primary Breast Tumors

### Paired-End RNA-Seq Analysis

Total RNA was prepared from 8 each fresh frozen estrogen receptor positive (ER+), ERBB2 enriched (HER2+), and triple negative (TN) breast tumors. Tumors were macrodissected to remove normal tissue. RNA quality was determined using an Agilent Bioanalyzer (RIN>7.9 for all samples), and cDNA libraries were prepared and sequenced (50 nt paired-end) on the Illumina GAIIx, as described elsewhere (Sun et al., PLoS ONE, 6:e17490 (2011)) to a depth of 20-50 M end pairs per sample (Table 7). The quantity of the fusion transcripts were calculated as the number of fusion encompassing reads per million aligned reads. Normal tissue mRNA-Seq data (50-base paired-end, 73-80 million read pairs per sample) from the Body Map 2.0 project were obtained from ArrayExpress (http://www.ebi.ac.uk/arrayexpress, query ID: E-MTAB-513). Paired-end sequence data from non-transformed human mammary epithelial cells (Asmann et al. Nucleic Acids Res., 39(15):e100 (2011)) were re-analyzed as described herein.

TABLE 7
Alignment summaries for individual tumors.

| | | | | One | | | |
|---|---|---|---|---|---|---|---|
| | | Both | | End Mapped | One End | One End | |
| | | Ends | | to Junction, the | Mapped to | Mapped to | |
| | Total | Mapped | Both Ends | Other End | Genome, the | Junction, the | |
| Tumor | Number of | to Exon | Mapped to | Mapped to | Other End Not | Other End | |
| ID | Read Pairs | Junctions | Genome | Genome | Mapped | Not Mapped | Sample Description |
| s_25 | 19,633,880 | 782,526 | 11,349,512 | 2,298,248 | 1,098,701 | 162,327 | HER2+ Breast Tumor |
| s_26 | 19,510,963 | 742,955 | 11,691,267 | 2,660,440 | 830,003 | 134,309 | HER2+ Breast Tumor |
| s_27 | 19,965,809 | 862,416 | 11,681,914 | 3,022,891 | 958,518 | 167,122 | HER2+ Breast Tumor |
| s_28 | 19,326,720 | 729,475 | 11,350,258 | 2,709,067 | 942,473 | 146,291 | HER2+ Breast Tumor |
| s_29 | 19,287,844 | 668,668 | 10,136,081 | 2,644,347 | 1,427,405 | 181,342 | HER2+ Breast Tumor |
| s_30 | 19,872,605 | 806,772 | 11,013,118 | 2,943,293 | 1,426,803 | 249,954 | HER2+ Breast Tumor |
| s_31 | 17,399,880 | 662,680 | 9,975,682 | 2,409,195 | 811,389 | 122,519 | HER2+ Breast Tumor |
| s_32 | 19,167,067 | 804,355 | 10,062,209 | 2,874,016 | 1,040,725 | 179,568 | HER2+ Breast Tumor |
| s_33 | 52,989,065 | 1,442,285 | 32,211,986 | 6,370,244 | 2,926,974 | 384,138 | ER+ Breast Tumor |
| s_34 | 47,666,820 | 1,481,381 | 28,330,271 | 6,340,808 | 3,099,048 | 455,741 | ER+ Breast Tumor |
| s_35 | 49,814,344 | 1,598,163 | 27,010,487 | 6,074,822 | 5,687,392 | 859,744 | ER+ Breast Tumor |
| s_36 | 50,734,654 | 1,349,033 | 23,322,513 | 5,046,612 | 8,806,497 | 1,335,350 | ER+ Breast Tumor |
| s_37 | 52,954,073 | 1,887,348 | 27,674,967 | 6,846,678 | 5,605,759 | 977,738 | ER+ Breast Tumor |
| s_38 | 51,724,496 | 2,235,084 | 27,914,085 | 8,148,819 | 2,675,758 | 465,731 | ER+ Breast Tumor |
| s_39 | 51,548,133 | 1,833,333 | 28,399,341 | 6,920,007 | 2,742,863 | 435,097 | ER+ Breast Tumor |
| s_40 | 44,112,005 | 1,916,730 | 25,100,264 | 6,332,273 | 2,451,822 | 418,968 | ER+ Breast Tumor |
| s_41 | 21,550,821 | 1,060,261 | 11,731,299 | 4,208,639 | 749,366 | 152,413 | Normal Breast Primary |
| | | | | | | | Culture |
| s_42 | 21,353,151 | 1,094,923 | 11,523,049 | 3,743,587 | 943,898 | 157,786 | Normal Breast Primary |
| | | | | | | | Culture |
| s_43 | 20,924,924 | 1,045,589 | 11,120,605 | 4,111,792 | 771,947 | 152,238 | Normal Breast Primary |
| | | | | | | | Culture |
| s_44 | 22,510,790 | 1,149,387 | 12,209,115 | 4,544,155 | 678,941 | 143,978 | Normal Breast Primary |
| | | | | | | | Culture |
| s_45 | 21,057,269 | 958,317 | 11,470,882 | 3,815,264 | 735,812 | 139,739 | Normal Breast Primary |
| | | | | | | | Culture |
| s_46 | 24,033,748 | 1,146,880 | 13,264,678 | 4,594,952 | 887,587 | 169,999 | Normal Breast Primary |
| | | | | | | | Culture |
| s_47 | 21,682,601 | 1,083,301 | 11,919,091 | 3,769,336 | 907,219 | 178,754 | Normal Breast Primary |
| | | | | | | | Culture |
| s_48 | 20,257,198 | 945,339 | 11,137,380 | 3,802,035 | 754,667 | 143,117 | Normal Breast Primary |
| | | | | | | | Culture |
| s_49 | 27,742,773 | 1,194,950 | 14,219,774 | 3,643,071 | 1,802,090 | 281,820 | Triple Negative Breast |
| | | | | | | | Tumor |
| s_50 | 26,038,478 | 922,741 | 15,502,762 | 3,686,465 | 1,091,731 | 155,837 | Triple Negative Breast |
| | | | | | | | Tumor |
| s_51 | 25,538,716 | 1,110,680 | 13,090,688 | 4,133,936 | 1,939,284 | 357,700 | Triple Negative Breast |
| | | | | | | | Tumor |
| s_52 | 22,224,358 | 773,848 | 12,782,913 | 3,121,694 | 937,044 | 139,107 | Triple Negative Breast |
| | | | | | | | Tumor |
| s_53 | 21,271,234 | 1,123,178 | 10,145,277 | 4,547,699 | 1,580,560 | 343,052 | Triple Negative Breast |
| | | | | | | | Tumor |
| s_54 | 25,238,796 | 724,527 | 12,992,429 | 2,910,508 | 2,842,085 | 329,963 | Triple Negative Breast |
| | | | | | | | Tumor |
| s_55 | 22,588,795 | 733,892 | 12,319,173 | 3,006,438 | 1,913,594 | 263,909 | Triple Negative Breast |
| | | | | | | | Tumor |
| s_56 | 28,685,711 | 966,103 | 15,650,142 | 3,271,160 | 1,834,194 | 228,476 | Triple Negative Breast |
| | | | | | | | Tumor |
| s_25 | 100.00% | 3.99% | 57.81% | 11.71% | 5.60% | 0.83% | HER2+ Breast Tumor |
| s_26 | 100.00% | 3.81% | 59.92% | 13.64% | 4.25% | 0.69% | HER2+ Breast Tumor |
| s_27 | 100.00% | 4.32% | 58.51% | 15.14% | 4.80% | 0.84% | HER2+ Breast Tumor |
| s_28 | 100.00% | 3.77% | 58.73% | 14.02% | 4.88% | 0.76% | HER2+ Breast Tumor |
| s_29 | 100.00% | 3.47% | 52.55% | 13.71% | 7.40% | 0.94% | HER2+ Breast Tumor |
| s_30 | 100.00% | 4.06% | 55.42% | 14.81% | 7.18% | 1.26% | HER2+ Breast Tumor |
| s_31 | 100.00% | 3.81% | 57.33% | 13.85% | 4.66% | 0.70% | HER2+ Breast Tumor |
| s_32 | 100.00% | 4.20% | 52.50% | 14.99% | 5.43% | 0.94% | HER2+ Breast Tumor |
| s_33 | 100.00% | 2.72% | 60.79% | 12.02% | 5.52% | 0.72% | ER+ Breast Tumor |
| s_34 | 100.00% | 3.11% | 59.43% | 13.30% | 6.50% | 0.96% | ER+ Breast Tumor |
| s_35 | 100.00% | 3.21% | 54.22% | 12.19% | 11.42% | 1.73% | ER+ Breast Tumor |
| s_36 | 100.00% | 2.66% | 45.97% | 9.95% | 17.36% | 2.63% | ER+ Breast Tumor |
| s_37 | 100.00% | 3.56% | 52.26% | 12.93% | 10.59% | 1.85% | ER+ Breast Tumor |
| s_38 | 100.00% | 4.32% | 53.97% | 15.75% | 5.17% | 0.90% | ER+ Breast Tumor |
| s_39 | 100.00% | 3.56% | 55.09% | 13.42% | 5.32% | 0.84% | ER+ Breast Tumor |
| s_40 | 100.00% | 4.35% | 56.90% | 14.35% | 5.56% | 0.95% | ER+ Breast Tumor |
| s_41 | 100.00% | 4.92% | 54.44% | 19.53% | 3.48% | 0.71% | Normal Breast Primary |
| | | | | | | | Culture |
| s_42 | 100.00% | 5.13% | 53.96% | 17.53% | 4.42% | 0.74% | Normal Breast Primary |
| | | | | | | | Culture |
| s_43 | 100.00% | 5.00% | 53.15% | 19.65% | 3.69% | 0.73% | Normal Breast Primary |
| | | | | | | | Culture |
| s_44 | 100.00% | 5.11% | 54.24% | 20.19% | 3.02% | 0.64% | Normal Breast Primary |
| | | | | | | | Culture |
| s_45 | 100.00% | 4.55% | 54.47% | 18.12% | 3.49% | 0.66% | Normal Breast Primary |
| | | | | | | | Culture |
| s_46 | 100.00% | 4.77% | 55.19% | 19.12% | 3.69% | 0.71% | Normal Breast Primary |
| | | | | | | | Culture |
| s_47 | 100.00% | 5.00% | 54.97% | 17.38% | 4.18% | 0.82% | Normal Breast Primary |
| | | | | | | | Culture |
| s_48 | 100.00% | 4.67% | 54.98% | 18.77% | 3.73% | 0.71% | Normal Breast Primary |
| | | | | | | | Culture |
| s_49 | 100.00% | 4.31% | 51.26% | 13.13% | 6.50% | 1.02% | Triple Negative Breast |
| | | | | | | | Tumor |
| s_50 | 100.00% | 3.54% | 59.54% | 14.16% | 4.19% | 0.60% | Triple Negative Breast |
| | | | | | | | Tumor |
| s_51 | 100.00% | 4.35% | 51.26% | 16.19% | 7.59% | 1.40% | Triple Negative Breast |
| | | | | | | | Tumor |
| s_52 | 100.00% | 3.48% | 57.52% | 14.05% | 4.22% | 0.63% | Triple Negative Breast |
| | | | | | | | Tumor |
| s_53 | 100.00% | 5.28% | 47.69% | 21.38% | 7.43% | 1.61% | Triple Negative Breast |
| | | | | | | | Tumor |
| s_54 | 100.00% | 2.87% | 51.48% | 11.53% | 11.26% | 1.31% | Triple Negative Breast |
| | | | | | | | Tumor |
| s_55 | 100.00% | 3.25% | 54.54% | 13.31% | 8.47% | 1.17% | Triple Negative Breast |
| | | | | | | | Tumor |
| s_56 | 100.00% | 3.37% | 54.56% | 11.40% | 6.39% | 0.80% | Triple Negative Breast |
| | | | | | | | Tumor |

### Identification of Fusion Transcripts

End pairs were aligned to human genome build 36 using Burrows-Wheeler Aligner (BWA) (Li and Durbin, Bioinformatics, 25:1754-60 (2009)). The aligned SAM files were sorted according to read IDs using SAMtools (Li et al., Bioinformatics, 25:2078-9 (2009)). The fusion transcripts were identified using SnowShoes-FTD (Asmann et al. Nucleic Acids Res., 39(15):e100 (2011)) version 2.0, which has higher sensitivity without increasing false discovery rate, compared to version 1.0.

### Fusion Encompassing Versus Fusion Spanning Reads

Fusion encompassing reads (Maher et al., Proc. Natl. Acad. Sci. USA, 106(30):12353-8 (2009)) contained 50 nucleotides from each end which map to different fusion partners. Fusion spanning reads included one end that maps within one of the two fusion partners and a second end that spans the junction between the two different fusion partners. Sentinel fusion transcripts were defined as those detected in a single tumor with 3 or more unique, tiling fusion encompassing read pairs plus 2 or more unique, tiling fusion spanning reads. Moreover, alignment of these reads must allow unambiguous assignment of directionality (5′ to 3′) of the two fusion partners. The initial analysis of fusion transcripts in breast cancer cell lines indicated that sentinel transcripts are predicted with very high accuracy. See, Example 1. A select subset of sentinel transcripts from the breast tumors was validated.

### Private Versus Redundant Fusion Transcripts

A private fusion transcript was detected in only one tumor sample. All private transcripts, by definition, had sentinel properties. Redundant transcripts were detected in two or more tumors. A redundant transcript must exhibit sentinel properties in at least one tumor.

### Tumor-Specific Fusion Transcripts

Fusion transcripts in breast tumors were filtered to remove all candidates that were also detected in either one of the control datasets: the HMEC or Body Map data. This approach was based on the assumption that such candidates represent either annotation or alignment errors or arise from germ line rearrangement polymorphisms (Hillmer et al., Genome Res., 21:665-75 (2011)).

### Results

131 sentinel fusion transcripts were detected in 24 tumors (Table 8). The majority of the fusion transcripts arose from interchromosomal fusions (104/131). Six fusion transcripts were expressed as multiple isoforms in tumors (labeled with a “+” in Table 8). The majority of the fusion transcripts were ‘private’, expressed in only one tumor sample. However, 45 sentinel transcripts were redundant, as evidenced by detection in two or more tumors (labeled with a “$” in Table 8). Redundancy was dependent upon depth of sequence. Therefore, some of the private transcripts could emerge as redundant if greater depth of sequence were obtained.

TABLE 8
Fusion transcripts in primary breast cancers.

| | | | | | | | | | | # titling Junction | |
|---|---|---|---|---|---|---|---|---|---|---|---|
| | | | | | Potential | | | | # unique Titling | Reads in | |
| | | Fusion pair | FUSION GENE | | Fusion | | | | Junction Reads in all | current | |
| Row # | Sample | Alphabetical | directional | Type | Mechanism | Fusion Strand | Total pairs | Exon Boundary Fusion | Samples | sample | SEQ ID NO: |
| 1*/$ | s_56 | AATK_USP32 | AATK->USP32 | intra- | D OR T | − | 0.23902 | YES | 4 | 3 | 453 |
| | | | | chr | | | | | | | |
| 2$ | s_55 | AATK_USP32 | AATK->USP32 | intra- | D OR T | − | 0.11663 | YES | 4 | 1 | 454 |
| | | | | chr | | | | | | | |
| 3* | s_26 | ABCA10_TP53I13 | TP53I13-> | intra- | I AND (D | + | 0.16049 | YES | 2 | 2 | 455 |
| | | | ABCA10 | chr | OR T) | | | | | | |
| 4*/$ | s_53 | ABCA2_FLNA | FLNA->ABCA2 | inter- | T | − | 0.14901 | NO | 2 | 2 | 456 |
| | | | | chr | | | | | | | |
| 5$ | s_36 | ABCA2_FLNA | | | | | 0.01437 | | | | |
| 6* | s_36 | ABCC5_EIF4G1 | EIF4G1-> | intra- | I AND T | + | 0.08623 | NO | 3 | 3 | 457 |
| | | | ABCC5 | chr | | | | | | | |
| 7* | s_29 | ACACA_CALR | CALR-> | inter- | I AND T | + | 0.10524 | NO | 3 | 3 | 458 |
| | | | ACACA | chr | | | | | | | |
| 8*/$ | s_55 | ACTB_APOL1 | APOL1->ACTB | inter- | I AND T | + | 0.08747 | NO | 4 | 4 | 459 |
| | | | | chr | | | | | | | |
| 9$ | s_49 | ACTB_APOL1 | | | | | 0.02488 | | | | |
| 10$ | s_54 | ACTB_APOL1 | | | | | 0.02745 | | | | |
| 11*/$/+ | s_56 | ACTB_C20orf112 | ACTB-> | inter- | T | − | 0.07171 | NO | 2 | 2 | 460 |
| | | | C20orf112 | chr | | | | | | | |
| 12*/$/+ | s_56 | ACTB_C20orf112 | ACTB-> | inter- | T | − | 0.07171 | NO | 2 | 2 | 461 |
| | | | >C20orf112 | chr | | | | | | | |
| 13$ | s_51 | ACTB_C20orf112 | | | | | 0.02566 | | | | |
| 14*/$ | s_54 | ACTB_H1F0 | H1F0->ACTB | inter- | I AND T | + | 0.08236 | NO | 3 | 3 | 462 |
| | | | | chr | | | | | | | |
| 15$ | s_25 | ACTB_H1F0 | | | | | 0.03320 | | | | |
| 16$ | s_30 | ACTB_H1F0 | | | | | 0.03205 | | | | |
| 17$ | s_35 | ACTB_H1F0 | | | | | 0.01317 | | | | |
| 18$ | s_38 | ACTB_H1F0 | | | | | 0.01254 | | | | |
| 19$ | s_51 | ACTB_H1F0 | | | | | 0.02566 | | | | |
| 20$ | s_53 | ACTB_H1F0 | | | | | 0.02980 | | | | |
| 21*/$ | s_34 | ACTB_NDUFS6 | NDUFS6-> | inter- | I AND T | + | 0.03955 | NO | 4 | 4 | 463 |
| | | | ACTB | chr | | | | | | | |
| 22$ | s_53 | ACTB_NDUFS6 | | | | | 0.08940 | | | | |
| 23*/$ | s_50 | ACTB_OGT | OGT->ACTB | inter- | I AND T | + | 0.07234 | NO | 7 | 7 | 464 |
| | | | | chr | | | | | | | |
| 24$ | s_29 | ACTB_OGT | | | | | 0.03508 | | | | |
| 25* | s_49 | ACTB_SLC34A2 | SLC34A2-> | inter- | I AND T | + | 0.09950 | NO | 5 | 5 | 465 |
| | | | ACTB | chr | | | | | | | |
| 26*/$ | s_53 | ACTG1_PPP1R12C | ACTG1-> | inter- | T | − | 0.17881 | NO | 3 | 3 | 466 |
| | | | PPP1R12C | chr | | | | | | | |
| 27$ | s_38 | ACTG1_PPP1R12C | | | | | 0.01254 | | | | |
| 28$ | s_55 | ACTG1_PPP1R12C | | | | | 0.02916 | | | | |
| 29$ | s_56 | ACTG1_PPP1R12C | | | | | 0.07171 | | | | |
| 30* | s_51 | ADCY9_C16orf5 | ADCY9-> | intra- | T | − | 0.23096 | YES | 3 | 3 | 467 |
| | | | C16orf5 | chr | | | | | | | |
| 31*/$ | s_39 | ADD3_FTL | FTL->ADD3 | inter- | T | + | 0.03872 | NO | 2 | 2 | 468 |
| | | | | chr | | | | | | | |
| 32$ | s_29 | ADD3_FTL | | | | | 0.03508 | | | | |
| 33*/+ | s_35 | AEBP1_THRA | AEBP1->THRA | inter- | T | + | 0.03952 | NO | 2 | 2 | 469 |
| | | | | chr | | | | | | | |
| 34+ | s_35 | AEBP1_THRA | AEBP1-THRA | inter- | T | Can Not | 0.03952 | NO | 2 | 2 | 470 |
| | | | | chr | | Determine | | | | | |
| 35* | s_33 | AMD1_IGFBP5 | IGFBP5-> | inter- | I AND T | − | 0.07198 | NO | 2 | 2 | 471 |
| | | | AMD1 | chr | | | | | | | |
| 36* | s_51 | ANKHD1_ITGAV | ITGAV-> | inter- | T | + | 0.66722 | YES | 9 | 9 | 472 |
| | | | ANKHD1 | chr | | | | | | | |
| 37* | s_30 | ANP32E_MYST4 | ANP32E-> | inter- | I AND T | − | 0.12819 | NO | 4 | 4 | 473 |
| | | | MYST4 | chr | | | | | | | |
| 38* | s_34 | APOOL_DCAF8 | APOOL-> | inter- | I AND T | + | 0.05273 | NO | 5 | 5 | 474 |
| | | | DCAF8 | chr | | | | | | | |
| 39* | s_34 | ARIH2_TMEM119 | TMEM119-> | inter- | I AND T | − | 0.05273 | NO | 13 | 13 | 475 |
| | | | ARIH2 | chr | | | | | | | |
| 40* | s_27 | ARL2_CAPN1 | CAPN1->ARL2 | intra- | T | + | 0.74395 | YES | 24 | 24 | 476 |
| | | | | chr | | | | | | | |
| 41*/$ | s_30 | ARL3_MTF2 | MTF2->ARL3 | inter- | I AND T | + | 0.48072 | YES | 34 | 15 | 477 |
| | | | | chr | | | | | | | |
| 42*/$ | s_52 | ARL3_MTF2 | MTF2->ARL3 | inter- | I AND T | + | 0.58084 | YES | 34 | 19 | 478 |
| | | | | chr | | | | | | | |
| 43* | s_33 | ASAP1_MALAT1 | ASAP1-> | inter- | I AND T | − | 0.03599 | NO | 3 | 3 | 479 |
| | | | MALAT1 | chr | | | | | | | |
| 44*/$ | s_40 | BASP1_COL1A1 | COL1A1-> | inter- | I AND T | − | 0.07187 | NO | 7 | 7 | 480 |
| | | | BASP1 | chr | | | | | | | |
| 45$ | s_33 | BASP1_COL1A1 | | | | | 0.01200 | | | | |
| 46$ | s_54 | BASP1_COL1A1 | | | | | 0.05490 | | | | |
| 47$ | s_55 | BASP1_COL1A1 | | | | | 0.02916 | | | | |
| 48*/$ | s_35 | BAT2L2_COL3A1 | BAT2L2-> | inter- | T | + | 0.05269 | NO | 14 | 14 | 481 |
| | | | COL3A1 | chr | | | | | | | |
| 49$ | s_31 | BAT2L2_COL3A1 | | | | | 0.03700 | | | | |
| 50$ | s_37 | BAT2L2_COL3A1 | | | | | 0.02519 | | | | |
| 51$ | s_39 | BAT2L2_COL3A1 | | | | | 0.01291 | | | | |
| 52$ | s_52 | BAT2L2_COL3A1 | | | | | 0.02904 | | | | |
| 53$ | s_54 | BAT2L2_COL3A1 | | | | | 0.02745 | | | | |
| 54* | s_54 | C2orf56_SAMD4B | C2orf56-> | inter- | T | + | 0.08236 | NO | 2 | 2 | 482 |
| | | | SAMD4B | chr | | | | | | | |
| 55* | s_31 | C8orf46_GPATCH8 | GPATCH8-> | inter- | I AND T | − | 0.36997 | YES | 2 | 2 | 483 |
| | | | C8orf46 | chr | | | | | | | |
| 56* | s_54 | CAT_PDHX | PDHX->CAT | intra- | T | + | 0.46669 | YES | 5 | 5 | 484 |
| | | | | chr | | | | | | | |
| 57*/+ | s_50 | CD24_GPAA1 | GPAA1->CD24 | inter- | I AND T | + | 0.07234 | NO | 3 | 3 | 485 |
| | | | | chr | | | | | | | |
| 58*/+ | s_50 | CD24_GPAA1 | GPAA1->CD24 | inter- | I AND T | + | 0.07234 | NO | 2 | 2 | 486 |
| | | | | chr | | | | | | | |
| 59*/$ | s_40 | CD68_NEAT1 | CD68->NEAT1 | inter- | T | + | 0.05750 | NO | 3 | 3 | 487 |
| | | | | chr | | | | | | | |
| 60$ | s_27 | CD68_NEAT1 | | | | | 0.03100 | | | | |
| 61$ | s_33 | CD68_NEAT1 | | | | | 0.01200 | | | | |
| 62$ | s_39 | CD68_NEAT1 | | | | | 0.01291 | | | | |
| 63*/$ | s_39 | CD68_PSAP | CD68->PSAP | inter- | I AND T | + | 0.05162 | NO | 9 | 8 | 488 |
| | | | | chr | | | | | | | |
| 64*/$ | s_39 | CD68_PSAP | CD68->PSAP | inter- | I AND T | + | 0.05162 | NO | 6 | 6 | 489 |
| | | | | chr | | | | | | | |
| 65$ | s_29 | CD68_PSAP | | | | | 0.24555 | | | | |
| 66$ | s_38 | CD68_PSAP | | | | | 0.01254 | | | | |
| 67$ | s_49 | CD68_PSAP | | | | | 0.12438 | | | | |
| 68$ | s_54 | CD68_PSAP | | | | | 0.05490 | | | | |
| 69*/$ | s_54 | CD74_MBD6 | CD74->MBD6 | inter- | I AND T | − | 0.13726 | NO | 19 | 19 | 490 |
| | | | | chr | | | | | | | |
| 70$ | s_56 | CD74_MBD6 | | | | | 0.04780 | | | | |
| 71* | s_53 | CDK4_UBA1 | CDK4->UBA1 | inter- | I AND T | − | 0.08940 | NO | 6 | 6 | 491 |
| | | | | chr | | | | | | | |
| 72* | s_54 | CIRBP_UGP2 | CIRBP->UGP2 | inter- | T | + | 0.10981 | NO | 2 | 2 | 492 |
| | | | | chr | | | | | | | |
| 73* | s_40 | COL14A1_DNAJA2 | DNAJA2-> | inter- | I AND T | − | 0.05750 | NO | 2 | 2 | 493 |
| | | | COL14A1 | chr | | | | | | | |
| 74* | s_35 | COL16A1_COL3A1 | COL3A1-> | inter- | I AND T | + | 0.03952 | NO | 2 | 2 | 494 |
| | | | COL16A1 | chr | | | | | | | |
| 75*/$ | s_37 | COL1A1_EPN1 | EPN1-> | inter- | I AND T | + | 0.03778 | NO | 7 | 7 | 495 |
| | | | COL1A1 | chr | | | | | | | |
| 76$ | s_28 | COL1A1_EPN1 | | | | | 0.03261 | | | | |
| 77$ | s_33 | COL1A1_EPN1 | | | | | 0.01200 | | | | |
| 78$ | s_35 | COL1A1_EPN1 | | | | | 0.01317 | | | | |
| 79$ | s_54 | COL1A1_EPN1 | | | | | 0.05490 | | | | |
| 80* | s_54 | COL1A1_FGD2 | COL1A1-> | inter- | I AND T | − | 0.08236 | NO | 2 | 2 | 496 |
| | | | FGD2 | chr | | | | | | | |
| 81*/$ | s_40 | COL1A1_FMNL3 | COL1A1-> | inter- | T | − | 0.04312 | NO | 3 | 3 | 497 |
| | | | FMNL3 | chr | | | | | | | |
| 82$ | s_35 | COL1A1_FMNL3 | | | | | 0.01317 | | | | |
| 83* | s_35 | COL1A1_GORASP2 | COL1A1-> | inter- | I AND T | − | 0.05269 | NO | 4 | 4 | 498 |
| | | | GORASP2 | chr | | | | | | | |
| 84* | s_40 | COL1A1_HEATR5A | HEATR5A-> | inter- | T | − | 0.05750 | NO | 2 | 2 | 499 |
| | | | COL1A1 | chr | | | | | | | |
| 85*/$ | s_35 | COL1A2_LAMP2 | COL1A2-> | inter- | I AND T | + | 0.03952 | NO | 2 | 2 | 500 |
| | | | LAMP2 | chr | | | | | | | |
| 86$ | s_37 | COL1A2_LAMP2 | | | | | 0.01259 | | | | |
| 87$ | s_54 | COL1A2_LAMP2 | | | | | 0.02745 | | | | |
| 88*/$ | s_40 | COL3A1_DCLK1 | DCLK1-> | inter- | I AND T | − | 0.05750 | NO | 4 | 4 | 501 |
| | | | COL3A1 | chr | | | | | | | |
| 89$ | s_35 | COL3A1_DCLK1 | | | | | 0.01317 | | | | |
| 90* | s_40 | COL3A1_POLD3 | POLD3-> | inter- | T | + | 0.05750 | NO | 2 | 2 | 502 |
| | | | COL3A1 | chr | | | | | | | |
| 91*/$ | s_40 | COL3A1_SPATS2L | SPATS2L-> | intra- | T | + | 0.05750 | NO | 11 | 11 | 503 |
| | | | COL3A1 | chr | | | | | | | |
| 92$ | s_38 | COL3A1_SPATS2L | | | | | 0.01254 | | | | |
| 93$ | s_49 | COL3A1_SPATS2L | | | | | 0.02488 | | | | |
| 94* | s_35 | COL3A1_ZNF43 | COL3A1-> | inter- | I AND T | + | 0.03952 | NO | 2 | 2 | 504 |
| | | | ZNF43 | chr | | | | | | | |
| 95* | s_52 | CPNE3_IFI27 | IFI27->CPNE3 | inter- | T | + | 0.08713 | NO | 7 | 7 | 505 |
| | | | | chr | | | | | | | |
| 96* | s_34 | CRNKL1_RHOBTB3 | RHOBTB3-> | inter- | I AND T | + | 0.05273 | NO | 2 | 2 | 506 |
| | | | CRNKL1 | chr | | | | | | | |
| 97* | s_53 | CTSD_EPHA2 | EPHA2->CTSD | inter- | T | − | 0.08940 | NO | 2 | 2 | 507 |
| | | | | chr | | | | | | | |
| 98*/$ | s_53 | CTSD_GNB2 | GNB2->CTSD | inter- | I AND T | + | 0.14901 | NO | 2 | 2 | 508 |
| | | | | chr | | | | | | | |
| 99$ | s_54 | CTSD_GNB2 | | | | | 0.02745 | | | | |
| 100* | s_53 | CTSD_LTBP4 | LTBP4->CTSD | inter- | I AND T | + | 0.14901 | NO | 4 | 4 | 509 |
| | | | | chr | | | | | | | |
| 101* | s_53 | CTSD_PACSIN3 | PACSIN3-> | intra- | D OR T | − | 0.08940 | NO | 2 | 2 | 510 |
| | | | CTSD | chr | | | | | | | |
| 102*/$ | s_53 | CTSD_PLXNA1 | PLXNA1-> | inter- | I AND T | + | 0.11920 | NO | 4 | 4 | 511 |
| | | | CTSD | chr | | | | | | | |
| 103$ | s_38 | CTSD_PLXNA1 | | | | | 0.01254 | | | | |
| 104* | s_53 | CTSD_PRKAR1B | CTSD-> | inter- | T | − | 0.14901 | NO | 18 | 18 | 512 |
| | | | PRKAR1B | chr | | | | | | | |
| 105* | s_53 | CTSD_TMEM109 | TMEM109-> | intra- | I AND T | + | 0.14901 | NO | 4 | 4 | 513 |
| | | | CTSD | chr | | | | | | | |
| 106* | s_26 | CTSS_GOLPH3L | GOLPH3L-> | intra- | T | − | 0.80247 | YES | 5 | 5 | 514 |
| | | | CTSS | chr | | | | | | | |
| 107* | s_33 | CTTN_NCRNA00201 | CTTN-> | inter- | I AND T | + | 0.03599 | NO | 3 | 3 | 515 |
| | | | NCRNA00201 | chr | | | | | | | |
| 108* | s_29 | CWC25_ROBO2 | CWC25-> | inter- | I AND T | − | 1.75396 | YES | 12 | 12 | 516 |
| | | | ROBO2 | chr | | | | | | | |
| 109* | s_39 | CYB561_YWHAG | YWHAG-> | inter- | T | − | 0.03872 | NO | 2 | 2 | 517 |
| | | | CYB561 | chr | | | | | | | |
| 110*/$ | s_40 | CYB5R3_TXNIP | CYB5R3-> | inter- | I AND T | − | 0.04312 | NO | 2 | 2 | 518 |
| | | | TXNIP | chr | | | | | | | |
| 111$ | s_35 | CYB5R3_TXNIP | | | | | 0.01317 | | | | |
| 112*/$ | s_40 | DCN_VPS35 | VPS35->DCN | inter- | T | − | 0.05750 | NO | 6 | 6 | 519 |
| | | | | chr | | | | | | | |
| 113$ | s_28 | DCN_VPS35 | | | | | 0.03261 | | | | |
| 114*/+ | s_29 | DIDO1_REPS1 | DIDO1-> | inter- | T | − | 1.19269 | YES | 19 | 19 | 520 |
| | | | REPS1 | chr | | | | | | | |
| 115*/+ | s_29 | DIDO1_REPS1 | DIDO1-> | inter- | T | − | 1.19269 | YES | 2 | 2 | 521 |
| | | | REPS1 | chr | | | | | | | |
| 116*/$ | s_50 | DNM2_PIN1 | DNM2->PIN1 | intra- | T | + | 0.07234 | YES | 3 | 3 | 522 |
| | | | | chr | | | | | | | |
| 117$ | s_38 | DNM2_PIN1 | | | | | 0.01254 | | | | |
| 118*/$ | s_34 | EIF4G2_RAB8A | RAB8A-> | inter- | I AND T | + | 0.03955 | NO | 4 | 4 | 523 |
| | | | EIF4G2 | chr | | | | | | | |
| 119$ | s_52 | EIF4G2_RAB8A | | | | | 0.05808 | | | | |
| 120*/$ | s_38 | ELAC1_SMAD4 | ELAC1-> | intra- | D OR T | + | 0.03762 | YES | 2 | 2 | 524 |
| | | | SMAD4 | chr | | | | | | | |
| 121$ | s_27 | ELAC1_SMAD4 | | | | | 0.03100 | | | | |
| 122$ | s_29 | ELAC1_SMAD4 | | | | | 0.03508 | | | | |
| 123$ | s_33 | ELAC1_SMAD4 | | | | | 0.01200 | | | | |
| 124$ | s_35 | ELAC1_SMAD4 | | | | | 0.01317 | | | | |
| 125$ | s_37 | ELAC1_SMAD4 | | | | | 0.03778 | | | | |
| 126$ | s_39 | ELAC1_SMAD4 | | | | | 0.01291 | | | | |
| 127$ | s_40 | ELAC1_SMAD4 | | | | | 0.02875 | | | | |
| 128*/$ | s_33 | ELF3_SLC39A6 | ELF3-> | inter- | I AND T | + | 0.03599 | NO | 2 | 2 | 525 |
| | | | SLC39A6 | chr | | | | | | | |
| 129$ | s_35 | ELF3_SLC39A6 | | | | | 0.01317 | | | | |
| 130* | s_56 | ELN_NCOR2 | NCOR2->ELN | inter- | I AND T | − | 0.07171 | NO | 2 | 2 | 526 |
| | | | | chr | | | | | | | |
| 131* | s_51 | EMP2_KRT81 | KRT81->EMP2 | inter- | T | − | 0.10265 | NO | 3 | 3 | 527 |
| | | | | chr | | | | | | | |
| 132* | s_27 | FAM3B_GLI3 | GLI3->FAM3B | inter- | I AND T | − | 0.65096 | YES | 4 | 4 | 528 |
| | | | | chr | | | | | | | |
| 133* | s_53 | FLNA_SBF1 | SBF1->FLNA | inter- | T | − | 0.20861 | NO | 12 | 12 | 529 |
| | | | | chr | | | | | | | |
| 134* | s_51 | GAPDH_KRT13 | GAPDH-> | inter- | I AND T | + | 0.30795 | NO | 2 | 2 | 530 |
| | | | KRT13 | chr | | | | | | | |
| 135* | s_52 | GAPDH_MRPS18B | GAPDH-> | inter- | T | + | 0.11617 | NO | 4 | 4 | 531 |
| | | | MRPS18B | chr | | | | | | | |
| 136*/$ | s_56 | GATA3_RHOB | RHOB-> | inter- | T | + | 0.21512 | NO | 12 | 12 | 532 |
| | | | GATA3 | chr | | | | | | | |
| 137$ | s_33 | GATA3_RHOB | | | | | 0.01200 | | | | |
| 138$ | s_55 | GATA3_RHOB | | | | | 0.05831 | | | | |
| 139*/+ | s_50 | GEMIN7_SLC39A14 | GEMIN7-> | inter- | T | + | 0.67516 | YES | 5 | 5 | 533 |
| | | | SLC39A14 | chr | | | | | | | |
| 140*/+ | s_50 | GEMIN7_SLC39A14 | GEMIN7-> | inter- | T | + | 0.67516 | YES | 2 | 2 | 534 |
| | | | SLC39A14 | chr | | | | | | | |
| 141* | s_55 | GNB1_TRH | GNB1->TRH | inter- | I AND T | − | 0.11663 | NO | 2 | 2 | 535 |
| | | | | chr | | | | | | | |
| 142* | s_56 | GNB4_PTMA | PTMA->GNB4 | inter- | I AND T | + | 0.07171 | NO | 12 | 12 | 536 |
| | | | | chr | | | | | | | |
| 143* | s_32 | GPR128_TFG | TFG->GPR128 | intra- | T | + | 0.80135 | YES | 9 | 9 | 537 |
| | | | | chr | | | | | | | |
| 144* | s_56 | HDLBP_NTN1 | NTN1->HDLBP | inter- | I AND T | + | 0.07171 | NO | 2 | 2 | 538 |
| | | | | chr | | | | | | | |
| 145*/$ | s_54 | HLA-E_TSPAN14 | TSPAN14-> | inter- | T | + | 0.08236 | NO | 4 | 1 | 539 |
| | | | HLA-E | chr | | | | | | | |
| 146*/$ | s_56 | HLA-E_TSPAN14 | TSPAN14-> | inter- | T | + | 0.07171 | NO | 4 | 3 | 540 |
| | | | HLA-E | chr | | | | | | | |
| 147* | s_36 | HMGN3_PAQR8 | HMGN3-> | intra- | I AND (D | − | 0.11498 | YES | 3 | 3 | 541 |
| | | | PAQR8 | chr | OR T) | | | | | | |
| 148* | s_30 | HNRNPH1_VAPA | HNRNPH1-> | inter- | I AND T | − | 0.12819 | NO | 2 | 2 | 542 |
| | | | VAPA | chr | | | | | | | |
| 149* | s_34 | HNRNPU_TES | TES-> | inter- | I AND T | + | 0.03955 | NO | 3 | 3 | 543 |
| | | | HNRNPU | chr | | | | | | | |
| 150* | s_33 | HSP90AB1_PCGF2 | HSP90AB1-> | inter- | I AND T | + | 0.03599 | NO | 2 | 2 | 544 |
| | | | PCGF2 | chr | | | | | | | |
| 151*/$ | s_38 | IGF2_MALAT1 | MALAT1-> | intra- | I AND T | + | 0.03762 | NO | 4 | 1 | 545 |
| | | | IGF2 | chr | | | | | | | |
| 152$ | s_28 | IGF2_MALAT1 | | | | | 0.03261 | | | | |
| 153$ | s_29 | IGF2_MALAT1 | | | | | 0.03508 | | | | |
| 154$ | s_34 | IGF2_MALAT1 | IGF2-MALAT1 | intra- | I AND (D | Can Not | 0.10546 | NO | 4 | 3 | 546 |
| | | | | chr | OR T) | Determine | | | | | |
| 155* | s_33 | IGFBP5_RAB3IP | RAB3IP-> | inter- | I AND T | + | 0.03599 | NO | 7 | 7 | 547 |
| | | | IGFBP5 | chr | | | | | | | |
| 156* | s_40 | IGFBP7_MAF | MAF->IGFBP7 | inter- | T | − | 0.04312 | NO | 2 | 2 | 548 |
| | | | | chr | | | | | | | |
| 157*/$ | s_36 | IGLL5_LOC96610 | LOC96610-> | intra- | D OR T | + | 0.43117 | NO | 51 | 1 | 549 |
| | | | IGLL5 | chr | | | | | | | |
| 158*/$ | s_49 | IGLL5_LOC96610 | LOC96610-> | intra- | D OR T | + | 6.99014 | NO | 51 | 50 | 550 |
| | | | IGLL5 | chr | | | | | | | |
| 159$ | s_26 | IGLL5_LOC96610 | | | | | 0.70618 | | | | |
| 160$ | s_27 | IGLL5_LOC96610 | | | | | 0.27898 | | | | |
| 161$ | s_28 | IGLL5_LOC96610 | | | | | 0.32609 | | | | |
| 162$ | s_29 | IGLL5_LOC96610 | | | | | 0.59635 | | | | |
| 163$ | s_31 | IGLL5_LOC96610 | | | | | 2.18284 | | | | |
| 164$ | s_32 | IGLL5_LOC96610 | | | | | 0.41810 | | | | |
| 165$ | s_35 | IGLL5_LOC96610 | | | | | 0.55326 | | | | |
| 166$ | s_37 | IGLL5_LOC96610 | | | | | 0.23929 | | | | |
| 167$ | s_38 | IGLL5_LOC96610 | | | | | 0.96567 | | | | |
| 168$ | s_40 | IGLL5_LOC96610 | | | | | 4.93033 | | | | |
| 169$ | s_50 | IGLL5_LOC96610 | | | | | 7.81259 | | | | |
| 170$ | s_51 | IGLL5_LOC96610 | | | | | 0.64156 | | | | |
| 171$ | s_54 | IGLL5_LOC96610 | | | | | 3.15700 | | | | |
| 172$ | s_55 | IGLL5_LOC96610 | | | | | 0.05831 | | | | |
| 173$ | s_56 | IGLL5_LOC96610 | | | | | 0.23902 | | | | |
| 174* | s_49 | IGLL5_SFTPC | SFTPC->IGLL5 | inter- | T | + | 0.07463 | NO | 2 | 2 | 551 |
| | | | | chr | | | | | | | |
| 175* | s_55 | IRX3_USF2 | USF2->IRX3 | inter- | I AND T | + | 0.08747 | NO | 2 | 2 | 552 |
| | | | | chr | | | | | | | |
| 176* | s_53 | ITGA3_KHK | ITGA3->KHK | inter- | T | + | 0.08940 | NO | 3 | 3 | 553 |
| | | | | chr | | | | | | | |
| 177* | s_26 | JOSD1_RPS19BP1 | JOSD1-> | intra- | T | − | 0.57778 | YES | 4 | 4 | 554 |
| | | | RPS19BP1 | chr | | | | | | | |
| 178*/$ | s_38 | KCTD1_LOC728606 | LOC728606-> | intra- | D OR T | − | 0.13795 | YES | 6 | 2 | 555 |
| | | | KCTD1 | chr | | | | | | | |
| 179*/$ | s_55 | KCTD1_LOC728606 | LOC728606-> | intra- | D OR T | − | 0.46652 | YES | 6 | 4 | 556 |
| | | | KCTD1 | chr | | | | | | | |
| 180$ | s_26 | KCTD1_LOC728606 | | | | | 0.06420 | | | | |
| 181$ | s_28 | KCTD1_LOC728606 | | | | | 0.03261 | | | | |
| 182$ | s_33 | KCTD1_LOC728606 | | | | | 0.03599 | | | | |
| 183$ | s_34 | KCTD1_LOC728606 | | | | | 0.26364 | | | | |
| 184$ | s_39 | KCTD1_LOC728606 | | | | | 0.01291 | | | | |
| 185$ | s_56 | KCTD1_LOC728606 | | | | | 0.54975 | | | | |
| 186* | s_31 | KCTD3_TXNDC16 | KCTD3-> | inter- | I AND T | + | 0.66595 | YES | 4 | 4 | 557 |
| | | | TXNDC16 | chr | | | | | | | |
| 187* | s_34 | KIAA1217_SERPINA1 | SERPINA1-> | inter- | I AND T | − | 0.05273 | NO | 3 | 3 | 558 |
| | | | KIAA1217 | chr | | | | | | | |
| 188*/$ | s_51 | KRT18_PLEC | KRT18->PLEC | inter- | I AND T | + | 0.10265 | NO | 2 | 2 | 559 |
| | | | | chr | | | | | | | |
| 189$ | s_53 | KRT18_PLEC | | | | | 0.11920 | | | | |
| 190* | s_51 | KRT4_RPL8 | RPL8->KRT4 | inter- | T | − | 0.07699 | NO | 2 | 2 | 560 |
| | | | | chr | | | | | | | |
| 191* | s_26 | LAMB3_RALGPS2 | RALGPS2-> | intra- | I AND (D | + | 0.25679 | YES | 6 | 6 | 561 |
| | | | LAMB3 | chr | OR T) | | | | | | |
| 192*/$ | s_54 | LGMN_NAP1L1 | LGMN-> | inter- | T | − | 0.10981 | YES | 2 | 2 | 562 |
| | | | NAP1L1 | chr | | | | | | | |
| 193$ | s_29 | LGMN_NAP1L1 | | | | | 0.03508 | | | | |
| 194* | s_33 | LRIG1_SLC39A6 | SLC39A6-> | inter- | T | − | 0.04798 | NO | 3 | 3 | 563 |
| | | | LRIG1 | chr | | | | | | | |
| 195*/$ | s_33 | MALAT1_PTP4A2 | PTP4A2-> | inter- | I AND T | − | 0.04798 | NO | 2 | 2 | 564 |
| | | | MALAT1 | chr | | | | | | | |
| 196$ | s_51 | MALAT1_PTP4A2 | | | | | 0.05132 | | | | |
| 197$ | s_55 | MALAT1_PTP4A2 | | | | | 0.02916 | | | | |
| 198*/$ | s_33 | MALAT1_TAX1BP1 | TAX1BP1-> | inter- | T | + | 0.04798 | NO | 2 | 2 | 565 |
| | | | | MALAT1 | chr | | | | | | |
| 199$ | s_39 | MALAT1_TAX1BP1 | | | | | 0.02581 | | | | |
| 200* | s_33 | MAPK1IP1L_XPO1 | MAPK1IP1L-> | inter- | I AND T | + | 0.03599 | NO | 2 | 2 | 566 |
| | | | XPO1 | chr | | | | | | | |
| 201*/$ | s_34 | MGP_NCRNA00188 | MGP-> | inter- | I AND T | − | 0.05273 | NO | 3 | 3 | 567 |
| | | | NCRNA00188 | chr | | | | | | | |
| 202$ | s_37 | MGP_NCRNA00188 | | | | | 0.01259 | | | | |
| 203$ | s_38 | MGP_NCRNA00188 | | | | | 0.01254 | | | | |
| 204* | s_33 | MGP_REPS2 | MGP->REPS2 | inter- | I AND T | − | 0.03599 | NO | 2 | 2 | 568 |
| | | | | chr | | | | | | | |
| 205* | s_50 | MKKS_PCNX | PCNX->MKKS | inter- | I AND T | + | 0.55460 | YES | 4 | 4 | 569 |
| | | | | chr | | | | | | | |
| 206* | s_53 | MRPL4_SLC16A3 | SLC16A3-> | inter- | T | + | 0.08940 | NO | 30 | 30 | 570 |
| | | | MRPL4 | chr | | | | | | | |
| 207* | s_40 | MRPL52_USP22 | MRPL52-> | inter- | I AND T | + | 0.04312 | NO | 3 | 3 | 571 |
| | | | USP22 | chr | | | | | | | |
| 208*/$ | s_29 | MUCL1_RPL23 | RPL23-> | inter- | I AND T | − | 0.10524 | NO | 4 | 4 | 572 |
| | | | MUCL1 | chr | | | | | | | |
| 209$ | s_27 | MUCL1_RPL23 | | | | | 0.06200 | | | | |
| 210$ | s_38 | MUCL1_RPL23 | | | | | 0.02508 | | | | |
| 211$ | s_51 | MUCL1_RPL23 | | | | | 0.02566 | | | | |
| 212* | s_54 | NAV2_WDFY1 | NAV2-> | inter- | I AND T | + | 0.10981 | NO | 3 | 3 | 573 |
| | | | WDFY1 | chr | | | | | | | |
| 213* | s_49 | NPLOC4_PDE6G | NPLOC4-> | intra- | T | − | 1.29355 | YES | 10 | 10 | 574 |
| | | | PDE6G | chr | | | | | | | |
| 214* | s_31 | OLA1_ORMDL3 | OLA1-> | inter- | T | − | 0.55496 | YES | 2 | 2 | 575 |
| | | | ORMDL3 | chr | | | | | | | |
| 215* | s_36 | PAQR5_THSD4 | THSD4-> | intra- | T | + | 0.21558 | YES | 3 | 3 | 576 |
| | | | PAQR5 | chr | | | | | | | |
| 216* | s_55 | PDIA3_YWHAG | YWHAG-> | inter- | I AND T | − | 0.08747 | NO | 4 | 4 | 577 |
| | | | PDIA3 | chr | | | | | | | |
| 217* | s_56 | PIKFYVE_TMEM119 | PIKFYVE-> | inter- | I AND T | + | 0.07171 | NO | 7 | 7 | 578 |
| | | | TMEM119 | chr | | | | | | | |
| 218* | s_53 | PKM2_SEMA4C | SEMA4C-> | inter- | T | − | 0.08940 | NO | 4 | 4 | 579 |
| | | | PKM2 | chr | | | | | | | |
| 219* | s_53 | PLEC_PLEKHM2 | PLEC-> | inter- | I AND T | − | 0.08940 | NO | 2 | 2 | 580 |
| | | | PLEKHM2 | chr | | | | | | | |
| 220* | s_53 | PLEC_RPS15 | RPS15->PLEC | inter- | I AND T | + | 0.20861 | NO | 4 | 4 | 581 |
| | | | | chr | | | | | | | |
| 221* | s_40 | POSTN_TM9SF3 | POSTN-> | inter- | T | − | 0.04312 | NO | 3 | 3 | 582 |
| | | | TM9SF3 | chr | | | | | | | |
| 222* | s_40 | POSTN_TRIM33 | POSTN-> | inter- | T | − | 0.04312 | NO | 2 | 2 | 583 |
| | | | TRIM33 | chr | | | | | | | |
| 223* | s_49 | PROM1_TAPT1 | PROM1-> | intra- | T | − | 0.24876 | YES | 2 | 2 | 584 |
| | | | TAPT1 | chr | | | | | | | |
| 224* | s_31 | RBM6_SLC38A3 | RBM6-> | intra- | D OR T | + | 0.14799 | YES | 2 | 2 | 585 |
| | | | SLC38A3 | chr | | | | | | | |
| 225* | s_29 | RNASE1_TEP1 | TEP1-> | intra- | T | − | 0.10524 | YES | 2 | 2 | 586 |
| | | | RNASE1 | chr | | | | | | | |
| 226*/$ | s_34 | RNF11_STC2 | STC2->RNF11 | inter- | I AND T | − | 0.05273 | NO | 2 | 2 | 587 |
| | | | | chr | | | | | | | |
| 227$ | s_37 | RNF11_STC2 | | | | | 0.02519 | | | | |
| 228*/$ | s_51 | RPL19_RPS16 | RPL19-> | inter- | I AND T | + | 0.17964 | NO | 2 | 2 | 589 |
| | | | RPS16 | chr | | | | | | | |
| 229$ | s_31 | RPL19_RPS16 | | | | | 0.03700 | | | | |
| 230$ | s_52 | RPL19_RPS16 | | | | | 0.05808 | | | | |
| 231*/ | s_51 | RPS16_TMSB10 | TMSB10-> | inter- | I AND T | + | 0.59023 | NO | 4 | 4 | 590 |
| | | | RPS16 | chr | | | | | | | |
| 232$ | s_29 | RPS16_TMSB10 | | | | | 0.03508 | | | | |
| 233$ | s_32 | RPS16_TMSB10 | | | | | 0.03484 | | | | |
| 234$ | s_53 | RPS16_TMSB10 | | | | | 0.05960 | | | | |
| 235* | s_33 | SFI1_YPEL1 | SFI1->YPEL1 | intra- | I AND T | + | 0.05998 | YES | 2 | 2 | 591 |
| | | | | chr | | | | | | | |
| 236* | s_55 | SLC9A3R1_TNRC18 | TNRC18-> | inter- | I AND T | − | 0.08747 | NO | 2 | 2 | 592 |
| | | | SLC9A3R1 | chr | | | | | | | |
| 237*/$ | s_51 | SOCS5_TTC7A | TTC7A-> | intra- | T | + | 0.23096 | YES | 5 | 5 | 593 |
| | | | SOCS5 | chr | | | | | | | |
| 238$ | s_40 | SOCS5_TTC7A | | | | | 0.02875 | | | | |
| 239$ | s_53 | SOCS5_TTC7A | | | | | 0.05960 | | | | |
| 240*/$/+ | s_35 | SPARC_TRPS1 | SPARC-> | inter- | T | − | 0.05269 | NO | 10 | 10 | 594 |
| | | | TRPS1 | chr | | | | | | | |
| 241*/$/+ | s_35 | SPARC_TRPS1 | SPARC-> | inter- | T | − | 0.05269 | NO | 4 | 4 | 595 |
| | | | TRPS1 | chr | | | | | | | |
| 242$ | s_27 | SPARC_TRPS1 | | | | | 0.03100 | | | | |
| 243$ | s_33 | SPARC_TRPS1 | | | | | 0.01200 | | | | |
| 244$ | s_39 | SPARC_TRPS1 | | | | | 0.01291 | | | | |
| 245* | s_36 | SRPK1_UBR2 | UBR2->SRPK1 | intra- | I AND T | + | 1.32225 | YES | 8 | 8 | 596 |
| | | | | chr | | | | | | | |
| 246*/$ | s_52 | YWHAZ_ZBTB33 | YWHAZ-> | inter- | I AND T | − | 0.11617 | NO | 2 | 2 | 597 |
| | | | ZBTB33 | chr | | | | | | | |
| 247$ | s_54 | YWHAZ_ZBTB33 | | | | | 0.02745 | | | | |

| Row # | Exon1 | Exon2 | Sample ID |
|---|---|---|---|
| 1 | E14:chr17:AATK:NM_001080395:76754332:76754467:− | E33:chr17:USP32:NM_032582:55777623:55777751:− | Triple Negative Breast |
| | | | Tumor |
| 2 | E14:chr17:AATK:NM_001080395:76754332:76754467:− | E33:chr17:USP32:NM_032582:55777623:55777751:− | Triple Negative Breast |
| | | | Tumor |
| 3 | E17:chr17:ABCA10:NM_080282:64683141:64683249:− | E6:chr17:TP53I13:NM_138349:24923285:24923841:+ | HER2+ Breast Tumor |
| 4 | E1:chr9:ABCA2:NM_212533:139021506:139022237:− | E3:chrX:FLNA:NM_001110556:153231210:153231429:− | Triple Negative Breast |
| | | | Tumor |
| 5 | | | ER+ Breast Tumor |
| 6 | E1:chr3:ABCC5:NM_005688:185120417:185121883:− | E25:chr3:EIF4G1:NM_182917:185528311:185528484:+ | ER+ Breast Tumor |
| 7 | E1:chr17:ACACA:NM_198839:32516039:32518487:− | E9:chr19:CALR:NM_004343:12915526:12916304:+ | HER2+ Breast Tumor |
| 8 | E1:chr7:ACTB:NM_001101:5533304:5534048:− | E6:chr22:APOL1:NM_001136540:34991142:34993522:+ | Triple Negative Breast |
| | | | Tumor |
| 9 | | | Triple Negative Breast |
| | | | Tumor |
| 10 | | | Triple Negative Breast |
| | | | Tumor |
| 11 | E1:chr7:ACTB:NM_001101:5533304:5534048:− | E1:chr20:C20orf112:NM_080616:30494522:30499280:− | Triple Negative Breast |
| | | | Tumor |
| 12 | E1:chr7:ACTB:NM_001101:5533304:5534048:− | E1:chr20:C20orf112:NM_080616:30494522:30499280:− | Triple Negative Breast |
| | | | Tumor |
| 13 | | | Triple Negative Breast |
| | | | Tumor |
| 14 | E3:chr7:ACTB:NM_001101:5534437:5534876:− | E1:chr22:H1F0:NM_005318:36531059:36533389:+ | Triple Negative Breast |
| | | | Tumor |
| 15 | | | HER2+ Breast Tumor |
| 16 | | | HER2+ Breast Tumor |
| 17 | | | ER+ Breast Tumor |
| 18 | | | ER+ Breast Tumor |
| 19 | | | Triple Negative Breast |
| | | | Tumor |
| 20 | | | Triple Negative Breast |
| | | | Tumor |
| 21 | E1:chr7:ACTB:NM_001101:5533304:5534048:− | E4:chr5:NDUFS6:NM_004553:1868964:1869163:+ | ER+ Breast Tumor |
| 22 | | | Triple Negative Breast |
| | | | Tumor |
| 23 | E1:chr7:ACTB:NM_001101:5533304:5534048:− | E22:chrX:OGT:NM_181672:70710194:70712472:+ | Triple Negative Breast |
| | | | Tumor |
| 24 | | | HER2+ Breast Tumor |
| 25 | E3:chr7:ACTB:NM_001101:5534437:5534876:− | E13:chr4:SLC34A2:NM_006424:25286854:25289466:+ | Triple Negative Breast |
| | | | Tumor |
| 26 | E1:chr17:ACTG1:NM_001614:77091593:77092454:− | E1:chr19:PPP1R12C:NM_017607:60294092:60294738:− | Triple Negative Breast |
| | | | Tumor |
| 27 | | | ER+ Breast Tumor |
| 28 | | | Triple Negative Breast |
| | | | Tumor |
| 29 | | | Triple Negative Breast |
| | | | Tumor |
| 30 | E10:chr16:ADCY9:NM_001116:4103751:4105487:− | E5:chr16:C16orf5:NM_013399:4504576:4504666:− | Triple Negative Breast |
| | | | Tumor |
| 31 | E14:chr10:ADD3:NM_001121:111883073:111885313:+ | E4:chr19:FTL:NM_000146:54161651:54161948:+ | ER+ Breast Tumor |
| 32 | | | HER2+ Breast Tumor |
| 33 | E18:chr7:AEBP1:NM_001129:44118681:44119033:+ | E1:chr17:THRA:NM_001190918:35472593:35472871:+ | ER+ Breast Tumor |
| 34 | E1:chr7:AEBP1:NM_001129:44110484:44111042:+ | E1:chr17:THRA:NM_001190918:35472593:35472871:+ | ER+ Breast Tumor |
| 35 | E1:chr6:AMD1:NM_001634:111302679:111303111:+ | E1:chr2:IGFBP5:NM_000599:217245072:217249850:− | ER+ Breast Tumor |
| 36 | E25:chr5:ANKHD1:NM_017747:139883834:139884009:+ | E15:chr2:ITGAV:NM_001145000:187229218:187229373:+ | Triple Negative Breast |
| | | | Tumor |
| 37 | E1:chr1:ANP32E:NM_030920:148457341:148459687:− | E18:chr10:MYST4:NM_012330:76458252:76462645:+ | HER2+ Breast Tumor |
| 38 | E9:chrX:APOOL:NM_198450:84229251:84234980:+ | E2:chr1:DCAF8:NR_028106:158480373:158480448:− | ER+ Breast Tumor |
| 39 | E3:chr3:ARIH2:NM_006321:48939898:48940250:+ | E1:chr12:TMEM119:NM_181724:107507750:107510302:− | ER+ Breast Tumor |
| 40 | E5:chr11:ARL2:NM_001667:64545768:64546232:+ | E7:chr11:CAPN1:NM_005186:64711261:64711345:+ | HER2+ Breast Tumor |
| 41 | E5:chr10:ARL3:NM_004311:104455092:104455236:− | E1:chr1:MTF2:NM_001164391:93317379:93317676:+ | HER2+ Breast Tumor |
| 42 | E5:chr10:ARL3:NM_004311:104455092:104455236:− | E1:chr1:MTF2:NM_001164391:93317379:93317676:+ | Triple Negative Breast |
| | | | Tumor |
| 43 | E1:chr8:ASAP1:NM_018482:131133534:131136233:− | E1:chr11:MALAT1:NR_002819:65021808:65030513:+ | ER+ Breast Tumor |
| 44 | E2:chr5:BASP1:NM_006317:17328316:17329943:+ | E1:chr17:COL1A1:NM_000088:45616455:45618008:− | ER+ Breast Tumor |
| 45 | | | ER+ Breast Tumor |
| 46 | | | Triple Negative Breast |
| | | | Tumor |
| 47 | | | Triple Negative Breast |
| | | | Tumor |
| 48 | E34:chr1:BAT2L2:NM_015172:169827348:169829273:+ | E48:chr2:COL3A1:NM_000090:189581894:189582192:+ | ER+ Breast Tumor |
| 49 | | | HER2+ Breast Tumor |
| 50 | | | ER+ Breast Tumor |
| 51 | | | ER+ Breast Tumor |
| 52 | | | Triple Negative Breast |
| | | | Tumor |
| 53 | | | Triple Negative Breast |
| | | | Tumor |
| 54 | E8:chr2:C2orf56:NM_001083946:37328781:37329807:+ | E5:chr19:SAMD4B:NM_018028:44539168:44539569:+ | Triple Negative Breast |
| | | | Tumor |
| 55 | E2:chr8:C8orf46:NM_152765:67571225:67571281:+ | E6:chr17:GPATCH8:NM_001002909:39897365:39897438:− | HER2+ Breast Tumor |
| 56 | E10:chr11:CAT:NM_001752:34442227:34442358:+ | E2:chr11:PDHX:NM_001166158:34909526:34909607:+ | Triple Negative Breast |
| | | | Tumor |
| 57 | E1:chrY:CD24:NM_013230:19611913:19614093:− | E4:chr8:GPAA1:NM_003801:145210604:145210752:+ | Triple Negative Breast |
| | | | Tumor |
| 58 | E1:chrY:CD24:NM_013230:19611913:19614093:− | E4:chr8:GPAA1:NM_003801:145210604:145210752:+ | Triple Negative Breast |
| | | | Tumor |
| 59 | E6:chr17:CD68:NM_001040059:7425419:7426153:+ | E1:chr11:NEAT1:NR_028272:64946844:64950577:+ | ER+ Breast Tumor |
| 60 | | | HER2+ Breast Tumor |
| 61 | | | ER+ Breast Tumor |
| 62 | | | ER+ Breast Tumor |
| 63 | E6:chr17:CD68:NM_001040059:7425419:7426153:+ | E5:chr10:PSAP:NM_001042466:73249476:73249663:− | ER+ Breast Tumor |
| 64 | E6:chr17:CD68:NM_001040059:7425419:7426153:+ | E5:chr10:PSAP:NM_001042466:73249476:73249663:− | ER+ Breast Tumor |
| 65 | | | HER2+ Breast Tumor |
| 66 | | | ER+ Breast Tumor |
| 67 | | | Triple Negative Breast |
| | | | Tumor |
| 68 | | | Triple Negative Breast |
| | | | Tumor |
| 69 | E1:chr5:CD74:NM_001025158:149761392:149762006:− | E7:chr12:MBD6:NM_052897:56206615:56207277:+ | Triple Negative Breast |
| | | | Tumor |
| 70 | | | Triple Negative Breast |
| | | | Tumor |
| 71 | E1:chr12:CDK4:NM_000075:56428269:56428667:− | E1:chrX:UBA1:NM_003334:46938144:46938367:+ | Triple Negative Breast |
| | | | Tumor |
| 72 | E7:chr19:CIRBP:NM_001280:1223425:1224171:+ | E1:chr2:UGP2:NM_006759:63922517:63922842:+ | Triple Negative Breast |
| | | | Tumor |
| 73 | E48:chr8:COL14A1:NM_021110:121452571:121453454:+ | E1:chr16:DNAJA2:NM_005880:45546774:45548633:− | ER+ Breast Tumor |
| 74 | E19:chr1:COL16A1:NM_001856:31904224:31904269:− | E5:chr2:COL3A1:NM_000090:189560029:189560110:+ | ER+ Breast Tumor |
| 75 | E7:chr17:COL1A1:NM_000088:45620235:45620343:− | E11:chr19:EPN1:NM_001130071:60898325:60898945:+ | ER+ Breast Tumor |
| 76 | | | HER2+ Breast Tumor |
| 77 | | | ER+ Breast Tumor |
| 78 | | | ER+ Breast Tumor |
| 79 | | | Triple Negative Breast |
| | | | Tumor |
| 80 | E3:chr17:COL1A1:NM_000088:45618676:45618867:− | E5:chr6:FGD2:NM_173558:37089362:37089519:+ | Triple Negative Breast |
| | | | Tumor |
| 81 | E1:chr17:COL1A1:NM_000088:45616455:45618008:− | E1:chr12:FMNL3:NM_175736:48317990:48325953:− | ER+ Breast Tumor |
| 82 | | | ER+ Breast Tumor |
| 83 | E2:chr17:COL1A1:NM_000088:45618137:45618380:− | E3:chr2:GORASP2:NM_015530:171514294:171514498:+ | ER+ Breast Tumor |
| 84 | E51:chr17:COL1A1:NM_000088:45633770:45633999:− | E1:chr14:HEATR5A:NM_015473:30830744:30832569:− | ER+ Breast Tumor |
| 85 | E52:chr7:COL1A2:NM_000089:93897494:93898480:+ | E1:chrX:LAMP2:NM_013995:119454376:119457176:− | ER+ Breast Tumor |
| 86 | | | ER+ Breast Tumor |
| 87 | | | Triple Negative Breast |
| | | | Tumor |
| 88 | E48:chr2:COL3A1:NM_000090:189581894:189582192:+ | E1:chr13:DCLK1:NM_004734:35241122:35246836:− | ER+ Breast Tumor |
| 89 | | | ER+ Breast Tumor |
| 90 | E51:chr2:COL3A1:NM_000090:189584598:189585717:+ | E12:chr11:POLD3:NM_006591:74029256:74031413:+ | ER+ Breast Tumor |
| 91 | E51:chr2:COL3A1:NM_000090:189584598:189585717:+ | E13:chr2:SPATS2L:NM_001100423:201050603:201055231:+ | ER+ Breast Tumor |
| 92 | | | ER+ Breast Tumor |
| 93 | | | Triple Negative Breast |
| | | | Tumor |
| 94 | E51:chr2:COL3A1:NM_000090:189584598:189585717:+ | E1:chr19:ZNF43:NM_003423:21779591:21784449:− | ER+ Breast Tumor |
| 95 | E17:chr8:CPNE3:NM_003909:87639631:87642842:+ | E5:chr14:IFI27:NM_005532:93652531:93652786:+ | Triple Negative Breast |
| | | | Tumor |
| 96 | E12:chr20:CRNKL1:NM_016652:19977983:19978075:− | E12:chr5:RHOBTB3:NM_014899:95154518:95157827:+ | ER+ Breast Tumor |
| 97 | E1:chr11:CTSD:NM_001909:1730560:1731476:− | E1:chr1:EPHA2:NM_004431:16323419:16324402:− | Triple Negative Breast |
| | | | Tumor |
| 98 | E5:chr11:CTSD:NM_001909:1735129:1735362:− | E10:chr7:GNB2:NM_005273:100114253:100114727:+ | Triple Negative Breast |
| | | | Tumor |
| 99 | | | Triple Negative Breast |
| | | | Tumor |
| 100 | E4:chr11:CTSD:NM_001909:1732711:1732834:− | E30:chr19:LTBP4:NM_001042545:45827140:45827565:+ | Triple Negative Breast |
| | | | Tumor |
| 101 | E1:chr11:CTSD:NM_001909:1730560:1731476:− | E1:chr11:PACSIN3:NM_001184974:47155649:47156173:− | Triple Negative Breast |
| | | | Tumor |
| 102 | E1:chr11:CTSD:NM_001909:1730560:1731476:− | E31:chr3:PLXNA1:NM_032242:128235454:128238925:+ | Triple Negative Breast |
| | | | Tumor |
| 103 | | | ER+ Breast Tumor |
| 104 | E4:chr11:CTSD:NM_001909:1732711:1732834:− | E1:chr7:PRKAR1B:NM_002735:555359:556765:− | Triple Negative Breast |
| | | | Tumor |
| 105 | E9:chr11:CTSD:NM_001909:1741597:1741798:− | E4:chr11:TMEM109:NM_024092:60445821:60447489:+ | Triple Negative Breast |
| | | | Tumor |
| 106 | E1:chr1:CTSS:NM_004079:148969175:148972245:− | E2:chr1:GOLPH3L:NM_018178:148900913:148901028:− | HER2+ Breast Tumor |
| 107 | E18:chr11:CTTN:NM_005231:69958779:69960338:+ | E1:chr1:NCRNA00201:NR_026778:243070563:243075269:− | ER+ Breast Tumor |
| 108 | E9:chr17:CWC25:NM_017748:34230679:34230852:− | E6:chr3:ROBO2:NM_001128929:77678178:77678303:+ | HER2+ Breast Tumor |
| 109 | E1:chr17:CYB561:NM_001017917:58863396:58865687:− | E1:chr7:YWHAG:NM_012479:75794043:75797486:− | ER+ Breast Tumor |
| 110 | E1:chr22:CYB5R3:NM_001129819:41343790:41345895:− | E8:chr1:TXNIP:NM_006472:144152539:144153985:+ | ER+ Breast Tumor |
| 111 | | | ER+ Breast Tumor |
| 112 | E6:chr12:DCN:NM_001920:90082512:90082625:− | E1:chr16:VPS35:NM_018206:45251089:45252064:− | ER+ Breast Tumor |
| 113 | | | HER2+ Breast Tumor |
| 114 | E5:chr20:DIDO1:NM_022105:61016006:61016203:− | E11:chr6:REPS1:NM_031922:139289230:139289311:− | HER2+ Breast Tumor |
| 115 | E5:chr20:DIDO1:NM_022105:61016006:61016203:− | E10:chr6:REPS1:NM_001128617:139283866:139283954:− | HER2+ Breast Tumor |
| 116 | E11:chr19:DNM2:NM_004945:10770161:10770248:+ | E2:chr19:PIN1:NM_006221:9810111:9810324:+ | Triple Negative Breast |
| | | | Tumor |
| 117 | | | ER+ Breast Tumor |
| 118 | E22:chr11:EIF4G2:NM_001418:10786827:10787158:− | E8:chr19:RAB8A:NM_005370:16104021:16105445:+ | ER+ Breast Tumor |
| 119 | | | Triple Negative Breast |
| | | | Tumor |
| 120 | E2:chr18:ELAC1:NM_018696:46754764:46754929:+ | E2:chr18:SMAD4:NM_005359:46827287:46827663:+ | ER+ Breast Tumor |
| 121 | | | HER2+ Breast Tumor |
| 122 | | | HER2+ Breast Tumor |
| 123 | | | ER+ Breast Tumor |
| 124 | | | ER+ Breast Tumor |
| 125 | | | ER+ Breast Tumor |
| 126 | | | ER+ Breast Tumor |
| 127 | | | ER+ Breast Tumor |
| 128 | E9:chr1:ELF3:NM_004433:200250959:200252938:+ | E4:chr18:SLC39A6:NM_001099406:31950634:31950740:− | ER+ Breast Tumor |
| 129 | | | ER+ Breast Tumor |
| 130 | E28:chr7:ELN:NM_000501:73115575:73115635:+ | E11:chr12:NCOR2:NM_006312:123390504:123390703:− | Triple Negative Breast |
| | | | Tumor |
| 131 | E1:chr16:EMP2:NM_001424:10529780:10534450:− | E1:chr12:KRT81:NM_002281:50965963:50966544:− | Triple Negative Breast |
| | | | Tumor |
| 132 | E7:chr21:FAM3B:NM_058186:41642388:41642521:+ | E14:chr7:GLI3:NM_000168:42229253:42229419:− | HER2+ Breast Tumor |
| 133 | E1:chrX:FLNA:NM_001110556:153230093:153230598:− | E1:chr22:SBF1:NM_002972:49230298:49232535:− | Triple Negative Breast |
| | | | Tumor |
| 134 | E9:chr12:GAPDH:NM_002046:6517527:6517797:+ | E7:chr17:KRT13:NM_002274:36914833:36915391:− | Triple Negative Breast |
| | | | Tumor |
| 135 | E9:chr12:GAPDH:NM_002046:6517527:6517797:+ | E7:chr6:MRPS18B:NM_014046:30701257:30702153:+ | Triple Negative Breast |
| | | | Tumor |
| 136 | E1:chr10:GATA3:NM_002051:8136672:8136860:+ | E1:chr2:RHOB:NM_004040:20510315:20512682:+ | Triple Negative Breast |
| | | | Tumor |
| 137 | | | ER+ Breast Tumor |
| 138 | | | Triple Negative Breast |
| | | | Tumor |
| 139 | E2:chr19:GEMIN7:NM_001007270:50275004:50275127:+ | E2:chr8:SLC39A14:NM_001135154:22318153:22318438:+ | Triple Negative Breast |
| | | | Tumor |
| 140 | E1:chr19:GEMIN7:NM_001007270:50274357:50274377:+ | E2:chr8:SLC39A14:NM_001135154:22318153:22318438:+ | Triple Negative Breast |
| | | | Tumor |
| 141 | E1:chr1:GNB1:NM_002074:1706588:1708352:− | E3:chr3:TRH:NM_007117:131178231:131179466:+ | Triple Negative Breast |
| | | | Tumor |
| 142 | E1:chr3:GNB4:NM_021629:180596569:180601801:− | E5:chr2:PTMA:NM_001099285:232285757:232286494:+ | Triple Negative Breast |
| | | | Tumor |
| 143 | E2:chr3:GPR128:NM_032787:101831131:101831245:+ | E3:chr3:TFG:NM_001007565:101921508:101921592:+ | HER2+ Breast Tumor |
| 144 | E10:chr2:HDLBP:NM_203346:241827689:241827908:− | E7:chr17:NTN1:NM_004822:9083681:9088042:+ | Triple Negative Breast |
| | | | Tumor |
| 145 | E8:chr6:HLA-E:NM_005516:30568504:30569960:+ | E6:chr10:TSPAN14:NM_001128309:82267640:82272371:+ | Triple Negative Breast |
| | | | Tumor |
| 146 | E8:chr6:HLA-E:NM_005516:30568504:30569960:+ | E6:chr10:TSPAN14:NM_001128309:82267640:82272371:+ | Triple Negative Breast |
| | | | Tumor |
| 147 | E6:chr6:HMGN3:NM_138730:80000981:80001174:− | E2:chr6:PAQR8:NM_133367:52375918:52380534:+ | ER+ Breast Tumor |
| 148 | E6:chr5:HNRNPH1:NM_005520:178977120:178977256:− | E7:chr18:VAPA:NM_003574:9944049:9950018:+ | HER2+ Breast Tumor |
| 149 | E1:chr1:HNRNPU:NM_031844:243080224:243084428:− | E7:chr7:TES:NM_152829:115684583:115686073:+ | ER+ Breast Tumor |
| 150 | E11:chr6:HSP90AB1:NM_007355:44328759:44329093:+ | E1:chr17:PCGF2:NM_007144:34143675:34145379:− | ER+ Breast Tumor |
| 151 | E1:chr11:IGF2:NM_000612:2106922:2111029:− | E1:chr11:MALAT1:NR_002819:65021808:65030513:+ | ER+ Breast Tumor |
| 152 | | | HER2+ Breast Tumor |
| 153 | | | HER2+ Breast Tumor |
| 154 | E1:chr11:IGF2:NM_000612:2106922:2111029:− | E1:chr11:MALAT1:NR_002819:65021808:65030513:+ | ER+ Breast Tumor |
| 155 | E1:chr2:IGFBP5:NM_000599:217245072:217249850:− | E9:chr12:RAB3IP:NM_001024647:68495410:68503251:+ | ER+ Breast Tumor |
| 156 | E5:chr4:IGFBP7:NM_001553:57670799:57671296:− | E1:chr16:MAF:NM_001031804:78185246:78192123:− | ER+ Breast Tumor |
| 157 | E2:chr22:IGLL5:NM_001178126:21565879:21565998:+ | E11:chr22:LOC96610:NR_027293:21007018:21007324:+ | ER+ Breast Tumor |
| 158 | E2:chr22:IGLL5:NM_001178126:21565879:21565998:+ | E11:chr22:LOC96610:NR_027293:21007018:21007324:+ | Triple Negative Breast |
| | | | Tumor |
| 159 | | | HER2+ Breast Tumor |
| 160 | | | HER2+ Breast Tumor |
| 161 | | | HER2+ Breast Tumor |
| 162 | | | HER2+ Breast Tumor |
| 163 | | | HER2+ Breast Tumor |
| 164 | | | HER2+ Breast Tumor |
| 165 | | | ER+ Breast Tumor |
| 166 | | | ER+ Breast Tumor |
| 167 | | | ER+ Breast Tumor |
| 168 | | | ER+ Breast Tumor |
| 169 | | | Triple Negative Breast |
| | | | Tumor |
| 170 | | | Triple Negative Breast |
| | | | Tumor |
| 171 | | | Triple Negative Breast |
| | | | Tumor |
| 172 | | | Triple Negative Breast |
| | | | Tumor |
| 173 | | | Triple Negative Breast |
| | | | Tumor |
| 174 | E2:chr22:IGLL5:NR_033661:21567554:21568011:+ | E2:chr8:SFTPC:NM_001172357:22076031:22076190:+ | Triple Negative Breast |
| | | | Tumor |
| 175 | E4:chr16:IRX3:NM_024336:52877196:52877879:− | E4:chr19:USF2:NM_003367:40452545:40452746:+ | Triple Negative Breast |
| | | | Tumor |
| 176 | E25:chr17:ITGA3:NM_005501:45521472:45522848:+ | E1:chr2:KHK:NM_000221:27163114:27163723:+ | Triple Negative Breast |
| | | | Tumor |
| 177 | E4:chr22:JOSD1:NM_014876:37425753:37426405:− | E3:chr22:RPS19BP1:NM_194326:38258345:38258474:− | HER2+ Breast Tumor |
| 178 | E4:chr18:KCTD1:NM_001142730:22335033:22335212:− | E2:chr18:LOC728606:NR_024259:22537353:22537600:− | ER+ Breast Tumor |
| 179 | E4:chr18:KCTD1:NM_001142730:22335033:22335212:− | E2:chr18:LOC728606:NR_024259:22537353:22537600:− | Triple Negative Breast |
| | | | Tumor |
| 180 | | | HER2+ Breast Tumor |
| 181 | | | HER2+ Breast Tumor |
| 182 | | | ER+ Breast Tumor |
| 183 | | | ER+ Breast Tumor |
| 184 | | | ER+ Breast Tumor |
| 185 | | | Triple Negative Breast |
| | | | Tumor |
| 186 | E8:chr1:KCTD3:NM_016121:213819874:213819965:+ | E5:chr14:TXNDC16:NM_020784:51993557:51993642:− | HER2+ Breast Tumor |
| 187 | E1:chr10:KIAA1217:NM_019590:24537725:24538198:+ | E1:chr14:SERPINA1:NM_001002236:93912836:93914730:− | ER+ Breast Tumor |
| 188 | E7:chr12:KRT18:NM_199187:51632169:51632393:+ | E2:chr8:PLEC:NM_000445:145068659:145072040:− | Triple Negative Breast |
| | | | Tumor |
| 189 | | | Triple Negative Breast |
| | | | Tumor |
| 190 | E8:chr12:KRT4:NM_002272:51491813:51492028:− | E2:chr8:RPL8:NM_000973:145986543:145986659:− | Triple Negative Breast |
| | | | Tumor |
| 191 | E10:chr1:LAMB3:NM_001017402:207865615:207865994:− | E16:chr1:RALGPS2:NM_152663:177129676:177129782:+ | HER2+ Breast Tumor |
| 192 | E14:chr14:LGMN:NM_001008530:92277159:92277277:− | E5:chr12:NAP1L1:NM_139207:74730577:74730700:− | Triple Negative Breast |
| | | | Tumor |
| 193 | | | HER2+ Breast Tumor |
| 194 | E19:chr3:LRIG1:NM_015541:66633303:66633535:− | E9:chr18:SLC39A6:NM_012319:31960179:31960977:− | ER+ Breast Tumor |
| 195 | E1:chr11:MALAT1:NR_002819:65021808:65030513:+ | E1:chr1:PTP4A2:NM_080391:32146379:32147148:− | ER+ Breast Tumor |
| 196 | | | Triple Negative Breast |
| | | | Tumor |
| 197 | | | Triple Negative Breast |
| | | | Tumor |
| 198 | E1:chr11:MALAT1:NR_002819:65021808:65030513:+ | E17:chr7:TAX1BP1:NM_001079864:27834771:27835911:+ | ER+ Breast Tumor |
| 199 | | | ER+ Breast Tumor |
| 200 | E4:chr14:MAPK1IP1L:NM_144578:54601086:54606665:+ | E24:chr2:XPO1:NM_003400:61614410:61614542:− | ER+ Breast Tumor |
| 201 | E1:chr12:MGP:NM_001190839:14925381:14926481:− | E4:chr17:NCRNA00188:NR_027159:16285406:16286063:+ | ER+ Breast Tumor |
| 202 | | | ER+ Breast Tumor |
| 203 | | | ER+ Breast Tumor |
| 204 | E1:chr12:MGP:NM_001190839:14925381:14926481:− | E18:chrX:REPS2:NM_004726:17075456:17081324:+ | ER+ Breast Tumor |
| 205 | E4:chr20:MKKS:NM_170784:10341177:10342579:− | E7:chr14:PCNX:NM_014982:70525036:70525169:+ | Triple Negative Breast |
| | | | Tumor |
| 206 | E8:chr19:MRPL4:NM_146388:10230284:10231736:+ | E4:chr17:SLC16A3:NM_001042423:77788302:77789058:+ | Triple Negative Breast |
| | | | Tumor |
| 207 | E4:chr14:MRPL52:NM_181307:22373220:22374086:+ | E1:chr17:USP22:NM_015276:20843497:20846978:− | ER+ Breast Tumor |
| 208 | E3:chr12:MUCL1:NM_058173:53536820:53536943:+ | E1:chr17:RPL23:NM_000978:34259846:34259993:− | HER2+ Breast Tumor |
| 209 | | | HER2+ Breast Tumor |
| 210 | | | ER+ Breast Tumor |
| 211 | | | Triple Negative Breast |
| | | | Tumor |
| 212 | E38:chr11:NAV2:NM_145117:20096254:20099723:+ | E1:chr2:WDFY1:NM_020830:224448308:224451691:− | Triple Negative Breast |
| | | | Tumor |
| 213 | E6:chr17:NPLOC4:NM_017921:77166406:77166567:− | E2:chr17:PDE6G:NR_026872:77229079:77229120:− | Triple Negative Breast |
| | | | Tumor |
| 214 | E8:chr2:OLA1:NM_001011708:174796006:174796134:− | E3:chr17:ORMDL3:NM_139280:35333808:35334004:− | HER2+ Breast Tumor |
| 215 | E4:chr15:PAQR5:NM_001104554:67459275:67459403:+ | E6:chr15:THSD4:NM_024817:69491079:69491216:+ | ER+ Breast Tumor |
| 216 | E1:chr15:PDIA3:NM_005313:41825881:41826196:+ | E1:chr7:YWHAG:NM_012479:75794043:75797486:− | Triple Negative Breast |
| | | | Tumor |
| 217 | E42:chr2:PIKFYVE:NM_015040:208928158:208931720:+ | E1:chr12:TMEM119:NM_181724:107507750:107510302:− | Triple Negative Breast |
| | | | Tumor |
| 218 | E6:chr15:PKM2:NM_182470:70288015:70288286:− | E1:chr2:SEMA4C:NM_017789:96889199:96890919:− | Triple Negative Breast |
| | | | Tumor |
| 219 | E2:chr8:PLEC:NM_000445:145068659:145072040:− | E1:chr1:PLEKHM2:NM_015164:15883413:15883700:+ | Triple Negative Breast |
| | | | Tumor |
| 220 | E1:chr8:PLEC:NM_000445:145061308:145068551:− | E3:chr19:RPS15:NM_001018:1391017:1391252:+ | Triple Negative Breast |
| | | | Tumor |
| 221 | E1:chr13:POSTN:NM_006475:37034719:37035507:− | E1:chr10:TM9SF3:NM_020123:98267856:98272077:− | ER+ Breast Tumor |
| 222 | E1:chr13:POSTN:NM_006475:37034719:37035507:− | E1:chr1:TRIM33:NM_015906:114736921:114742005:− | ER+ Breast Tumor |
| 223 | E15:chr4:PROM1:NM_001145849:15617258:15617411:− | E2:chr4:TAPT1:NM_153365:15777353:15777514:− | Triple Negative Breast |
| | | | Tumor |
| 224 | E1:chr3:RBM6:NM_005777:49952480:49952662:+ | E2:chr3:SLC38A3:NM_006841:50226585:50226737:+ | HER2+ Breast Tumor |
| 225 | E1:chr14:RNASE1:NM_198235:20339354:20340092:− | E38:chr14:TEP1:NM_007110:19925903:19926062:− | HER2+ Breast Tumor |
| 226 | E1:chr1:RNF11:NM_014372:51474532:51475139:+ | E1:chr5:STC2:NM_003714:172674331:172677858:− | ER+ Breast Tumor |
| 227 | | | ER+ Breast Tumor |
| 228 | E2:chr17:RPL19:NM_000981:34610991:34611098:+ | E4:chr19:RPS16:NM_001020:44618086:44618188:− | Triple Negative Breast |
| | | | Tumor |
| 229 | | | HER2+ Breast Tumor |
| 230 | | | Triple Negative Breast |
| | | | Tumor |
| 231 | E4:chr19:RPS16:NM_001020:44618086:44618188:− | E3:chr2:TMSB10:NM_021103:84987023:84987310:+ | Triple Negative Breast |
| | | | Tumor |
| 232 | | | HER2+ Breast Tumor |
| 233 | | | HER2+ Breast Tumor |
| 234 | | | Triple Negative Breast |
| | | | Tumor |
| 235 | E5:chr22:SFI1:NM_001007467:30272846:30272957:+ | E4:chr22:YPEL1:NM_013313:20394916:20395197:− | ER+ Breast Tumor |
| 236 | E1:chr17:SLC9A3R1:NM_004252:70256357:70257021:+ | E2:chr7:TNRC18:NM_001080495:5315031:5315106:− | Triple Negative Breast |
| | | | Tumor |
| 237 | E2:chr2:SOCS5:NM_014011:46839161:46843431:+ | E1:chr2:TTC7A:NM_020458:47021816:47022368:+ | Triple Negative Breast |
| | | | Tumor |
| 238 | | | ER+ Breast Tumor |
| 239 | | | Triple Negative Breast |
| | | | Tumor |
| 240 | E1:chr5:SPARC:NM_003118:151021201:151023353:− | E7:chr8:TRPS1:NM_014112:116749945:116750402:− | ER+ Breast Tumor |
| 241 | E5:chr5:SPARC:NM_003118:151029417:151029538:− | E7:chr8:TRPS1:NM_014112:116749945:116750402:− | ER+ Breast Tumor |
| 242 | | | HER2+ Breast Tumor |
| 243 | | | ER+ Breast Tumor |
| 244 | | | ER+ Breast Tumor |
| 245 | E3:chr6:SRPK1:NM_003137:35918289:35918359:− | E1:chr6:UBR2:NM_001184801:42639737:42640113:+ | ER+ Breast Tumor |
| 246 | E1:chr8:YWHAZ:NM_001135700:101999980:102002156:− | E3:chrX:ZBTB33:NM_001184742:119271296:119276279:+ | Triple Negative Breast |
| | | | Tumor |
| 247 | | | Triple Negative Breast |
| | | | Tumor |

In the “Row #” column for Table 8, sentinel transcripts are identified with an * symbol; redundant transcripts are identified with a $ symbol; and transcripts that are expressed as multiple isoforms are identified with a + symbol.

### Tumor Subtype Distribution of Fusion Transcripts

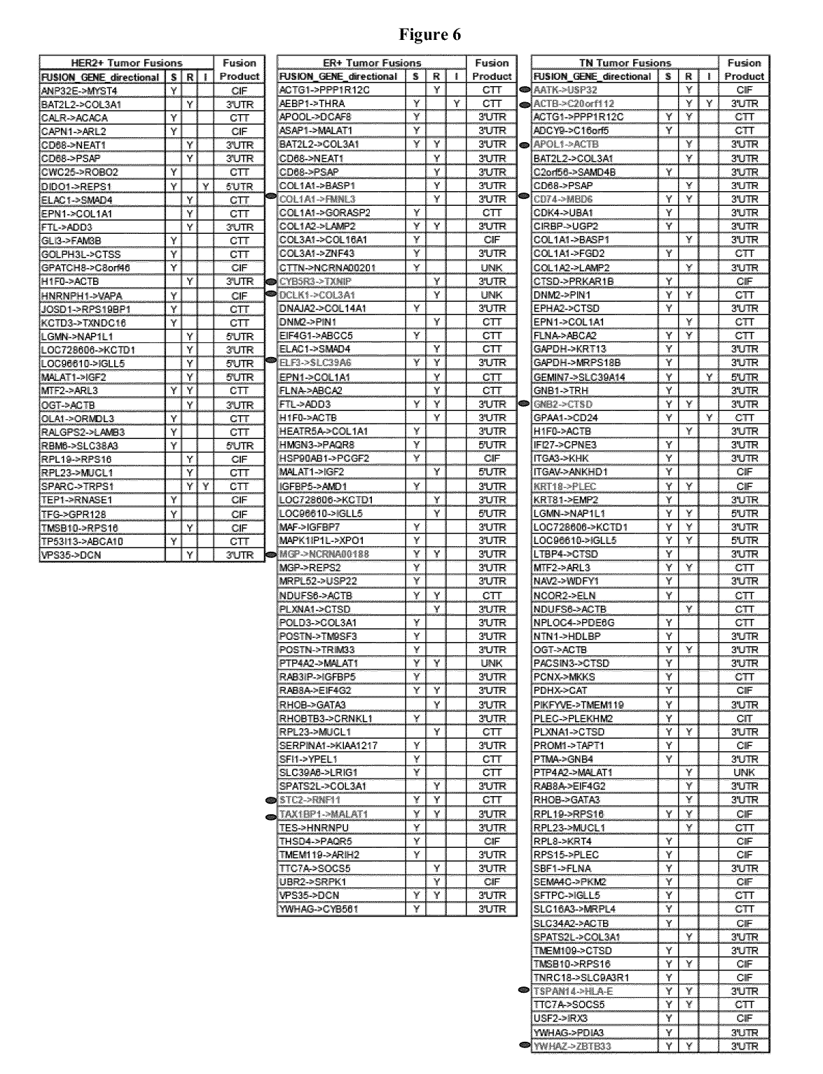

Every tumor expressed at least one redundant fusion transcript, with a range of 1-13 redundant transcripts/tumor (Table 9). Among the redundant transcripts, seven were uniquely expressed in ER+ tumors and eight in TN tumors (labeled with oval symbols in FIG. 6), but no redundant transcript was exclusively expressed in HER2+ tumors. Private transcripts were detected at a range of 0-12/tumor (Table 9). ER+ and TN tumors expressed similar numbers of fusion transcripts, whereas HER2+ tumors expressed significantly fewer fusions (Table 9). However, a few HER2+ tumors expressed levels of fusions that were comparable to those observed in ER+ or TN tumors (see, e.g., HER2+ tumor s_{—}29 in Table 8). It is possible that the expression of large numbers of fusion transcripts is indicative of a subset of HER2+ tumors that have unusually high genomic instability, with implications for therapeutic response. Fusion transcripts represented a heretofore underappreciated class of genomic features that may have considerable potential as biomarkers or therapeutic targets in breast cancer.

TABLE 9
Distribution of fusion transcripts among tumors subtypes. Tumor subtype-
specific incidence was abstracted from Table 8. Statistical analysis was performed by
ANOVA.

| | | | Number of | | | Number of | Subtype | Fusions |
|---|---|---|---|---|---|---|---|---|
| | | | Genes in | | Range | Genes in | Specific | with |
| Tumor | Private | Range Private | Private | Redundant | Redundant. | Redundant | Redundant | Multiple |
| Subtype | Fusions | Fusions/Tumor | Fusions | Fusions | Fusions/Tumor | Fusions | Fusions | Isoforms |
| All | 86 | 0 to 12 | 149 | 45 | 1 to 13 | 76 | — | 6 |
| Tumors | | | | | | | | |
| HER2 | 17⁽¹⁾ | 0 to 5 | 34 | 19⁽²⁾ | 1 to 9 | 33 | 0 | 1 |
| Tumors | | | | | | | | |
| ER+ | 30 | 0 to 9 | 51 | 32 | 2 to 12 | 55 | 7 | 2 |
| Tumors | | | | | | | | |
| TN | 39 | 2 to 12 | 68 | 32 | 3 to 13 | 53 | 8 | 3 |
| Tumors | | | | | | | | |

⁽¹⁾p = 0.25 re. ER+, p = 0.036 re. TN
⁽²⁾p = 0.006 re. ER+, p = 0.02 re. TN

### Chromosomal Distribution of Fusion Transcript Partners

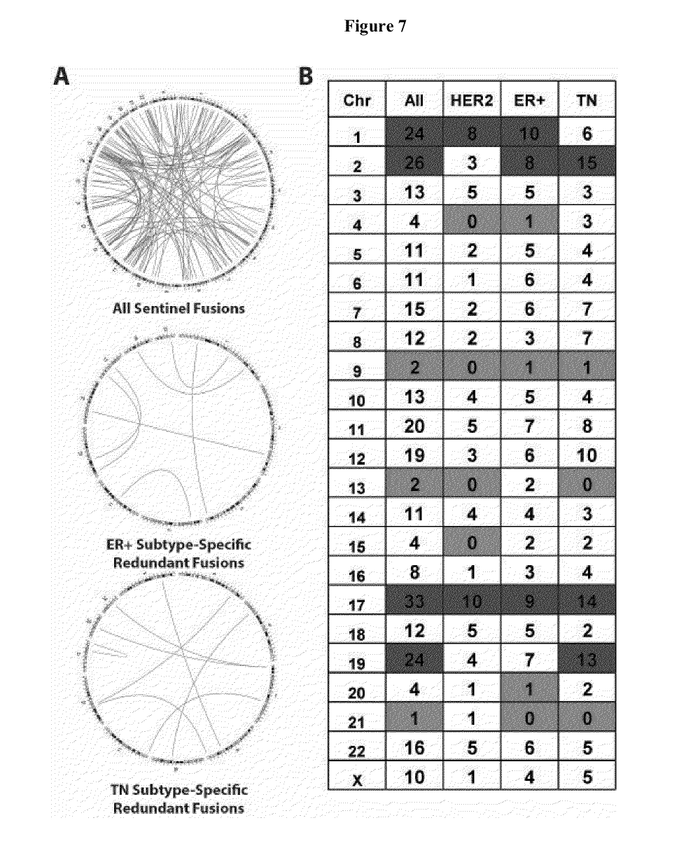

The chromosomal mapping distribution of the sentinel fusions was clearly non-random (FIG. 7A). A disproportionately large number of fusion transcript partners were located on chromosomes 1, 2, 17, and 19 (FIG. 7B), whereas relatively few fusion transcript partners are located on chromosomes 4, 9, 13, 15, 20, and 21. It was difficult, because of the relatively small numbers, to make any rigorous conclusions with respect to tumor-subtype-specific distribution of fusion transcripts. However, chromosome 19 appeared to be a ‘hot spot’ for TN tumors. Circos plots of ER+ specific and TN specific redundant fusion gene partners (FIG. 7A) indicated that there is a subtype-specific fusion transcript geography, suggesting a functional link between breast tumor subtype and formation of fusion transcripts. The observation that HER2+ tumors, as a group, express significantly fewer fusion transcripts was consistent with this hypothesis.

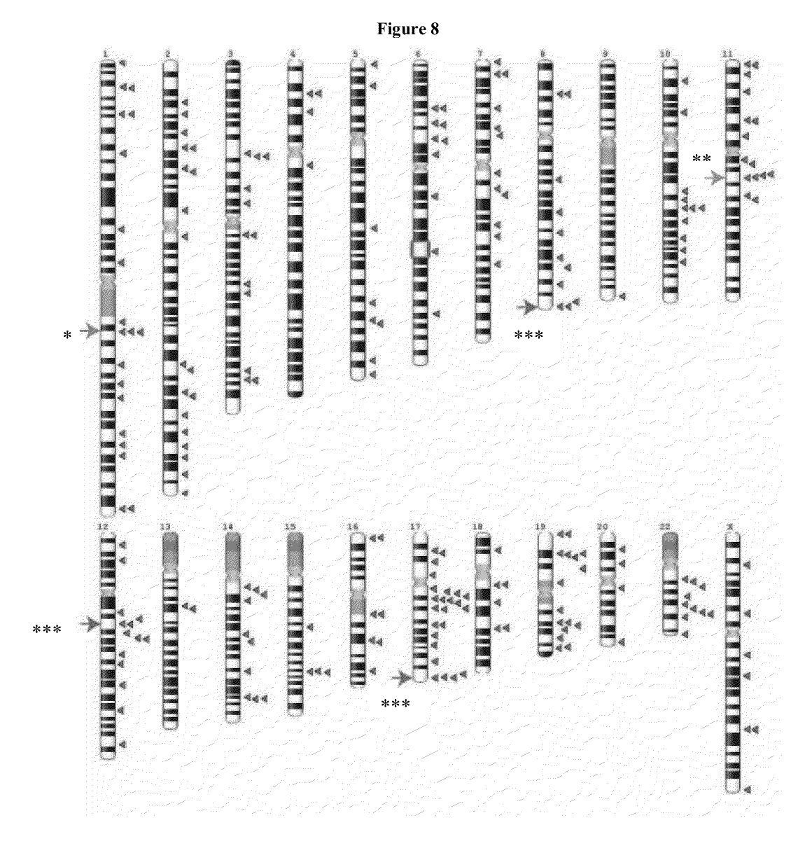

A number of distinct clusters emerged when the fusion partner genes were mapped to genomic loci (FIG. 8). Two major clusters were observed on chromosome 17, mapping to 17q21-q23, and 17q25. Both of these regions are well-known to undergo copy number variation in breast cancer. All of the chromosome 19 fusion partners in TN tumors mapped to clusters located in the vicinity of 19p13 or 19q13. One large cluster of genes at 11q13.1-q13.4 was restricted to ER+ tumors (arrow in FIG. 8 labeled with two asterisks), a small cluster of genes at 1q21.2-q21.3 was restricted to HER2+ tumors (arrow in FIG. 8 labeled with one asterisk), and genes that clustered at 8q24.3, 12q13.13, and 17q25.1-q25.3 were restricted to TN tumors (arrows in FIG. 8 labeled with three asterisks).

Limited data from genomic analysis of both breast cancer cell lines (Edgren et al., Genome Biol., 12:R6 (2011)) and tumors (Inaki et al., Genome Res., 21:676-87 (2011); and Stephens et al., Nature, 462:1005-10 (2009)) indicate that genomic rearrangement is the primary mechanism whereby most fusion transcripts are generated. Furthermore, review of the array comparative genomic hybridization (aCGH) data on breast cancer revealed that many of the fusion partners that were identified map to regions that are known to undergo copy number gain or loss in breast tumors. This correlation was evident when one considers chromosome 17, which contained 33 genes that contributed to fusion transcripts. Among these genes, six mapped to a cluster at 17q12, 5 to 17q21, and 6 to 17q25. All three of these loci are known to undergo copy number variation in breast cancer (Stephens et al., Nature, 462:1005-10 (2009); Adelaide et al., Cancer Res., 67:11565-75 (2007); Andre et al., Clin. Cancer Res., 15:441-51 (2009); and Bae et al., World J. Surg. Oncol., 8:32 (2010)). The distribution of fusion partners on chromosome 19 was even more striking. All of the genes map to either 19p12-p13 or 19q13. Both aCGH and genome wide association data indicated that these two regions are important in breast cancer, particularly the triple negative subtype (Antoniou et al., Nat. Genet., 42:885-92 (2010); and Yang et al., Genes Chromosomes Cancer, 41:250-6 (2004)). Based on these considerations, most of the fusion transcripts appeared to arise due to chromosomal rearrangements and therefore marked areas of local chromosomal instability.

### Structure and Potential Functional Significance of Predicted Fusion Transcript Products

SnowShoes_FTD assembled the predicted nucleotide sequences of the candidate fusion transcripts and translated that sequence into the predicted amino acid sequences of the putative fusion proteins (Table 10). Fusion transcripts in breast cancer cell lines fall into several broad categories based on the location with the transcription unit wherein the fusion occurs. A small number of fusions occurred in 5′ UTR regions (FIG. 6), placing the coding sequence of the 3′ fusion partner under the control of the promoter from the 5′ fusion partner. A ‘promoter swap’ event of this sort was associated with ERBB2 overexpression in a breast cancer cell line derived from a HER2+ tumor.

TABLE 10
Predicted nucleotide sequence of candidate fusion transcripts and predicted amino acid sequence of translations products.

| # | FUSION | Transcripts | In frame | Junction Point Mutations | Boundary Exon 5′ Gene |
|---|---|---|---|---|---|
| 1 | KCTD3->TXNDC16 | NM_016121->NM_001160047 | | | E8: chr1: 213819874-213819965 |
| 2 | KCTD3->TXNDC16 | NM_016121->NM_020784 | | | E8: chr1: 213819874-213819965 |
| 3 | ITGB4->ACTB | NM_000213->NM_001101 | | | E153: chr17: 71261440-71261650 |
| 4 | ITGB4->ACTB | NM_000213->NM_001101 | | | E153: chr17: 71261440-71261650 |
| 5 | PDHX->CAT | NM_003477->NM_001752 | YES | | E2: chr11: 34909526-34909607 |
| 6 | PDHX->CAT | NM_001135024->NM_001752 | YES | | E2: chr11: 34909526-34909607 |
| 7 | PDHX->CAT | NM_001166158->NM_001752 | YES | | E2: chr11: 34909526-34909607 |
| 8 | EPN1->COL1A1 | NM_013333->NM_000088 | | | E20: chr19: 60898325-60898945 |
| 9 | EPN1->COL1A1 | NM_001130072->NM_000088 | | | E22: chr19: 60898325-60898945 |
| 10 | EPN1->COL1A1 | NM_001130071->NM_000088 | | | E22: chr19: 60898325-60898945 |
| 11 | CWC25->ROBO2 | NM_017748->NM_002942 | | | E2: chr17: 34230679-34230852 |
| 12 | CWC25->ROBO2 | NM_017748->NM_001128929 | YES | AGC->AAC(S->N) | E2: chr17: 34230679-34230852 |
| 13 | LTBP4->CTSD | NM_003573->NM_001909 | YES | | E33: chr19: 45827140-45827565 |
| 14 | LTBP4->CTSD | NM_001042544->NM_001909 | YES | | E33: chr19: 45827140-45827565 |
| 15 | LTBP4->CTSD | NM_001042545->NM_001909 | YES | | E30: chr19: 45827140-45827565 |
| 16 | MIR1204->PVT1 | NR_031609->NR_003367 | YES | | E1: chr8: 128877389-128877456 |
| 17 | MIR1204->PVT1 | NR_031609->NR_003367 | YES | | E1: chr8: 128877389-128877456 |
| 18 | MIR1204->PVT1 | NR_031609->NR_003367 | YES | | E1: chr8: 128877389-128877456 |
| 19 | MIR1204->PVT1 | NR_031609->NR_003367 | YES | | E1: chr8: 128877389-128877456 |
| 20 | MIR1204->PVT1 | NR_031609->NR_003367 | YES | | E1: chr8: 128877389-128877456 |
| 21 | MIR1204->PVT1 | NR_031609->NR_003367 | YES | | E1: chr8: 128877389-128877456 |
| 22 | MIR1204->PVT1 | NR_031609->NR_003367 | YES | | E1: chr8: 128877389-128877456 |
| 23 | MIR1204->PVT1 | NR_031609->NR_003367 | YES | | E1: chr8: 128877389-128877456 |
| 24 | PTMA->SDC4 | NM_001099285->NM_002999 | YES | | E40: chr2: 232285757-232286494 |
| 25 | PTMA->SDC4 | NM_002823->NM_002999 | YES | | E40: chr2: 232285757-232286494 |
| 26 | SERINC2->KRT5 | NM_178865->NM_000424 | | | E7: chr1: 31674411-31674502 |
| 27 | NPLOC4->PDE6G | NM_017921->NM_002602 | | | E12: chr17: 77166406-77166567 |
| 28 | NPLOC4->PDE6G | NM_017921->NR_026872 | (part of | | E12: chr17: 77166406-77166567 |
| | | | NPLOC4) | | |
| 29 | SFN->CTSD | NM_006142->NM_001909 | YES | | E2: chr1: 27062219-27063534 |
| 30 | KRT7->KRT14 | NM_005556->NM_000526 | YES | | E54: chr12: 50928641-50928976 |
| 31 | FKBP1A->SDCBP2 | NM_000801->NM_080489 | YES | | E2: chr20: 1321477-1321525 |
| 32 | FKBP1A->SDCBP2 | NM_054014->NM_080489 | | | E2: chr20: 1321477-1321525 |
| 33 | FKBP1A->SDCBP2 | NM_000801->NM_080489 | | | E2: chr20: 1321477-1321525 |
| 34 | FKBP1A->SDCBP2 | NM_054014->NM_080489 | | | E2: chr20: 1321477-1321525 |
| 35 | GLI3->FAM3B | NM_000168->NM_206964 | | | E2: chr7: 42229253-42229419 |
| 36 | GLI3->FAM3B | NM_000168->NM_058186 | | | E2: chr7: 42229253-42229419 |
| 37 | KRT7->ACTB | NM_005556->NM_001101 | YES | | E52: chr12: 50925462-50925683 |
| 38 | ILF3->KRT5 | NM_012218->NM_000424 | YES | | E38: chr19: 10659021-10659384 |
| 39 | ILF3->KRT5 | NM_017620->NM_000424 | YES | | E38: chr19: 10659021-10659384 |
| 40 | SLC39A6->LRIG1 | NM_012319->NM_015541 | | | E2: chr18: 31960179-31960977 |
| 41 | COL1A1->FMNL3 | NM_000088->NM_175736 | YES | | E51: chr17: 45616455-45618008 |
| 42 | COL1A1->FMNL3 | NM_000088->NM_198900 | YES | | E51: chr17: 45616455-45618008 |
| 43 | COL1A2->MAZ | NM_000089->NM_002383 | | | E620: chr7: 93894435-93894543 |
| 44 | COL1A2->MAZ | NM_000089->NM_001042539 | | | E620: chr7: 93894435-93894543 |
| 45 | PTRF->TAPBP | NM_012232->NM_003190 | YES | | E2: chr17: 37807994-37810932 |
| 46 | PTRF->TAPBP | NM_012232->NM_172209 | YES | | E2: chr17: 37807994-37810932 |
| 47 | LOC96610->IGLL5 | NR_027293->NM_001178126 | NOT Evaluated | | E11: chr22: 21007018-21007324 |
| 48 | LOC96610->IGLL5 | NR_027293->NM_001178126 | NOT Evaluated | | E11: chr22: 21007018-21007324 |
| 49 | VPS35->DCN | NM_018206->NM_133506 | YES | | E17: chr16: 45251089-45252064 |
| 50 | VPS35->DCN | NM_018206->NM_133503 | YES | | E17: chr16: 45251089-45252064 |
| 51 | VPS35->DCN | NM_018206->NM_001920 | YES | | E17: chr16: 45251089-45252064 |
| 52 | VPS35->DCN | NM_018206->NM_133506 | YES | | E17: chr16: 45251089-45252064 |
| 53 | VPS35->DCN | NM_018206->NM_133503 | YES | | E17: chr16: 45251089-45252064 |
| 54 | VPS35->DCN | NM_018206->NM_001920 | YES | | E17: chr16: 45251089-45252064 |
| 55 | GAPDH->KRT13 | NM_002046->NM_153490 | YES | | E108: chr12: 6517527-6517797 |
| 56 | GAPDH->KRT13 | NM_002046->NM_002274 | YES | | E108: chr12: 6517527-6517797 |
| 57 | SPATS2L->COL3A1 | NM_015535->NM_000090 | YES | | E13: chr2: 201050603-201055231 |
| 57 | SPATS2L->COL3A1 | NM_001100422->NM_000090 | YES | | E13: chr2: 201050603-201055231 |
| 59 | SPATS2L->COL3A1 | NM_001100424->NM_000090 | YES | | E12: chr2: 201050603-201055231 |
| 60 | SPATS2L->COL3A1 | NM_001100423->NM_000090 | YES | | E13: chr2: 201050603-201055231 |
| 61 | YWHAG->CYB561 | NM_012479->NM_001017917 | YES | | E2: chr7: 75794043-75797486 |
| 62 | YWHAG->CYB561 | NM_012479->NM_001017916 | YES | | E2: chr7: 75794043-75797486 |
| 63 | YWHAG->CYB561 | NM_012479->NM_001915 | YES | | E2: chr7: 75794043-75797486 |
| 64 | LASP1->ACTN1 | NM_006148->NM_001102 | YES | | E7: chr17: 34328383-34331548 |
| 65 | LASP1->ACTN1 | NM_006148->NM_001130004 | YES | | E7: chr17: 34328383-34331548 |
| 66 | LASP1->ACTN1 | NM_006148->NM_001130005 | YES | | E7: chr17: 34328383-34331548 |
| 67 | ANP32E->MYST4 | NM_030920->NM_012330 | YES | | E7: chr1: 148457341-148459687 |
| 68 | ANP32E->MYST4 | NM_001136478->NM_012330 | YES | | E6: chr1: 148457341-148459687 |
| 69 | ANP32E->MYST4 | NM_001136479->NM_012330 | YES | | E7: chr1: 148457341-148459687 |
| 70 | COL1A1->BASP1 | NM_000088->NM_006317 | YES | | E51: chr17: 45616455-45618008 |
| 71 | COL1A1->MBD6 | NM_000088->NM_052897 | YES | | E51: chr17: 45616455-45618008 |
| 72 | TSPAN14->HLA-E | NM_030927->NM_005516 | YES | | E9: chr10: 82267640-82272371 |
| 73 | TSPAN14->HLA-E | NM_001128309->NM_005516 | YES | | E6: chr10: 82267640-82272371 |
| 74 | TSPAN14->HLA-E | NM_030927->NM_005516 | YES | | E9: chr10: 82267640-82272371 |
| 75 | TSPAN14->HLA-E | NM_001128309->NM_005516 | YES | | E6: chr10: 82267640-82272371 |
| 76 | COL1A1->PLEC | NM_000088->NM_201383 | | | E44: chr17: 45620455-45620509 |
| 77 | COL1A1->PLEC | NM_000088->NM_201384 | (part of | | E44: chr17: 45620455-45620509 |
| | | | COL1A1) | | |
| 78 | COL1A1->PLEC | NM_000088->NM_000445 | (part of | | E44: chr17: 45620455-45620509 |
| | | | COL1A1) | | |
| 79 | COL1A1->PLEC | NM_000088->NM_201381 | | | E44: chr17: 45620455-45620509 |
| 80 | COL1A1->PLEC | NM_000088->NM_201382 | | | E44: chr17: 45620455-45620509 |
| 81 | COL1A1->PLEC | NM_000088->NM_201380 | | | E44: chr17: 45620455-45620509 |
| 82 | COL1A1->PLEC | NM_000088->NM_201378 | | | E44: chr17: 45620455-45620509 |
| 83 | COL1A1->PLEC | NM_000088->NM_201379 | | | E44: chr17: 45620455-45620509 |
| 84 | COL1A1->PLEC | NM_000088->NM_201383 | | | E44: chr17: 45620455-45620509 |
| 85 | COL1A1->PLEC | NM_000088->NM_201384 | | | E44: chr17: 45620455-45620509 |
| 86 | COL1A1->PLEC | NM_000088->NM_000445 | | | E44: chr17: 45620455-45620509 |
| 87 | MMP14->ACTB | NM_004995->NM_001101 | | | E7: chr14: 22383419-22383558 |
| 88 | KRT5->HNRNPA2B1 | NM_000424->NM_002137 | YES | | E9: chr12: 51194625-51195291 |
| 89 | KRT5->HNRNPA2B1 | NM_000424->NM_031243 | YES | | E9: chr12: 51194625-51195291 |
| 90 | FN1->YWHAG | NM_002026->NM_012479 | (part of FN1) | | E37: chr2: 215948181-215948361 |
| 91 | FN1->YWHAG | NM_212482->NM_012479 | (part of FN1) | | E38: chr2: 215948181-215948361 |
| 92 | FN1->YWHAG | NM_212474->NM_012479 | (part of FN1) | | E36: chr2: 215948181-215948361 |
| 93 | FN1->YWHAG | NM_212476->NM_012479 | (part of FN1) | | E36: chr2: 215948181-215948361 |
| 94 | FN1->YWHAG | NM_212478->NM_012479 | (part of FN1) | | E37: chr2: 215948181-215948361 |
| 95 | ATN1->KRT14 | NM_001940->NM_000526 | YES | TTT->CCT(F->P) | E17: chr12: 6917904-6918601 |
| 96 | ATN1->KRT14 | NM_001007026->NM_000526 | YES | TTT->CCT(F->P) | E17: chr12: 6917904-6918601 |
| 97 | ALDOA->KRT5 | NM_184043->NM_000424 | NOT Evaluated | | E29: chr16: 29986055-29986188 |
| 98 | ALDOA->KRT5 | NM_184041->NM_000424 | NOT Evaluated | | E29: chr16: 29986055-29986188 |
| 99 | ALDOA->KRT5 | NM_001127617->NM_000424 | NOT Evaluated | | E29: chr16: 29986055-29986188 |
| 100 | ALDOA->KRT5 | NM_000034->NM_000424 | NOT Evaluated | | E49: chr16: 29986055-29986188 |
| 101 | CD74->MBD6 | NM_001025158->NM_052897 | YES | | E6: chr5: 149761392-149762006 |
| 102 | CALR->ZFP36L1 | NM_004343->NM_004926 | YES | | E72: chr19: 12915526-12916304 |
| 103 | ZFP36L1->CALR | NM_004926->NM_004343 | YES | | E2: chr14: 68324127-68326962 |
| 104 | CALR->ZFP36L1 | NM_004343->NM_004926 | YES | | E72: chr19: 12915526-12916304 |
| 105 | SAMD4B->COL1A1 | NM_018028->NM_000088 | | | E39: chr19: 44558129-44558369 |
| 106 | SAMD4B->COL1A1 | NM_018028->NM_000088 | | | E39: chr19: 44558129-44558369 |
| 107 | COL4A2->COL1A1 | NM_001846->NM_000088 | | | E79: chr13: 109930567-109930738 |
| 108 | COL4A2->COL1A1 | NM_001846->NM_000088 | YES | GAG->AAG(E->K) | E91: chr13: 109953730-109953829 |
| 109 | RPS15->PLEC | NM_001018->NM_201381 | YES | ATG->GAG(M->E) | E3: chr19: 1391017-1391252 |
| 110 | RPS15->PLEC | NM_001018->NM_201382 | YES | ATG->GAG(M->E) | E3: chr19: 1391017-1391252 |
| 111 | RPS15->PLEC | NM_001018->NM_201380 | YES | ATG->GAG(M->E) | E3: chr19: 1391017-1391252 |
| 112 | RPS15->PLEC | NM_001018->NM_201378 | YES | ATG->GAG(M->E) | E3: chr19: 1391017-1391252 |
| 113 | RPS15->PLEC | NM_001018->NM_201379 | YES | ATG->GAG(M->E) | E3: chr19: 1391017-1391252 |
| 114 | RPS15->PLEC | NM_001018->NM_201383 | YES | ATG->GAG(M->E) | E3: chr19: 1391017-1391252 |
| 115 | RPS15->PLEC | NM_001018->NM_201384 | YES | ATG->GAG(M->E) | E3: chr19: 1391017-1391252 |
| 116 | RPS15->PLEC | NM_001018->NM_000445 | YES | ATG->GAG(M->E) | E3: chr19: 1391017-1391252 |
| 117 | EPHA2->CTSD | NM_004431->NM_001909 | YES | | E17: chr1: 16323419-16324402 |
| 118 | IFI27->CPNE3 | NM_001130080->NM_003909 | YES | | E5: chr14: 93652531-93652786 |
| 119 | IFI27->CPNE3 | NM_005532->NM_003909 | YES | | E5: chr14: 93652531-93652786 |
| 120 | SLC16A3->MRPL4 | NM_004207->NM_146388 | | | E4: chr17: 77788302-77789058 |
| 121 | SLC16A3->MRPL4 | NM_001042422->NM_146388 | | | E4: chr17: 77788302-77789058 |
| 122 | SLC16A3->MRPL4 | NM_001042423->NM_146388 | | | E4: chr17: 77788302-77789058 |
| 123 | KRT18->PLEC | NM_199187->NM_201381 | YES | CTG->GTG(L->V) | E7: chr12: 51632169-51632393 |
| 124 | KRT18->PLEC | NM_199187->NM_201382 | YES | CTG->GTG(L->V) | E7: chr12: 51632169-51632393 |
| 125 | KRT18->PLEC | NM_199187->NM_201380 | YES | CTG->GTG(L->V) | E7: chr12: 51632169-51632393 |
| 126 | KRT18->PLEC | NM_199187->NM_201378 | YES | CTG->GTG(L->V) | E7: chr12: 51632169-51632393 |
| 127 | KRT18->PLEC | NM_199187->NM_201379 | YES | CTG->GTG(L->V) | E7: chr12: 51632169-51632393 |
| 128 | KRT18->PLEC | NM_199187->NM_201383 | YES | CTG->GTG(L->V) | E7: chr12: 51632169-51632393 |
| 129 | KRT18->PLEC | NM_199187->NM_201384 | YES | CTG->GTG(L->V) | E7: chr12: 51632169-51632393 |
| 130 | KRT18->PLEC | NM_199187->NM_000445 | YES | CTG->GTG(L->V) | E7: chr12: 51632169-51632393 |
| 131 | KRT18->PLEC | NM_000224->NM_201381 | YES | CTG->GTG(L->V) | E6: chr12: 51632169-51632393 |
| 132 | KRT18->PLEC | NM_000224->NM_201382 | YES | CTG->GTG(L->V) | E6: chr12: 51632169-51632393 |
| 133 | KRT18->PLEC | NM_000224->NM_201380 | YES | CTG->GTG(L->V) | E6: chr12: 51632169-51632393 |
| 134 | KRT18->PLEC | NM_000224->NM_201378 | YES | CTG->GTG(L->V) | E6: chr12: 51632169-51632393 |
| 135 | KRT18->PLEC | NM_000224->NM_201379 | YES | CTG->GTG(L->V) | E6: chr12: 51632169-51632393 |
| 136 | KRT18->PLEC | NM_000224->NM_201383 | YES | CTG->GTG(L->V) | E6: chr12: 51632169-51632393 |
| 137 | KRT18->PLEC | NM_000224->NM_201384 | YES | CTG->GTG(L->V) | E6: chr12: 51632169-51632393 |
| 138 | KRT18->PLEC | NM_000224->NM_000445 | YES | CTG->GTG(L->V) | E6: chr12: 51632169-51632393 |
| 139 | C2orf56->SAMD4B | NM_144736->NM_018028 | YES | | E10: chr2: 37328781-37329807 |
| 140 | C2orf56->SAMD4B | NM_001083946->NM_018028 | YES | | E8: chr2: 37328781-37329807 |
| 141 | POSTN->TRIM33 | NM_001135935->NM_015906 | YES | | E21: chr13: 37034719-37035507 |
| 142 | POSTN->TRIM33 | NM_001135935->NM_033020 | YES | | E21: chr13: 37034719-37035507 |
| 143 | POSTN->TRIM33 | NM_006475->NM_015906 | YES | | E23: chr13: 37034719-37035507 |
| 144 | POSTN->TRIM33 | NM_006475->NM_033020 | YES | | E23: chr13: 37034719-37035507 |
| 145 | POSTN->TRIM33 | NM_001135934->NM_015906 | YES | | E21: chr13: 37034719-37035507 |
| 146 | POSTN->TRIM33 | NM_001135934->NM_033020 | YES | | E21: chr13: 37034719-37035507 |
| 147 | POSTN->TRIM33 | NM_001135936->NM_015906 | YES | | E20: chr13: 37034719-37035507 |
| 148 | POSTN->TRIM33 | NM_001135936->NM_033020 | YES | | E20: chr13: 37034719-37035507 |
| 149 | GPAA1->CD24 | NM_003801->NM_013230 | | | E4: chr8: 145210604-145210752 |
| 150 | GPAA1->CD24 | NM_003801->NM_013230 | YES | CTG->GTG(L->V) | E4: chr8: 145210604-145210752 |
| 151 | DNM2->PIN1 | NM_004945->NM_006221 | | | E11: chr19: 10770161-10770248 |
| 152 | DNM2->PIN1 | NM_001190716->NM_006221 | | | E11: chr19: 10770161-10770248 |
| 153 | DNM2->PIN1 | NM_001005360->NM_006221 | | | E11: chr19: 10770161-10770248 |
| 154 | DNM2->PIN1 | NM_001005361->NM_006221 | | | E11: chr19: 10770161-10770248 |
| 155 | DNM2->PIN1 | NM_001005362->NM_006221 | YES | CTG->GTG(L->V) | E11: chr19: 10770161-10770248 |
| 156 | KRT5->KRT14 | NM_000424->NM_000526 | YES | | E9: chr12: 51194625-51195291 |
| 157 | COL3A1->COL1A1 | NM_000090->NM_000088 | | | E609: chr2: 189581894-189582192 |
| 158 | COL18A1->SPARC | NM_130444->NM_003118 | | | E35: chr21: 45749475-45749620 |
| 159 | COL18A1->SPARC | NM_130445->NM_003118 | | | E36: chr21: 45749475-45749620 |
| 160 | COL18A1->SPARC | NM_030582->NM_003118 | | | E35: chr21: 45749475-45749620 |
| 161 | SPARC->COL18A1 | NM_003118->NM_130444 | YES | | E2: chr5: 151035885-151035955 |
| 162 | SPARC->COL18A1 | NM_003118->NM_130445 | YES | | E2: chr5: 151035885-151035955 |
| 163 | SPARC->COL18A1 | NM_003118->NM_030582 | YES | | E2: chr5: 151035885-151035955 |
| 164 | COL18A1->SPARC | NM_130444->NM_003118 | | | E35: chr21: 45749475-45749620 |
| 165 | COL18A1->SPARC | NM_130445->NM_003118 | | | E36: chr21: 45749475-45749620 |
| 166 | COL18A1->SPARC | NM_030582->NM_003118 | | | E35: chr21: 45749475-45749620 |
| 167 | COL18A1->SPARC | NM_130444->NM_003118 | | | E35: chr21: 45749475-45749620 |
| 168 | COL18A1->SPARC | NM_130445->NM_003118 | | | E36: chr21: 45749475-45749620 |
| 169 | COL18A1->SPARC | NM_030582->NM_003118 | | | E35: chr21: 45749475-45749620 |
| 170 | IGFBP5->AMD1 | NM_000599->NM_001634 | YES | | E4: chr2: 217245072-217249850 |
| 171 | CPSF6->COL1A1 | NM_007007->NM_000088 | | | E5: chr12: 67937778-67937952 |
| 172 | CPSF6->COL1A1 | NM_007007->NM_000088 | | | E5: chr12: 67937778-67937952 |
| 173 | PRPF40A->RPL14 | NM_017892->NM_003973 | | | E10: chr2: 153241164-153241539 |
| 174 | PRPF40A->RPL14 | NM_017892->NM_001034996 | | | E10: chr2: 153241164-153241539 |
| 175 | RALGPS2->LAMB3 | NM_152663->NM_001017402 | | | E16: chr1: 177129676-177129782 |
| 176 | RALGPS2->LAMB3 | NM_152663->NM_001127641 | | | E16: chr1: 177129676-177129782 |
| 177 | RALGPS2->LAMB3 | NM_152663->NM_000228 | | | E16: chr1: 177129676-177129782 |
| 178 | COL1A1->FGD2 | NM_000088->NM_173558 | | | E49: chr17: 45618676-45618867 |
| 179 | CTTN->NCRNA00201 | NM_005231->NR_026778 | YES | | E36: chr11: 69958779-69960338 |
| 180 | FBLIM1->F3 | NM_017556->NM_001178096 | YES | | E9: chr1: 15983629-15985671 |
| 181 | FBLIM1->F3 | NM_017556->NM_001993 | YES | | E9: chr1: 15983629-15985671 |
| 182 | FBLIM1->F3 | NM_001024216-> | YES | | E5: chr1: 15983629-15985671 |
| | | NM_001178096 | | | |
| 183 | FBLIM1->F3 | NM_001024216->NM_001993 | YES | | E5: chr1: 15983629-15985671 |
| 184 | GAPDH->MRPS18B | NM_002046->NM_014046 | YES | | E108: chr12: 6517527-6517797 |
| 185 | HSP90AB1->PCGF2 | NM_007355->NM_007144 | YES | | E71: chr6: 44328759-44329093 |
| 186 | RPS2->HRAS | NM_002952->NM_001130442 | YES | | E2: chr16: 1954450-1954630 |
| 187 | RPS2->HRAS | NM_002952->NM_005343 | | | E2: chr16: 1954450-1954630 |
| 188 | RPS2->HRAS | NM_002952->NM_176795 | | | E2: chr16: 1954450-1954630 |
| 189 | RNF213->KRT5 | NM_020914->NM_000424 | YES | | E138: chr17: 75981739-75984680 |
| 190 | PRINS->KIAA1217 | NR_023388->NM_001098500 | NOT Evaluated | | E2: chr10: 24584056-24584981 |
| 191 | PRINS->KIAA1217 | NR_023388->NM_001098501 | NOT Evaluated | | E2: chr10: 24584056-24584981 |
| 192 | PRINS->KIAA1217 | NR_023388->NM_019590 | NOT Evaluated | | E2: chr10: 24584056-24584981 |
| 193 | KRT14->NOTCH2 | NM_000526->NM_024408 | | | E6: chr17: 36993012-36993233 |
| 194 | UBR2->SRPK1 | NM_001184801->NR_034069 | (part of UBR2) | | E1: chr6: 42639737-42640113 |
| 195 | UBR2->SRPK1 | NM_001184801->NM_003137 | YES | | E1: chr6: 42639737-42640113 |
| 196 | UBR2->SRPK1 | NM_015255->NR_034069 | (part of UBR2) | | E1: chr6: 42639737-42640113 |
| 197 | UBR2->SRPK1 | NM_015255->NM_003137 | YES | | E1: chr6: 42639737-42640113 |
| 198 | GEMIN7->SLC39A14 | NM_001007270->NM_015359 | YES | | E1: chr19: 50274357-50274377 |
| 199 | GEMIN7->5LC39A14 | NM_001007270->NM_001128431 | YES | | E1: chr19: 50274357-50274377 |
| 200 | GEMIN7->5LC39A14 | NM_001007270->NM_001135154 | YES | | E1: chr19: 50274357-50274377 |
| 201 | GEMIN7->5LC39A14 | NM_001007270->NM_001135153 | YES | | E1: chr19: 50274357-50274377 |
| 202 | GEMIN7->SLC39A14 | NM_024707->NM_015359 | YES | | E1: chr19: 50274357-50274377 |
| 203 | GEMIN7->SLC39A14 | NM_024707->NM_001128431 | YES | | E1: chr19: 50274357-50274377 |
| 204 | GEMIN7->SLC39A14 | NM_024707->NM_001135154 | YES | | E1: chr19: 50274357-50274377 |
| 205 | GEMIN7->SLC39A14 | NM_024707->NM_001135153 | YES | | E1: chr19: 50274357-50274377 |
| 206 | GEMIN7->5LC39A14 | NM_001007270->NM_015359 | YES | | E2: chr19: 50275004-50275127 |
| 207 | GEMIN7->5LC39A14 | NM_001007270->NM_001128431 | YES | | E2: chr19: 50275004-50275127 |
| 208 | GEMIN7->5LC39A14 | NM_001007270->NM_001135154 | YES | | E2: chr19: 50275004-50275127 |
| 209 | GEMIN7->5LC39A14 | NM_001007270->NM_001135153 | YES | | E2: chr19: 50275004-50275127 |
| 210 | GEMIN7->5LC39A14 | NM_024707->NM_015359 | YES | | E2: chr19: 50275004-50275127 |
| 211 | GEMIN7->5LC39A14 | NM_024707->NM_001128431 | YES | | E2: chr19: 50275004-50275127 |
| 212 | GEMIN7->5LC39A14 | NM_024707->NM_001135154 | YES | | E2: chr19: 50275004-50275127 |
| 213 | GEMIN7->5LC39A14 | NM_024707->NM_001135153 | YES | | E2: chr19: 50275004-50275127 |
| 214 | IRF2BP2->ACTB | NM_182972->NM_001101 | YES | | E2: chr1: 232806637-232810221 |
| 215 | IRF2BP2->ACTB | NM_001077397->NM_001101 | YES | | E2: chr1: 232806637-232810221 |
| 216 | TMSB10->RPS16 | NM_021103->NM_001020 | YES | GGC->ACC(G->T) | E6: chr2: 84987023-84987310 |
| 217 | LOC728606->KCTD1 | NR_024259->NM_001142730 | YES | | E1: chr18: 22537353-22537600 |
| 218 | LOC728606->KCTD1 | NR_024259->NM_001136205 | YES | | E1: chr18: 22537353-22537600 |
| 219 | LOC728606->KCTD1 | NR_024259->NM_198991 | YES | | E1: chr18: 22537353-22537600 |
| 220 | LOC728606->KCTD1 | NR_024259->NM_001142730 | YES | | E1: chr18: 22537353-22537600 |
| 221 | LOC728606->KCTD1 | NR_024259->NM_001136205 | YES | | E1: chr18: 22537353-22537600 |
| 222 | LOC728606->KCTD1 | NR_024259->NM_198991 | YES | | E1: chr18: 22537353-22537600 |
| 223 | PALLD->KRT5 | NM_001166110->NM_000424 | YES | | E2: chr4: 170035448-170036120 |
| 224 | PALLD->KRT5 | NM_001166110->NM_000424 | YES | | E2: chr4: 170035448-170036120 |
| 225 | AEBP1->THRA | NM_001129->NM_199334 | | | E39: chr7: 44118681-44119033 |
| 226 | AEBP1->THRA | NM_001129->NM_003250 | | | E39: chr7: 44118681-44119033 |
| 227 | AEBP1->THRA | NM_001129->NM_001190918 | | | E39: chr7: 44118681-44119033 |
| 228 | FLNA->ABCA2 | NM_001110556->NM_001606 | (part of FLNA) | | E46: chrX: 153231210-153231429 |
| 229 | FLNA->ABCA2 | NM_001110556->NM_212533 | (part of FLNA) | | E46: chrX: 153231210-153231429 |
| 230 | FLNA->ABCA2 | NM_001456->NM_001606 | (part of FLNA) | | E45: chrX: 153231210-153231429 |
| 231 | FLNA->ABCA2 | NM_001456->NM_212533 | (part of FLNA) | | E45: chrX: 153231210-153231429 |
| 232 | FTL->ADD3 | NM_000146->NM_019903 | YES | | E8: chr19: 54161651-54161948 |
| 233 | FTL->ADD3 | NM_000146->NM_016824 | YES | | E8: chr19: 54161651-54161948 |
| 234 | FTL->ADD3 | NM_000146->NM_001121 | YES | | E8: chr19: 54161651-54161948 |
| 235 | CYB5R3->TXNIP | NM_001171660->NM_006472 | YES | | E9: chr22: 41343790-41345895 |
| 236 | CYB5R3->TXNIP | NM_001171661->NM_006472 | YES | | E10: chr22: 41343790-41345895 |
| 237 | CYB5R3->TXNIP | NM_007326->NM_006472 | YES | | E9: chr22: 41343790-41345895 |
| 238 | CYB5R3->TXNIP | NM_001129819->NM_006472 | YES | | E9: chr22: 41343790-41345895 |
| 239 | CYB5R3->TXNIP | NM_000398->NM_006472 | YES | | E9: chr22: 41343790-41345895 |
| 240 | FTH1->TNFAIP2 | NM_002032->NM_006291 | NOT Evaluated | | E1: chr11: 61491359-61491708 |
| 241 | MRPL52->U5P22 | NM_178336->NM_015276 | YES | | E5: chr14: 22373220-22374086 |
| 242 | MRPL52->U5P22 | NM_180982->NM_015276 | YES | | E5: chr14: 22373220-22374086 |
| 243 | MRPL52->U5P22 | NM_181306->NM_015276 | YES | | E5: chr14: 22373220-22374086 |
| 244 | MRPL52->U5P22 | NM_181305->NM_015276 | YES | | E4: chr14: 22373220-22374086 |
| 245 | MRPL52->U5P22 | NM_181304->NM_015276 | YES | | E5: chr14: 22373220-22374086 |
| 246 | MRPL52->U5P22 | NM_181307->NM_015276 | YES | | E4: chr14: 22373220-22374086 |
| 247 | PLXNA1->CTSD | NM_032242->NM_001909 | YES | | E31: chr3: 128235454-128238925 |
| 248 | COL3A1->COL16A1 | NM_000090->NM_001856 | YES | | E566: chr2: 189560029-189560110 |
| 249 | SLC9A3R1- | NM_004252->NR_024445 | YES | | E18: chr17: 70276201-70277093 |
| | >LOC100128003 | | | | |
| 250 | KRT6A->PIK3R2 | M_005554->NM_005027 | | | E9: chr12: 51167224-51168006 |
| 251 | SBF1->FLNA | NM_002972->NM_001110556 | YES | | E41: chr22: 49230298-49232535 |
| 252 | SBF1->FLNA | NM_002972->NM_001456 | YES | | E41: chr22: 49230298-49232535 |
| 253 | CAV1->MMP2 | NM_001753->NM_004530 | YES | | E3: chr7: 115986235-115988474 |
| 254 | CAV1->MMP2 | NM_001753->NM_001127891 | YES | | E3: chr7: 115986235-115988474 |
| 255 | CAV1->MMP2 | NM_001172895->NM_004530 | YES | | E3: chr7: 115986235-115988474 |
| 256 | CAV1->MMP2 | NM_001172895->NM_001127891 | YES | | E3: chr7: 115986235-115988474 |
| 257 | CAV1->MMP2 | NM_001172896->NM_004530 | YES | | E2: chr7: 115986235-115988474 |
| 258 | CAV1->MMP2 | NM_001172896->NM_001127891 | YES | | E2: chr7: 115986235-115988474 |
| 259 | CAV1->MMP2 | NM_001172897->NM_004530 | YES | | E3: chr7: 115986235-115988474 |
| 260 | CAV1->MMP2 | NM_001172897->NM_001127891 | YES | | E3: chr7: 115986235-115988474 |
| 261 | CTSD->HOMER3 | NM_001909->NM_001145722 | YES | | E9: chr11: 1730560-1731476 |
| 262 | COL1A2->YAP1 | NM_000089->NM_001195044 | | | E624: chr7: 93897494-93898480 |
| 263 | COL1A2->YAP1 | NM_000089->NM_001130145 | | | E624: chr7: 93897494-93898480 |
| 264 | COL1A2->YAP1 | NM_000089->NM_006106 | | | E624: chr7: 93897494-93898480 |
| 265 | COL1A2->YAP1 | NM_000089->NM_001195045 | | | E624: chr7: 93897494-93898480 |
| 266 | TTC7A->SOCS5 | NM_020458->NM_014011 | YES | INSERTION: GATTTTATAATC(DFII) | E1: chr2: 47021816-47022368 |
| 267 | TTC7A->SOCS5 | NM_020458->NM_144949 | YES | INSERTION: GATTTTATAATC(DFII) | E1: chr2: 47021816-47022368 |
| 268 | USF2->IRX3 | NM_003367->NM_024336 | YES | | E14: chr19: 40452545-40452746 |
| 269 | RPL23->MUCL1 | NM_000978->NM_058173 | | | E5: chr17: 34259846-34259993 |
| 270 | SRRM2->SPARC | NM_016333->NM_003118 | YES | | E60: chr16: 2760859-2761414 |
| 271 | SRRM2->SPARC | NM_016333->NM_003118 | YES | | E60: chr16: 2760859-2761414 |
| 272 | DNAJA2->COL14A1 | NM_005880->NM_021110 | YES | | E9: chr16: 45546774-45548633 |
| 273 | KRT81->ACTB | NM_002281->NM_001101 | YES | | E9: chr12: 50965963-50966544 |
| 274 | FTH1->KCTD12 | NM_002032->NM_138444 | YES | | E1: chr11: 61491359-61491708 |
| 275 | COL1A1->TBC1D9B | NM_000088->NM_198868 | YES | | E50: chr17: 45618137-45618380 |
| 276 | COL1A1->TBC1D9B | NM_000088->NM_015043 | YES | | E50: chr17: 45618137-45618380 |
| 277 | RPL19->RPS16 | NM_000981->NM_001020 | YES | | E2: chr17: 34610991-34611098 |
| 278 | MTF2->ARL3 | NM_007358->NM_004311 | | | E1: chr1: 93317379-93317676 |
| 279 | MTF2->ARL3 | NM_001164393->NM_004311 | NOT Evaluated | | E1: chr1: 93317379-93317676 |
| 280 | MTF2->ARL3 | NM_001164392->NM_004311 | | | E1: chr1: 93317379-93317676 |
| 281 | MTF2->ARL3 | NM_001164391->NM_004311 | NOT Evaluated | | E1: chr1: 93317379-93317676 |
| 282 | MTF2->ARL3 | NM_007358->NM_004311 | | | E1: chr1: 93317379-93317676 |
| 283 | MTF2->ARL3 | NM_001164393->NM_004311 | NOT Evaluated | | E1: chr1: 93317379-93317676 |
| 284 | MTF2->ARL3 | NM_001164392->NM_004311 | | | E1: chr1: 93317379-93317676 |
| 285 | MTF2->ARL3 | NM_001164391->NM_004311 | NOT Evaluated | | E1: chr1: 93317379-93317676 |
| 286 | SFTPC->IGLL5 | NM_003018->NR_033661 | | | E2: chr8: 22076031-22076190 |
| 287 | SFTPC->IGLL5 | NM_003018->NM_001178126 | | | E2: chr8: 22076031-22076190 |
| 288 | SFTPC->IGLL5 | NM_001172410->NR_033661 | | | E2: chr8: 22076031-22076190 |
| 289 | SFTPC->IGLL5 | NM_001172410-> | | | E2: chr8: 22076031-22076190 |
| | | NM_001178126 | | | |
| 290 | SFTPC->IGLL5 | NM_001172357->NR_033661 | | | E2: chr8: 22076031-22076190 |
| 291 | SFTPC->IGLL5 | NM_001172357-> | | | E2: chr8: 22076031-22076190 |
| | | NM_001178126 | | | |
| 292 | C6orf147->KHDC1 | NR_027005->NM_030568 | YES | | E3: chr6: 74058441-74058484 |
| 293 | PRR4->TAS2R20 | NM_001098538->NM_176889 | (part of PRR4) | | E2: chr12: 11090885-11091054 |
| 294 | PRR4->TAS2R20 | NM_001098538->NM_176889 | (part of PRR4) | | E2: chr12: 11090885-11091054 |
| 295 | PRR4->TAS2R20 | NM_001098538->NM_176889 | (part of PRR4) | | E2: chr12: 11090885-11091054 |
| 296 | PRR4->TAS2R20 | NM_001098538->NM_176889 | (part of PRR4) | | E2: chr12: 11090885-11091054 |
| 297 | ACTN4->ACTB | NM_004924->NM_001101 | YES | | E21: chr19: 43911753-43913010 |
| 298 | ACTN4->ACTB | NM_004924->NM_001101 | YES | | E21: chr19: 43911753-43913010 |
| 299 | ACTN4->ACTB | NM_004924->NM_001101 | YES | | E21: chr19: 43911753-43913010 |
| 300 | ACTN4->ACTB | NM_004924->NM_001101 | YES | | E21: chr19: 43911753-43913010 |
| 301 | ACTN4->ACTB | NM_004924->NM_001101 | YES | | E21: chr19: 43911753-43913010 |
| 302 | MGP->NCRNA00188 | NM_000900->NR_027163 | YES | | E4: chr12: 14925381-14926481 |
| 303 | MGP->NCRNA00188 | NM_000900->NR_027162 | YES | | E4: chr12: 14925381-14926481 |
| 304 | MGP->NCRNA00188 | NM_000900->NR_027165 | YES | | E4: chr12: 14925381-14926481 |
| 305 | MGP->NCRNA00188 | NM_000900->NR_027164 | YES | | E4: chr12: 14925381-14926481 |
| 306 | MGP->NCRNA00188 | NM_000900->NR_027170 | YES | | E4: chr12: 14925381-14926481 |
| 307 | MGP->NCRNA00188 | NM_000900->NR_027160 | YES | | E4: chr12: 14925381-14926481 |
| 308 | MGP->NCRNA00188 | NM_000900->NR_027159 | YES | | E4: chr12: 14925381-14926481 |
| 309 | MGP->NCRNA00188 | NM_000900->NR_027158 | YES | | E4: chr12: 14925381-14926481 |
| 310 | MGP->NCRNA00188 | NM_000900->NR_027169 | YES | | E4: chr12: 14925381-14926481 |
| 311 | MGP->NCRNA00188 | NM_000900->NR_027168 | YES | | E4: chr12: 14925381-14926481 |
| 312 | MGP->NCRNA00188 | NM_000900->NR_027167 | YES | | E4: chr12: 14925381-14926481 |
| 313 | MGP->NCRNA00188 | NM_000900->NR_027161 | YES | | E4: chr12: 14925381-14926481 |
| 314 | MGP->NCRNA00188 | NM_000900->NR_027667 | YES | | E4: chr12: 14925381-14926481 |
| 315 | MGP->NCRNA00188 | NM_000900->NR_027166 | YES | | E4: chr12: 14925381-14926481 |
| 316 | MGP->NCRNA00188 | NM_001190839->NR_027163 | YES | | E5: chr12: 14925381-14926481 |
| 317 | MGP->NCRNA00188 | NM_001190839->NR_027162 | YES | | E5: chr12: 14925381-14926481 |
| 318 | MGP->NCRNA00188 | NM_001190839->NR_027165 | YES | | E5: chr12: 14925381-14926481 |
| 319 | MGP->NCRNA00188 | NM_001190839->NR_027164 | YES | | E5: chr12: 14925381-14926481 |
| 320 | MGP->NCRNA00188 | NM_001190839->NR_027170 | YES | | E5: chr12: 14925381-14926481 |
| 321 | MGP->NCRNA00188 | NM_001190839->NR_027160 | YES | | E5: chr12: 14925381-14926481 |
| 322 | MGP->NCRNA00188 | NM_001190839->NR_027159 | YES | | E5: chr12: 14925381-14926481 |
| 323 | MGP->NCRNA00188 | NM_001190839->NR_027158 | YES | | E5: chr12: 14925381-14926481 |
| 324 | MGP->NCRNA00188 | NM_001190839->NR_027169 | YES | | E5: chr12: 14925381-14926481 |
| 325 | MGP->NCRNA00188 | NM_001190839->NR_027168 | YES | | E5: chr12: 14925381-14926481 |
| 326 | MGP->NCRNA00188 | NM_001190839->NR_027167 | YES | | E5: chr12: 14925381-14926481 |
| 327 | MGP->NCRNA00188 | NM_001190839->NR_027161 | YES | | E5: chr12: 14925381-14926481 |
| 328 | MGP->NCRNA00188 | NM_001190839->NR_027667 | YES | | E5: chr12: 14925381-14926481 |
| 329 | MGP->NCRNA00188 | NM_001190839->NR_027166 | YES | | E5: chr12: 14925381-14926481 |
| 330 | PALLD->CBR4 | NM_016081->NM_032783 | YES | | E21: chr4: 170083938-170086183 |
| 331 | PALLD->CBR4 | NM_016081->NM_032783 | YES | | E21: chr4: 170083938-170086183 |
| 332 | NDUFS6->ACTB | NM_004553->NM_001101 | (part of | | E4: chr5: 1868964-1869163 |
| | | | NDUFS6) | | |
| 333 | COL1A2->ACTG1 | NM_000089->NM_001614 | | | E613: chr7: 93891583-93891691 |
| 334 | GNB2->CTSD | NM_005273->NM_001909 | YES | | E10: chr7: 100114253-100114727 |
| 335 | DIDO1->REPS1 | NM_033081->NM_031922 | NOT Evaluated | | E2: chr20: 61016006-61016203 |
| 336 | DIDO1->REPS1 | NM_033081->NM_001128617 | NOT Evaluated | | E2: chr20: 61016006-61016203 |
| 337 | DIDO1->REPS1 | NM_001193369->NM_031922 | NOT Evaluated | | E2: chr20: 61016006-61016203 |
| 338 | DIDO1->REPS1 | NM_001193369->NM_001128617 | NOT Evaluated | | E2: chr20: 61016006-61016203 |
| 339 | DIDO1->REPS1 | NM_001193370->NM_031922 | NOT Evaluated | | E2: chr20: 61016006-61016203 |
| 340 | DIDO1->REPS1 | NM_001193370->NM_001128617 | NOT Evaluated | | E2: chr20: 61016006-61016203 |
| 341 | DIDO1->REPS1 | NM_080797->NM_031922 | NOT Evaluated | | E2: chr20: 61016006-61016203 |
| 342 | DIDO1->REPS1 | NM_080797->NM_001128617 | NOT Evaluated | | E2: chr20: 61016006-61016203 |
| 343 | DIDO1->REPS1 | NM_080796->NM_031922 | NOT Evaluated | | E2: chr20: 61016006-61016203 |
| 344 | DIDO1->REPS1 | NM_080796->NM_001128617 | NOT Evaluated | | E2: chr20: 61016006-61016203 |
| 345 | DIDO1->REPS1 | NM_022105->NM_031922 | NOT Evaluated | | E2: chr20: 61016006-61016203 |
| 346 | DIDO1->REPS1 | NM_022105->NM_001128617 | NOT Evaluated | | E2: chr20: 61016006-61016203 |
| 347 | DIDO1->REPS1 | NM_033081->NM_031922 | NOT Evaluated | | E2: chr20: 61016006-61016203 |
| 348 | DIDO1->REPS1 | NM_033081->NM_001128617 | NOT Evaluated | | E2: chr20: 61016006-61016203 |
| 349 | DIDO1->REPS1 | NM_001193369->NM_031922 | NOT Evaluated | | E2: chr20: 61016006-61016203 |
| 350 | DIDO1->REPS1 | NM_001193369->NM_001128617 | NOT Evaluated | | E2: chr20: 61016006-61016203 |
| 351 | DIDO1->REPS1 | NM_001193370->NM_031922 | NOT Evaluated | | E2: chr20: 61016006-61016203 |
| 352 | DIDO1->REPS1 | NM_001193370->NM_001128617 | NOT Evaluated | | E2: chr20: 61016006-61016203 |
| 353 | DIDO1->REPS1 | NM_080797->NM_031922 | NOT Evaluated | | E2: chr20: 61016006-61016203 |
| 354 | DIDO1->REPS1 | NM_080797->NM_001128617 | NOT Evaluated | | E2: chr20: 61016006-61016203 |
| 355 | DIDO1->REPS1 | NM_080796->NM_031922 | NOT Evaluated | | E2: chr20: 61016006-61016203 |
| 356 | DIDO1->REPS1 | NM_080796->NM_001128617 | NOT Evaluated | | E2: chr20: 61016006-61016203 |
| 357 | DIDO1->REPS1 | NM_022105->NM_031922 | NOT Evaluated | | E2: chr20: 61016006-61016203 |
| 358 | DIDO1->REPS1 | NM_022105->NM_001128617 | NOT Evaluated | | E2: chr20: 61016006-61016203 |
| 359 | MALAT1->IGF2 | NR_002819->NM_001007139 | NOT Evaluated | | E9: chr11: 65021808-65030513 |
| 360 | MALAT1->IGF2 | NR_002819->NM_001127598 | NOT Evaluated | | E9: chr11: 65021808-65030513 |
| 361 | MALAT1->IGF2 | NR_002819->NM_000612 | NOT Evaluated | | E9: chr11: 65021808-65030513 |
| 362 | CALD1->COL1A1 | NM_033157->NM_000088 | YES | | E8: chr7: 134282798-134283060 |
| 363 | CALD1->COL1A1 | NM_033138->NM_000088 | YES | | E8: chr7: 134282798-134283060 |
| 364 | CALD1->COL1A1 | NM_004342->NM_000088 | YES | | E7: chr7: 134282798-134283060 |
| 365 | CALD1->COL1A1 | NM_033140->NM_000088 | YES | | E5: chr7: 134282798-134283060 |
| 366 | CALD1->COL1A1 | NM_033139->NM_000088 | YES | | E6: chr7: 134282798-134283060 |
| 367 | MYH9->KRT6B | NM_002473->NM_005555 | | | E39: chr22: 35010394-35010503 |
| 368 | APOOL->DCAF8 | NM_198450->NM_015726 | YES | | E9: chrX: 84229251-84234980 |
| 369 | APOOL->DCAF8 | NM_198450->NR_028104 | YES | | E9: chrX: 84229251-84234980 |
| 370 | APOOL->DCAF8 | NM_198450->NR_028103 | YES | | E9: chrX: 84229251-84234980 |
| 371 | APOOL->DCAF8 | NM_198450->NR_028105 | YES | | E9: chrX: 84229251-84234980 |
| 372 | APOOL->DCAF8 | NM_198450->NR_028106 | YES | | E9: chrX: 84229251-84234980 |
| 373 | PACSIN3->CTSD | NM_016223->NM_001909 | YES | | E11: chr11: 47155649-47156173 |
| 374 | PACSIN3->CTSD | NM_001184975->NM_001909 | YES | | E11: chr11: 47155649-47156173 |
| 375 | PACSIN3->CTSD | NM_001184974->NM_001909 | YES | | E11: chr11: 47155649-47156173 |
| 376 | SOX4->KRT5 | NM_003107->NM_000424 | YES | | E2: chr6: 21701950-21706828 |
| 377 | HEATR5A->COL1A1 | NM_015473->NM_000088 | YES | | E30: chr14: 30830744-30832569 |
| 378 | TFG->GPR128 | NM_001007565->NM_032787 | YES | | E3: chr3: 101921508-101921592 |
| 379 | TFG->GPR128 | NM_006070->NM_032787 | YES | | E3: chr3: 101921508-101921592 |
| 380 | TFG->GPR128 | NM_001195479->NM_032787 | YES | | E3: chr3: 101921508-101921592 |
| 381 | TFG->GPR128 | NM_001195478->NM_032787 | YES | | E3: chr3: 101921508-101921592 |
| 382 | METTL10->FAM53B | NM_212554->NM_014661 | YES | | E7: chr10: 126437395-126439062 |
| 383 | METTL10->FAM53B | NM_212554->NM_014661 | YES | | E7: chr10: 126437395-126439062 |
| 384 | METTL10->FAM53B | NM_212554->NM_014661 | YES | | E7: chr10: 126437395-126439062 |
| 385 | NUFIP2->KRT5 | NM_020772->NM_000424 | YES | | E4: chr17: 24606979-24615735 |
| 386 | NUFIP2->KRT5 | NM_020772->NM_000424 | YES | | E4: chr17: 24606979-24615735 |
| 387 | CIRBP->UGP2 | NM_001280->NM_006759 | YES | | E7: chr19: 1223425-1224171 |
| 388 | JOSD1->RPS19BP1 | NM_014876->NM_194326 | | | E1: chr22: 37425753-37426405 |
| 389 | COL1A2->TSIX | NM_000089->NR_003255 | YES | | E624: chr7: 93897494-93898480 |
| 390 | C9orf86->PPP1R14B | NM_024718->NM_138689 | YES | INSERTION: CAGGCCCCGGCGGCCGCC(QAPAAA) | E15: chr9: 138854594-138855460 |
| 391 | C9orf86->PPP1R14B | NM_001173988->NM_138689 | YES | INSERTION: CAGGCCCCGGCGGCCGCC(QAPAAA) | E15: chr9: 138854594-138855460 |
| 392 | AATK->USP32 | NM_001080395->NM_032582 | YES | | E1: chr17: 76754332-76754467 |
| 393 | AATK->USP32 | NM_001080395->NM_032582 | YES | | E1: chr17: 76754332-76754467 |
| 394 | DAB2IP->KRT5 | NM_138709->NM_000424 | | | E10: chr9: 123574706-123575595 |
| 395 | DAB2IP->KRT5 | NM_032552->NM_000424 | | | E12: chr9: 123574706-123575595 |
| 396 | ADCY9->C16orf5 | NM_001116->NM_013399 | | INSERTION: GCCCTGCCTGTTCCCTGTCCATCCAG | E2: chr16: 4103751-4105487 |
| | | | | GCCAGCAGCTGAAGGAGCCTCACCTGCCTCCCTT | |
| | | | | CTCTGAGTAGCACGGATTTGAGGAGAAGCAGCGA | |
| | | | | AG(ALPVPCPSRPAAEGASPASLL*VARI*GEAAK) | |
| 397 | RAB3IP->IGFBP5 | NM_175625->NM_000599 | YES | | E10: chr12: 68495410-68503251 |
| 398 | RAB3IP->IGFBP5 | NM_175624->NM_000599 | YES | | E10: chr12: 68495410-68503251 |
| 399 | RAB3IP->IGFBP5 | NM_022456->NM_000599 | YES | | E11: chr12: 68495410-68503251 |
| 400 | RAB3IP->IGFBP5 | NM_175623->NM_000599 | YES | | E11: chr12: 68495410-68503251 |
| 401 | RAB3IP->IGFBP5 | NM_001024647->NM_000599 | YES | | E9: chr12: 68495410-68503251 |
| 402 | RAB3IP->IGFBP5 | NM_175625->NM_000599 | YES | | E10: chr12: 68495410-68503251 |
| 403 | RAB3IP->IGFBP5 | NM_175624->NM_000599 | YES | | E10: chr12: 68495410-68503251 |
| 404 | RAB3IP->IGFBP5 | NM_022456->NM_000599 | YES | | E1: chr12: 68495410-68503251 |
| 405 | RAB3IP->IGFBP5 | NM_175623->NM_000599 | YES | | E11: chr12: 68495410-68503251 |
| 406 | RAB3IP->IGFBP5 | NM_001024647->NM_000599 | YES | | E9: chr12: 68495410-68503251 |
| 407 | MALAT1->DST | NR_002819->NM_001723 | NOT Evaluated | | E9: chr11: 65021808-65030513 |
| 408 | HOOK3->FNTA | NM_032410->NM_002027 | YES | TGG->CTG(W->L) | E17: chr8: 42976406-42976441 |
| 409 | HOOK3->FNTA | NM_032410->NR_033698 | | | E17: chr8: 42976406-42976441 |
| 410 | KRT81->EMP2 | NM_002281->NM_001424 | YES | | E9: chr12: 50965963-50966544 |
| 411 | TPD52->MRPS28 | NM_001025253->NM_014018 | YES | | E7: chr8: 81117409-81117458 |
| 412 | TPD52->MRPS28 | NM_005079->NM_014018 | YES | | E5: chr8: 81117409-81117458 |
| 413 | TPD52->MRPS28 | NM_001025252->NM_014018 | YES | | E5: chr8: 81117409-81117458 |
| 414 | CTSD->PRKAR1B | NM_001909->NM_001164758 | YES | CTC->GTC(L->V) | E6: chr11: 1732711-1732834 |
| 415 | CTSD->PRKAR1B | NM_001909->NM_001164761 | YES | CTC->GTC(L->V) | E6: chr11: 1732711-1732834 |
| 416 | CTSD->PRKAR1B | NM_001909->NM_001164762 | YES | CTC->GTC(L->V) | E6: chr11: 1732711-1732834 |
| 417 | CTSD->PRKAR1B | NM_001909->NM_001164759 | YES | CTC->GTC(L->V) | E6: chr11: 1732711-1732834 |
| 418 | CTSD->PRKAR1B | NM_001909->NM_001164760 | YES | CTC->GTC(L->V) | E6: chr11: 1732711-1732834 |
| 419 | CTSD->PRKAR1B | NM_001909->NM_002735 | YES | CTC->GTC(L->V) | E6: chr11: 1732711-1732834 |
| 420 | ASAP1->MALAT1 | NM_018482->NR_002819 | YES | | E29: chr8: 131133534-131136233 |
| 421 | SRRM2->HSP90AB1 | NM_016333->NM_007355 | YES | AAA->TAA(K->*) | E57: chr16: 2758998-2759286 |
| 422 | PROM1->TAPT1 | NM_006017->NM_153365 | YES | GGG->GTG(G->V) | E12: chr4: 15617258-15617411 |
| 423 | PROM1->TAPT1 | NM_001145850->NM_153365 | YES | GGG->GTG(G->V) | E12: chr4: 15617258-15617411 |
| 424 | PROM1->TAPT1 | NM_001145849->NM_153365 | YES | GGG->GTG(G->V) | E12: chr4: 15617258-15617411 |
| 425 | PROM1->TAPT1 | NM_001145847->NM_153365 | YES | GGG->GTG(G->V) | E12: chr4: 15617258-15617411 |
| 426 | PROM1->TAPT1 | NM_001145852->NM_153365 | YES | GGG->GTG(G->V) | E11: chr4: 15617258-15617411 |
| 427 | PROM1->TAPT1 | NM_001145851->NM_153365 | YES | GGG->GTG(G->V) | E11: chr4: 15617258-15617411 |
| 428 | PROM1->TAPT1 | NM_001145848->NM_153365 | YES | GGG->GTG(G->V) | E12: chr4: 15617258-15617411 |
| 429 | RCC2->MARCKS | NM_018715->NM_002356 | | | E2: chr1: 17637312-17637605 |
| 430 | RPL8->KRT4 | NM_033301->NM_002272 | YES | ACC->GCC(T->A) | E5: chr8: 145986543-145986659 |
| 431 | RPL8->KRT4 | NM_000973->NM_002272 | YES | ACC->GCC(T->A) | E5: chr8: 145986543-145986659 |
| 432 | CD68->NEAT1 | NM_001251->NR_028272 | YES | | E12: chr17: 7425419-7426153 |
| 433 | CD68->NEAT1 | NM_001040059->NR_028272 | YES | | E12: chr17: 7425419-7426153 |
| 434 | PLEKHO2-> | NM_025201->NM_182703 | YES | | E5: chr15: 62940728-62940827 |
| | ANKDD1A | | | | |
| 435 | PLEKHO2->ANKDD1A | NM_001195059->NM_182703 | YES | | E4: chr15: 62940728-62940827 |
| 436 | PLEKHO2->ANKDD1A | NM_025201->NM_182703 | YES | | E5: chr15: 62940728-62940827 |
| 437 | PLEKHO2->ANKDD1A | NM_001195059->NM_182703 | YES | | E4: chr15: 62940728-62940827 |
| 438 | PCNX->MKKS | NM_014982->NM_018848 | | | E7: chr14: 70525036-70525169 |
| 439 | PCNX->MKKS | NM_014982->NM_170784 | | | E7: chr14: 70525036-70525169 |
| 440 | SPARC->TRPS1 | NM_003118->NM_014112 | | | E6: chr5: 151029417-151029538 |
| 441 | SPARC->TRPS1 | NM_003118->NM_014112 | YES | | E10: chr5: 151021201-151023353 |
| 442 | FLNA->UBXN6 | NM_001110556->NM_025241 | YES | | E48: chrX: 153230093-153230598 |
| 443 | FLNA->UBXN6 | NM_001456->NM_025241 | YES | | E47: chrX: 153230093-153230598 |
| 444 | WDR82->CNN2 | NM_025222->NM_201277 | YES | | E9: chr3: 52263477-52266575 |
| 445 | WDR82->CNN2 | NM_025222->NM_004368 | YES | | E9: chr3: 52263477-52266575 |
| 446 | WDR82->CNN2 | NM_025222->NM_201277 | YES | | E9: chr3: 52263477-52266575 |
| 447 | WDR82->CNN2 | NM_025222->NM_004368 | YES | | E9: chr3: 52263477-52266575 |
| 448 | TMEM119->ARIH2 | NM_181724->NM_006321 | YES | | E2: chr12: 107507750-107510302 |
| 449 | GNB1->TRH | NM_002074->NM_007117 | YES | | E12: chr1: 1706588-1708352 |
| 450 | ELF3->SLC39A6 | NM_001114309->NM_012319 | YES | | E9: chr1: 200250959-200252938 |
| 451 | ELF3->SLC39A6 | NM_001114309-> | YES | | E9: chr1: 200250959-200252938 |
| | | NM_001099406 | | | |
| 452 | ELF3->SLC39A6 | NM_004433->NM_012319 | YES | | E9: chr1: 200250959-200252938 |
| 453 | ELF3->SLC39A6 | NM_004433->NM_001099406 | YES | | E9: chr1: 200250959-200252938 |
| 454 | KRT7->KRT17 | NM_005556->NM_000422 | YES | INSERTION: CTCCTCTCCAGCCCTTCTCCTGTGTGCCTGC | E53: chr12: 50928227-50928262 |
| | | | | CTCCTGCCGCCGCCACC(LLSSPSPVCLPPAAAT) | |
| 455 | GAPDH->ILF3 | NM_002046->NM_153464 | | | E107: chr12: 6517010-6517423 |
| 456 | GAPDH->ILF3 | NM_002046->NM_012218 | | | E107: chr12: 6517010-6517423 |
| 457 | GAPDH->ILF3 | NM_002046->NM_017620 | | | E107: chr12: 6517010-6517423 |
| 458 | GAPDH->ILF3 | NM_002046->NM_004516 | | | E107: chr12: 6517010-6517423 |
| 459 | GAPDH->ILF3 | NM_002046->NM_001137673 | | | E107: chr12: 6517010-6517423 |
| 460 | BAT2L2->COL3A1 | NM_015172->NM_000090 | YES | | E34: chr1: 169827348-169829273 |
| 461 | CAPN1->ARL2 | NM_005186->NM_001667 | YES | | E7: chr11: 64711261-64711345 |
| 462 | IGLL5->B2M | NR_033661->NM_004048 | NA | | E6: chr22: 21567554-21568011 |
| 463 | IGLL5->B2M | NM_001178126->NM_004048 | YES | | E12: chr22: 21567554-21568011 |
| 464 | ENO1->ACTG1 | NM_001428->NM_001614 | YES | | E10: chr1: 8845880-8845989 |
| 465 | COL1A1->KLK6 | NM_000088->NM_002774 | YES | INSERTION: GGCGGACAAAGCCCGATTGTTCCTGGGCCC | E50: chr17: 45618137-45618380 |
| | | | | TTTCCCCATCGCGCCTGGGCCTGCTCCCCAGCCCGGGG | |
| | | | | CAGGGGCGGGGGCCAGTGTGGTGACACACGCTGTAGC | |
| | | | | TGTCTCCCCGGCTGGCTGGCTCGCTCTCTCCTGGGGAC | |
| | | | | ACAGAGGTCGGCAGGCAGCACACAGAGGGACCTACGG | |
| | | | | GCAGCTGTTCCTTCCCCCGACTCAAGAATCCCCGGAGC | |
| | | | | CCGGAGGCCTGCAGCAGGAGCGGCC(GGQSPIVPGPFP | |
| | | | | HRAWACSPARGRGGGQCGDTRCSCLPGWLARSLLGTQR | |
| | | | | SAGSTQRDLRAAVPSPDSRIPGARRPAAGAA) | |
| 466 | RAB8A->EIF4G2 | NM_005370->NM_001042559 | YES | | E8: chr19: 16104021-16105445 |
| 467 | RAB8A->EIF4G2 | NM_005370->NM_001418 | YES | | E8: chr19: 16104021-16105445 |
| 468 | LMNA->FTL | NM_170708->NM_000146 | YES | INSERTION: TATCTGGGACCTGCCAGCA | E11: chr1: 154375494-154376502 |
| | | | | CCGTTTTTGTGGTTAGCTCCTTCTTGCC | |
| | | | | AACCAAC(YLGPASTVFVVSSFLPTN) | |
| 469 | LMNA->FTL | NM_170707->NM_000146 | YES | INSERTION: TATCTGGGACCTGCCAGCA | E12: chr1: 154375494-154376502 |
| | | | | CCGTTTTTGTGGTTAGCTCCTTCTTGCC | |
| | | | | AACCAAC(YLGPASTVFVVSSFLPTN) | |
| 470 | COL1A2->LAMP2 | NM_000089->NM_013995 | YES | | E624: chr7: 93897494-93898480 |
| 471 | ALDOA->RPS16 | NM_184043->NM_001020 | | | E34: chr16: 29988320-29988495 |
| 472 | ALDOA->RPS16 | NM_184041->NM_001020 | | | E34: chr16: 29988320-29988495 |
| 473 | ALDOA->RPS16 | NM_001127617->NM_001020 | | | E34: chr16: 29988320-29988495 |
| 474 | ALDOA->RPS16 | NM_000034->NM_001020 | | | E54: chr16: 29988320-29988495 |
| 475 | ALDOA->RPS16 | NM_184043->NM_001020 | | | E34: chr16: 29988320-29988495 |
| 476 | ALDOA->RPS16 | NM_184041->NM_001020 | | | E34: chr16: 29988320-29988495 |
| 477 | ALDOA->RPS16 | NM_001127617->NM_001020 | | | E34: chr16: 29988320-29988495 |
| 478 | ALDOA->RPS16 | NM_000034->NM_001020 | | | E54: chr16: 29988320-29988495 |
| 479 | ELAC1->SMAD4 | NM_018696->NM_005359 | | | E2: chr18: 46754764-46754929 |
| 480 | RPS5->ACTB | NM_001009->NM_001101 | YES | | E12: chr19: 63597860-63597983 |
| 481 | CALR->ACACA | NM_004343->NM_198838 | (part of CALR) | | E72: chr19: 12915526-12916304 |
| 482 | CALR->ACACA | NM_004343->NM_198837 | (part of CALR) | | E72: chr19: 12915526-12916304 |
| 483 | CALR->ACACA | NM_004343->NM_198836 | (part of CALR) | | E72: chr19: 12915526-12916304 |
| 484 | CALR->ACACA | NM_004343->NM_198839 | (part of CALR) | | E72: chr19: 12915526-12916304 |
| 485 | CALR->ACACA | NM_004343->NM_198834 | (part of CALR) | | E72: chr19: 12915526-12916304 |
| 486 | HNRNPH1->VAPA | NM_005520->NM_194434 | YES | GTC->ATC(V->I) | E9: chr5: 178977120-178977256 |
| 487 | HNRNPH1->VAPA | NM_005520->NM_003574 | YES | GTC->ATC(V->I) | E9: chr5: 178977120-178977256 |
| 488 | SLC34A2->ACTB | NM_006424->NM_001101 | YES | GAG->AGG(E->R) | E13: chr4: 25286854-25289466 |
| 489 | FAM129B->PXN | NM_022833->NM_025157 | | | E14: chr9: 129307438-129309531 |
| 490 | FAM129B->PXN | NM_022833->NM_002859 | | | E14: chr9: 129307438-129309531 |
| 491 | FAM129B->PXN | NM_022833->NM_001080855 | | | E14: chr9: 129307438-129309531 |
| 492 | FAM129B->PXN | NM_001035534->NM_025157 | | | E14: chr9: 129307438-129309531 |
| 493 | FAM129B->PXN | NM_001035534->NM_002859 | | | E14: chr9: 129307438-129309531 |
| 494 | FAM129B->PXN | NM_001035534->NM_001080855 | | | E14: chr9: 129307438-129309531 |
| 495 | OLA1->ORMDL3 | NM_013341->NM_139280 | | | E4: chr2: 174796006-174796134 |
| 496 | OLA1->ORMDL3 | NM_001011708->NM_139280 | YES | | E3: chr2: 174796006-174796134 |
| 497 | OGT->ACTB | NM_181673->NM_001101 | YES | | E22: chrX: 70710194-70712472 |
| 498 | OGT->ACTB | NM_181672->NM_001101 | YES | | E22: chrX: 70710194-70712472 |
| 499 | OGT->ACTB | NM_181673->NM_001101 | YES | | E22: chrX: 70710194-70712472 |
| 500 | OGT->ACTB | NM_181672->NM_001101 | YES | | E22: chrX: 70710194-70712472 |
| 501 | COL3A1->ZNF43 | NM_000090->NM_003423 | YES | | E612: chr2: 189584598-189585717 |
| 502 | TEP1->RNASE1 | NM_007110->NM_198235 | YES | INSERTION: GGGCTTTTCTGGGAAA | E18: chr14: 19925903-19926062 |
| | | | | GTGAGGCCACC(GLFWESEAT) | |
| 503 | TEP1->RNASE1 | NM_007110->NM_198234 | YES | INSERTION: GGGCTTTTCTGGGAAA | E18: chr14: 19925903-19926062 |
| | | | | GTGAGGCCACC(GLFWESEAT) | |
| 504 | TEP1->RNASE1 | NM_007110->NM_198232 | YES | INSERTION: GGGCTTTTCTGGGAAA | E18: chr14: 19925903-19926062 |
| | | | | GTGAGGCCACC(GLFWESEAT) | |
| 505 | TEP1->RNASE1 | NM_007110->NM_002933 | YES | INSERTION: GGGCTTTTCTGGGAAA | E18: chr14: 19925903-19926062 |
| | | | | GTGAGGCCACC(GLFWESEAT) | |
| 506 | GAPDH->ACTG1 | NM_002046->NM_001614 | YES | | E108: chr12: 6517527-6517797 |
| 507 | RPL14->GLS | NM_003973->NM_014905 | | | E54: chr3: 40478433-40478863 |
| 508 | RPL14->GLS | NM_001034996->NM_014905 | | | E54: chr3: 40478433-40478863 |
| 509 | TAX1BP1->MALAT1 | NM_001079864->NR_002819 | YES | | E34: chr7: 27834771-27835911 |
| 510 | TAX1BP1->MALAT1 | NM_006024->NR_002819 | YES | | E34: chr7: 27834771-27835911 |
| 511 | SERPINA1->KIAA1217 | NM_001002235->NM_001098501 | YES | | E5: chr14: 93912836-93914730 |
| 512 | SERPINA1->KIAA1217 | NM_001002235->NM_019590 | YES | | E5: chr14: 93912836-93914730 |
| 513 | SERPINA1->KIAA1217 | NM_001127705->NM_001098501 | YES | | E7: chr14: 93912836-93914730 |
| 514 | SERPINA1->KIAA1217 | NM_001127705->NM_019590 | YES | | E7: chr14: 93912836-93914730 |
| 515 | SERPINA1->KIAA1217 | NM_001002236->NM_001098501 | YES | | E7: chr14: 93912836-93914730 |
| 516 | SERPINA1->KIAA1217 | NM_001002236->NM_019590 | YES | | E7: chr14: 93912836-93914730 |
| 517 | SERPINA1->KIAA1217 | NM_001127707->NM_001098501 | YES | | E6: chr14: 93912836-93914730 |
| 518 | SERPINA1->KIAA1217 | NM_001127707->NM_019590 | YES | | E6: chr14: 93912836-93914730 |
| 519 | SERPINA1->KIAA1217 | NM_001127706->NM_001098501 | YES | | E6: chr14: 93912836-93914730 |
| 520 | SERPINA1->KIAA1217 | NM_001127706->NM_019590 | YES | | E6: chr14: 93912836-93914730 |
| 521 | SERPINA1->KIAA1217 | NM_001127702->NM_001098501 | YES | | E6: chr14: 93912836-93914730 |
| 522 | SERPINA1->KIAA1217 | NM_001127702->NM_019590 | YES | | E6: chr14: 93912836-93914730 |
| 523 | SERPINA1->KIAA1217 | NM_001127701->NM_001098501 | YES | | E7: chr14: 93912836-93914730 |
| 524 | SERPINA1->KIAA1217 | NM_001127701->NM_019590 | YES | | E7: chr14: 93912836-93914730 |
| 525 | SERPINA1->KIAA1217 | NM_001127700->NM_001098501 | YES | | E5: chr14: 93912836-93914730 |
| 526 | SERPINA1->KIAA1217 | NM_001127700->NM_019590 | YES | | E5: chr14: 93912836-93914730 |
| 527 | SERPINA1->KIAA1217 | NM_001127703->NM_001098501 | YES | | E7: chr14: 93912836-93914730 |
| 528 | SERPINA1->KIAA1217 | NM_001127703->NM_019590 | YES | E7: chr14: 93912836-93914730 | |
| 529 | SERPINA1->KIAA1217 | NM_001127704->NM_001098501 | YES | E7: chr14: 93912836-93914730 | |
| 530 | SERPINA1->KIAA1217 | NM_001127704->NM_019590 | YES | E7: chr14: 93912836-93914730 | |
| 531 | SERPINA1->KIAA1217 | NM_000295->NM_001098501 | YES | E5: chr14: 93912836-93914730 | |
| 532 | SERPINA1->KIAA1217 | NM_000295->NM_019590 | YES | E5: chr14: 93912836-93914730 | |
| 533 | HMGN3->PAQR8 | NM_004242->N M 133367 | YES | INSERTION: GTTGCATACCCTGTCCTGAGGGCGCGG | E1: chr6: 80000981-80001174 |
| | | | | CACGGAGTGCATGCGGGCCGCTGC(VAYPVLRARHG | |
| | | | | VHAGRC) | |
| 534 | HMGN3->PAQR8 | NM_138730->NM_133367 | YES | INSERTION: GTTGCATACCCTGTCCTGAGGGCGCGG | E1: chr6: 80000981-80001174 |
| | | | | CACGGAGTGCATGCGGGCCGCTGC(VAYPVLRARHG | |
| | | | | VHAGRC) | |
| 535 | RPL14->EP400 | NM_003973->NM_015409 | | | E54: chr3: 40478433-40478863 |
| 536 | RPL14->EP400 | NM_001034996->NM_015409 | | | E54: chr3: 40478433-40478863 |
| 537 | GPATCH8->C8orf46 | NM_001002909->NM_152765 | YES | | E3: chr17: 39897365-39897438 |
| 538 | GPATCH8->C8orf46 | NR_036474->NM_152765 | YES | | E4: chr17: 39897365-39897438 |
| 539 | PTRF->COL1A1 | NM_012232->NM_000088 | YES | | E2: chr17: 37807994-37810932 |
| 540 | CDK4->UBA1 | NM_000075->NM_003334 | YES | | E8: chr12: 56428269-56428667 |
| 541 | GAPDH->IRAK1 | NM_002046->NM_001569 | YES | | E108: chr12: 6517527-6517797 |
| 542 | GAPDH->IRAK1 | NM_002046->NM_001025243 | YES | | E108: chr12: 6517527-6517797 |
| 543 | GAPDH->IRAK1 | NM_002046->NM_001025242 | YES | | E108: chr12: 6517527-6517797 |
| 544 | CD68->PSAP | NM_001251->NM_001042465 | YES | | E12: chr17: 7425419-7426153 |
| 545 | CD68->PSAP | NM_001251->NM_002778 | YES | | E12: chr17: 7425419-7426153 |
| 546 | CD68->PSAP | NM_001251->NM_001042466 | YES | | E12: chr17: 7425419-7426153 |
| 547 | CD68->PSAP | NM_001040059->NM_001042465 | YES | | E12: chr17: 7425419-7426153 |
| 548 | CD68->PSAP | NM_001040059->NM_002778 | YES | | E12: chr17: 7425419-7426153 |
| 549 | CD68->PSAP | NM_001040059->NM_001042466 | YES | | E12: chr17: 7425419-7426153 |
| 550 | CD68->PSAP | NM_001251->NM_001042465 | YES | | E12: chr17: 7425419-7426153 |
| 551 | CD68->P5AP | NM_001251->NM_002778 | YES | | E12: chr17: 7425419-7426153 |
| 552 | CD68->P5AP | NM_001251->NM_001042466 | YES | | E12: chr17: 7425419-7426153 |
| 553 | CD68->PSAP | NM_001040059->NM_001042465 | YES | | E12: chr17: 7425419-7426153 |
| 554 | CD68->PSAP | NM_001040059->NM_002778 | YES | | E12: chr17: 7425419-7426153 |
| 555 | CD68->PSAP | NM_001040059->NM_001042466 | YES | | E12: chr17: 7425419-7426153 |
| 556 | APOL1->ACTB | NM_003661->NM_001101 | YES | | E6: chr22: 34991142-34993522 |
| 557 | APOL1->ACTB | NM_145343->NM_001101 | YES | | E7: chr22: 34991142-34993522 |
| 558 | APOL1->ACTB | NM_001136541->NM_001101 | YES | | E5: chr22: 34991142-34993522 |
| 559 | APOL1->ACTB | NM_001136540->NM_001101 | YES | | E6: chr22: 34991142-34993522 |
| 560 | WRB->SH3BGR | NM_004627->NM_007341 | YES | | E3: chr21: 39685564-39685632 |
| 561 | WRB->SH3BGR | NM_004627->NM_001001713 | | | E3: chr21: 39685564-39685632 |
| 562 | WRB->SH3BGR | NM_001146218->NM_007341 | YES | | E3: chr21: 39685564-39685632 |
| 563 | WRB->SH3BGR | NM_001146218->NM_001001713 | | | E3: chr21: 39685564-39685632 |
| 564 | WRB->SH3BGR | NM_004627->NM_007341 | YES | | E3: chr21: 39685564-39685632 |
| 565 | WRB->SH3BGR | NM_004627->NM_001001713 | | | E3: chr21: 39685564-39685632 |
| 566 | WRB->SH3BGR | NM_001146218->NM_007341 | YES | | E3: chr21: 39685564-39685632 |
| 567 | WRB->SH3BGR | NM_001146218->NM_001001713 | | | E3: chr21: 39685564-39685632 |
| 568 | WRB->SH3BGR | NM_004627->NM_007341 | YES | | E3: chr21: 39685564-39685632 |
| 569 | WRB->SH3BGR | NM_004627->NM_001001713 | | | E3: chr21: 39685564-39685632 |
| 570 | WRB->SH3BGR | NM_001146218->NM_007341 | YES | | E3: chr21: 39685564-39685632 |
| 571 | WRB->SH3BGR | NM_001146218->NM_001001713 | | | E3: chr21: 39685564-39685632 |
| 572 | ITGA3->KHK | NM002204->NM006488 | YES | | E52: chr17: 45521472-45522848 |
| 573 | ITGA3->KHK | NM002204->NM000221 | YES | | E52: chr17: 45521472-45522848 |
| 574 | ITGA3->KHK | NM_005501->NM_006488 | | INSERTION: CCTCCCACGCGGAGGAGGAGCCAGGGCAGCTGGGAGCGGGGA | E50: chr17: 45521472-45522848 |
| | | | | CACCATCCTCCTGGATAAGAGGCAGAGGCCGGGAGGAACCCCGTCAGCCGG | |
| | | | | GCGGGCAGGAAGCTCTGGGAGTAGCCT(PPTRRRSQGSWERGHHPPG*EAEA | |
| | | | | GRNPVSRAGRKLWE*P) | |
| 575 | ITGA3->KHK | NM_005501->NM_000221 | | INSERTION: CCTCCCACGCGGAGGAGGAGCCAGGGCAGCTGGGAGCGGGGA | E50: chr17: 45521472-45522848 |
| | | | | CACCATCCTCCTGGATAAGAGGCAGAGGCCGGGAGGAACCCCGTCAGCCGG | |
| | | | | GCGGGCAGGAAGCTCTGGGAGTAGCCT(PPTRRRSQGSWERGHHPPG*EAEA | |
| | | | | GRNPVSRAGRKLWE*P) | |
| 576 | BDKRB2->BDKRB1 | NM_000623->NM_000710 | | | E2: chr14: 95773163-95773271 |
| 577 | BDKRB2->BDKRB1 | NM_000623->NM_000710 | | | E2: chr14: 95773163-95773271 |
| 578 | RPL14->MPRIP | NM_003973->NM_015134 | | | E54: chr3: 40478433-40478863 |
| 579 | RPL14->MPRIP | NM_003973->NM_201274 | | | E54: chr3: 40478433-40478863 |
| 580 | RPL14->MPRIP | NM_001034996->NM_015134 | | | E54: chr3: 40478433-40478863 |
| 581 | RPL14->MPRIP | NM_001034996->NM_201274 | | | E54: chr3: 40478433-40478863 |
| 582 | PIKFYVE->TMEM119 | NM_015040->NM_181724 | YES | | E42: chr2: 208928158-208931720 |
| 583 | TMEM109->CTSD | NM_024092->NM_001909 | YES | | E4: chr11: 60445821-60447489 |
| 584 | SREBF1->IGFBP5 | NM_004176->NM_000599 | YES | | E19: chr17: 17655392-17656890 |
| 585 | SREBF1->IGFBP5 | NM_001005291->NM_000599 | YES | | E20: chr17: 17655392-17656890 |
| 586 | SREBF1->IGFBP5 | NM_004176->NM_000599 | YES | | E19: chr17: 17655392-17656890 |
| 587 | SREBF1->IGFBP5 | NM_001005291->NM_000599 | YES | | E20: chr17: 17655392-17656890 |
| 588 | MGP->REPS2 | NM_000900->NM_001080975 | YES | | E4: chr12: 14925381-14926481 |
| 589 | MGP->REPS2 | NM_000900->NM_004726 | YES | | E4: chr12: 14925381-14926481 |
| 590 | MGP->REPS2 | NM_001190839-> | YES | | E5: chrl 2: 14925381-14926481 |
| 590 | | NM_001080975 | | | |
| 591 | MGP->REPS2 | NM_001190839->NM_004726 | YES | | E5: chr12: 14925381-14926481 |
| 592 | AKT2->ACTB | NM_001626->NM_001101 | YES | | E14: chr19: 45428063-45431698 |
| 593 | AKT2->ACTB | NM_001626->NM_001101 | YES | | E14: chr19: 45428063-45431698 |
| 594 | SBF1->FAM 129B | NM_002972->NM_022833 | YES | | E41: chr22: 49230298-49232535 |
| 595 | SBF1->FAM 129B | NM_002972->NM_001035534 | YES | | E41: chr22: 49230298-49232535 |
| 596 | SBF1->FAM 129B | NM_002972->NM_022833 | YES | | E41: chr22: 49230298-49232535 |
| 597 | SBF1->FAM 129B | NM_002972->NM_001035534 | YES | | E41: chr22: 49230298-49232535 |
| 598 | RHOBTB3->CRNKL1 | NM_014899->NM_016652 | YES | | E12: chr5: 95154518-95157827 |
| 599 | ACTG1->PPP1R12C | NM_001614->NM_017607 | YES | | E6: chr17: 77091593-77092454 |
| 600 | POSTN->TM9SF3 | NM_001135935->NM_020123 | YES | | E21: chr13: 37034719-37035507 |
| 601 | POSTN->TM9SF3 | NM_006475->NM_020123 | YES | | E23: chr13: 37034719-37035507 |
| 602 | POSTN->TM9SF3 | NM_001135934->NM_020123 | YES | | E21: chr13: 37034719-37035507 |
| 603 | POSTN->TM9SF3 | NM_001135936->NM_020123 | YES | | E20: chr13: 37034719-37035507 |
| 604 | CLTA->PKP3 | NM_001833->NM_007183 | | | E1: chr9: 36180852-36181270 |
| 605 | CLTA->PKP3 | NM_001076677->NM_007183 | | | E1: chr9: 36180852-36181270 |
| 606 | CLTA->PKP3 | NM_001184761->NM_007183 | | | E1: chr9: 36180852-36181270 |
| 607 | CLTA->PKP3 | NM_001184760->NM_007183 | | | E1: chr9: 36180852-36181270 |
| 608 | CLTA->PKP3 | NM_007096->NM_007183 | | | E1: chr9: 36180852-36181270 |
| 609 | CLTA->PKP3 | NM_001184762->NM_007183 | | | E1: chr9: 36180852-36181270 |
| 610 | NTN1->HDLBP | NM_004822->NM_005336 | YES | | E7: chr17: 9083681-9088042 |
| 611 | NTN1->HDLBP | NM_004822->NM_203346 | YES | | E7: chr17: 9083681-9088042 |
| 612 | HACL1->COLQ | NM_012260->NM_005677 | | | E16: chr3: 15579868-15580055 |
| 613 | HACL1->COLQ | NM_012260->NM_080538 | | | E16: chr3: 15579868-15580055 |
| 614 | HACL1->COLQ | NM_012260->NM_080539 | | | E16: chr3: 15579868-15580055 |
| 615 | HACL1->COLQ | NM_012260->NM_005677 | | | E16: chr3: 15579868-15580055 |
| 616 | HACL1->COLQ | NM_012260->NM_080538 | | | E16: chr3: 15579868-15580055 |
| 617 | HACL1->COLQ | NM_012260->NM_080539 | | | E16: chr3: 15579868-15580055 |
| 618 | SHAN K3->TPT1 | NM_001080420->NM_003295 | YES | | E23: chr22: 49516014-49518507 |
| 619 | COL1A1->TIMP2 | NM_000088->NM_003255 | YES | | E51: chr17: 45616455-45618008 |
| 620 | FLNA->GPS1 | NM_001110556->NM_212492 | YES | | E48: chrX: 153230093-153230598 |
| 621 | FLNA->GPS1 | NM_001456->NM_212492 | YES | | E47: chrX: 153230093-153230598 |
| 622 | YWHAG->PDIA3 | NM_012479->NM_005313 | YES | | E2: chr7: 75794043-75797486 |
| 623 | YWHAG->PDIA3 | NM_012479->NM_005313 | YES | | E2: chr7: 75794043-75797486 |
| 624 | MAPK1IP1L->XPO1 | NM_144578->NM_003400 | YES | | E4: chr14: 54601086-54606665 |
| 625 | TP53I13->ABCA10 | NM_138349->NM_080282 | | | E6: chr17: 24923285-24923841 |
| 626 | COL1A1->GORASP2 | NM_000088->NM_015530 | | | E50: chr17: 45618137-45618380 |
| 627 | COL1A1->GORASP2 | NM_000088->NM_015530 | | | E50: chr17: 45618137-45618380 |
| 628 | COL1A2->ACTB | NM_000089->NM_001101 | | | E613: chr7: 93891583-93891691 |
| 629 | LGMN->NAP1L1 | NM_001008530->NM_004537 | NOT Evaluated | | E2: chr14: 92277159-92277277 |
| 630 | LGMN->NAP1L1 | NM_001008530->NM_139207 | NOT Evaluated | | E2: chr14: 92277159-92277277 |
| 631 | CTBS->GNG5 | NM_004388->NM_005274 | YES | | E6: chr1: 84801527-84801689 |
| 632 | CTBS->GNG5 | NM_004388->NM_005274 | YES | | E6: chr1: 84801527-84801689 |
| 633 | CTBS->GNG5 | NM_004388->NM_005274 | YES | | E6: chr1: 84801527-84801689 |
| 634 | CTBS->GNG5 | NM_004388->NM_005274 | YES | | E6: chr1: 84801527-84801689 |
| 635 | CTBS->GNG5 | NM_004388->NM_005274 | YES | | E6: chr1: 84801527-84801689 |
| 636 | CTBS->GNG5 | NM_004388->NM_005274 | YES | | E6: chr1: 84801527-84801689 |
| 637 | CTBS->GNG5 | NM_004388->NM_005274 | YES | | E6: chr1: 84801527-84801689 |
| 638 | CTBS->GNG5 | NM_004388->NM_005274 | YES | | E6: chr1: 84801527-84801689 |
| 639 | CTBS->GNG5 | NM_004388->NM_005274 | YES | | E6: chr1: 84801527-84801689 |
| 640 | CTBS->GNG5 | NM_004388->NM_005274 | YES | | E6: chr1: 84801527-84801689 |
| 641 | CTBS->GNG5 | NM_004388->NM_005274 | YES | | E6: chr1: 84801527-84801689 |
| 642 | ITGB4->KRT6A | NM_000213->NM_005554 | | | E137: chr17: 71244997-71245120 |
| 643 | ITGB4->KRT6A | NM_001005619->NM_005554 | | | E94: chr17: 71244997-71245120 |
| 644 | ITGB4->KRT6A | NM_001005731->NM_005554 | | | E95: chr17: 71244997-71245120 |
| 645 | KRT7->PKM2 | NM_005556->NM_002654 | | | E49: chr12: 50918730-50918826 |
| 646 | KRT7->PKM2 | NM_005556->NM_182471 | | | E49: chr12: 50918730-50918826 |
| 647 | KRT7->PKM2 | NM_005556->NM_182470 | | | E49: chr12: 50918730-50918826 |
| 648 | KRT7->PKM2 | NM_005556->NM_002654 | (part of KRT7) | | E49: chr12: 50918730-50918826 |
| 649 | KRT7->PKM2 | NM_005556->NM_182471 | (part of KRT7) | | E49: chr12: 50918730-50918826 |
| 650 | KRT7->PKM2 | NM_005556->NM_182470 | (part of KRT7) | | E49: chr12: 50918730-50918826 |
| 651 | TNRC18->SLC9A3R1 | NM_001080495->NM_004252 | YES | | E29: chr7: 5315031-5315106 |
| 652 | PTMA->GNB4 | NM_001099285->NM_021629 | YES | | E40: chr2: 232285757-232286494 |
| 653 | PTMA->GNB4 | NM_002823->NM_021629 | YES | | E40: chr2: 232285757-232286494 |
| 654 | GALNT8->KCNA6 | NM_017417->NM_002235 | | | E10: chr12: 4744805-4744973 |
| 655 | GALNT8->KCNA6 | NM_017417->NM_002235 | | | E10: chr12: 4744805-4744973 |
| 656 | GALNT8->KCNA6 | NM_017417->NM_002235 | | | E10: chr12: 4744805-4744973 |
| 657 | RBM6->SLC38A3 | NM_001167582->NM_006841 | YES | | E1: chr3: 49952480-49952662 |
| 658 | RBM6->SLC38A3 | NM_005777->NM_006841 | YES | | E1: chr3: 49952480-49952662 |
| 659 | ITGB4->KRT14 | NM_000213->NM_000526 | | | E159: chr17: 71264875-71264986 |
| 660 | ITGB4->KRT14 | NM_001005619->NM_000526 | | | E116: chr17: 71264875-71264986 |
| 661 | ITGB4->KRT14 | NM_001005731->NM_000526 | | | E116: chr17: 71264875-71264986 |
| 662 | RHOB->GATA3 | NM_004040->NM_002051 | YES | | E2: chr2: 20510315-20512682 |
| 663 | RHOB->GATA3 | NM_004040->NM_001002295 | YES | | E2: chr2: 20510315-20512682 |
| 664 | RPS5->ACTG1 | NM_001009->NM_001614 | (part of RPS5) | | E11: chr19: 63597675-63597774 |
| 665 | TES->HNRNPU | NM_015641->NM_031844 | YES | | E7: chr7: 115684583-115686073 |
| 666 | TES->HNRNPU | NM_015641->NM_004501 | YES | | E7: chr7: 115684583-115686073 |
| 667 | TES->HNRNPU | NM_152829->NM_031844 | YES | | E7: chr7: 115684583-115686073 |
| 668 | TES->HNRNPU | NM_152829->NM_004501 | YES | | E7: chr7: 115684583-115686073 |
| 669 | PLEC->PLEKHM2 | NM_201381->NM_015164 | | | E31: chr8: 145068659-145072040 |
| 670 | PLEC->PLEKHM2 | NM_201382->NM_015164 | | | E31: chr8: 145068659-145072040 |
| 671 | PLEC->PLEKHM2 | NM_201380->NM_015164 | | | E31: chr8: 145068659-145072040 |
| 672 | PLEC->PLEKHM2 | NM_201378->NM_015164 | | | E31: chr8: 145068659-145072040 |
| 673 | PLEC->PLEKHM2 | NM_201379->NM_015164 | | | E31: chr8: 145068659-145072040 |
| 674 | PLEC->PLEKHM2 | NM_201383->NM_015164 | | | E31: chr8: 145068659-145072040 |
| 675 | PLEC->PLEKHM2 | NM_201384->NM_015164 | | | E31: chr8: 145068659-145072040 |
| 676 | PLEC->PLEKHM2 | NM_000445->NM_015164 | | | E32: chr8: 145068659-145072040 |
| 677 | STC2->RNF11 | NM_003714->NM_014372 | | | E4: chr5: 172674331-172677858 |
| 678 | MT2A->KRT5 | NM_005953->NM_000424 | NOT Evaluated | | E4: chr16: 55199978-55200096 |
| 679 | MT2A->KRT5 | NM_005953->NM_000424 | NOT Evaluated | | E4: chr16: 55199978-55200096 |
| 680 | THSD4->PAQR5 | NM_024817->NM_017705 | YES | | E6: chr15: 69491079-69491216 |
| 681 | THSD4->PAQR5 | NM_024817->NM_001104554 | YES | | E6: chr15: 69491079-69491216 |
| 682 | FUS->ACTB | NM_004960->NM_001101 | YES | | E15: chr16: 31110220-31113691 |
| 683 | FUS->ACTB | NR_028388->NM_001101 | NOT Evaluated | | E14: chr16: 31110220-31113691 |
| 684 | FUS->ACTB | NM_001170937->NM_001101 | YES | | E15: chr16: 31110220-31113691 |
| 685 | FUS->ACTB | NM_001170634->NM_001101 | YES | | E15: chr16: 31110220-31113691 |
| 686 | ACTB->C20orf112 | NM_001101->NM_080616 | YES | | E6: chr7: 5533304-5534048 |
| 687 | ACTB->C200rf112 | NM_001101->NM_080616 | YES | | E6: chr7: 5533304-5534048 |
| 688 | ACTB->PMEPA1 | NM_001101->NM_199171 | YES | | E6: chr7: 5533304-5534048 |
| 689 | ACTB->PMEPA1 | NM_001101->NM_199169 | YES | | E6: chr7: 5533304-5534048 |
| 690 | ACTB->PMEPA1 | NM_001101->NM_199170 | YES | | E6: chr7: 5533304-5534048 |
| 691 | ACTB->PMEPA1 | NM_001101->NM_020182 | YES | | E6: chr7: 5533304-5534048 |
| 692 | RPL14->ATXN1 | NM_003973->NM_000332 | YES | CTG->GCG(L->A) | E54: chr3: 40478433-40478863 |
| 693 | RPL14->ATXN1 | NM_003973->NM_001128164 | YES | CTG->GCG(L->A) | E54: chr3: 40478433-40478863 |
| 694 | RPL14->ATXN1 | NM_001034996->NM_000332 | YES | CTG->GCG(L->A) | E54: chr3: 40478433-40478863 |
| 695 | RPL14->ATXN1 | NM_001034996->NM_001128164 | YES | CTG->GCG(L->A) | E54: chr3: 40478433-40478863 |
| 696 | RPL14->ATXN1 | NM_003973->NM_000332 | | | E54: chr3: 40478433-40478863 |
| 697 | RPL14->ATXN1 | NM_003973->NM_001128164 | | | E54: chr3: 40478433-40478863 |
| 698 | RPL14->ATXN1 | NM_001034996->NM_000332 | | | E54: chr3: 40478433-40478863 |
| 699 | RPL14->ATXN1 | NM_001034996->NM_001128164 | | | E54: chr3: 40478433-40478863 |
| 700 | RPL14->ATXN1 | NM_003973->NM_000332 | | | E54: chr3: 40478433-40478863 |
| 701 | RPL14->ATXN1 | NM_003973->NM_001128164 | | | E54: chr3: 40478433-40478863 |
| 702 | RPL14->ATXN1 | NM_001034996->NM_000332 | | | E54: chr3: 40478433-40478863 |
| 703 | RPL14->ATXN1 | NM_001034996->NM_001128164 | | | E54: chr3: 40478433-40478863 |
| 704 | SFI1->YPEL1 | NM_014775->NM_013313 | | | E5: chr22: 30272846-30272957 |
| 705 | SFI1->YPEL1 | NM_001007467->NM_013313 | | | E5: chr22: 30272846-30272957 |
| 706 | CNOT6->MICAL2 | NM_015455->NM_014632 | YES | | E1: chr5: 179854022-179854369 |
| 707 | CNOT6->MICAL2 | NM_015455->NM_014632 | YES | | E1: chr5: 179854022-179854369 |
| 708 | KRT17->PKM2 | NM_000422->NM_002654 | | | E4: chr17: 37031370-37031532 |
| 709 | KRT17->PKM2 | NM_000422->NM_182471 | | | E4: chr17: 37031370-37031532 |
| 710 | KRT17->PKM2 | NM_000422->NM_182470 | | | E4: chr17: 37031370-37031532 |
| 711 | EEF1DP3->FRY | NR_027062->NM_023037 | NOT Evaluated | | E2: chr13: 31418145-31418318 |
| 712 | EEF1DP3->FRY | NR_027062->NM_023037 | NOT Evaluated | | E2: chr13: 31418145-31418318 |
| 713 | EEF1DP3->FRY | NR_027062->NM_023037 | NOT_Evaluated | | E2: chr13: 31418145-31418318 |
| 714 | EEF1DP3->FRY | NR_027062->NM_023037 | NOT Evaluated | | E2: chr13: 31418145-31418318 |
| 715 | EEF1DP3->FRY | NR_027062->NM_023037 | NOT Evaluated | | E2: chr13: 31418145-31418318 |
| 716 | KRT15->KRT6A | NM_002275->NM_005554 | YES | | E8: chr17: 36923523-36923898 |
| 717 | ITGAV->ANKHD1 | NM_002210->NM_017747 | YES | | E17: chr2: 187229218-187229373 |
| 718 | ITGAV->ANKHD1 | NM_001145000->NM_017747 | YES | | E15: chr2: 187229218-187229373 |
| 719 | ITGAV->ANKHD1 | NM_001144999->NM_017747 | YES | | E17: chr2: 187229218-187229373 |
| 720 | KRT5->VCP | NM_000424->NM_007126 | YES | | E9: chr12: 51194625-51195291 |
| 721 | TMED2->ACTB | NM_006815->NM_001101 | YES | | E4: chr12: 122647104-122648641 |
| 722 | TMED2->ACTB | NM_006815->NM_001101 | YES | | E4: chr12: 122647104-122648641 |
| 723 | TNS4->KRT5 | NM_032865->NM_000424 | YES | | E13: chr17: 35885605-35887507 |
| 724 | TNS4->KRT5 | NM_032865->NM_000424 | YES | | E13: chr17: 35885605-35887507 |
| 725 | TPM4->CD24 | NM_001145160->NM_013230 | YES | | E18: chr19: 16073073-16074813 |
| 726 | TPM4->CD24 | NM_003290->NM_013230 | YES | | E16: chr19: 16073073-16074813 |
| 727 | MAF->IGFBP7 | NM_001031804->NM_001553 | YES | | E1: chr16: 78185246-78192123 |
| 728 | POLD3->COL3A1 | NM_006591->NM_000090 | YES | | E12: chr11: 74029256-74031413 |
| 729 | ATP1A1->KRT17 | NM_001160233->NM_000422 | NOT Evaluated | | E1: chr1: 116718011-116718386 |
| 730 | GAPDH->CD24 | NM_002046->NM_013230 | YES | | E108: chr12: 6517527-6517797 |
| 731 | EIF4G1->ABCC5 | NM_001194946->NM_005688 | (part of | | E27: chr3: 185528311-185528484 |
| | | | EIF4G1) | | |
| 732 | EIF4G1->ABCC5 | NM_001194947->NM_005688 | (part of | | E26: chr3: 185528311-185528484 |
| | | | EIF4G1) | | |
| 733 | EIF4G1->ABCC5 | NM_198242->NM_005688 | (part of | | E22: chr3: 185528311-185528484 |
| | | | EIF4G1) | | |
| 734 | EIF4G1->ABCC5 | NM_198244->NM_005688 | (part of | | E23: chr3: 185528311-185528484 |
| | | | EIF4G1) | | |
| 735 | EIF4G1->ABCC5 | NM_198241->NM_005688 | (part of | | E26: chr3: 185528311-185528484 |
| | | | EIF4G1) | | |
| 736 | EIF4G1->ABCC5 | NM_182917->NM_005688 | (part of | | E25: chr3: 185528311-185528484 |
| | | | EIF4G1) | | |
| 737 | EIF4G1->ABCC5 | NM_004953->NM_005688 | (part of | | E19: chr3: 185528311-185528484 |
| | | | EIF4G1) | | |
| 738 | HSP90AB1->KRT6A | NM_007355->NM_005554 | YES | INSERTION: GCAGCTCT | E70: chr6: 44327713-44327982 |
| | | | | CTCATCTCCTGGAACC | |
| | | | | (AALSSPGT) | |
| 739 | RRN3P3->CDR2 | NR_027460->NM_001802 | YES | | E5: chr16: 22348617-22348770 |
| 740 | RRN3P3->CDR2 | NR_027460->NM_001802 | YES | | E5: chr16: 22348617-22348770 |
| 741 | MALAT1->ACTG1 | NR_002819->NM_001614 | NOT Evaluated | | E9: chr11: 65021808-65030513 |
| 742 | MALAT1->ACTG1 | NR_002819->NM_001614 | NOT Evaluated | | E9: chr11: 65021808-65030513 |
| 743 | MALAT1->ACTG1 | NR_002819->NM_001614 | NOT Evaluated | | E9: chr11: 65021808-65030513 |
| 744 | MALAT1->ACTG1 | NR_002819->NM_001614 | NOT Evaluated | | E9: chr11: 65021808-65030513 |
| 745 | COL1A1->CD276 | NM_000088->NM_025240 | | | E44: chr17: 45620455-45620509 |
| 746 | COL1A1->CD276 | NM_000088->NM_001024736 | | | E44: chr17: 45620455-45620509 |
| 747 | COL1A1->CD276 | NM_000088->NM_025240 | | | E44: chr17: 45620455-45620509 |
| 748 | COL1A1->CD276 | NM_000088->NM_001024736 | | | E44: chr17: 45620455-45620509 |
| 749 | SLC26A2->CD24 | NM_000112->NM_013230 | YES | | E3: chr5: 149340048-149347156 |
| 750 | MTG1->LOC619207 | NM_138384->NR_002934 | | | E9: chr10: 135066185-135066267 |
| 751 | MTG1->LOC619207 | NM_138384->NR_002934 | | | E9: chr10: 135066185-135066267 |
| 752 | MTG1->LOC619207 | NM_138384->NR_002934 | | | E9: chr10: 135066185-135066267 |
| 753 | YWHAZ->ZBTB33 | NM_001135700->NM_006777 | YES | | E6: chr8: 101999980-102002156 |
| 754 | YWHAZ->ZBTB33 | NM_001135700->NM_001184742 | YES | | E6: chr8: 101999980-102002156 |
| 755 | YWHAZ->ZBTB33 | NM_003406->NM_006777 | YES | | E6: chr8: 101999980-102002156 |
| 756 | YWHAZ->ZBTB33 | NM_003406->NM_001184742 | YES | | E6: chr8: 101999980-102002156 |
| 757 | YWHAZ->ZBTB33 | NM_145690->NM_006777 | YES | | E6: chr8: 101999980-102002156 |
| 758 | YWHAZ->ZBTB33 | NM_145690->NM_001184742 | YES | | E6: chr8: 101999980-102002156 |
| 759 | YWHAZ->ZBTB33 | NM_001135699->NM_006777 | YES | | E6: chr8: 101999980-102002156 |
| 760 | YWHAZ->ZBTB33 | NM_001135699->NM_001184742 | YES | | E6: chr8: 101999980-102002156 |
| 761 | YWHAZ->ZBTB33 | NM_001135702->NM_006777 | YES | | E6: chr8: 101999980-102002156 |
| 762 | YWHAZ->ZBTB33 | NM_001135702->NM_001184742 | YES | | E6: chr8: 101999980-102002156 |
| 763 | YWHAZ->ZBTB33 | NM_001135701->NM_006777 | YES | | E6: chr8: 101999980-102002156 |
| 764 | YWHAZ->ZBTB33 | NM_001135701->NM_001184742 | YES | | E6: chr8: 101999980-102002156 |
| 765 | SEMA4C->PKM2 | NM_017789->NM_002654 | YES | | E15: chr2: 96889199-96890919 |
| 766 | SEMA4C->PKM2 | NM_017789->NM_182471 | YES | | E15: chr2: 96889199-96890919 |
| 767 | SEMA4C->PKM2 | NM_017789->NM_182470 | YES | | E15: chr2: 96889199-96890919 |
| 768 | ALDOA->TAGLN2 | NM_184043->NM_003564 | (part of | | E35: chr16: 29988651-29988851 |
| | | | ALDOA) | | |
| 769 | ALDOA->TAGLN2 | NM_184041->NM_003564 | (part of | | E35: chr16: 29988651-29988851 |
| | | | ALDOA) | | |
| 770 | ALDOA->TAGLN2 | NM_001127617->NM_003564 | (part of | | E35: chr16: 29988651-29988851 |
| | | | ALDOA) | | |
| 771 | ALDOA->TAGLN2 | NM_000034->NM_003564 | (part of | | E55: chr16: 29988651-29988851 |
| | | | ALDOA) | | |
| 772 | NCOR2->ELN | NM_001077261->NM_001081754 | | | E38: chr12: 123390504-123390703 |
| 773 | NCOR2->ELN | NM_001077261->NM_001081753 | | | E38: chr12: 123390504-123390703 |
| 774 | NCOR2->ELN | NM_001077261->NM_001081755 | | | E38: chr12: 123390504-123390703 |
| 775 | NCOR2->ELN | NM_001077261->NM_001081752 | | | E38: chr12: 123390504-123390703 |
| 776 | NCOR2->ELN | NM_001077261->NM_000501 | | | E38: chr12: 123390504-123390703 |
| 777 | NCOR2->ELN | NM_006312->NM_001081754 | | | E39: chr12: 123390504-123390703 |
| 778 | NCOR2->ELN | NM_006312->NM_001081753 | | | E39: chr12: 123390504-123390703 |
| 779 | NCOR2->ELN | NM_006312->NM_001081755 | | | E39: chr12: 123390504-123390703 |
| 780 | NCOR2->ELN | NM_006312->NM_001081752 | | | E39: chr12: 123390504-123390703 |
| 781 | NCOR2->ELN | NM_006312->NM_000501 | | | E39: chr12: 123390504-123390703 |
| 782 | HLA-A->ARF1 | NM_002116->NM_001658 | | | E15: chr6: 30020989-30021037 |
| 783 | HLA-A->ARF1 | NM_002116->NM_001024226 | | | E15: chr6: 30020989-30021037 |
| 784 | HLA-A->ARF1 | NM_002116->NM_001024227 | | | E15: chr6: 30020989-30021037 |
| 785 | HLA-A->ARF1 | NM_002116->NM_001024228 | | | E15: chr6: 30020989-30021037 |
| 786 | COL1A1->YWHAG | NM_000088->NM_012479 | YES | | E51: chr17: 45616455-45618008 |
| 787 | NAV2->WDFY1 | NM_001111018->NM_020830 | YES | | E114: chr11: 20096254-20099723 |
| 788 | NAV2->WDFY1 | NM_145117->NM_020830 | YES | | E114: chr11: 20096254-20099723 |
| 789 | NAV2->WDFY1 | NM_182964->NM_020830 | YES | | E114: chr11: 20096254-20099723 |
| 790 | NAV2->WDFY1 | NM_001111019->NM_020830 | YES | | E54: chr11: 20096254-20099723 |
| 791 | H1F0->ACTB | NM_005318->NM_001101 | YES | | E3: chr22: 36531059-36533389 |
| 792 | GOLPH3L->CTSS | NM_018178->NM_004079 | | | E4: chr1: 148900913-148901028 |
| 793 | CALR->NCL | NM_004343->NM_005381 | | | E72: chr19: 12915526-12916304 |
| 794 | CALR->NCL | NM_004343->NM_005381 | | | E72: chr19: 12915526-12916304 |
| 795 | CALR->NCL | NM_004343->NM_005381 | | | E72: chr19: 12915526-12916304 |
| 796 | CALR->NCL | NM_004343->NM_005381 | | | E72: chr19: 12915526-12916304 |
| 797 | C9orf30->TMEFF1 | NM_080655->NM_003692 | YES | | E2: chr9: 102244008-102244459 |
| 798 | C9orf30->TMEFF1 | NM_080655->NM_003692 | YES | | E2: chr9: 102244008-102244459 |
| 799 | C9orf30->TMEFF1 | NM_080655->NM_003692 | YES | | E2: chr9: 102244008-102244459 |
| 800 | C9orf30->TMEFF1 | NM_080655->NM_003692 | YES | | E2: chr9: 102244008-102244459 |
| 801 | C9orf30->TMEFF1 | NM_080655->NM_003692 | YES | | E2: chr9: 102244008-102244459 |
| 802 | C9orf30->TMEFF1 | NM_080655->NM_003692 | YES | | E2: chr9: 102244008-102244459 |
| 803 | MYH9->COL1A1 | NM_002473->NM_000088 | | | E37: chr22: 35011649-35011773 |

| | | Fusion Transcript | |
|---|---|---|---|
| | | Coding Sequence | |
| # | Boundary Exon 3′ Gene | (SEQ ID:) | Fusion Protein Sequence (SEQ ID NO: or GenBank Accession No.) |
| 1 | E17: chr14: 51993557-51993642 | 597 | 1083 |
| 2 | E17: chr14: 51993557-51993642 | 598 | 1084 |
| 3 | E4: chr7: 5534437-5534876 | 599 | 1085 |
| 4 | E4: chr7: 5534437-5534876 | 600 | 1086 |
| 5 | E10: chr11: 34442227-34442358 | 601 | 1087 |
| 6 | E10: chr11: 34442227-34442358 | 602 | 1088 |
| 7 | E10: chr11: 34442227-34442358 | 603 | 1089 |
| 8 | E45: chr17: 45620235-45620343 | 604 | 1090 |
| 9 | E45: chr17: 45620235-45620343 | 605 | 1091 |
| 10 | E45: chr17: 45620235-45620343 | 606 | 1092 |
| 11 | E7: chr3: 77678178-77678303 | 607 | 1093 |
| 12 | E6: chr3: 77678178-77678303 | 608 | 1094 |
| 13 | E6: chr11: 1732711-1732834 | | the entire LTBP4 protein from NM_003573 |
| 14 | E6: chr11: 1732711-1732834 | | the entire LTBP4 protein from NM_001042544 |
| 15 | E6: chr11: 1732711-1732834 | | the entire LTBP4 protein from NM_001042545 |
| 16 | E3: chr8: 128972016-128972426 | 609 | Assuming: intact protein for NR_003367 |
| 17 | E3: chr8: 128972016-128972426 | 610 | Assuming: intact protein for NR_003367 |
| 18 | E3: chr8: 128972016-128972426 | 611 | Assuming: intact protein for NR_003367 |
| 19 | E3: chr8: 128972016-128972426 | 612 | Assuming: intact protein for NR_003367 |
| 20 | E3: chr8: 128972016-128972426 | 613 | Assuming: intact protein for NR_003367 |
| 21 | E3: chr8: 128972016-128972426 | 614 | Assuming: intact protein for NR_003367 |
| 22 | E2: chr8: 128936582-128936747 | 615 | Assuming: intact protein for NR_003367 |
| 23 | E2: chr8: 128936582-128936747 | 616 | Assuming: intact protein for NR_003367 |
| 24 | E5: chr20: 43387342-43389469 | | the entire PTMA protein from NM_001099285 |
| 25 | E5: chr20: 43387342-43389469 | | the entire PTMA protein from NM_002823 |
| 26 | E1: chr12: 51199792-51200510 | 617 | 1095 |
| 27 | E3: chr17: 77229079-77229120 | 618 | 1096 |
| 28 | E2: chr17: 77229079-77229120 | 619 | 1097 |
| 29 | E9: chr11: 1730560-1731476 | | the entire SFN protein from NM_006142 |
| 30 | E1: chr17: 36996087-36996673 | | the entire KRT7 protein from NM_005556 |
| 31 | E2: chr20: 1249006-1249079 | 620 | 1098 |
| 32 | E2: chr20: 1249006-1249079 | 621 | 1099 |
| 33 | E2: chr20: 1249006-1249079 | 622 | 1100 |
| 34 | E2: chr20: 1249006-1249079 | 623 | 1101 |
| 35 | E6: chr21: 41642388-41642521 | 624 | 1102 |
| 36 | E7: chr21: 41642388-41642521 | 625 | 1103 |
| 37 | E4: chr7: 5534437-5534876 | 626 | 1104 |
| 38 | E1: chr12: 51199792-51200510 | 627 | 1105 |
| 39 | E1: chr12: 51199792-51200510 | 628 | 1106 |
| 40 | E1: chr3: 66633303-66633535 | 629 | 1107 |
| 41 | E26: chr12: 48317990-48325953 | | the entire COL1A1 protein from NM_000088 |
| 42 | E25: chr12: 48317990-48325953 | | the entire COL1A1 protein from NM_000088 |
| 43 | E16: chr16: 29725355-29725715 | 630 | 1108 |
| 44 | E19: chr16: 29725355-29725715 | 631 | 1109 |
| 45 | E8: chr6: 33375451-33377526 | | the entire PTRF protein from NM_012232 |
| 46 | E7: chr6: 33375451-33377526 | | the entire PTRF protein from NM_012232 |
| 47 | E11: chr22: 21565879-21565998 | 632 | 1110 |
| 48 | E11: chr22: 21565879-21565998 | 633 | 1111 |
| 49 | E2: chr12: 90082512-90082625 | | the entire VPS35 protein from NM_018206 |
| 50 | E3: chr12: 90082512-90082625 | | the entire VPS35 protein from NM_018206 |
| 51 | E3: chr12: 90082512-90082625 | | the entire VPS35 protein from NM_018206 |
| 52 | E2: chr12: 90082512-90082625 | | the entire VPS35 protein from NM_018206 |
| 53 | E3: chr12: 90082512-90082625 | | the entire VPS35 protein from NM_018206 |
| 54 | E3: chr12: 90082512-90082625 | | the entire VPS35 protein from NM_018206 |
| 55 | E1: chr17: 36914833-36915391 | | the entire GAPDH protein from NM_002046 |
| 56 | E1: chr17: 36914833-36915391 | | the entire GAPDH protein from NM_002046 |
| 57 | E612: chr2: 189584598-189585717 | | the entire SPATS2L protein from NM_015535 |
| 57 | E612: chr2: 189584598-189585717 | | the entire SPATS2L protein from NM_001100422 |
| 59 | E612: chr2: 189584598-189585717 | | the entire SPATS2L protein from NM_001100424 |
| 60 | E612: chr2: 189584598-189585717 | | the entire SPATS2L protein from NM_001100423 |
| 61 | E6: chr17: 58863396-58865687 | | the entire YWHAG protein from NM_012479 |
| 62 | E6: chr17: 58863396-58865687 | | the entire YWHAG protein from NM_012479 |
| 63 | E6: chr17: 58863396-58865687 | | the entire YWHAG protein from NM_012479 |
| 64 | E1: chr14: 68515421-68515836 | | the entire LASP1 protein from NM_006148 |
| 65 | E1: chr14: 68515421-68515836 | | the entire LASP1 protein from NM_006148 |
| 66 | E1: chr14: 68515421-68515836 | | the entire LASP1 protein from NM_006148 |
| 67 | E18: chr10: 76458252-76462645 | 634 | 1112 |
| 68 | E18: chr10: 76458252-76462645 | 635 | 1113 |
| 69 | E18: chr10: 76458252-76462645 | 636 | 1114 |
| 70 | E2: chr5: 17328316-17329943 | | the entire COL1A1 protein from NM_000088 |
| 71 | E26: chr12: 56209209-56210198 | | the entire COL1A1 protein from NM_000088 |
| 72 | E8: chr6: 30568504-30569960 | | the entire TSPAN14 protein from NM_030927 |
| 73 | E8: chr6: 30568504-30569960 | | the entire TSPAN14 protein from NM_001128309 |
| 74 | E8: chr6: 30568504-30569960 | | the entire TSPAN14 protein from NM_030927 |
| 75 | E8: chr6: 30568504-30569960 | | the entire TSPAN14 protein from NM_001128309 |
| 76 | E1: chr8: 145088547-145088680 | 637 | 1115 |
| 77 | E256: chr8: 145088547-145088680 | 638 | 1116 |
| 78 | E264: chr8: 145088547-145088680 | 639 | 1117 |
| 79 | E20: chr8: 145076539-145076692 | 640 | 1118 |
| 80 | E20: chr8: 145076539-145076692 | 641 | 1119 |
| 81 | E20: chr8: 145076539-145076692 | 642 | 1120 |
| 82 | E20: chr8: 145076539-145076692 | 643 | 1121 |
| 83 | E20: chr8: 145076539-145076692 | 644 | 1122 |
| 84 | E20: chr8: 145076539-145076692 | 645 | 1123 |
| 85 | E20: chr8: 145076539-145076692 | 646 | 1124 |
| 86 | E21: chr8: 145076539-145076692 | 647 | 1125 |
| 87 | E4: chr7: 5534437-5534876 | 648 | 1126 |
| 88 | E8: chr7: 26199719-26199839 | 649 | 1127 |
| 89 | E9: chr7: 26199719-26199839 | 650 | 1128 |
| 90 | E2: chr7: 75794043-75797486 | 651 | 1129 |
| 91 | E2: chr7: 75794043-75797486 | 652 | 1130 |
| 92 | E2: chr7: 75794043-75797486 | 653 | 1131 |
| 93 | E2: chr7: 75794043-75797486 | 654 | 1132 |
| 94 | E2: chr7: 75794043-75797486 | 655 | 1133 |
| 95 | E1: chr17: 36996087-36996673 | 656 | 1134 |
| 96 | E1: chr17: 36996087-36996673 | 657 | 1135 |
| 97 | E1: chr12: 51199792-51200510 | 658 | 1136 |
| 98 | E1: chr12: 51199792-51200510 | 659 | 1137 |
| 99 | E1: chr12: 51199792-51200510 | 660 | 1138 |
| 100 | E1: chr12: 51199792-51200510 | 661 | 1139 |
| 101 | E20: chr12: 56206615-56207277 | | the entire CD74 protein from NM_001025158 |
| 102 | E2: chr14: 68324127-68326962 | | the entire CALR protein from NM_004343 |
| 103 | E72: chr19: 12915526-12916304 | | the entire ZFP36L1 protein from NM_004926 |
| 104 | E2: chr14: 68324127-68326962 | | the entire CALR protein from NM_004343 |
| 105 | E50: chr17: 45618137-45618380 | 662 | 1140 |
| 106 | E50: chr17: 45618137-45618380 | 663 | 1141 |
| 107 | E33: chr17: 45623176-45623284 | 664 | 1142 |
| 108 | E3: chr17: 45631915-45631950 | 665 | 1143 |
| 109 | E32: chr8: 145061308-145068551 | 666 | 1144 |
| 110 | E32: chr8: 145061308-145068551 | 667 | 1145 |
| 111 | E32: chr8: 145061308-145068551 | 668 | 1146 |
| 112 | E32: chr8: 145061308-145068551 | 669 | 1147 |
| 113 | E32: chr8: 145061308-145068551 | 670 | 1148 |
| 114 | E32: chr8: 145061308-145068551 | 671 | 1149 |
| 115 | E32: chr8: 145061308-145068551 | 672 | 1150 |
| 116 | E33: chr8: 145061308-145068551 | 673 | 1151 |
| 117 | E9: chr11: 1730560-1731476 | | the entire EPHA2 protein from NM_004431 |
| 118 | E17: chr8: 87639631-87642842 | | the entire IFI27 protein from NM_001130080 |
| 119 | E17: chr8: 87639631-87642842 | | the entire IFI27 protein from NM_005532 |
| 120 | E8: chr19: 10230284-10231736 | 674 | 1153 |
| 121 | E8: chr19: 10230284-10231736 | 675 | 1154 |
| 122 | E8: chr19: 10230284-10231736 | 676 | 1155 |
| 123 | E31: chr8: 145068659-145072040 | 677 | 1156 |
| 124 | E31: chr8: 145068659-145072040 | 678 | 1157 |
| 125 | E31: chr8: 145068659-145072040 | 679 | 1157 |
| 126 | E31: chr8: 145068659-145072040 | 680 | 1158 |
| 127 | E31: chr8: 145068659-145072040 | 681 | 1159 |
| 128 | E31: chr8: 145068659-145072040 | 682 | 1160 |
| 129 | E31: chr8: 145068659-145072040 | 683 | 1161 |
| 130 | E32: chr8: 145068659-145072040 | 684 | 1162 |
| 131 | E31: chr8: 145068659-145072040 | 685 | 1163 |
| 132 | E31: chr8: 145068659-145072040 | 686 | 1164 |
| 133 | E31: chr8: 145068659-145072040 | 687 | 1165 |
| 134 | E31: chr8: 145068659-145072040 | 688 | 1166 |
| 135 | E31: chr8: 145068659-145072040 | 689 | 1167 |
| 136 | E31: chr8: 145068659-145072040 | 690 | 1168 |
| 137 | E31: chr8: 145068659-145072040 | 691 | 1169 |
| 138 | E32: chr8: 145068659-145072040 | 692 | 1170 |
| 139 | E37: chr19: 44539168-44539569 | | the entire C2orf56 protein from NM_144736 |
| 140 | E37: chr19: 44539168-44539569 | | the entire C2orf56 protein from NM_001083946 |
| 141 | E20: chr1: 114736921-114742005 | | the entire POSTN protein from NM_001135935 |
| 142 | E19: chr1: 114736921-114742005 | | the entire POSTN protein from NM_001135935 |
| 143 | E20: chr1: 114736921-114742005 | | the entire POSTN protein from NM_006475 |
| 144 | E19: chr1: 114736921-114742005 | | the entire POSTN protein from NM_006475 |
| 145 | E20: chr1: 114736921-114742005 | | the entire POSTN protein from NM_001135934 |
| 146 | E19: chr1: 114736921-114742005 | | the entire POSTN protein from NM_001135934 |
| 147 | E20: chr1: 114736921-114742005 | | the entire POSTN protein from NM_001135936 |
| 148 | E19: chr1: 114736921-114742005 | | the entire POSTN protein from NM_001135936 |
| 149 | E1: chrY: 19611913-19614093 | 693 | 1171 |
| 150 | E1: chrY: 19611913-19614093 | 694 | 1172 |
| 151 | E2: chr19: 9810111-9810324 | 695 | 1173 |
| 152 | E2: chr19: 9810111-9810324 | 696 | 1174 |
| 153 | E2: chr19: 9810111-9810324 | 697 | 1175 |
| 154 | E2: chr19: 9810111-9810324 | 698 | 1176 |
| 155 | E2: chr19: 9810111-9810324 | 699 | 1177 |
| 156 | E1: chr17: 36996087-36996673 | 700 | 1178 |
| 157 | E5: chr17: 45631585-45631687 | 701 | 1179 |
| 158 | E2: chr5: 151035885-151035955 | 702 | 1180 |
| 159 | E2: chr5: 151035885-151035955 | 703 | 1181 |
| 160 | E2: chr5: 151035885-151035955 | 704 | 1182 |
| 161 | E35: chr21: 45749475-45749620 | 705 | 1183 |
| 162 | E36: chr21: 45749475-45749620 | 706 | 1184 |
| 163 | E35: chr21: 45749475-45749620 | 707 | 1185 |
| 164 | E2: chr5: 151035885-151035955 | 708 | 1186 |
| 165 | E2: chr5: 151035885-151035955 | 709 | 1187 |
| 166 | E2: chr5: 151035885-151035955 | 710 | 1188 |
| 167 | E2: chr5: 151035885-151035955 | 711 | 1189 |
| 168 | E2: chr5: 151035885-151035955 | 712 | 1190 |
| 169 | E2: chr5: 151035885-151035955 | 713 | 1191 |
| 170 | E10: chr6: 111302679-111303111 | | the entire IGFBP5 protein from NM_000599 |
| 171 | E39: chr17: 45621736-45621898 | 714 | 1192 |
| 172 | E39: chr17: 45621736-45621898 | 715 | 1193 |
| 173 | E54: chr3: 40478433-40478863 | 716 | 1194 |
| 174 | E54: chr3: 40478433-40478863 | 717 | 1195 |
| 175 | E13: chr1: 207865615-207865994 | 718 | 1196 |
| 176 | E14: chr1: 207865615-207865994 | 719 | 1197 |
| 177 | E14: chr1: 207865615-207865994 | 720 | 1198 |
| 178 | E5: chr6: 37089362-37089519 | 721 | 1199 |
| 179 | E2: chr1: 243070563-243075269 | | the entire CTTN protein from NM_005231 |
| 180 | E5: chr1: 94767319-94768740 | | the entire FBLIM1 protein from NM_017556 |
| 181 | E6: chr1: 94767319-94768740 | | the entire FBLIM1 protein from NM_017556 |
| 182 | E5: chr1: 94767319-94768740 | | the entire FBLIM1 protein from NM_001024216 |
| 183 | E6: chr1: 94767319-94768740 | | the entire FBLIM1 protein from NM_001024216 |
| 184 | E7: chr6: 30701257-30702153 | | the entire GAPDH protein from NM_002046 |
| 185 | E11: chr17: 34143675-34145379 | 722 | 1200 |
| 186 | E1: chr11: 525415-525550 | 723 | 1201 |
| 187 | E1: chr11: 525415-525550 | 724 | 1202 |
| 188 | E1: chr11: 525415-525550 | 725 | 1203 |
| 189 | E9: chr12: 51194625-51195291 | | the entire RNF213 protein from NM_020914 |
| 190 | E23: chr10: 24709803-24710002 | 726 | 1204 |
| 191 | E37: chr10: 24709803-24710002 | 727 | 1205 |
| 192 | E45: chr10: 24709803-24710002 | 728 | 1206 |
| 193 | E25: chr1: 120269450-120269956 | 729 | 1207 |
| 194 | E14: chr6: 35918289-35918359 | 730 | 1208 |
| 195 | E14: chr6: 35918289-35918359 | 731 | 1209 |
| 196 | E14: chr6: 35918289-35918359 | 732 | 1210 |
| 197 | E14: chr6: 35918289-35918359 | 733 | 1211 |
| 198 | E2: chr8: 22318153-22318438 | 734 | Assuming: intact protein for NM_015359 |
| 199 | E2: chr8: 22318153-22318438 | 735 | Assuming: intact protein for NM_001128431 |
| 200 | E2: chr8: 22318153-22318438 | 736 | Assuming: intact protein for NM_001135154 |
| 201 | E2: chr8: 22318153-22318438 | 737 | Assuming: intact protein for NM_001135153 |
| 202 | E2: chr8: 22318153-22318438 | 738 | Assuming: intact protein for NM_015359 |
| 203 | E2: chr8: 22318153-22318438 | 739 | Assuming: intact protein for NM_001128431 |
| 204 | E2: chr8: 22318153-22318438 | 740 | Assuming: intact protein for NM_001135154 |
| 205 | E2: chr8: 22318153-22318438 | 741 | Assuming: intact protein for NM_001135153 |
| 206 | E2: chr8: 22318153-22318438 | 742 | Assuming: intact protein for NM_015359 |
| 207 | E2: chr8: 22318153-22318438 | 743 | Assuming: intact protein for NM_001128431 |
| 208 | E2: chr8: 22318153-22318438 | 744 | Assuming: intact protein for NM_001135154 |
| 209 | E2: chr8: 22318153-22318438 | 745 | Assuming: intact protein for NM_001135153 |
| 210 | E2: chr8: 22318153-22318438 | 746 | Assuming: intact protein for NM_015359 |
| 211 | E2: chr8: 22318153-22318438 | 747 | Assuming: intact protein for NM_001128431 |
| 212 | E2: chr8: 22318153-22318438 | 748 | Assuming: intact protein for NM_001135154 |
| 213 | E2: chr8: 22318153-22318438 | 749 | Assuming: intact protein for NM_001135153 |
| 214 | E6: chr7: 5533304-5534048 | | the entire IRF2BP2 protein from NM_182972 |
| 215 | E6: chr7: 5533304-5534048 | | the entire IRF2BP2 protein from NM_001077397 |
| 216 | E2: chr19: 44618086-44618188 | 750 | 1212 |
| 217 | E2: chr18: 22335033-22335212 | | the entire LOC728606 protein from NR_024259 |
| 218 | E2: chr18: 22335033-22335212 | | the entire LOC728606 protein from NR_024259 |
| 219 | E3: chr18: 22335033-22335212 | | the entire LOC728606 protein from NR_024259 |
| 220 | E2: chr18: 22335033-22335212 | | the entire LOC728606 protein from NR_024259 |
| 221 | E2: chr18: 22335033-22335212 | | the entire LOC728606 protein from NR_024259 |
| 222 | E3: chr18: 22335033-22335212 | | the entire LOC728606 protein from NR_024259 |
| 223 | E1: chr12: 51199792-51200510 | 751 | 1213 |
| 224 | E1: chr12: 51199792-51200510 | 752 | 1214 |
| 225 | E1: chr17: 35472593-35472871 | 753 | 1215 |
| 226 | E1: chr17: 35472593-35472871 | 754 | 1216 |
| 227 | E1: chr17: 35472593-35472871 | 755 | 1217 |
| 228 | E48: chr9: 139021506-139022237 | 756 | 1218 |
| 229 | E48: chr9: 139021506-139022237 | 757 | 1219 |
| 230 | E48: chr9: 139021506-139022237 | 758 | 1220 |
| 231 | E48: chr9: 139021506-139022237 | 759 | 1221 |
| 232 | E14: chr10: 111883073-111885313 | | the entire FTL protein from NM_000146 |
| 233 | E15: chr10: 111883073-111885313 | | the entire FTL protein from NM_000146 |
| 234 | E14: chr10: 111883073-111885313 | | the entire FTL protein from NM_000146 |
| 235 | E32: chr1: 144152539-144153985 | | the entire CYB5R3 protein from NM_001171660 |
| 236 | E32: chr1: 144152539-144153985 | | the entire CYB5R3 protein from NM_001171661 |
| 237 | E32: chr1: 144152539-144153985 | | the entire CYB5R3 protein from NM_007326 |
| 238 | E32: chr1: 144152539-144153985 | | the entire CYB5R3 protein from NM_001129819 |
| 239 | E32: chr1: 144152539-144153985 | | the entire CYB5R3 protein from NM_000398 |
| 240 | E2: chr14: 102663094-102663719 | 760 | 1222 |
| 241 | E13: chr17: 20843497-20846978 | | the entire MRPL52 protein from NM_178336 |
| 242 | E13: chr17: 20843497-20846978 | | the entire MRPL52 protein from NM_180982 |
| 243 | E13: chr17: 20843497-20846978 | | the entire MRPL52 protein from NM_181306 |
| 244 | E13: chr17: 20843497-20846978 | | the entire MRPL52 protein from NM_181305 |
| 245 | E13: chr17: 20843497-20846978 | | the entire MRPL52 protein from NM_181304 |
| 246 | E13: chr17: 20843497-20846978 | | the entire MRPL52 protein from NM_181307 |
| 247 | E9: chr11: 1730560-1731476 | | the entire PLXNA1 protein from NM_032242 |
| 248 | E53: chr1: 31904224-31904269 | 761 | 1223 |
| 249 | E1: chr1: 2110341-2114884 | | the entire SLC9A3R1 protein from NM_004252 |
| 250 | E6: chr19: 18133088-18133305 | 762 | 1224 |
| 251 | E48: chrX: 153230093-153230598 | | the entire SBF1 protein from NM_002972 |
| 252 | E47: chrX: 153230093-153230598 | | the entire SBF1 protein from NM_002972 |
| 253 | E13: chr16: 54096751-54098087 | | the entire CAV1 protein from NM_001753 |
| 254 | E13: chr16: 54096751-54098087 | | the entire CAV1 protein from NM_001753 |
| 255 | E13: chr16: 54096751-54098087 | | the entire CAV1 protein from NM_001172895 |
| 256 | E13: chr16: 54096751-54098087 | | the entire CAV1 protein from NM_001172895 |
| 257 | E13: chr16: 54096751-54098087 | | the entire CAV1 protein from NM_001172896 |
| 258 | E13: chr16: 54096751-54098087 | | the entire CAV1 protein from NM_001172896 |
| 259 | E13: chr16: 54096751-54098087 | | the entire CAV1 protein from NM_001172897 |
| 260 | E13: chr16: 54096751-54098087 | | the entire CAV1 protein from NM_001172897 |
| 261 | E1: chr19: 18911659-18912113 | | the entire CTSD protein from NM_001909 |
| 262 | E8: chr11: 101605642-101609364 | 763 | 1225 |
| 263 | E9: chr11: 101605642-101609364 | 764 | 1226 |
| 264 | E7: chr11: 101605642-101609364 | 765 | 1227 |
| 265 | E9: chr11: 101605642-101609364 | 766 | 1228 |
| 266 | E2: chr2: 46839161-46843431 | 767 | 1229 |
| 267 | E2: chr2: 46839161-46843431 | 768 | 1230 |
| 268 | E1: chr16: 52877196-52877879 | 769 | 1231 |
| 269 | E7: chr12: 53536820-53536943 | 770 | 1232 |
| 270 | E10: chr5: 151021201-151023353 | | the entire SRRM2 protein from NM_016333 |
| 271 | E10: chr5: 151021201-151023353 | | the entire SRRM2 protein from NM_016333 |
| 272 | E48: chr8: 121452571-121453454 | | the entire DNAJA2 protein from NM_005880 |
| 273 | E6: chr7: 5533304-5534048 | | the entire KRT81 protein from NM_002281 |
| 274 | E1: chr13: 76352304-76358541 | 771 | 1233 |
| 275 | E1: chr5: 179267309-179267462 | 772 | 1234 |
| 276 | E1: chr5: 179267309-179267462 | 773 | 1235 |
| 277 | E2: chr19: 44618086-44618188 | 774 | 1236 |
| 278 | E2: chr10: 104455092-104455236 | 775 | 1237 |
| 279 | E2: chr10: 104455092-104455236 | 776 | 1238 |
| 280 | E2: chr10: 104455092-104455236 | 777 | 1239 |
| 281 | E2: chr10: 104455092-104455236 | 778 | 1240 |
| 282 | E2: chr10: 104455092-104455236 | 779 | 1241 |
| 283 | E2: chr10: 104455092-104455236 | 780 | 1242 |
| 284 | E2: chr10: 104455092-104455236 | 781 | 1243 |
| 285 | E2: chr10: 104455092-104455236 | 782 | 1244 |
| 286 | E6: chr22: 21567554-21568011 | 783 | 1245 |
| 287 | E12: chr22: 21567554-21568011 | 784 | 1246 |
| 288 | E6: chr22: 21567554-21568011 | 785 | 1247 |
| 289 | E12: chr22: 21567554-21568011 | 786 | 1248 |
| 290 | E6: chr22: 21567554-21568011 | 787 | 1249 |
| 291 | E12: chr22: 21567554-21568011 | 788 | 1250 |
| 292 | E2: chr6: 74008849-74008974 | | the entire C6orf147 protein from NR_027005 |
| 293 | E1: chr12: 11039827-11041741 | 789 | 1251 |
| 294 | E1: chr12: 11039827-11041741 | 790 | 1252 |
| 295 | E1: chr12: 11039827-11041741 | 791 | 1253 |
| 296 | E1: chr12: 11039827-11041741 | 792 | 1254 |
| 297 | E6: chr7: 5533304-5534048 | | the entire ACTN4 protein from NM_004924 |
| 298 | E6: chr7: 5533304-5534048 | | the entire ACTN4 protein from NM_004924 |
| 299 | E6: chr7: 5533304-5534048 | | the entire ACTN4 protein from NM_004924 |
| 300 | E6: chr7: 5533304-5534048 | | the entire ACTN4 protein from NM_004924 |
| 301 | E6: chr7: 5533304-5534048 | | the entire ACTN4 protein from NM_004924 |
| 302 | E12: chr17: 16285406-16286063 | | the entire MGP protein from NM_000900 |
| 303 | E10: chr17: 16285406-16286063 | | the entire MGP protein from NM_000900 |
| 304 | E10: chr17: 16285406-16286063 | | the entire MGP protein from NM_000900 |
| 305 | E12: chr17: 16285406-16286063 | | the entire MGP protein from NM_000900 |
| 306 | E12: chr17: 16285406-16286063 | | the entire MGP protein from NM_000900 |
| 307 | E10: chr17: 16285406-16286063 | | the entire MGP protein from NM_000900 |
| 308 | E8: chr17: 16285406-16286063 | | the entire MGP protein from NM_000900 |
| 309 | E8: chr17: 16285406-16286063 | | the entire MGP protein from NM_000900 |
| 310 | E10: chr17: 16285406-16286063 | | the entire MGP protein from NM_000900 |
| 311 | E10: chr17: 16285406-16286063 | | the entire MGP protein from NM_000900 |
| 312 | E10: chr17: 16285406-16286063 | | the entire MGP protein from NM_000900 |
| 313 | E8: chr17: 16285406-16286063 | | the entire MGP protein from NM_000900 |
| 314 | E10: chr17: 16285406-16286063 | | the entire MGP protein from NM_000900 |
| 315 | E10: chr17: 16285406-16286063 | | the entire MGP protein from NM_000900 |
| 316 | E12: chr17: 16285406-16286063 | | the entire MGP protein from NM_001190839 |
| 317 | E10: chr17: 16285406-16286063 | | the entire MGP protein from NM_001190839 |
| 318 | E10: chr17: 16285406-16286063 | | the entire MGP protein from NM_001190839 |
| 319 | E12: chr17: 16285406-16286063 | | the entire MGP protein from NM_001190839 |
| 320 | E12: chr17: 16285406-16286063 | | the entire MGP protein from NM_001190839 |
| 321 | E10: chr17: 16285406-16286063 | | the entire MGP protein from NM_001190839 |
| 322 | E8: chr17: 16285406-16286063 | | the entire MGP protein from NM_001190839 |
| 323 | E8: chr17: 16285406-16286063 | | the entire MGP protein from NM_001190839 |
| 324 | E10: chr17: 16285406-16286063 | | the entire MGP protein from NM_001190839 |
| 325 | E10: chr17: 16285406-16286063 | | the entire MGP protein from NM_001190839 |
| 326 | E10: chr17: 16285406-16286063 | | the entire MGP protein from NM_001190839 |
| 327 | E8: chr17: 16285406-16286063 | | the entire MGP protein from NM_001190839 |
| 328 | E10: chr17: 16285406-16286063 | | the entire MGP protein from NM_001190839 |
| 329 | E10: chr17: 16285406-16286063 | | the entire MGP protein from NM_001190839 |
| 330 | E1: chr4: 170167673-170168043 | | the entire PALLD protein from NM_016081 |
| 331 | E1: chr4: 170167673-170168043 | | the entire PALLD protein from NM_016081 |
| 332 | E6: chr7: 5533304-5534048 | 793 | 1255 |
| 333 | E4: chr17: 77092808-77093247 | 794 | 1256 |
| 334 | E5: chr11: 1735129-1735362 | | the entire GNB2 protein from NM_005273 |
| 335 | E10: chr6: 139289230-139289311 | 795 | 1257 |
| 336 | E19: chr6: 139289230-139289311 | 796 | NOT Calculated |
| 337 | E10: chr6: 139289230-139289311 | 797 | 1258 |
| 338 | E19: chr6: 139289230-139289311 | 798 | NOT Calculated |
| 339 | E10: chr6: 139289230-139289311 | 799 | 1259 |
| 340 | E19: chr6: 139289230-139289311 | 800 | NOT Calculated |
| 341 | E10: chr6: 139289230-139289311 | 801 | 1260 |
| 342 | E19: chr6: 139289230-139289311 | 802 | NOT Calculated |
| 343 | E10: chr6: 139289230-139289311 | 803 | 1261 |
| 344 | E19: chr6: 139289230-139289311 | 804 | NOT Calculated |
| 345 | E10: chr6: 139289230-139289311 | 805 | 1262 |
| 346 | E19: chr6: 139289230-139289311 | 806 | NOT Calculated |
| 347 | E11: chr6: 139283866-139283954 | 807 | 1263 |
| 348 | E10: chr6: 139283866-139283954 | 808 | 1264 |
| 349 | E11: chr6: 139283866-139283954 | 809 | 1265 |
| 350 | E10: chr6: 139283866-139283954 | 810 | 1266 |
| 351 | E11: chr6: 139283866-139283954 | 811 | 1267 |
| 352 | E10: chr6: 139283866-139283954 | 812 | 1268 |
| 353 | E11: chr6: 139283866-139283954 | 813 | 1269 |
| 354 | E10: chr6: 139283866-139283954 | 814 | 1270 |
| 355 | E11: chr6: 139283866-139283954 | 815 | 1271 |
| 356 | E10: chr6: 139283866-139283954 | 816 | 1272 |
| 357 | E11: chr6: 139283866-139283954 | 817 | 1273 |
| 358 | E10: chr6: 139283866-139283954 | 818 | 1274 |
| 359 | E5: chr11: 2106922-2111029 | 819 | NOT Calculated |
| 360 | E5: chr11: 2106922-2111029 | 820 | NOT Calculated |
| 361 | E4: chr11: 2106922-2111029 | 821 | NOT Calculated |
| 362 | E1: chr17: 45633770-45633999 | 822 | 1275 |
| 363 | E1: chr17: 45633770-45633999 | 823 | 1276 |
| 364 | E1: chr17: 45633770-45633999 | 824 | 1277 |
| 365 | E1: chr17: 45633770-45633999 | 825 | 1278 |
| 366 | E1: chr17: 45633770-45633999 | 826 | 1279 |
| 367 | E1: chr12: 51131589-51132177 | 827 | 1280 |
| 368 | E3: chr1: 158480373-158480448 | | the entire APOOL protein from NM_198450 |
| 369 | E2: chr1: 158480373-158480448 | | the entire APOOL protein from NM_198450 |
| 370 | E3: chr1: 158480373-158480448 | | the entire APOOL protein from NM_198450 |
| 371 | E3: chr1: 158480373-158480448 | | the entire APOOL protein from NM_198450 |
| 372 | E3: chr1: 158480373-158480448 | | the entire APOOL protein from NM_198450 |
| 373 | E9: chr11: 1730560-1731476 | | the entire PACSIN3 protein from NM_016223 |
| 374 | E9: chr11: 1730560-1731476 | | the entire PACSIN3 protein from NM_001184975 |
| 375 | E9: chr11: 1730560-1731476 | | the entire PACSIN3 protein from NM_001184974 |
| 376 | E1: chr12: 51199792-51200510 | 828 | 1281 |
| 377 | E1: chr17: 45633770-45633999 | | the entire HEATR5A protein from NM_015473 |
| 378 | E2: chr3: 101831131-101831245 | 829 | 1282 |
| 379 | E2: chr3: 101831131-101831245 | 830 | 1283 |
| 380 | E2: chr3: 101831131-101831245 | 831 | 1284 |
| 381 | E2: chr3: 101831131-101831245 | 832 | 1285 |
| 382 | E2: chr10: 126385194-126385446 | | the entire METTL10 protein from NM_212554 |
| 383 | E2: chr10: 126385194-126385446 | | the entire METTL10 protein from NM_212554 |
| 384 | E2: chr10: 126385194-126385446 | | the entire METTL10 protein from NM_212554 |
| 385 | E1: chr12: 51199792-51200510 | | the entire NUFIP2 protein from NM_020772 |
| 386 | E1: chr12: 51199792-51200510 | | the entire NUFIP2 protein from NM_020772 |
| 387 | E1: chr2: 63922517-63922842 | | the entire CIRBP protein from NM_001280 |
| 388 | E2: chr22: 38258345-38258474 | 833 | 1286 |
| 389 | E1: chrX: 72928764-72965791 | | the entire COL1A2 protein from NM_000089 |
| 390 | E1: chr11: 63770463-63770989 | 834 | 1287 |
| 391 | E1: chr11: 63770463-63770989 | 835 | 1288 |
| 392 | E2: chr17: 55777623-55777751 | 836 | 1289 |
| 393 | E2: chr17: 55777623-55777751 | 837 | 1290 |
| 394 | E1: chr12: 51199792-51200510 | 838 | 1291 |
| 395 | E1: chr12: 51199792-51200510 | 839 | 1292 |
| 396 | E2: chr16: 4504576-4504666 | 840 | 1293 |
| 397 | E4: chr2: 217245072-217249850 | | the entire RAB3IP protein from NM_175625 |
| 398 | E4: chr2: 217245072-217249850 | | the entire RAB3IP protein from NM_175624 |
| 399 | E4: chr2: 217245072-217249850 | | the entire RAB3IP protein from NM_022456 |
| 400 | E4: chr2: 217245072-217249850 | | the entire RAB3IP protein from NM_175623 |
| 401 | E4: chr2: 217245072-217249850 | | the entire RAB3IP protein from NM_001024647 |
| 402 | E4: chr2: 217245072-217249850 | | the entire RAB3IP protein from NM_175625 |
| 403 | E4: chr2: 217245072-217249850 | | the entire RAB3IP protein from NM_175624 |
| 404 | E4: chr2: 217245072-217249850 | | the entire RAB3IP protein from NM_022456 |
| 405 | E4: chr2: 217245072-217249850 | | the entire RAB3IP protein from NM_175623 |
| 406 | E4: chr2: 217245072-217249850 | | the entire RAB3IP protein from NM_001024647 |
| 407 | E24: chr6: 56587339-56590175 | 841 | NOT Calculated |
| 408 | E5: chr8: 43046480-43046607 | 842 | 1294 |
| 409 | E4: chr8: 43046480-43046607 | 843 | 1295 |
| 410 | E5: chr16: 10529780-10534450 | | the entire KRT81 protein from NM_002281 |
| 411 | E2: chr8: 81077788-81077970 | 844 | 1296 |
| 412 | E2: chr8: 81077788-81077970 | 845 | 1297 |
| 413 | E2: chr8: 81077788-81077970 | 846 | 1298 |
| 414 | E11: chr7: 555359-556765 | 847 | 1299 |
| 415 | E11: chr7: 555359-556765 | 848 | 1300 |
| 416 | E11: chr7: 555359-556765 | 849 | 1301 |
| 417 | E11: chr7: 555359-556765 | 850 | 1302 |
| 418 | E11: chr7: 555359-556765 | 851 | 1303 |
| 419 | E11: chr7: 555359-556765 | 852 | 1304 |
| 420 | E9: chr11: 65021808-65030513 | | the entire ASAP1 protein from NM_018482 |
| 421 | E66: chr6: 44326005-44326314 | 853 | 1305 |
| 422 | E13: chr4: 15777353-15777514 | 854 | 1306 |
| 423 | E13: chr4: 15777353-15777514 | 855 | 1307 |
| 424 | E13: chr4: 15777353-15777514 | 856 | 1308 |
| 425 | E13: chr4: 15777353-15777514 | 857 | 1309 |
| 426 | E13: chr4: 15777353-15777514 | 858 | 1310 |
| 427 | E13: chr4: 15777353-15777514 | 859 | 1311 |
| 428 | E13: chr4: 15777353-15777514 | 860 | 1312 |
| 429 | E1: chr6: 114285219-114285716 | 861 | 1313 |
| 430 | E2: chr12: 51491813-51492028 | 862 | 1314 |
| 431 | E2: chr12: 51491813-51492028 | 863 | 1315 |
| 432 | E3: chr11: 64946844-64950577 | | the entire CD68 protein from NM_001251 |
| 433 | E3: chr11: 64946844-64950577 | | the entire CD68 protein from NM_001040059 |
| 434 | E4: chr15: 63001172-63001271 | 864 | 1316 |
| 435 | E4: chr15: 63001172-63001271 | 865 | 1317 |
| 436 | E4: chr15: 63001172-63001271 | 866 | 1318 |
| 437 | E4: chr15: 63001172-63001271 | 867 | 1319 |
| 438 | E3: chr20: 10341177-10342579 | 868 | 1320 |
| 439 | E3: chr20: 10341177-10342579 | 869 | 1321 |
| 440 | E1: chr8: 116749945-116750402 | 870 | 1322 |
| 441 | E1: chr8: 116749945-116750402 | | the entire SPARC protein from NM_003118 |
| 442 | E1: chr19: 4408611-4408791 | | the entire FLNA protein from NM_001110556 |
| 443 | E1: chr19: 4408611-4408791 | | the entire FLNA protein from NM_001456 |
| 444 | E18: chr19: 988623-990064 | | the entire WDR82 protein from NM_025222 |
| 445 | E21: chr19: 988623-990064 | | the entire WDR82 protein from NM_025222 |
| 446 | E18: chr19: 988623-990064 | | the entire WDR82 protein from NM_025222 |
| 447 | E21: chr19: 988623-990064 | | the entire WDR82 protein from NM_025222 |
| 448 | E3: chr3: 48939898-48940250 | | the entire TMEM119 protein from NM_181724 |
| 449 | E3: chr3: 131178231-131179466 | | the entire GNB1 protein from NM_002074 |
| 450 | E6: chr18: 31950634-31950740 | | the entire ELF3 protein from NM_001114309 |
| 451 | E5: chr18: 31950634-31950740 | | the entire ELF3 protein from NM_001114309 |
| 452 | E6: chr18: 31950634-31950740 | | the entire ELF3 protein from NM_004433 |
| 453 | E5: chr18: 31950634-31950740 | | the entire ELF3 protein from NM_004433 |
| 454 | E1: chr17: 37033855-37034408 | 871 | 1323 |
| 455 | E1: chr19: 10625936-10626163 | 872 | 1324 |
| 456 | E21: chr19: 10625936-10626163 | 873 | 1325 |
| 457 | E21: chr19: 10625936-10626163 | 874 | 1326 |
| 458 | E1: chr19: 10625936-10626163 | 875 | 1327 |
| 459 | E1: chr19: 10625936-10626163 | 876 | 1328 |
| 460 | E609: chr2: 189581894-189582192 | | the entire BAT2L2 protein from NM_015172 |
| 461 | E5: chr11: 64545768-64546232 | 877 | 1329 |
| 462 | E8: chr15: 42797096-42797649 | | |
| 463 | E8: chr15: 42797096-42797649 | | the entire IGLL5 protein from NM_001178126 |
| 464 | E3: chr17: 77093523-77093763 | 878 | 1330 |
| 465 | E1: chr19: 56164557-56164741 | 879 | 1331 |
| 466 | E1: chr11: 10786827-10787158 | | the entire RAB8A protein from NM_005370 |
| 467 | E1: chr11: 10786827-10787158 | | the entire RAB8A protein from NM_005370 |
| 468 | E5: chr19: 54160377-54160678 | 880 | 1332 |
| 469 | E5: chr19: 54160377-54160678 | 881 | 1333 |
| 470 | E9: chrX: 119454376-119457176 | | the entire COL1A2 protein from NM_000089 |
| 471 | E3: chr19: 44616144-44616241 | 882 | 1334 |
| 472 | E3: chr19: 44616144-44616241 | 883 | 1335 |
| 473 | E3: chr19: 44616144-44616241 | 884 | 1336 |
| 474 | E3: chr19: 44616144-44616241 | 885 | 1337 |
| 475 | E3: chr19: 44616144-44616241 | 886 | 1338 |
| 476 | E3: chr19: 44616144-44616241 | 887 | 1339 |
| 477 | E3: chr19: 44616144-44616241 | 888 | 1340 |
| 478 | E3: chr19: 44616144-44616241 | 889 | 1341 |
| 479 | E2: chr18: 46827287-46827663 | 890 | 1342 |
| 480 | E6: chr7: 5533304-5534048 | | the entire RPS5 protein from NM_001009 |
| 481 | E55: chr17: 32516039-32518487 | 891 | 1343 |
| 482 | E54: chr17: 32516039-32518487 | 892 | 1344 |
| 483 | E56: chr17: 32516039-32518487 | 893 | 1345 |
| 484 | E60: chr17: 32516039-32518487 | 894 | 1346 |
| 485 | E56: chr17: 32516039-32518487 | 895 | 1347 |
| 486 | E6: chr18: 9944049-9950018 | 896 | 1348 |
| 487 | E7: chr18: 9944049-9950018 | 897 | 1349 |
| 488 | E4: chr7: 5534437-5534876 | 898 | 1350 |
| 489 | E2: chr12: 119146336-119146563 | 899 | 1351 |
| 490 | E2: chr12: 119146336-119146563 | 900 | 1352 |
| 491 | E2: chr12: 119146336-119146563 | 901 | 1353 |
| 492 | E2: chr12: 119146336-119146563 | 902 | 1354 |
| 493 | E2: chr12: 119146336-119146563 | 903 | 1355 |
| 494 | E2: chr12: 119146336-119146563 | 904 | 1356 |
| 495 | E2: chr17: 35333808-35334004 | 905 | 1357 |
| 496 | E2: chr17: 35333808-35334004 | 906 | 1358 |
| 497 | E6: chr7: 5533304-5534048 | | the entire OGT protein from NM_181673 |
| 498 | E6: chr7: 5533304-5534048 | | the entire OGT protein from NM_181672 |
| 499 | E6: chr7: 5533304-5534048 | | the entire OGT protein from NM_181673 |
| 500 | E6: chr7: 5533304-5534048 | | the entire OGT protein from NM_181672 |
| 501 | E4: chr19: 21779591-21784449 | | the entire COL3A1 protein from NM_000090 |
| 502 | E3: chr14: 20339354-20340092 | 907 | 1359 |
| 503 | E3: chr14: 20339354-20340092 | 908 | 1360 |
| 504 | E2: chr14: 20339354-20340092 | 909 | 1361 |
| 505 | E2: chr14: 20339354-20340092 | 910 | 1362 |
| 506 | E4: chr17: 77092808-77093247 | 911 | 1363 |
| 507 | E1: chr2: 191453791-191454441 | 912 | 1364 |
| 508 | E1: chr2: 191453791-191454441 | 913 | 1365 |
| 509 | E9: chr11: 65021808-65030513 | | the entire TAX1BP1 protein from NM_001079864 |
| 510 | E9: chr11: 65021808-65030513 | | the entire TAX1BP1 protein from NM_006024 |
| 511 | E35: chr10: 24537725-24538198 | | the entire SERPINA1 protein from NM_001002235 |
| 512 | E43: chr10: 24537725-24538198 | | the entire SERPINA1 protein from NM_001002235 |
| 513 | E35: chr10: 24537725-24538198 | | the entire SERPINA1 protein from NM_001127705 |
| 514 | E43: chr10: 24537725-24538198 | | the entire SERPINA1 protein from NM_001127705 |
| 515 | E35: chr10: 24537725-24538198 | | the entire SERPINA1 protein from NM_001002236 |
| 516 | E43: chr10: 24537725-24538198 | | the entire SERPINA1 protein from NM_001002236 |
| 517 | E35: chr10: 24537725-24538198 | | the entire SERPINA1 protein from NM_001127707 |
| 518 | E43: chr10: 24537725-24538198 | | the entire SERPINA1 protein from NM_001127707 |
| 519 | E35: chr10: 24537725-24538198 | | the entire SERPINA1 protein from NM_001127706 |
| 520 | E43: chr10: 24537725-24538198 | | the entire SERPINA1 protein from NM_001127706 |
| 521 | E35: chr10: 24537725-24538198 | | the entire SERPINA1 protein from NM_001127702 |
| 522 | E43: chr10: 24537725-24538198 | | the entire SERPINA1 protein from NM_001127702 |
| 523 | E35: chr10: 24537725-24538198 | | the entire SERPINA1 protein from NM_001127701 |
| 524 | E43: chr10: 24537725-24538198 | | the entire SERPINA1 protein from NM_001127701 |
| 525 | E35: chr10: 24537725-24538198 | | the entire SERPINA1 protein from NM_001127700 |
| 526 | E43: chr10: 24537725-24538198 | | the entire SERPINA1 protein from NM_001127700 |
| 527 | E35: chr10: 24537725-24538198 | | the entire SERPINA1 protein from NM_001127703 |
| 528 | E43: chr10: 24537725-24538198 | | the entire SERPINA1 protein from NM_001127703 |
| 529 | E35: chr10: 24537725-24538198 | | the entire SERPINA1 protein from NM_001127704 |
| 530 | E43: chr10: 24537725-24538198 | | the entire SERPINA1 protein from NM_001127704 |
| 531 | E35: chr10: 24537725-24538198 | | the entire SERPINA1 protein from NM_000295 |
| 532 | E43: chr10: 24537725-24538198 | | the entire SERPINA1 protein from NM_000295 |
| 533 | E2: chr6: 52375918-52380534 | 914 | 1366 |
| 534 | E2: chr6: 52375918-52380534 | 915 | 1367 |
| 535 | E47: chr12: 131112963-131113199 | 916 | 1368 |
| 536 | E47: chr12: 131112963-131113199 | 917 | 1369 |
| 537 | E2: chr8: 67571225-67571281 | 918 | 1370 |
| 538 | E2: chr8: 67571225-67571281 | | the entire GPATCH8 protein from NR_036474 |
| 539 | E51: chr17: 45616455-45618008 | | the entire PTRF protein from NM_012232 |
| 540 | E1: chrX: 46938144-46938367 | | the entire CDK4 protein from NM_000075 |
| 541 | E9: chrX: 152935081-152935289 | | the entire GAPDH protein from NM_002046 |
| 542 | E9: chrX: 152935081-152935289 | | the entire GAPDH protein from NM_002046 |
| 543 | E9: chrX: 152935081-152935289 | | the entire GAPDH protein from NM_002046 |
| 544 | E11: chr10: 73249476-73249663 | | the entire CD68 protein from NM_001251 |
| 545 | E10: chr10: 73249476-73249663 | | the entire CD68 protein from NM_001251 |
| 546 | E10: chr10: 73249476-73249663 | | the entire CD68 protein from NM_001251 |
| 547 | E11: chr10: 73249476-73249663 | | the entire CD68 protein from NM_001040059 |
| 548 | E10: chr10: 73249476-73249663 | | the entire CD68 protein from NM_001040059 |
| 549 | E10: chr10: 73249476-73249663 | | the entire CD68 protein from NM_001040059 |
| 550 | E11: chr10: 73249476-73249663 | | the entire CD68 protein from NM_001251 |
| 551 | E10: chr10: 73249476-73249663 | | the entire CD68 protein from NM_001251 |
| 552 | E10: chr10: 73249476-73249663 | | the entire CD68 protein from NM_001251 |
| 553 | E11: chr10: 73249476-73249663 | | the entire CD68 protein from NM_001040059 |
| 554 | E10: chr10: 73249476-73249663 | | the entire CD68 protein from NM_001040059 |
| 555 | E10: chr10: 73249476-73249663 | | the entire CD68 protein from NM_001040059 |
| 556 | E6: chr7: 5533304-5534048 | | the entire APOL1 protein from NM_003661 |
| 557 | E6: chr7: 5533304-5534048 | | the entire APOL1 protein from NM_145343 |
| 558 | E6: chr7: 5533304-5534048 | | the entire APOL1 protein from NM_001136541 |
| 559 | E6: chr7: 5533304-5534048 | | the entire APOL1 protein from NM_001136540 |
| 560 | E2: chr21: 39756170-39756356 | 919 | 1371 |
| 561 | E2: chr21: 39756170-39756356 | 920 | 1372 |
| 562 | E2: chr21: 39756170-39756356 | 921 | 1373 |
| 563 | E2: chr21: 39756170-39756356 | 922 | 1374 |
| 564 | E2: chr21: 39756170-39756356 | 923 | 1375 |
| 565 | E2: chr21: 39756170-39756356 | 924 | 1376 |
| 566 | E2: chr21: 39756170-39756356 | 925 | 1377 |
| 567 | E2: chr21: 39756170-39756356 | 926 | 1378 |
| 568 | E2: chr21: 39756170-39756356 | 927 | 1379 |
| 569 | E2: chr21: 39756170-39756356 | 928 | 1380 |
| 570 | E2: chr21: 39756170-39756356 | 929 | 1381 |
| 571 | E2: chr21: 39756170-39756356 | 930 | 1382 |
| 572 | E1: chr2: 27163114-27163723 | | the entire ITGA3 protein from NM_002204 |
| 573 | E1: chr2: 27163114-27163723 | | the entire ITGA3 protein from NM_002204 |
| 574 | E1: chr2: 27163114-27163723 | 931 | 1383 |
| 575 | E1: chr2: 27163114-27163723 | 932 | 1384 |
| 576 | E2: chr14: 95798741-95798860 | 933 | 1385 |
| 577 | E2: chr14: 95798741-95798860 | 934 | 1386 |
| 578 | E6: chr17: 16980257-16980489 | 935 | 1387 |
| 579 | E6: chr17: 16980257-16980489 | 936 | 1388 |
| 580 | E6: chr17: 16980257-16980489 | 937 | 1389 |
| 581 | E6: chr17: 16980257-16980489 | 938 | 1390 |
| 582 | E2: chr12: 107507750-107510302 | | the entire PIKFYVE protein from NM_015040 |
| 583 | E1: chr11: 1741597-1741798 | | the entire TMEM109 protein from NM_024092 |
| 584 | E4: chr2: 217245072-217249850 | | the entire SREBF1 protein from NM_004176 |
| 585 | E4: chr2: 217245072-217249850 | | the entire SREBF1 protein from NM_001005291 |
| 586 | E4: chr2: 217245072-217249850 | | the entire SREBF1 protein from NM_004176 |
| 587 | E4: chr2: 217245072-217249850 | | the entire SREBF1 protein from NM_001005291 |
| 588 | E18: chrX: 17075456-17081324 | | the entire MGP protein from NM_000900 |
| 589 | E18: chrX: 17075456-17081324 | | the entire MGP protein from NM_000900 |
| 590 | E18: chrX: 17075456-17081324 | | the entire MGP protein from NM_001190839 |
| 591 | E18: chrX: 17075456-17081324 | | the entire MGP protein from NM_001190839 |
| 592 | E6: chr7: 5533304-5534048 | | the entire AKT2 protein from NM_001626 |
| 593 | E6: chr7: 5533304-5534048 | | the entire AKT2 protein from NM_001626 |
| 594 | E14: chr9: 129307438-129309531 | | the entire SBF1 protein from NM_002972 |
| 595 | E14: chr9: 129307438-129309531 | | the entire SBF1 protein from NM_002972 |
| 596 | E1: chr9: 129370919-129371176 | | the entire SBF1 protein from NM_002972 |
| 597 | E56: chr9: 129370919-129371176 | | the entire SBF1 protein from NM_002972 |
| 598 | E4: chr20: 19977983-19978075 | | the entire RHOBTB3 protein from NM_014899 |
| 599 | E22: chr19: 60294092-60294738 | 939 | 1391 |
| 600 | E15: chr10: 98267856-98272077 | | the entire POSTN protein from NM_001135935 |
| 601 | E15: chr10: 98267856-98272077 | | the entire POSTN protein from NM_006475 |
| 602 | E15: chr10: 98267856-98272077 | | the entire POSTN protein from NM_001135934 |
| 603 | E15: chr10: 98267856-98272077 | | the entire POSTN protein from NM_001135936 |
| 604 | E3: chr11: 386813-387445 | 940 | 1392 |
| 605 | E3: chr11: 386813-387445 | 941 | 1393 |
| 606 | E3: chr11: 386813-387445 | 942 | 1394 |
| 607 | E3: chr11: 386813-387445 | 943 | 1395 |
| 608 | E3: chr11: 386813-387445 | 944 | 1396 |
| 609 | E3: chr11: 386813-387445 | 945 | 1397 |
| 610 | E19: chr2: 241827689-241827908 | | the entire NTN1 protein from NM_004822 |
| 611 | E19: chr2: 241827689-241827908 | | the entire NTN1 protein from NM_004822 |
| 612 | E2: chr3: 15506035-15506148 | 946 | 1398 |
| 613 | E2: chr3: 15506035-15506148 | 947 | 1399 |
| 614 | E2: chr3: 15506035-15506148 | 948 | 1400 |
| 615 | E2: chr3: 15506035-15506148 | 949 | 1401 |
| 616 | E2: chr3: 15506035-15506148 | 950 | 1402 |
| 617 | E2: chr3: 15506035-15506148 | 951 | 1403 |
| 618 | E1: chr13: 44813176-44813297 | | the entire SHANK3 protein from NM_001080420 |
| 619 | E5: chr17: 74360653-74363541 | | the entire COL1A1 protein from NM_000088 |
| 620 | E1: chr17: 77603051-77603624 | | the entire FLNA protein from NM_001110556 |
| 621 | E1: chr17: 77603051-77603624 | | the entire FLNA protein from NM_001456 |
| 622 | E1: chr15: 41825881-41826196 | | the entire YWHAG protein from NM_012479 |
| 623 | E1: chr15: 41825881-41826196 | | the entire YWHAG protein from NM_012479 |
| 624 | E2: chr2: 61614410-61614542 | | the entire MAPK1IP1L protein from NM_144578 |
| 625 | E24: chr17: 64683141-64683249 | 952 | 1404 |
| 626 | E3: chr2: 171514294-171514498 | 953 | 1405 |
| 627 | E3: chr2: 171514294-171514498 | 954 | 1406 |
| 628 | E4: chr7: 5534437-5534876 | 955 | 1407 |
| 629 | E12: chr12: 74730577-74730700 | 956 | 1408 |
| 630 | E12: chr12: 74730577-74730700 | 957 | 1409 |
| 631 | E3: chr1: 84740096-84740241 | 958 | 1410 |
| 632 | E3: chr1: 84740096-84740241 | 959 | 1411 |
| 633 | E3: chr1: 84740096-84740241 | 960 | 1412 |
| 634 | E3: chr1: 84740096-84740241 | 961 | 1413 |
| 635 | E3: chr1: 84740096-84740241 | 962 | 1414 |
| 636 | E3: chr1: 84740096-84740241 | 963 | 1415 |
| 637 | E3: chr1: 84740096-84740241 | 964 | 1416 |
| 638 | E3: chr1: 84740096-84740241 | 965 | 1417 |
| 639 | E3: chr1: 84740096-84740241 | 966 | 1418 |
| 640 | E3: chr1: 84740096-84740241 | 967 | 1419 |
| 641 | E3: chr1: 84740096-84740241 | 968 | 1420 |
| 642 | E9: chr12: 51167224-51168006 | 969 | 1421 |
| 643 | E9: chr12: 51167224-51168006 | 970 | 1422 |
| 644 | E9: chr12: 51167224-51168006 | 971 | 1423 |
| 645 | E5: chr15: 70289067-70289254 | 972 | 1424 |
| 646 | E5: chr15: 70289067-70289254 | 973 | 1425 |
| 647 | E5: chr15: 70289067-70289254 | 974 | 1426 |
| 648 | E11: chr15: 70278423-70279151 | 975 | 1427 |
| 649 | E11: chr15: 70278423-70279151 | 976 | 1428 |
| 650 | E11: chr15: 70278423-70279151 | 977 | 1429 |
| 651 | E13: chr17: 70256357-70257021 | 978 | 1430 |
| 652 | E10: chr3: 180596569-180601801 | | the entire PTMA protein from NM_001099285 |
| 653 | E10: chr3: 180596569-180601801 | | the entire PTMA protein from NM_002823 |
| 654 | E2: chr12: 4830164-4830538 | 979 | 1431 |
| 655 | E2: chr12: 4830164-4830538 | 980 | 1432 |
| 656 | E2: chr12: 4830164-4830538 | 981 | 1433 |
| 657 | E2: chr3: 50226585-50226737 | 982 | Assuming: intact protein for NM_006841 |
| 658 | E2: chr3: 50226585-50226737 | 983 | Assuming: intact protein for NM_006841 |
| 659 | E1: chr17: 36996087-36996673 | 984 | 1434 |
| 660 | E1: chr17: 36996087-36996673 | 985 | 1435 |
| 661 | E1: chr17: 36996087-36996673 | 986 | 1436 |
| 662 | E7: chr10: 8136672-8136860 | | the entire RHOB protein from NM_004040 |
| 663 | E7: chr10: 8136672-8136860 | | the entire RHOB protein from NM_004040 |
| 664 | E6: chr17: 77091593-77092454 | 987 | 1437 |
| 665 | E14: chr1: 243080224-243084428 | | the entire TES protein from NM_015641 |
| 666 | E14: chr1: 243080224-243084428 | | the entire TES protein from NM_015641 |
| 667 | E14: chr1: 243080224-243084428 | | the entire TES protein from NM_152829 |
| 668 | E14: chr1: 243080224-243084428 | | the entire TES protein from NM_152829 |
| 669 | E1: chr1: 15883413-15883700 | 988 | 1438 |
| 670 | E1: chr1: 15883413-15883700 | 989 | 1439 |
| 671 | E1: chr1: 15883413-15883700 | 990 | 1440 |
| 672 | E1: chr1: 15883413-15883700 | 991 | 1441 |
| 673 | E1: chr1: 15883413-15883700 | 992 | 1442 |
| 674 | E1: chr1: 15883413-15883700 | 993 | 1443 |
| 675 | E1: chr1: 15883413-15883700 | 994 | 1444 |
| 676 | E1: chr1: 15883413-15883700 | 995 | 1445 |
| 677 | E1: chr1: 51474532-51475139 | 996 | 1446 |
| 678 | E1: chr12: 51199792-51200510 | 997 | 1447 |
| 679 | E1: chr12: 51199792-51200510 | 998 | 1448 |
| 680 | E4: chr15: 67459275-67459403 | 999 | 1449 |
| 681 | E4: chr15: 67459275-67459403 | 1000 | 1450 |
| 682 | E6: chr7: 5533304-5534048 | | the entire FUS protein from NM_004960 |
| 683 | E6: chr7: 5533304-5534048 | 1001 | NOT Calculated |
| 684 | E6: chr7: 5533304-5534048 | | the entire FUS protein from NM_001170937 |
| 685 | E6: chr7: 5533304-5534048 | | the entire FUS protein from NM_001170634 |
| 686 | E8: chr20: 30494522-30499280 | | the entire ACTB protein from NM_001101 |
| 687 | E8: chr20: 30494522-30499280 | | the entire ACTB protein from NM_001101 |
| 688 | E4: chr20: 55656857-55661060 | | the entire ACTB protein from NM_001101 |
| 689 | E4: chr20: 55656857-55661060 | | the entire ACTB protein from NM_001101 |
| 690 | E4: chr20: 55656857-55661060 | | the entire ACTB protein from NM_001101 |
| 691 | E4: chr20: 55656857-55661060 | | the entire ACTB protein from NM_001101 |
| 692 | E8: chr6: 16434603-16436680 | 1002 | 1451 |
| 693 | E7: chr6: 16434603-16436680 | 1003 | 1452 |
| 694 | E8: chr6: 16434603-16436680 | 1004 | 1453 |
| 695 | E7: chr6: 16434603-16436680 | 1005 | 1454 |
| 696 | E8: chr6: 16434603-16436680 | 1006 | 1455 |
| 697 | E7: chr6: 16434603-16436680 | 1007 | 1456 |
| 698 | E8: chr6: 16434603-16436680 | 1008 | 1457 |
| 699 | E7: chr6: 16434603-16436680 | 1009 | 1458 |
| 700 | E8: chr6: 16434603-16436680 | 1010 | 1459 |
| 701 | E7: chr6: 16434603-16436680 | 1011 | 1460 |
| 702 | E8: chr6: 16434603-16436680 | 1012 | 1461 |
| 703 | E7: chr6: 16434603-16436680 | 1013 | 1462 |
| 704 | E2: chr22: 20394916-20395197 | 1014 | 1463 |
| 705 | E2: chr22: 20394916-20395197 | 1015 | 1464 |
| 706 | E2: chr11: 12116512-12116587 | 1016 | Assuming: intact protein for NM_014632 |
| 707 | E3: chr11: 12140201-12140542 | 1017 | Assuming: intact protein for NM_014632 |
| 708 | E2: chr15: 70298338-70298505 | 1018 | 1465 |
| 709 | E2: chr15: 70298338-70298505 | 1019 | 1466 |
| 710 | E2: chr15: 70298338-70298505 | 1020 | 1467 |
| 711 | E2: chr13: 31550970-31551170 | 1021 | 1468 |
| 712 | E2: chr13: 31550970-31551170 | 1022 | 1469 |
| 713 | E2: chr13: 31550970-31551170 | 1023 | 1470 |
| 714 | E2: chr13: 31550970-31551170 | 1024 | 1471 |
| 715 | E2: chr13: 31550970-31551170 | 1025 | 1472 |
| 716 | E9: chr12: 51167224-51168006 | 1026 | 1473 |
| 717 | E25: chr5: 139883834-139884009 | 1027 | 1474 |
| 718 | E25: chr5: 139883834-139884009 | 1028 | 1475 |
| 719 | E25: chr5: 139883834-139884009 | 1029 | 1476 |
| 720 | E13: chr9: 35050309-35050522 | | the entire KRT5 protein from NM_000424 |
| 721 | E6: chr7: 5533304-5534048 | | the entire TMED2 protein from NM_006815 |
| 722 | E6: chr7: 5533304-5534048 | | the entire TMED2 protein from NM_006815 |
| 723 | E1: chr12: 51199792-51200510 | | the entire TNS4 protein from NM_032865 |
| 724 | E1: chr12: 51199792-51200510 | | the entire TNS4 protein from NM_032865 |
| 725 | E1: chrY: 19611913-19614093 | | the entire TPM4 protein from NM_001145160 |
| 726 | E1: chrY: 19611913-19614093 | | the entire TPM4 protein from NM_003290 |
| 727 | E1: chr4: 57670799-57671296 | | the entire MAF protein from NM_001031804 |
| 728 | E612: chr2: 189584598-189585717 | | the entire POLD3 protein from NM_006591 |
| 729 | E1: chr17: 37033855-37034408 | 1030 | 1477 |
| 730 | E1: chrY: 19611913-19614093 | | the entire GAPDH protein from NM_002046 |
| 731 | E30: chr3: 185120417-185121883 | 1031 | 1478 |
| 732 | E30: chr3: 185120417-185121883 | 1032 | 1479 |
| 733 | E30: chr3: 185120417-185121883 | 1033 | 1480 |
| 734 | E30: chr3: 185120417-185121883 | 1034 | 1481 |
| 735 | E30: chr3: 185120417-185121883 | 1035 | 1482 |
| 736 | E30: chr3: 185120417-185121883 | 1036 | 1483 |
| 737 | E30: chr3: 185120417-185121883 | 1037 | 1484 |
| 738 | E1: chr12: 51172699-51173448 | 1038 | 1485 |
| 739 | E2: chr16: 22283723-22283836 | | the entire RRN3P3 protein from NR_027460 |
| 740 | E2: chr16: 22283723-22283836 | | the entire RRN3P3 protein from NR_027460 |
| 741 | E4: chr17: 77092808-77093247 | 1039 | 1486 |
| 742 | E4: chr17: 77092808-77093247 | 1040 | 1487 |
| 743 | E4: chr17: 77092808-77093247 | 1041 | 1488 |
| 744 | E4: chr17: 77092808-77093247 | 1042 | 1489 |
| 745 | E1: chr15: 71763674-71763854 | 1043 | 1490 |
| 746 | E1: chr15: 71763674-71763854 | 1044 | 1491 |
| 747 | E1: chr15: 71763674-71763854 | 1045 | 1492 |
| 748 | E1: chr15: 71763674-71763854 | 1046 | 1493 |
| 749 | E1: chrY: 19611913-19614093 | | the entire SLC26A2 protein from NM_000112 |
| 750 | E2: chr10: 135119730-135120048 | 1047 | 1494 |
| 751 | E2: chr10: 135119730-135120048 | 1048 | 1495 |
| 752 | E2: chr10: 135119730-135120048 | 1049 | 1496 |
| 753 | E2: chrX: 119271296-119276279 | | the entire YWHAZ protein from NM_001135700 |
| 754 | E3: chrX: 119271296-119276279 | | the entire YWHAZ protein from NM_001135700 |
| 755 | E2: chrX: 119271296-119276279 | | the entire YWHAZ protein from NM_003406 |
| 756 | E3: chrX: 119271296-119276279 | | the entire YWHAZ protein from NM_003406 |
| 757 | E2: chrX: 119271296-119276279 | | the entire YWHAZ protein from NM_145690 |
| 758 | E3: chrX: 119271296-119276279 | | the entire YWHAZ protein from NM_145690 |
| 759 | E2: chrX: 119271296-119276279 | | the entire YWHAZ protein from NM_001135699 |
| 760 | E3: chrX: 119271296-119276279 | | the entire YWHAZ protein from NM_001135699 |
| 761 | E2: chrX: 119271296-119276279 | | the entire YWHAZ protein from NM_001135702 |
| 762 | E3: chrX: 119271296-119276279 | | the entire YWHAZ protein from NM_001135702 |
| 763 | E2: chrX: 119271296-119276279 | | the entire YWHAZ protein from NM_001135701 |
| 764 | E3: chrX: 119271296-119276279 | | the entire YWHAZ protein from NM_001135701 |
| 765 | E6: chr15: 70288015-70288286 | 1050 | 1497 |
| 766 | E6: chr15: 70288015-70288286 | 1051 | 1498 |
| 767 | E6: chr15: 70288015-70288286 | 1052 | 1499 |
| 768 | E5: chr1: 158154526-158155355 | 1053 | 1500 |
| 769 | E5: chr1: 158154526-158155355 | 1054 | 1501 |
| 770 | E5: chr1: 158154526-158155355 | 1055 | 1502 |
| 771 | E5: chr1: 158154526-158155355 | 1056 | 1503 |
| 772 | E28: chr7: 73115575-73115635 | 1057 | 1504 |
| 773 | E27: chr7: 73115575-73115635 | 1058 | 1505 |
| 774 | E27: chr7: 73115575-73115635 | 1059 | 1506 |
| 775 | E26: chr7: 73115575-73115635 | 1060 | 1507 |
| 776 | E28: chr7: 73115575-73115635 | 1061 | 1508 |
| 777 | E28: chr7: 73115575-73115635 | 1062 | 1509 |
| 778 | E27: chr7: 73115575-73115635 | 1063 | 1510 |
| 779 | E27: chr7: 73115575-73115635 | 1064 | 1511 |
| 780 | E26: chr7: 73115575-73115635 | 1065 | 1512 |
| 781 | E28: chr7: 73115575-73115635 | 1066 | 1513 |
| 782 | E2: chr1: 226351401-226351586 | 1067 | 1514 |
| 783 | E2: chr1: 226351401-226351586 | 1068 | 1515 |
| 784 | E2: chr1: 226351401-226351586 | 1069 | 1516 |
| 785 | E2: chr1: 226351401-226351586 | 1070 | 1517 |
| 786 | E2: chr7: 75794043-75797486 | | the entire COL1A1 protein from NM_000088 |
| 787 | E12: chr2: 224448308-224451691 | | the entire NAV2 protein from NM_001111018 |
| 788 | E12: chr2: 224448308-224451691 | | the entire NAV2 protein from NM_145117 |
| 789 | E12: chr2: 224448308-224451691 | | the entire NAV2 protein from NM_182964 |
| 790 | E12: chr2: 224448308-224451691 | | the entire NAV2 protein from NM_001111019 |
| 791 | E4: chr7: 5534437-5534876 | | the entire H1F0 protein from NM_005318 |
| 792 | E8: chr1: 148969175-148972245 | 1071 | 1518 |
| 793 | E4: chr2: 232033623-232033821 | 1072 | 1519 |
| 794 | E4: chr2: 232033623-232033821 | 1073 | 1520 |
| 795 | E3: chr2: 232034494-232034972 | 1074 | 1521 |
| 796 | E3: chr2: 232034494-232034972 | 1075 | 1522 |
| 797 | E2: chr9: 102300867-102300977 | 1076 | 1523 |
| 798 | E2: chr9: 102300867-102300977 | 1077 | 1524 |
| 799 | E2: chr9: 102300867-102300977 | 1078 | 1525 |
| 800 | E2: chr9: 102300867-102300977 | 1079 | 1526 |
| 801 | E2: chr9: 102300867-102300977 | 1080 | 1527 |
| 802 | E2: chr9: 102300867-102300977 | 1081 | 1528 |
| 803 | E50: chr17: 45618137-45618380 | 1082 | 1529 |

The most common class of fusion transcripts in cell lines occurred within 3′-untranslated regions (3′UTRs). A similar distribution prevailed in primary breast tumors (FIG. 6). Such fusions resulted in the generation of full length coding sequences of the 5′ fusion partner, but altered the 3′ UTR sequence of such transcripts, with potential effects on stability and/or translational efficiency of the fusion transcript (Mayr et al., Science, 315:1576-9 (2007)).

The second broad class of chimaeric transcripts involved fusion within the coding regions. Some of these transcripts contained precise exon/exon junctions (column H of Table 8) and were assumed to be processed. However, the data did not discriminate between tumor-specific trans-splicing events and processing of a primary transcript that arises due to genomic rearrangement. The fusion junctions of many chimaeric transcripts did not correspond to known exon/exon boundaries. These may have arose due to trans-splicing at cryptic sites or, more likely, may represent novel exonic sequences derived from transcription of rearranged genes.

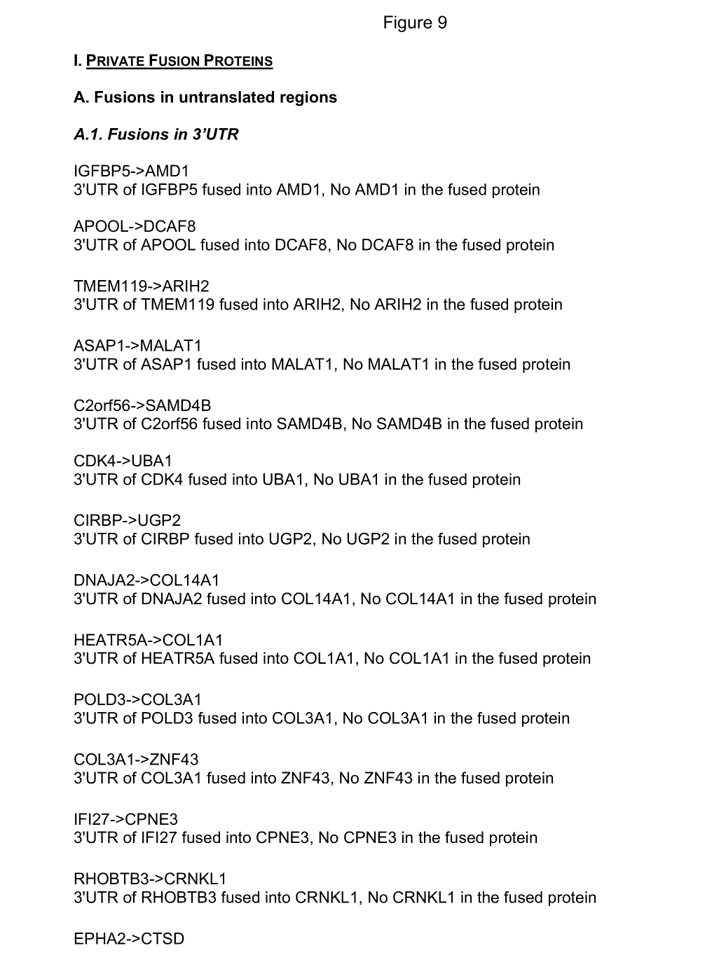

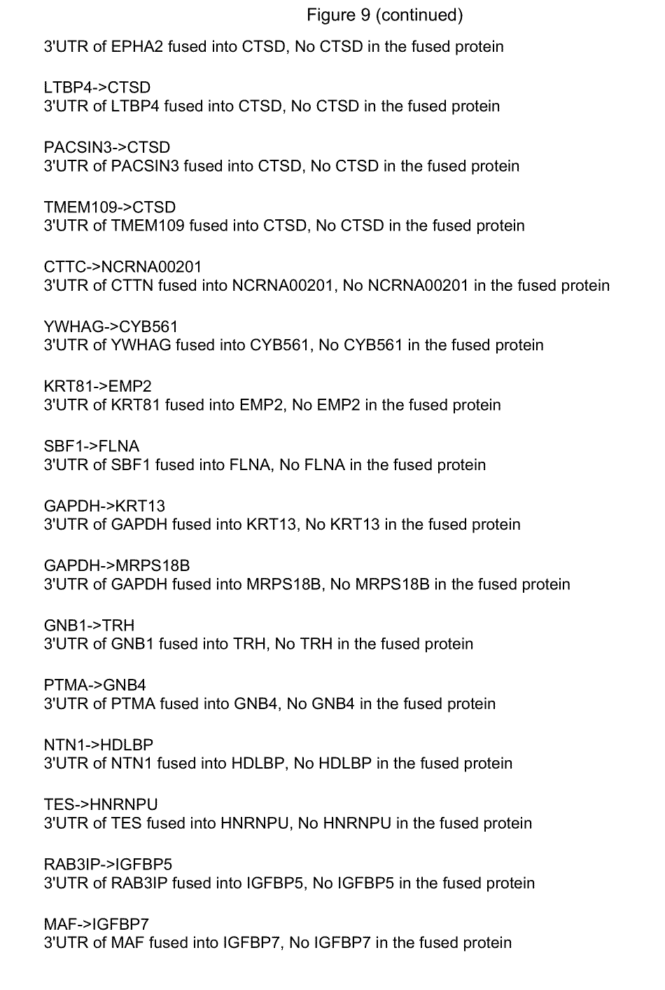

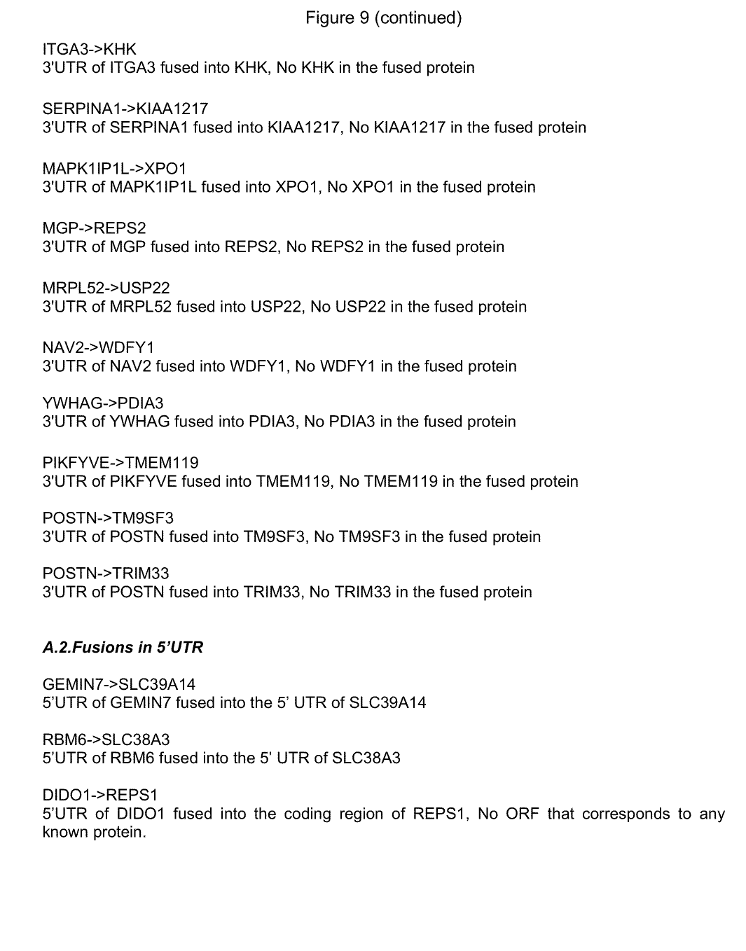

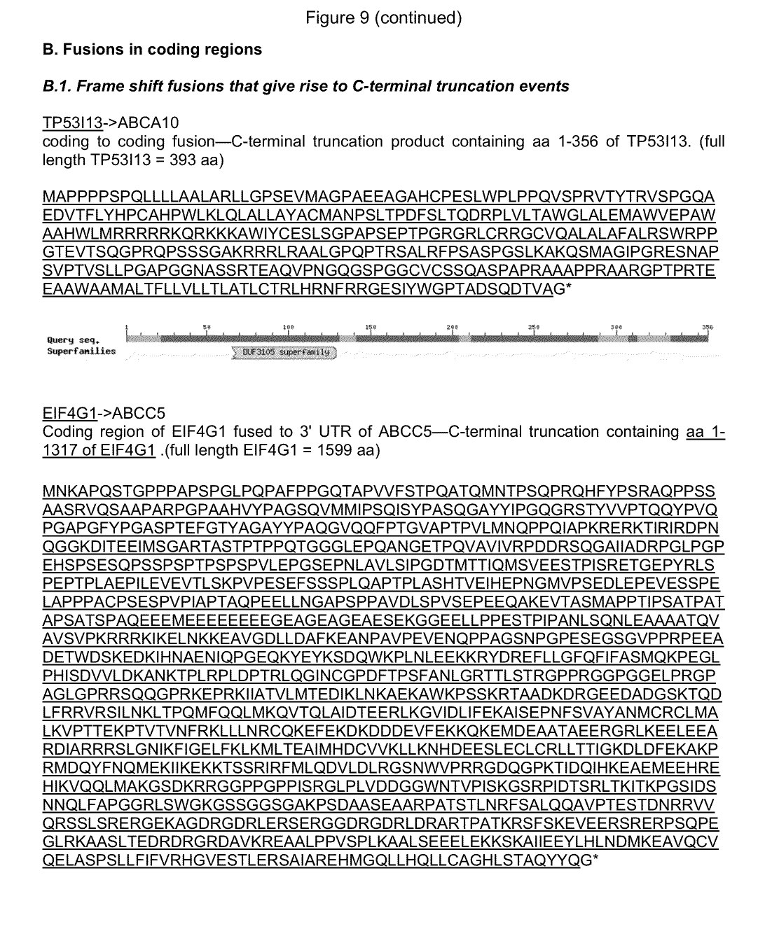

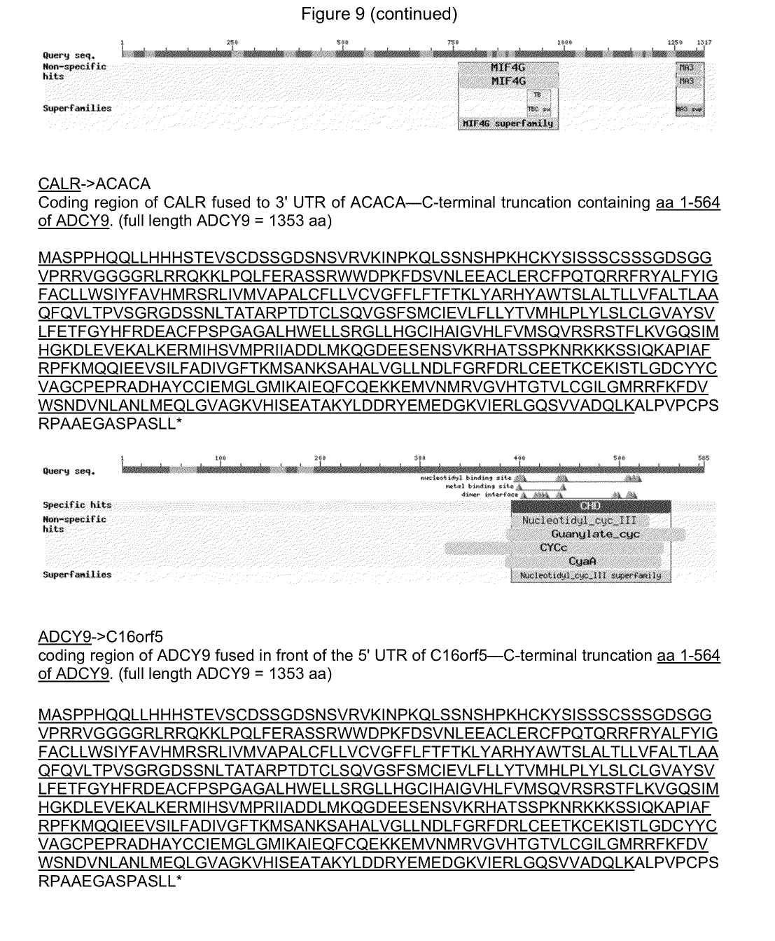

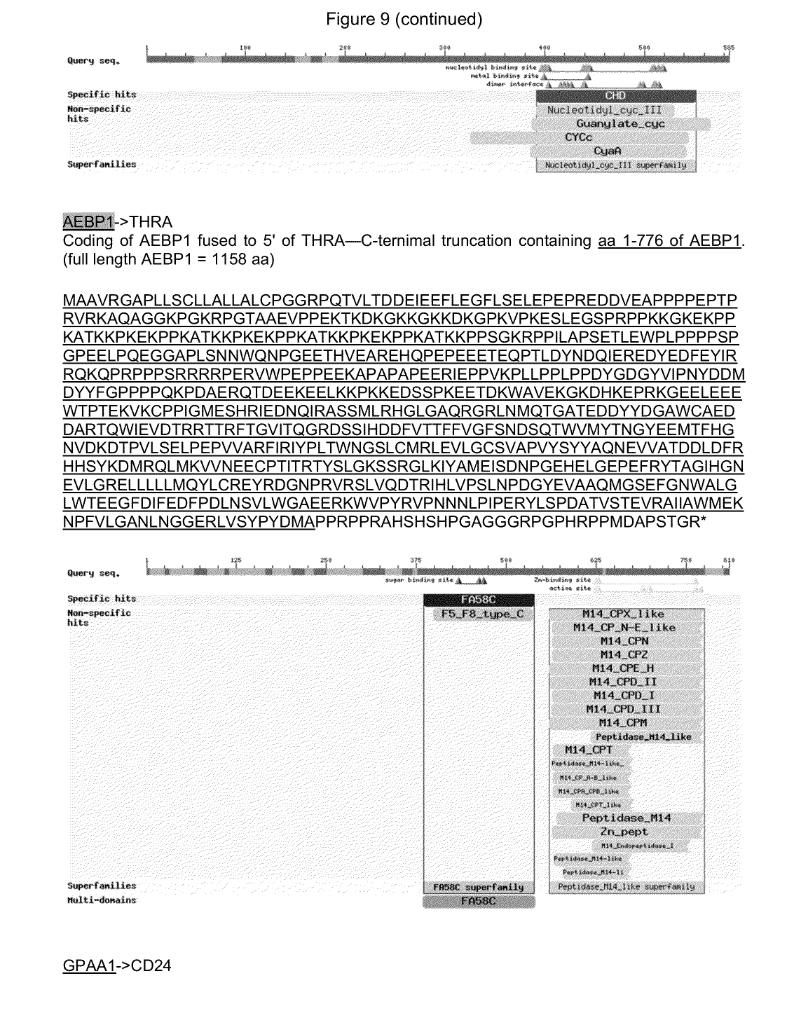

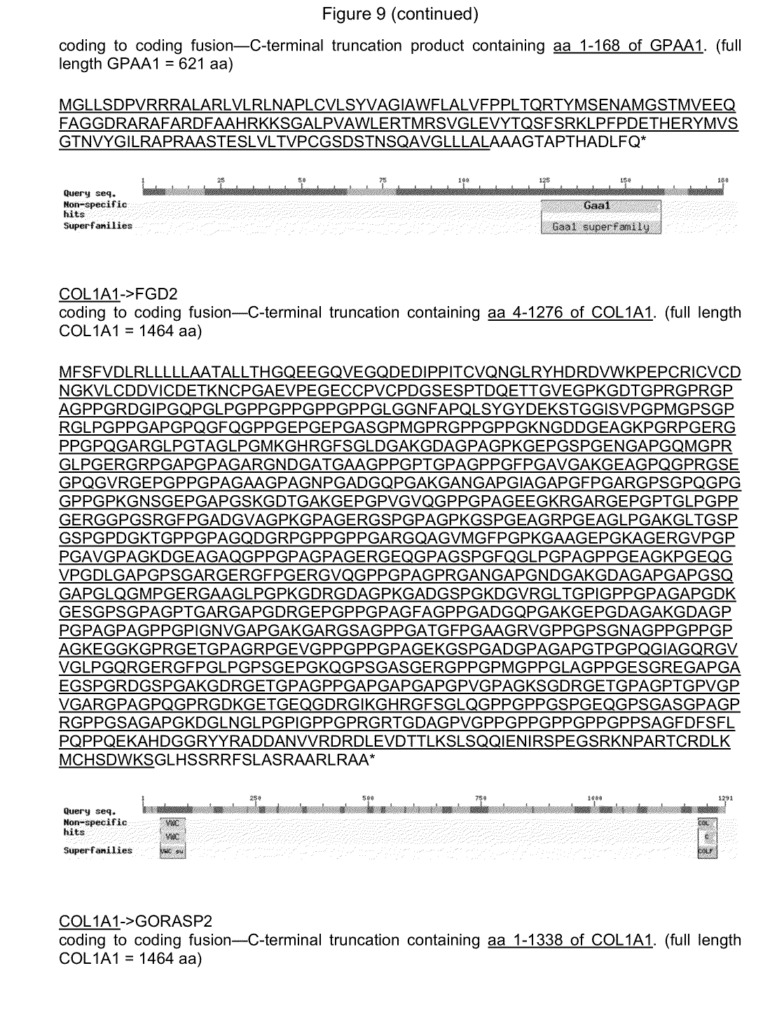

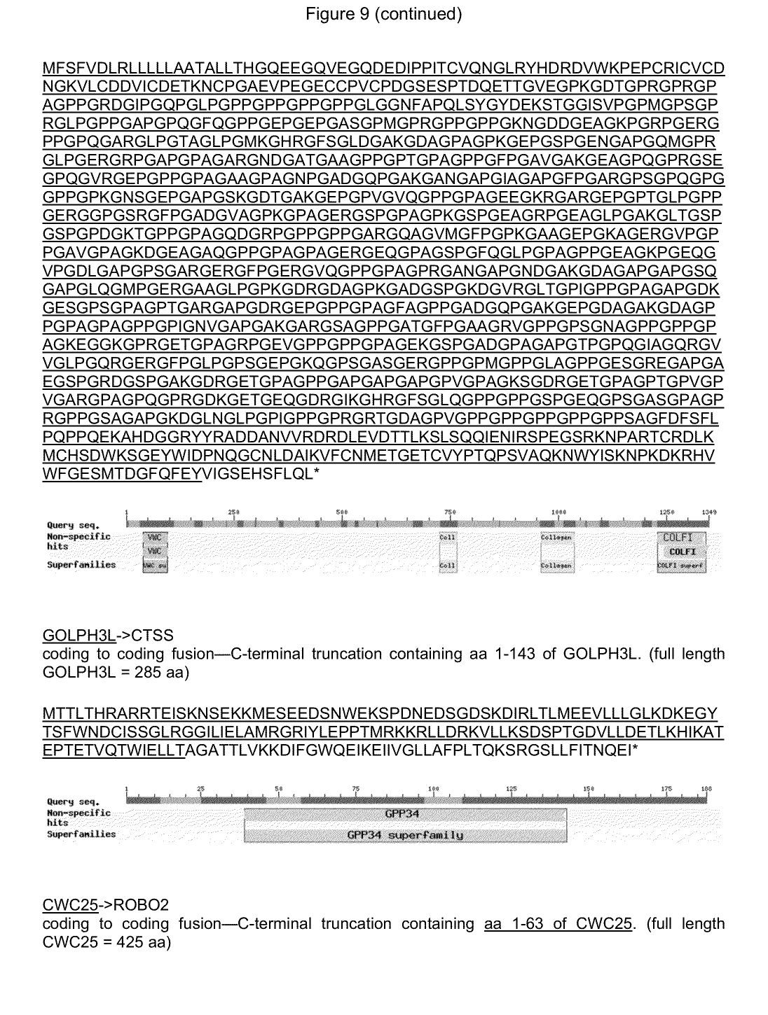

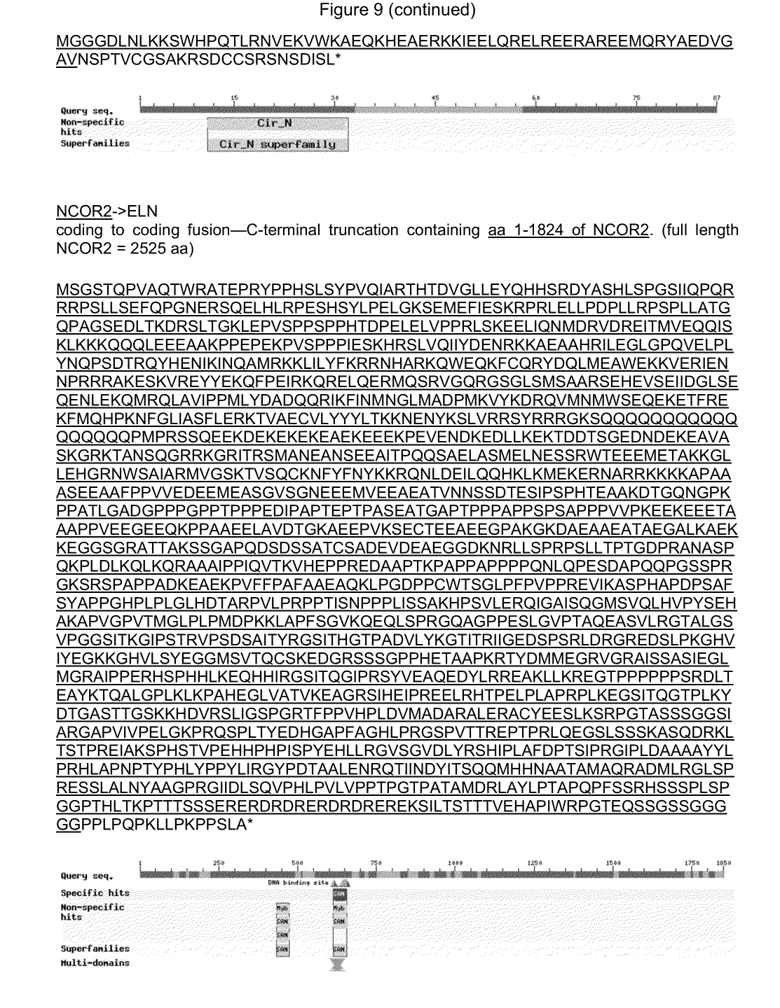

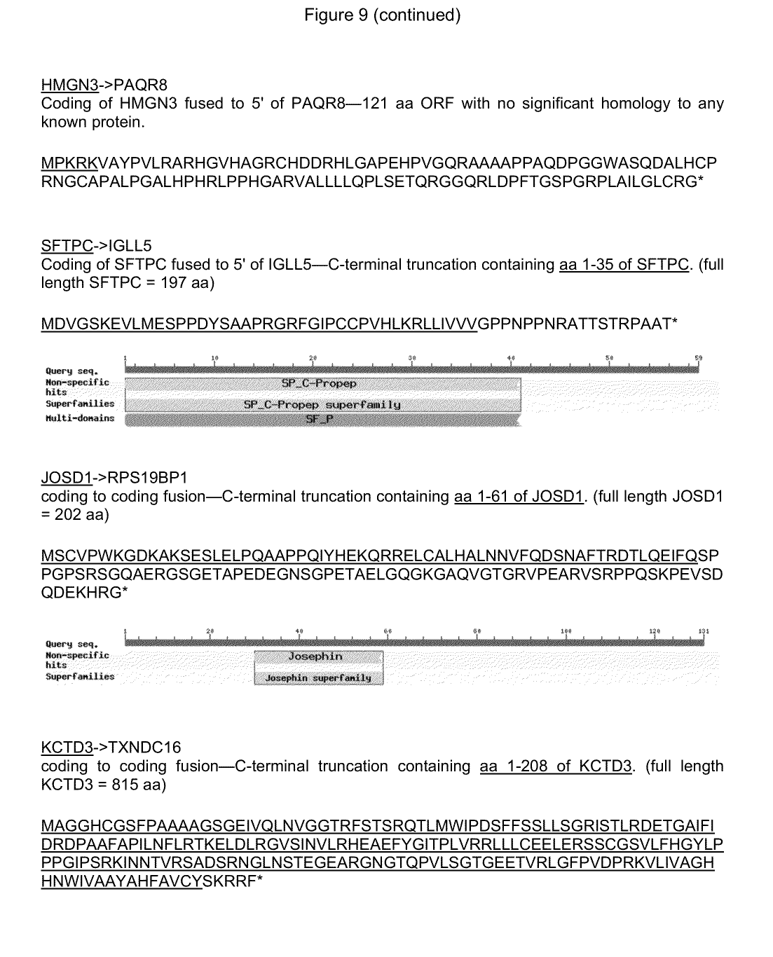

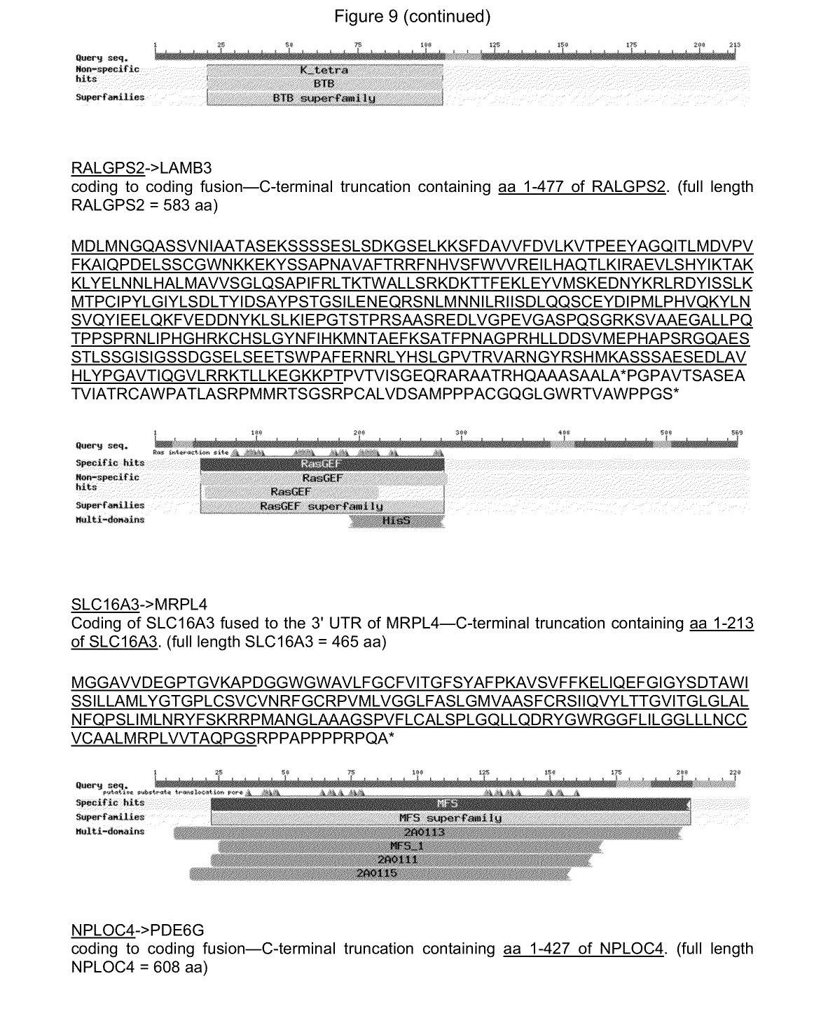

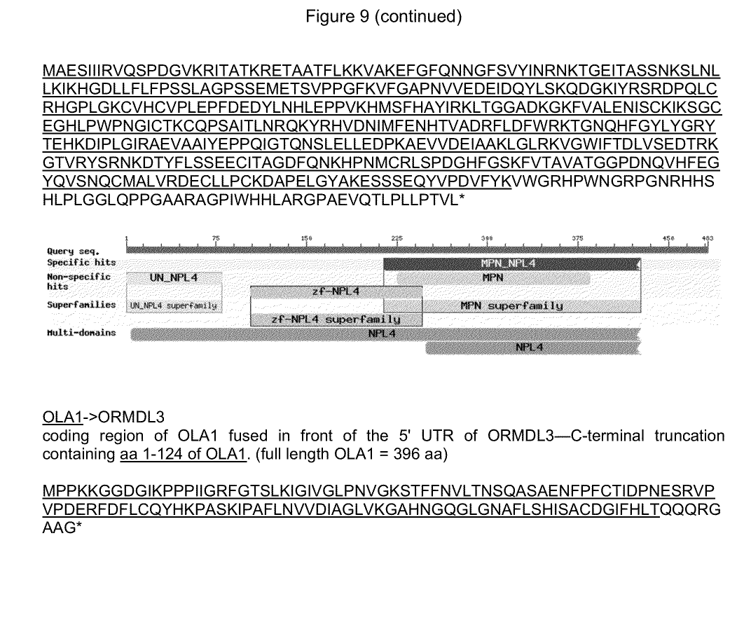

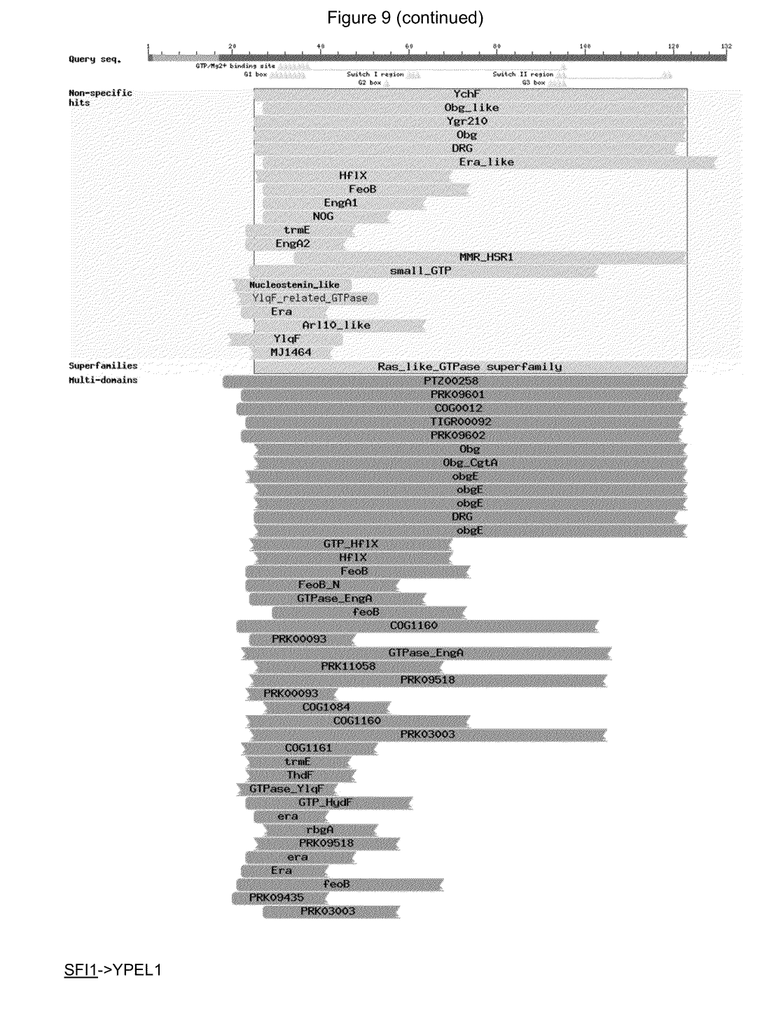

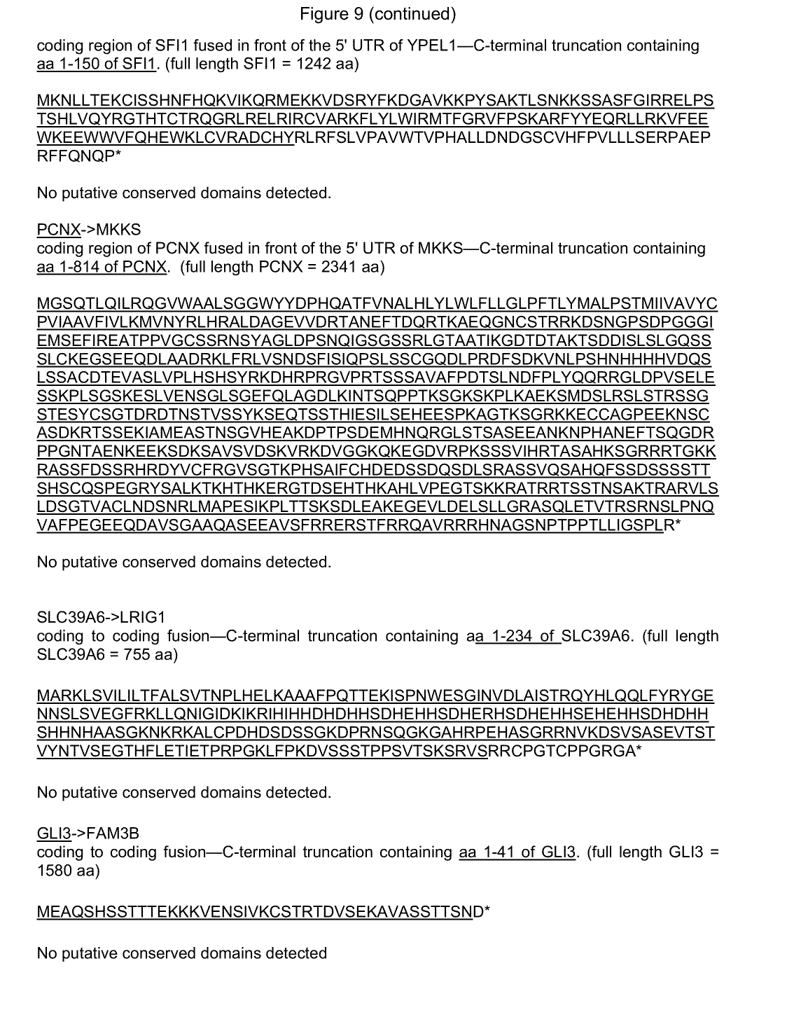

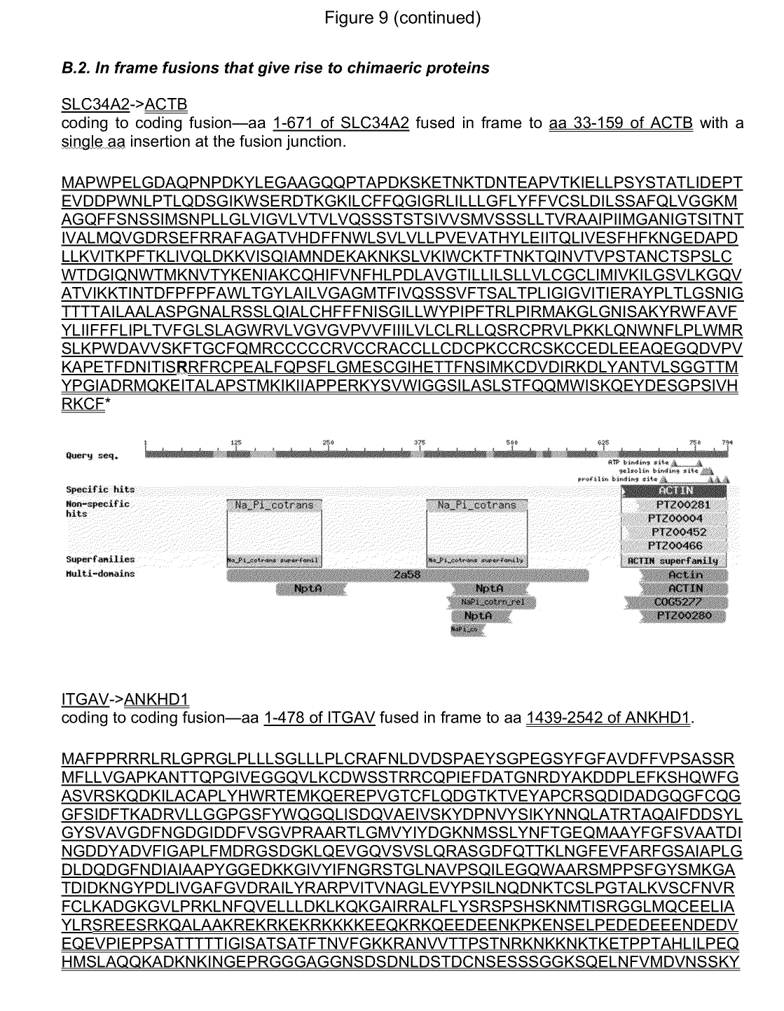

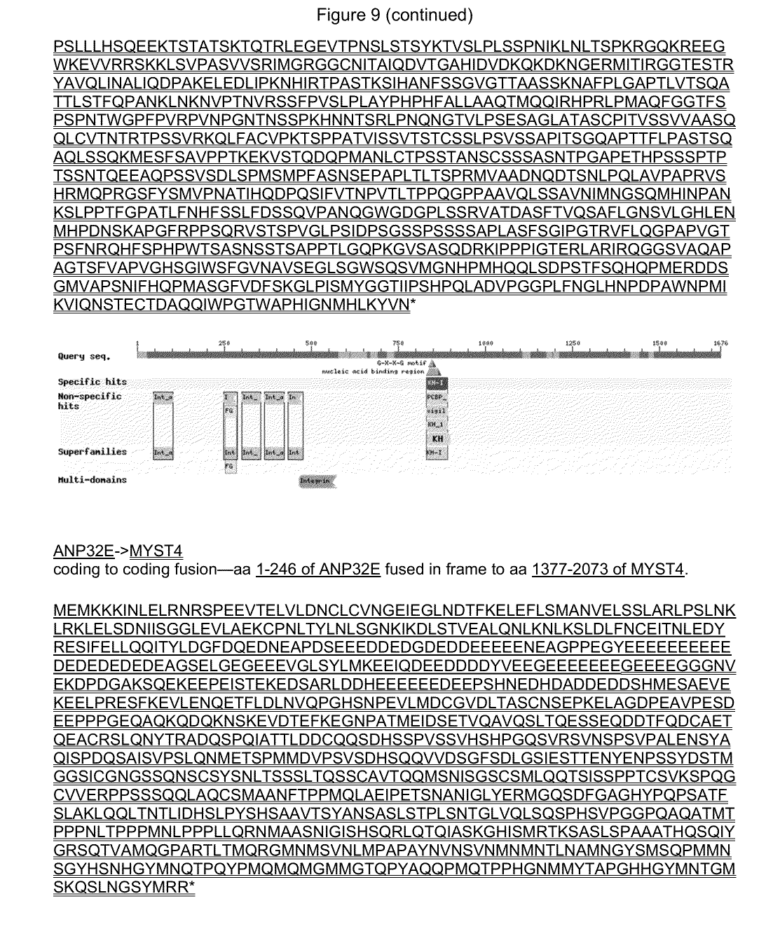

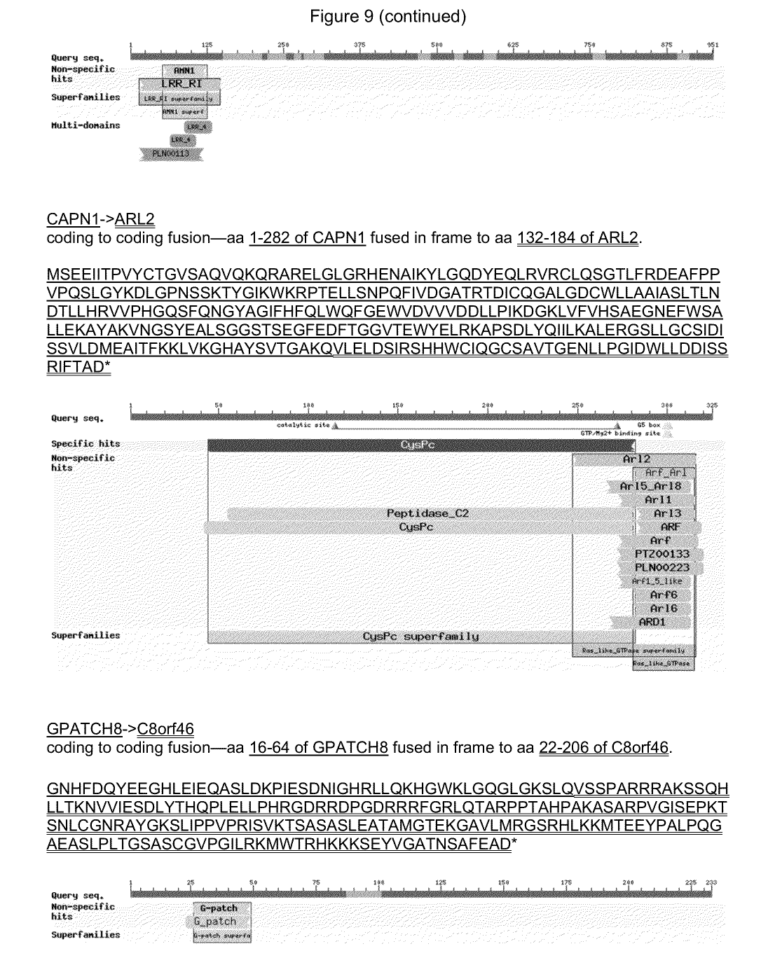

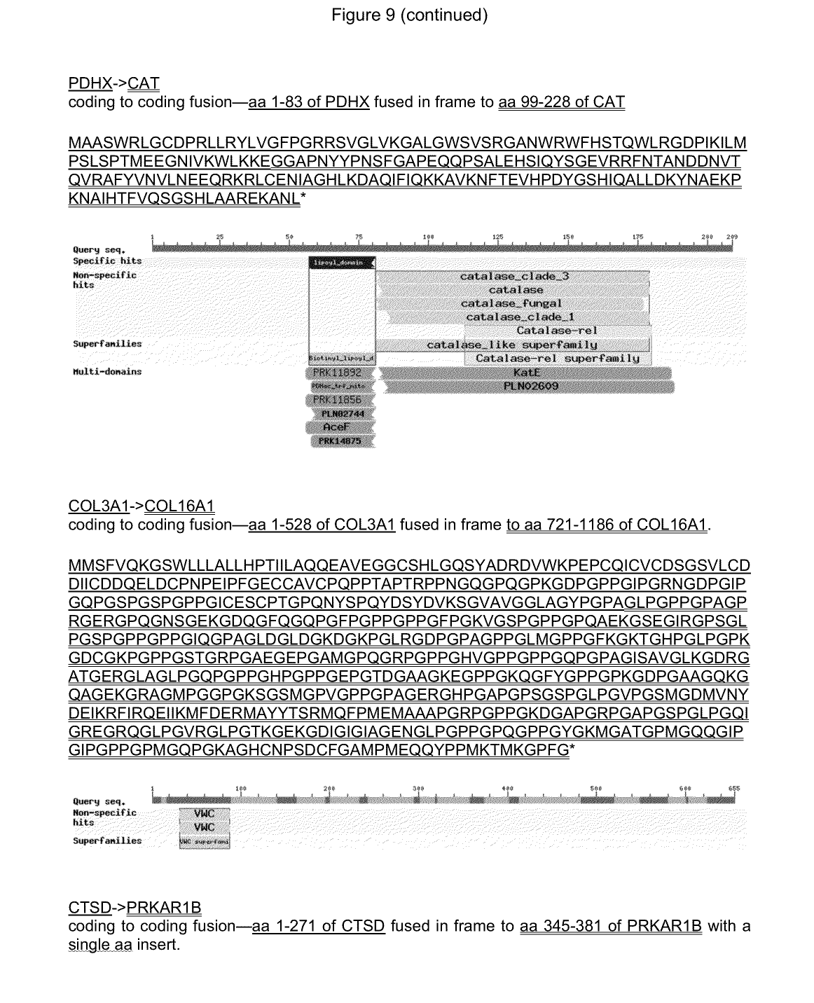

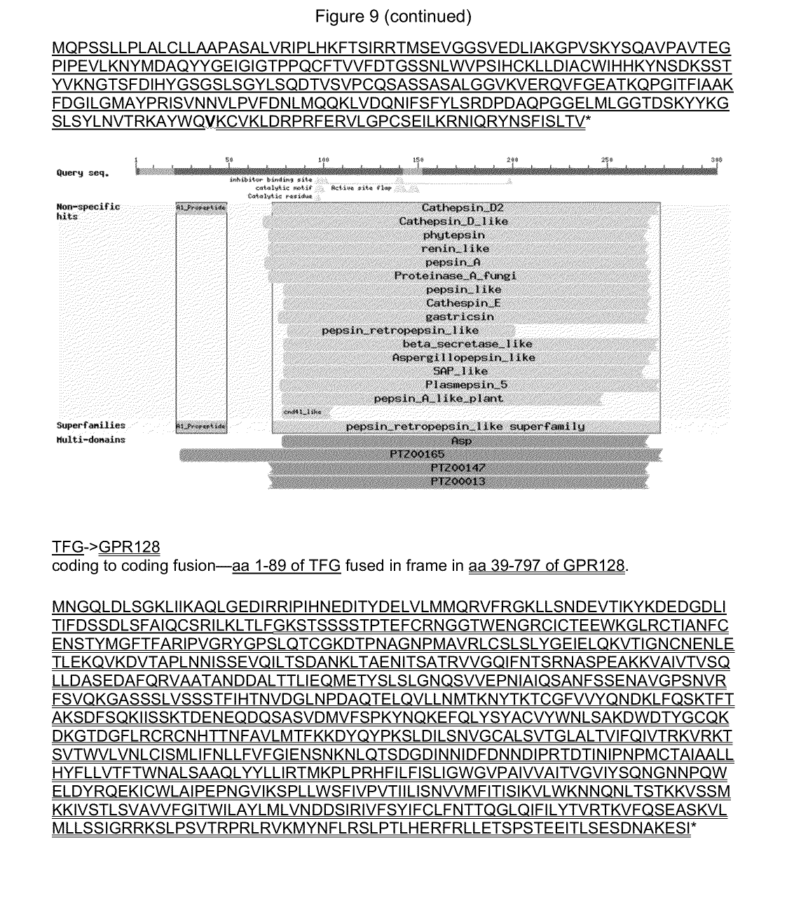

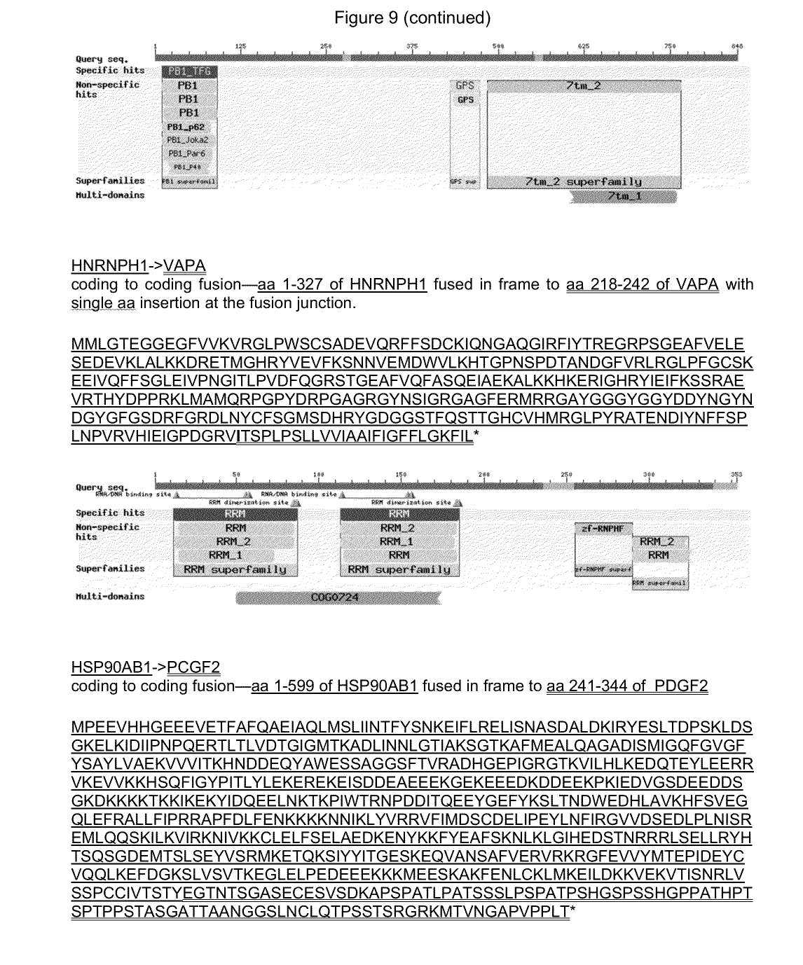

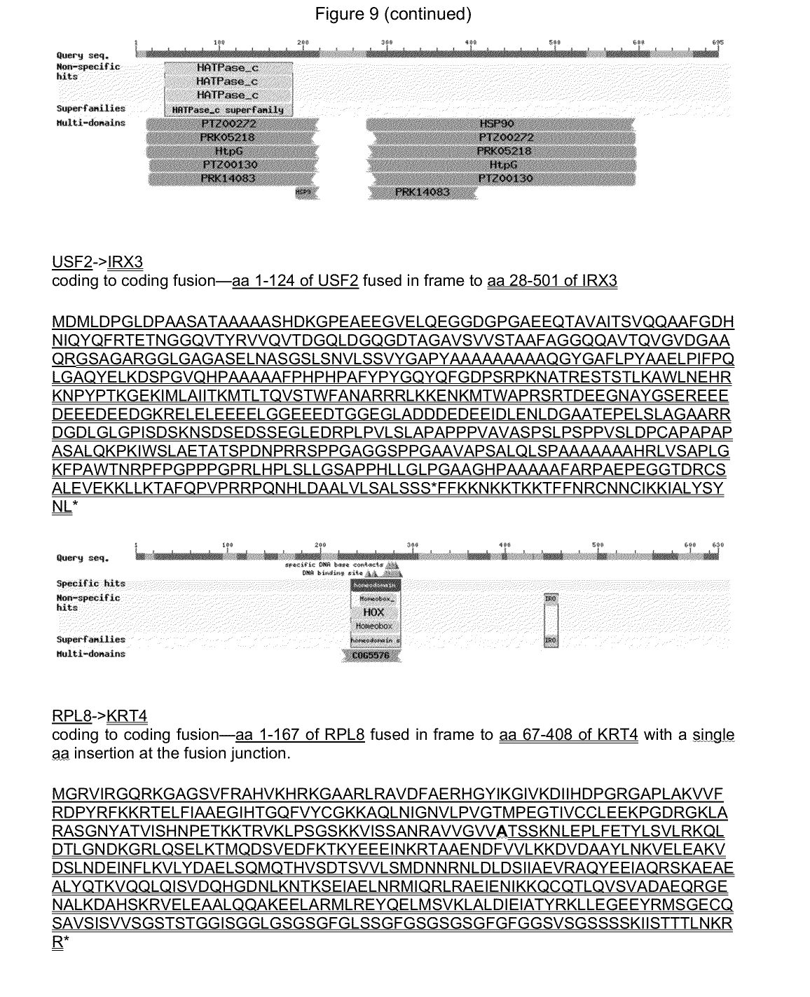

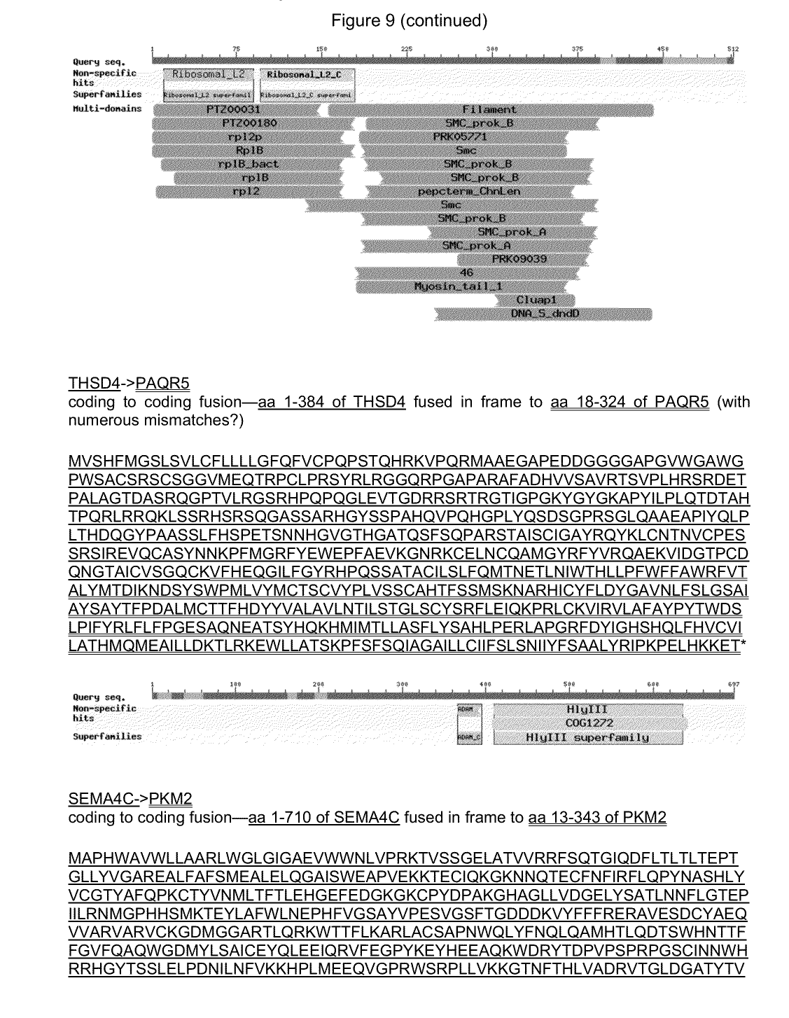

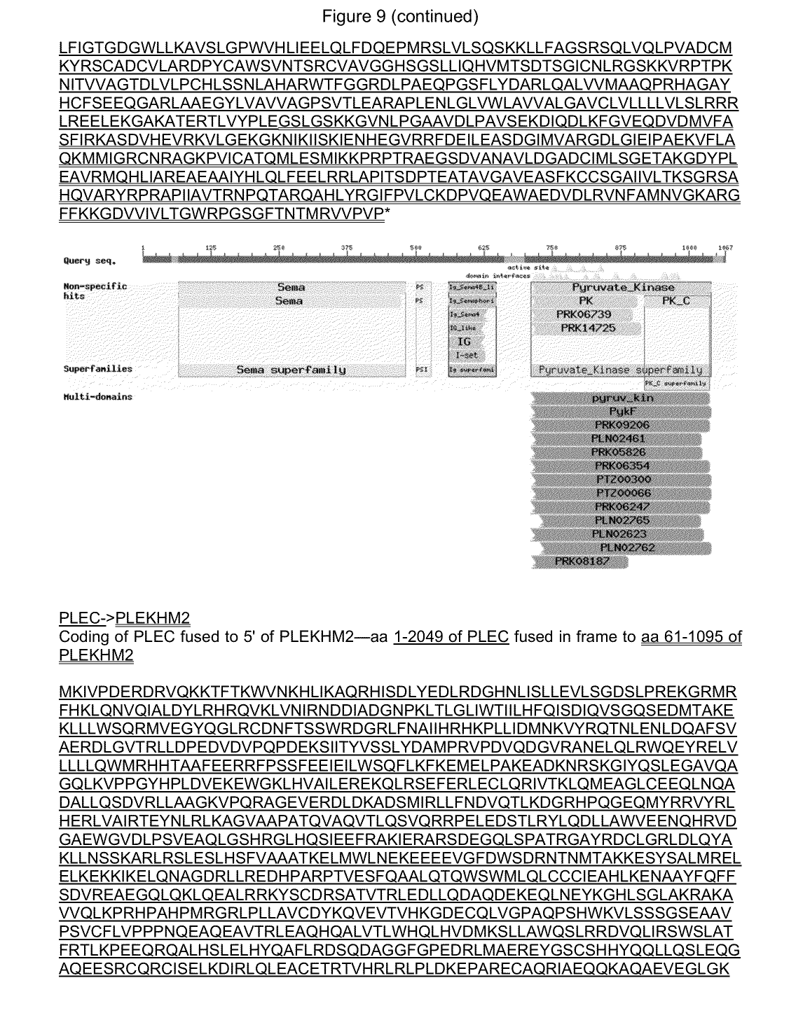

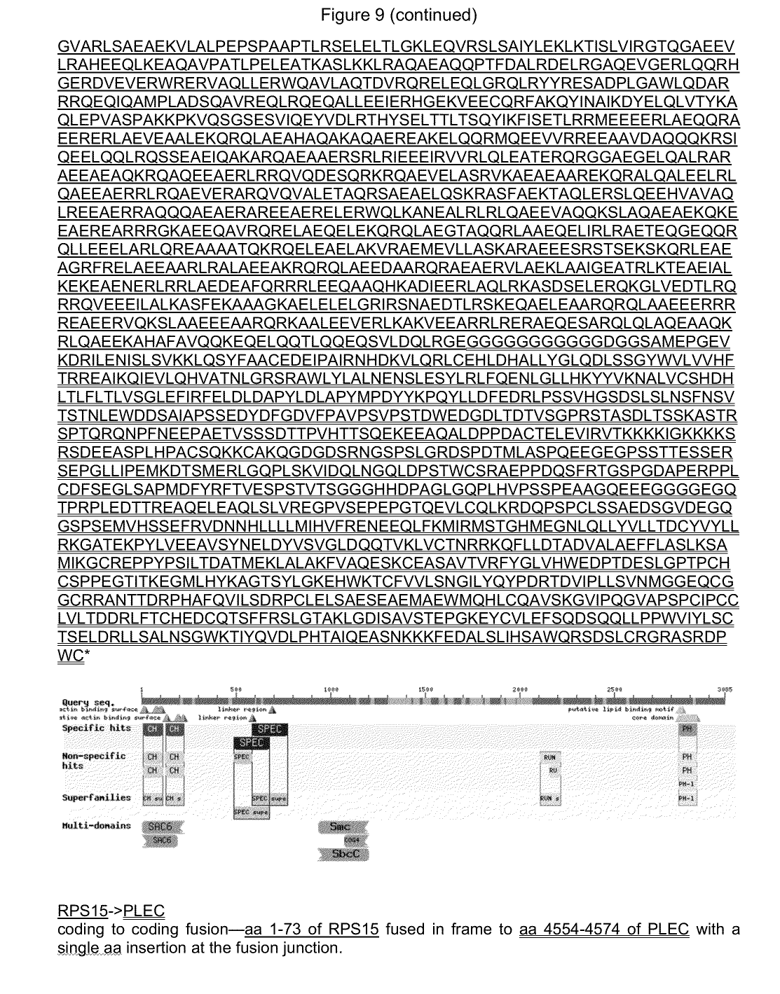

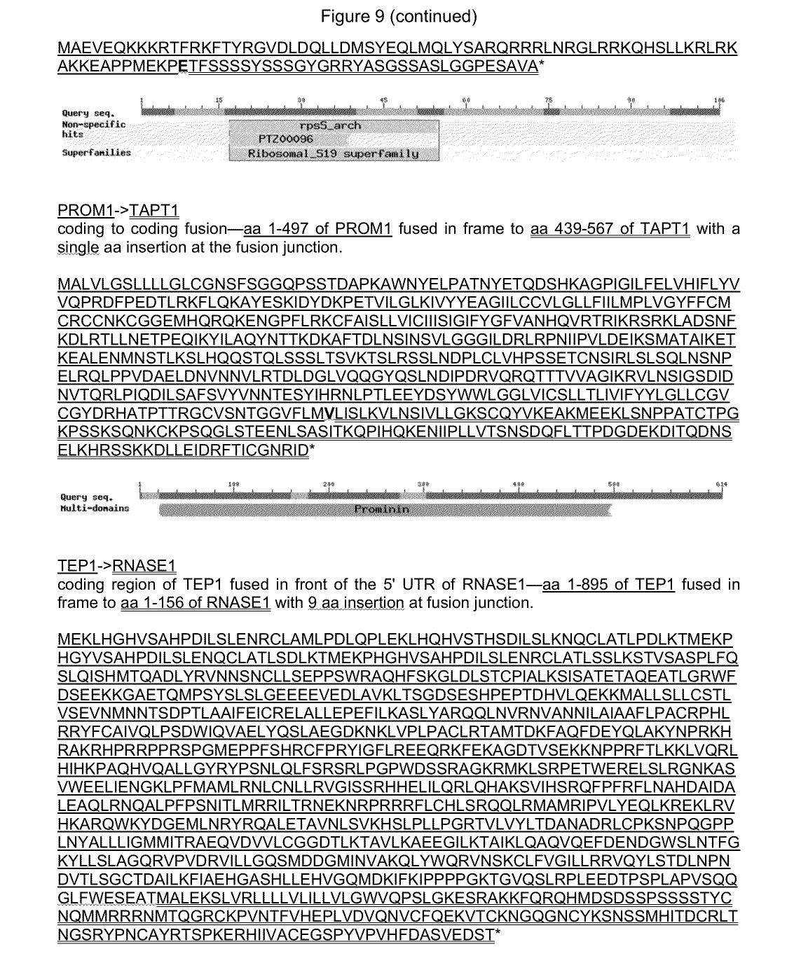

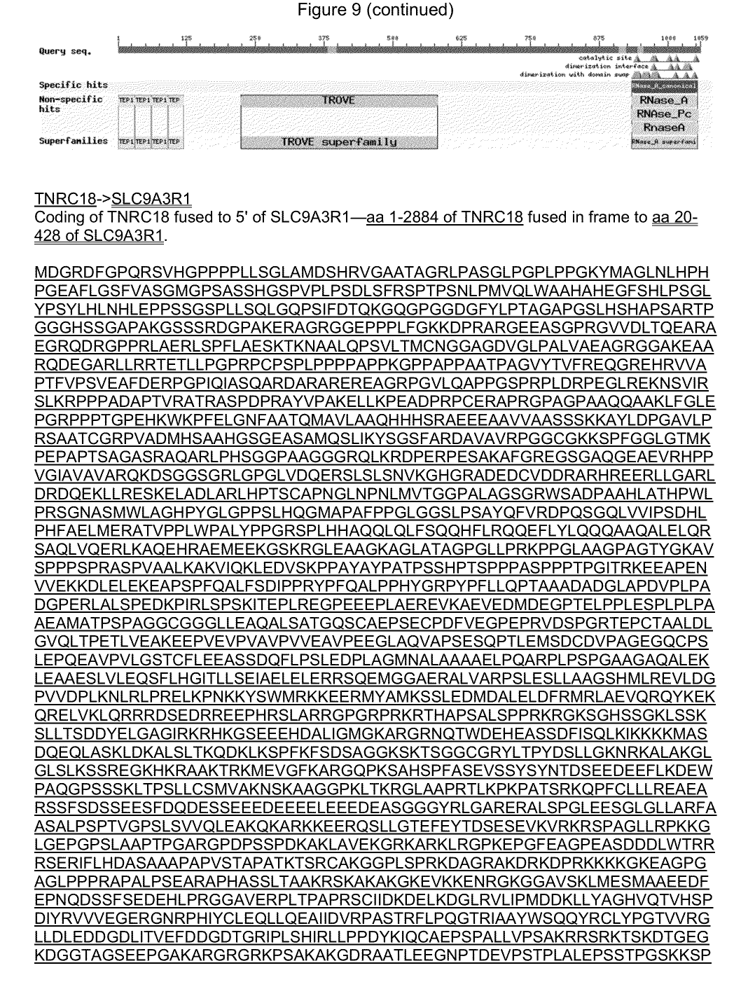

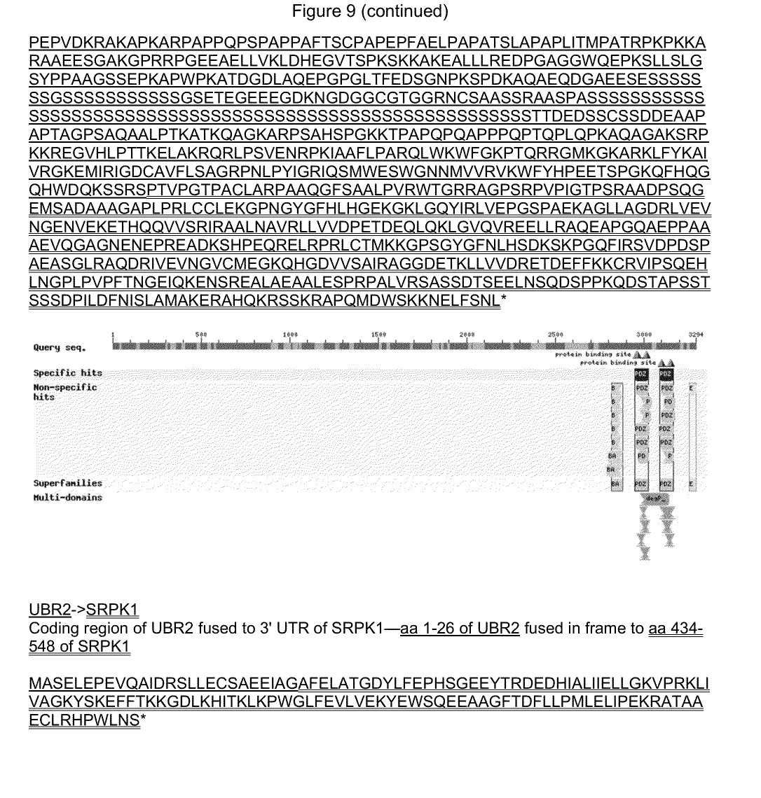

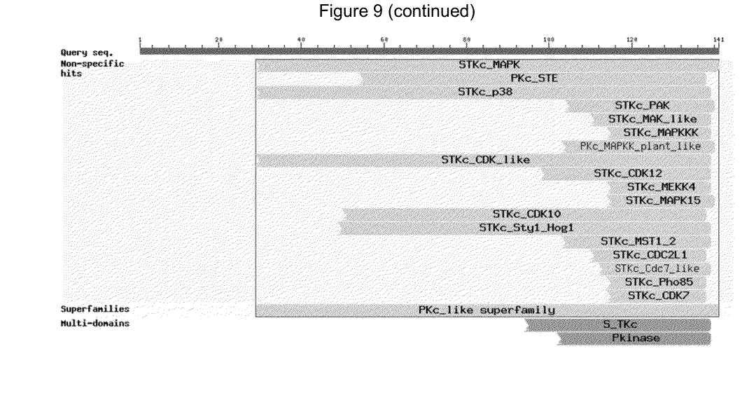

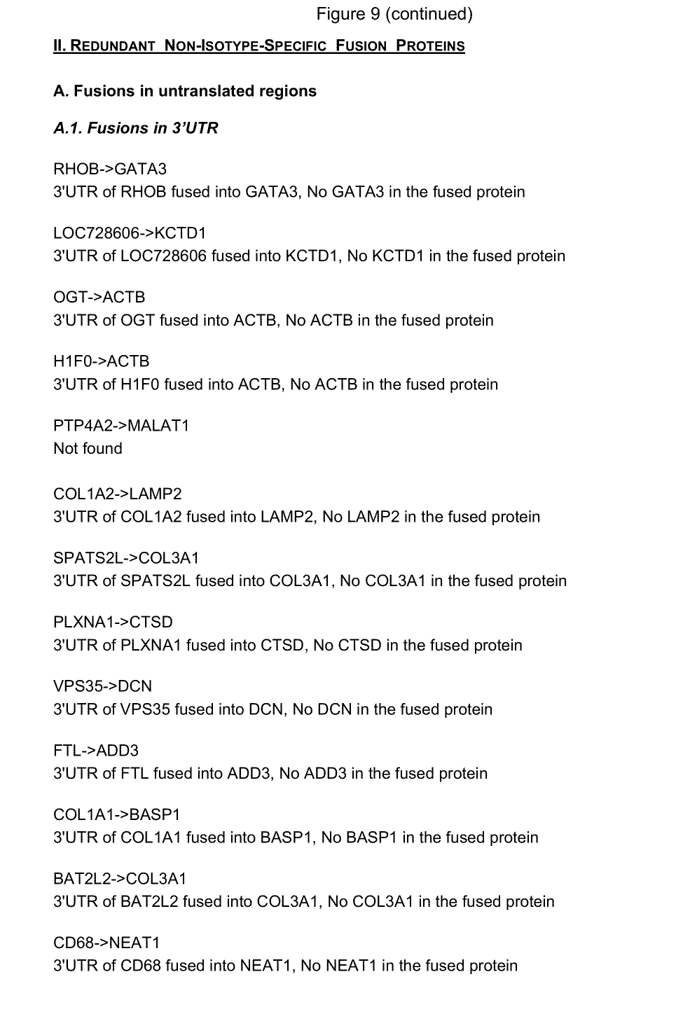

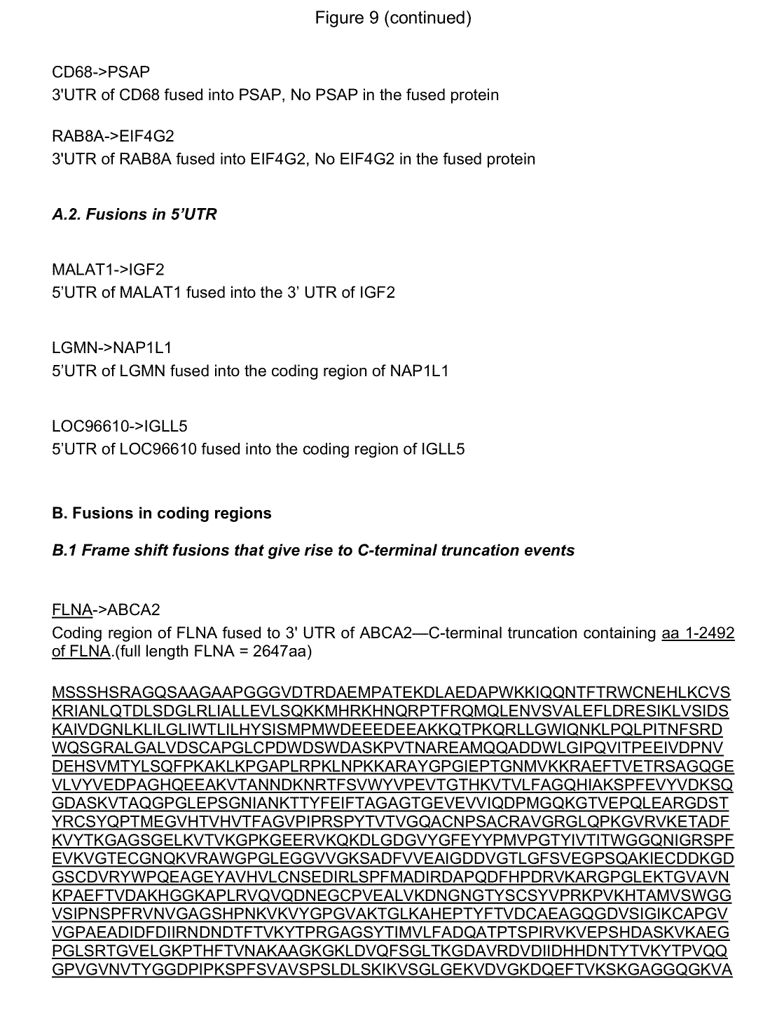

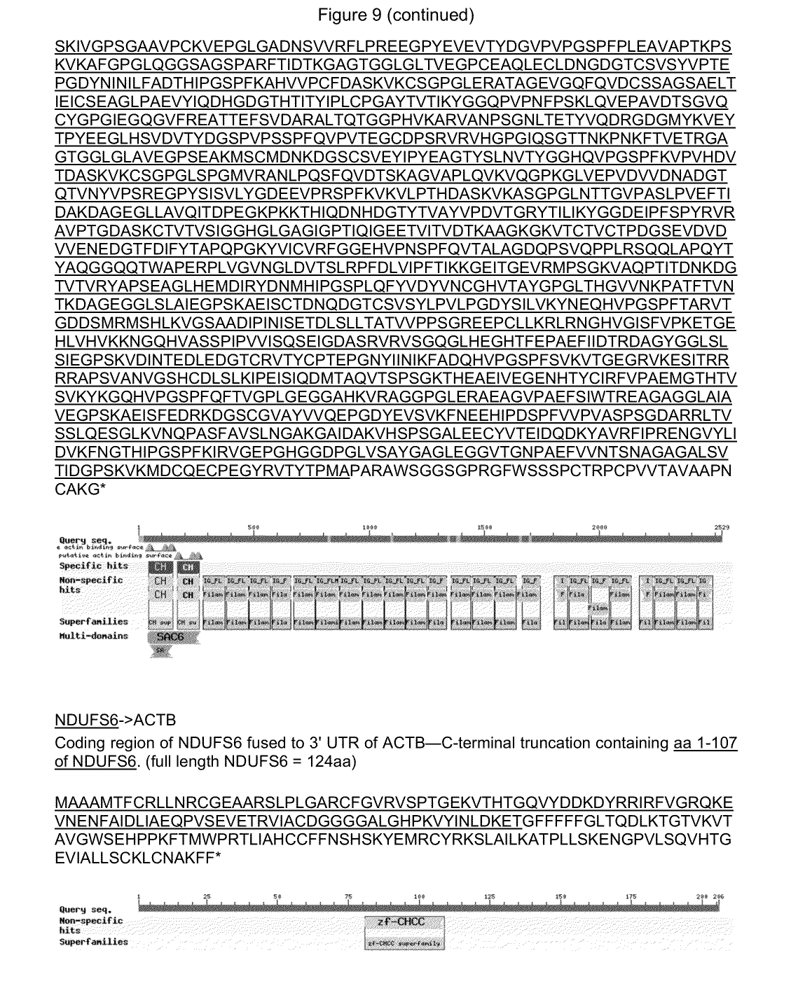

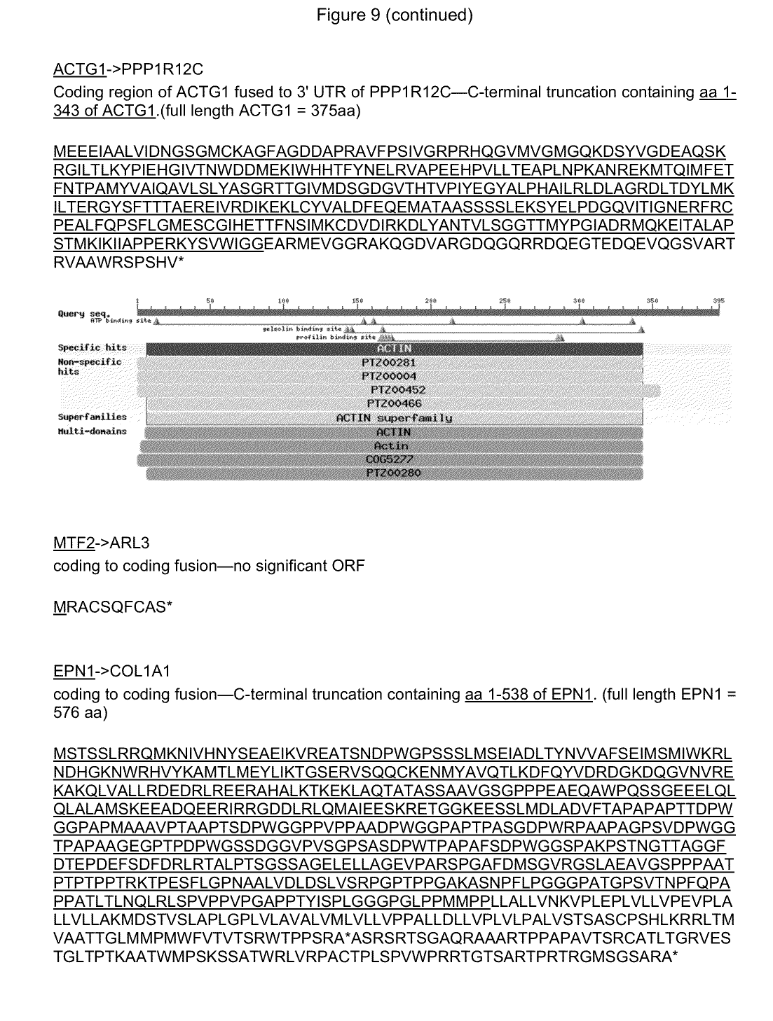

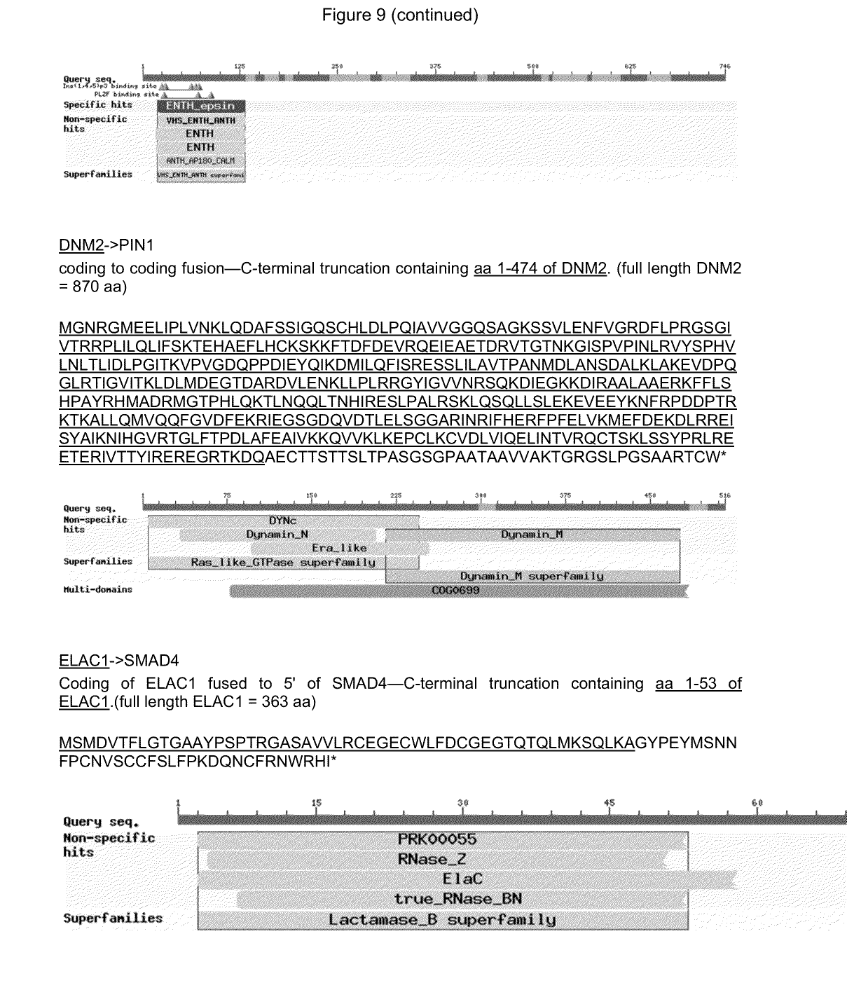

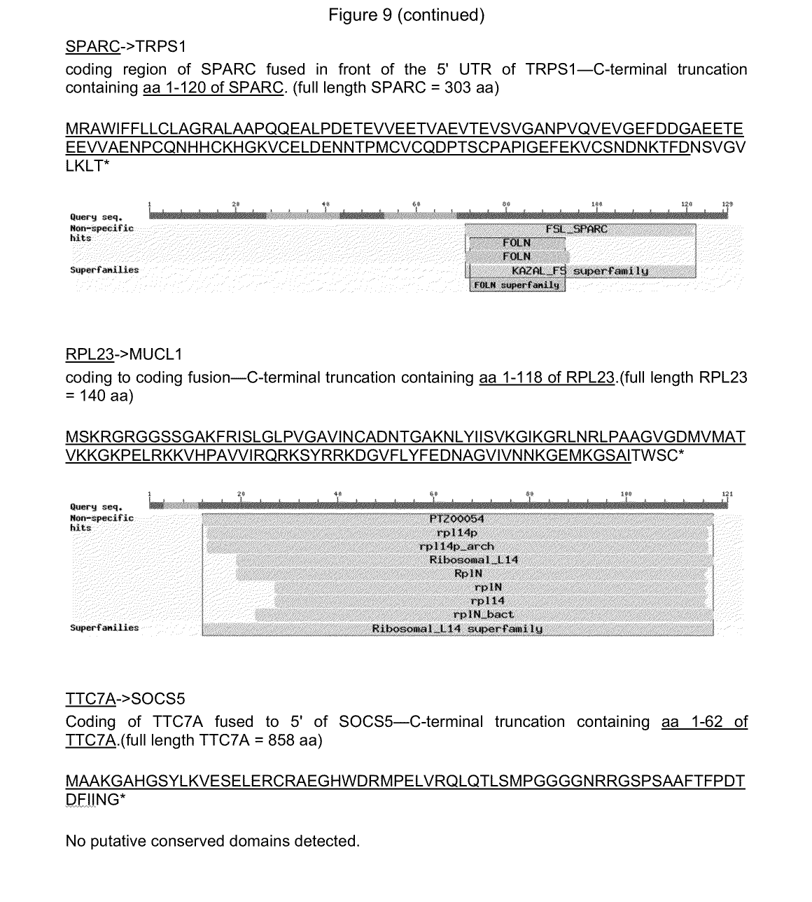

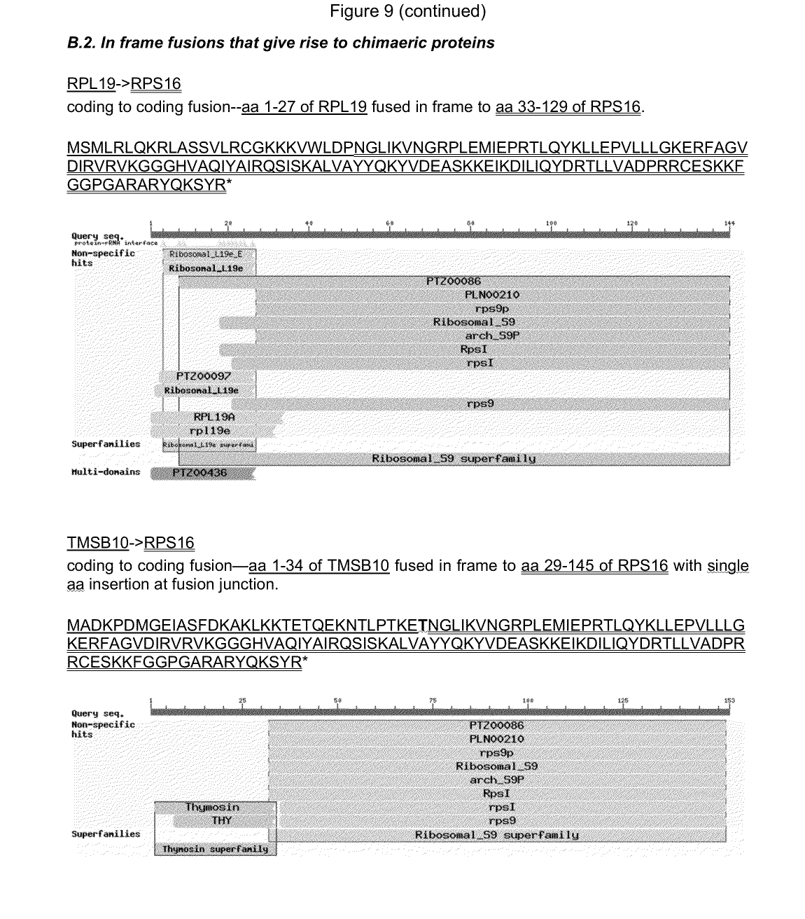

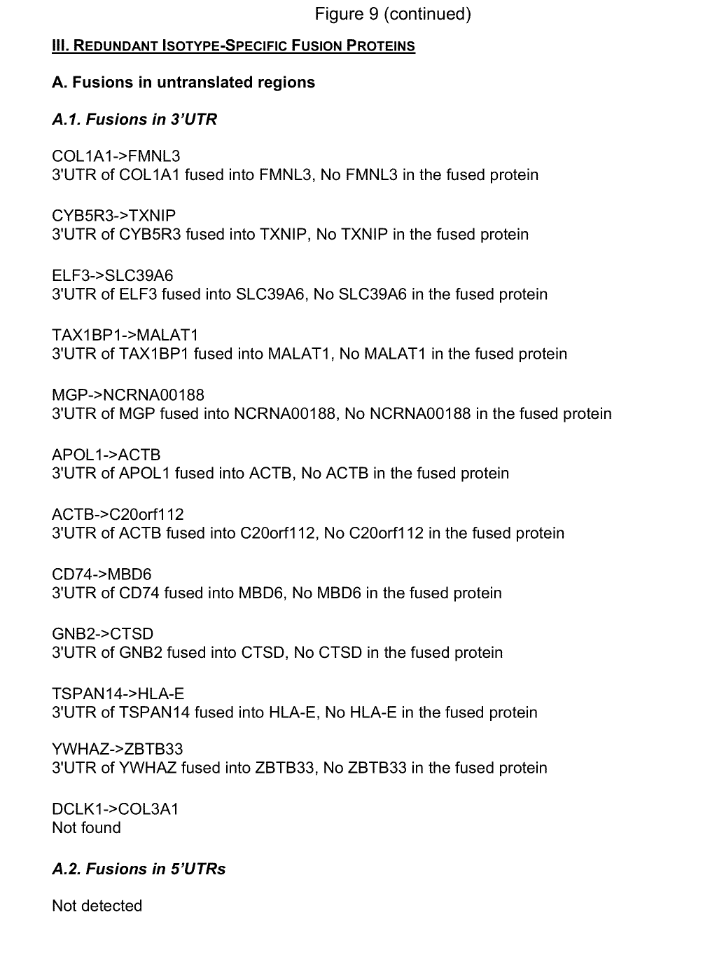

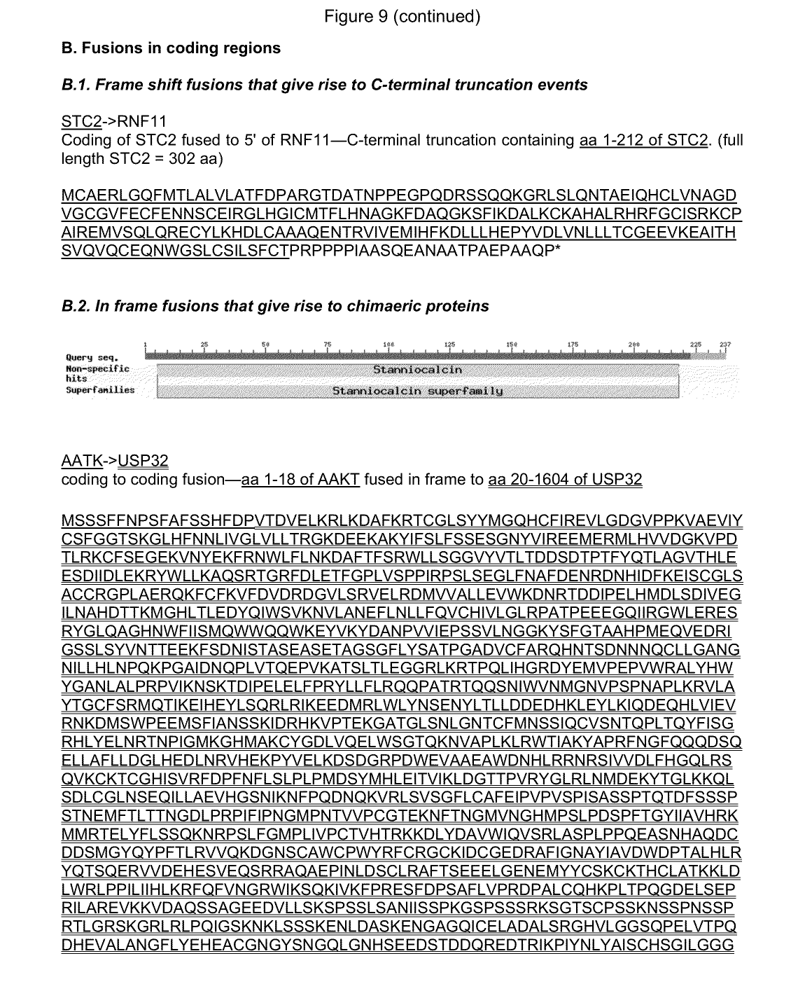

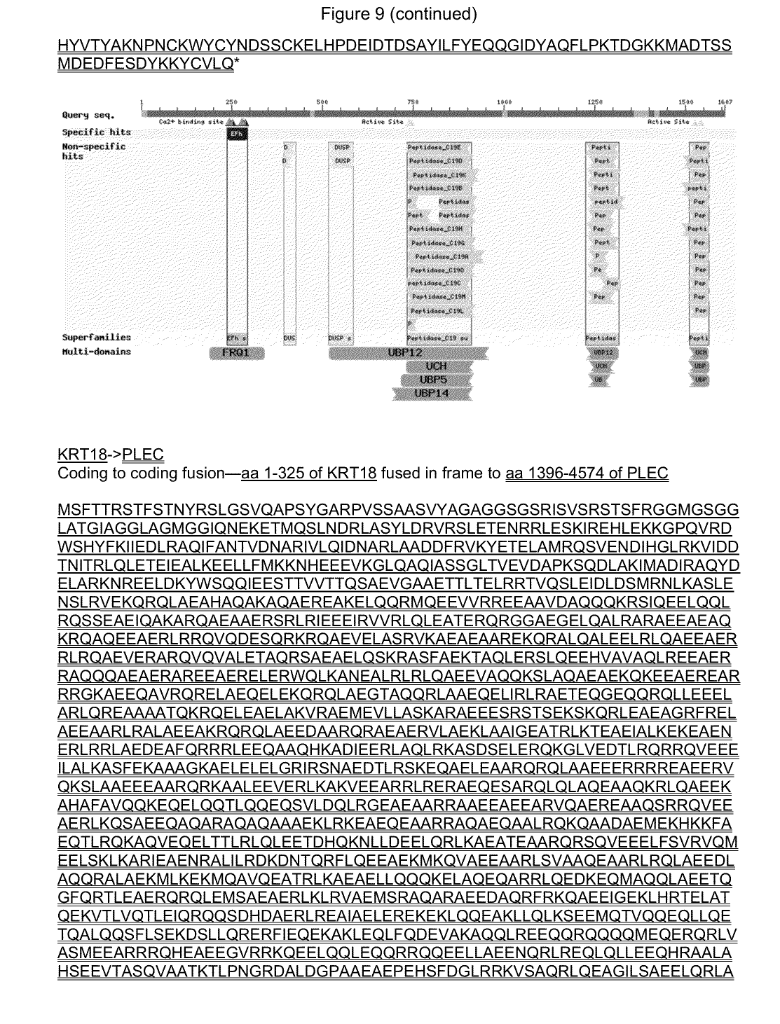

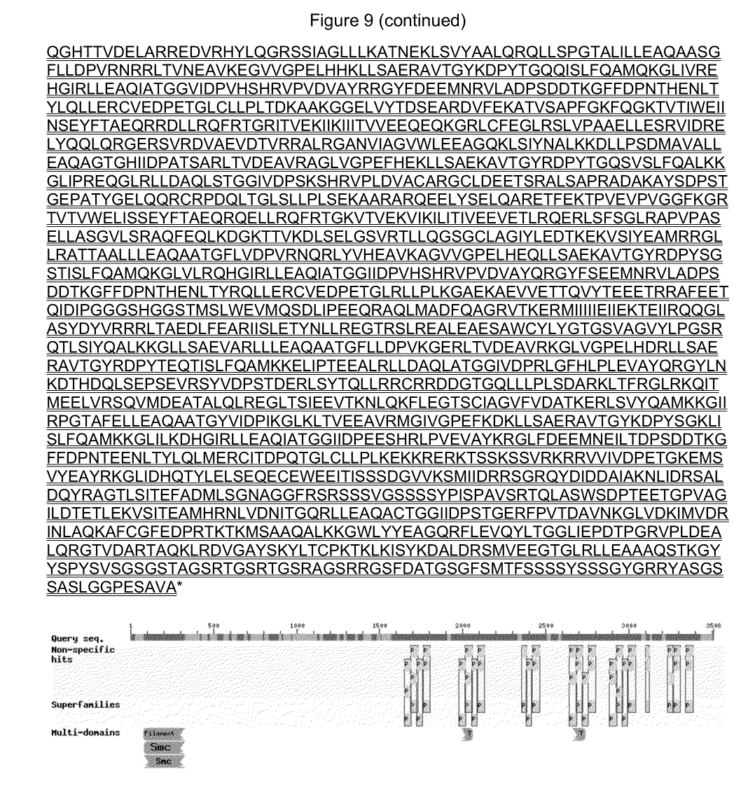

Coding sequence fusions fall into two classes. 25 fusion transcripts were identified that were predicted to give rise to chimaeric proteins, many of which contained functional domains from both fusion partners and might therefore be expected to have novel properties (CIF in FIG. 6). The deduced sequence and functional domains of all predicted fusion products was set forth in FIG. 9. By way of example, the TFG->GPR128 fusion transcript was predicted to encode a 848 amino acid protein in which the PB 1 protein-protein interaction domain of TFG (also known as the TRKT3 oncogene) is fused to the seven trans-membrane spanning domain of GPR128, with loss of the serine/threonine-rich N-terminal domain that is characteristic of this subclass of G-protein-coupled receptors. The potential regulatory effects of such a chimeric protein might be considerable, and the fact that these hypothetical signaling changes might devolve from a G-protein-coupled receptor makes this a potentially druggable target.

About half of the coding-to-coding fusions were predicted to result in frame shifts and carboxy-terminal truncation of the 5′ fusion partner (CTT in FIG. 6). To the extent to which such transcripts escape non-sense mediated degradation mechanisms, they would be predicted to encode N-terminal polypeptides that are deleted of C-terminal functional domains. For example, the ADCY9->C16orf5 fusion transcript was predicted to encode a polypeptide of 585 amino acids that includes the N-terminal nucleotide binding domain of adenylylate cyclase 9, but is deleted of the C-terminal nucleotide cyclase domain and therefore unlikely to have catalytic activity. However, the N-terminal fragment contained the intact dimerization domain of ADCY9 and might therefore function as a dominant negative inhibitor.

Taken together, the results provided herein demonstrate that a set of biomarkers (e.g., fusion genes) can be used to identify breast cancer.

Potential functions for particular fusion transcripts are listed in Table 11.

TABLE 11

| Fusion Transcript | Activity |
|---|---|
| AATK->USP32 | protein synthesis |
| TP53I13->ABCA10 | Drug resistance |
| FLNA->ABCA2 | Drug resistance |
| EIF4G1->ABCC5 | Drug resistance |
| CALR->ACACA | Drug resistance |
| APOL1->ACTB | cell motility/invasion |
| H1F0->ACTB | cell motility/invasion |
| NDUFS6->ACTB | cell motility/invasion |
| OGT->ACTB | cell motility/invasion |
| SLC34A2->ACTB | cell motility/invasion |
| ACTG1->PPP1R12C | cell signaling |
| FTL->ADD3 | cell motility/invasion |
| AEBP1->THRA | cell signaling |
| AEBP1-THRA | cell signaling |
| ITGAV->ANKHD1 | cell survival |
| ANP32E->MYST4 | gene regulation |
| APOOL->DCAF8 | protein synthesis |
| TMEM119->ARIH2 | protein synthesis |
| CAPN1->ARL2 | cell signaling |
| MTF2->ARL3 | cell signaling |
| ASAP1->MALAT1 | metastasis |
| BAT2L2->COL3A1 | cell motility/invasion |
| GPAA1->CD24 | immune surveillance |
| CD74->MBD6 | gene regulation |
| CDK4->UBA1 | protein synthesis |
| CIRBP->UGP2 | Drug resistance |
| DNAJA2->COL14A1 | cell motility/invasion |
| COL3A1->COL16A1 | cell motility/invasion |
| EPN1->COL1A1 | cell motility/invasion |
| COL1A1->FGD2 | cell signaling |
| COL1A1->FMNL3 | cell growth |
| COL1A1->GORASP2 | protein synthesis |
| HEATR5A->COL1A1 | cell motility/invasion |
| COL1A2->LAMP2 | metastasis |
| DCLK1->COL3A1 | cell motility/invasion |
| POLD3->COL3A1 | cell motility/invasion |
| SPATS2L->COL3A1 | cell motility/invasion |
| COL3A1->ZNF43 | gene regulation |
| RHOBTB3->CRNKL1 | gene regulation |
| EPHA2->CTSD | malignant transformation |
| GNB2->CTSD | malignant transformation |
| LTBP4->CTSD | malignant transformation |
| PACSIN3->CTSD | malignant transformation |
| PLXNA1->CTSD | malignant transformation |
| CTSD->PRKAR1B | gene regulation |
| TMEM109->CTSD | malignant transformation |
| GOLPH3L->CTSS | malignant transformation |
| CWC25->ROBO2 | cell motility/invasion |
| VPS35->DCN | cell growth |
| DIDO1->REPS1 | cell signaling |
| DNM2->PIN1 | malignant transformation |
| RAB8A->EIF4G2 | protein synthesis |
| ELAC1->SMAD4 | malignant transformation |
| NCOR2->ELN | cell motility/invasion |
| GLI3->FAM3B | cell viability |
| SBF1->FLNA | cell motility/invasion |
| GAPDH->KRT13 | cell motility/invasion |
| GAPDH->MRPS18B | protein synthesis |
| RHOB->GATA3 | gene regulation |
| GNB1->TRH | cell growth |
| PTMA->GNB4 | cell signaling |
| TFG->GPR128 | malignant transformation |
| TSPAN14->HLA-E | immune surveillance |
| HMGN3->PAQR8 | cell signaling |
| TES->HNRNPU | gene regulation |
| HSP90AB1->PCGF2 | malignant transformation |
| MALAT1->IGF2 | metastasis |
| IGF2-MALAT1 | metastasis |
| RAB3IP->IGFBP5 | cell growth |
| MAF->IGFBP7 | cell growth |
| USF2->IRX3 | cell differentiation |
| JOSD1->RPS19BP1 | protein synthesis |
| LOC728606->KCTD1 | metastasis |
| KRT18->PLEC | cell motility/invasion |
| RPL8->KRT4 | cell motility/invasion |
| RALGPS2->LAMB3 | malignant transformation |
| LGMN->NAP1L1 | gene regulation |
| SLC39A6->LRIG1 | cell growth |
| PTP4A2->MALAT1 | metastasis |
| TAX1BP1->MALAT1 | metastasis |
| MAPK1IP1L->XPO1 | Drug resistance |
| MGP->REPS2 | cell signaling |
| PCNX->MKKS | protein synthesis |
| SLC16A3->MRPL4 | protein synthesis |
| MRPL52->USP22 | protein synthesis |
| RPL23->MUCL1 | metastasis |
| NAV2->WDFY1 | cell signaling |
| NPLOC4->PDE6G | cell signaling |
| OLA1->ORMDL3 | cell signaling |
| THSD4->PAQR5 | cell signaling |
| YWHAG->PDIA3 | protein synthesis |
| SEMA4C->PKM2 | energy metabolism |
| PLEC->PLEKHM2 | metastasis |
| RPS15->PLEC | cell motility/invasion |
| POSTN->TRIM33 | cell growth |
| PROM1->TAPT1 | cell differentiation |
| TEP1->RNASE1 | gene regulation |
| STC2->RNF11 | malignant transformation |
| RPL19->RPS16 | protein synthesis |
| TMSB10->RPS16 | protein synthesis |
| SFI1->YPEL1 | cell differentiation |
| TTC7A->SOCS5 | immune surveillance |
| SPARC->TRPS1 | cell differentiation |
| UBR2->SRPK1 | gene regulation |
| YWHAZ->ZBTB33 | malignant transformation |

### Example 3

### Characterization of the ARID1A-MAST2 Fusion Transcript

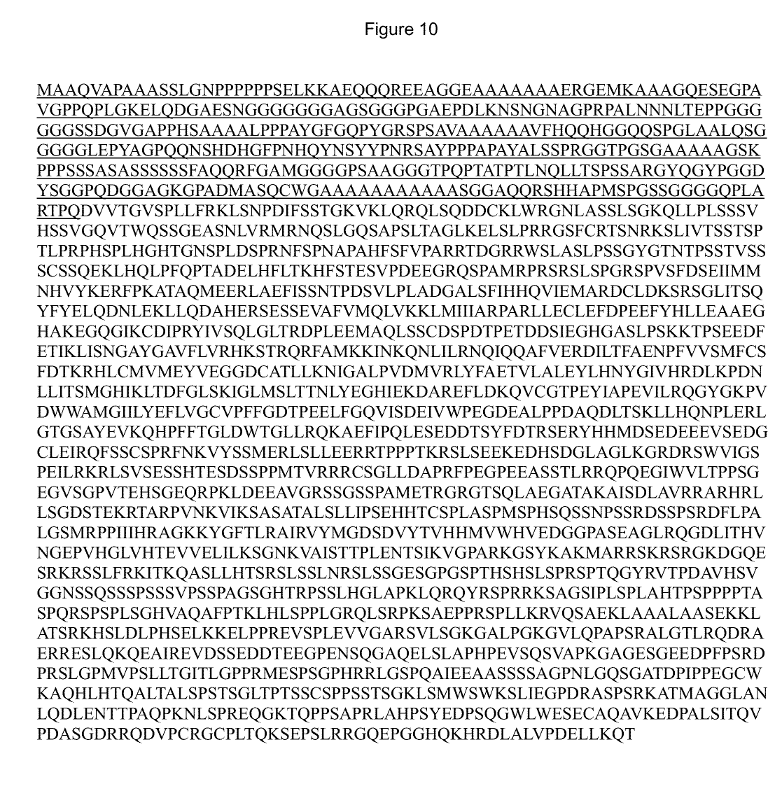

The ARID1A->MAST2 fusion encoded a 2118 amino acid chimeric polypeptide product that contained the complete kinase domain of the microtubule-associated serine/threonine protein kinase MAST2, but is deleted of amino terminal MAST2 sequences that may affect the activity of the kinase. The predicted amino acid sequence of the chimeric polypeptide is set forth in FIG. 10.

Specific RT-PCR primers that can discriminate between endogenous MAST2 and the ARID1A->MAST2 fusion transcript were designed (FIG. 11; lanes labeled “NT”). Lentiviral shRNA knockdown constructs were designed to attenuate expression of the fusion transcript. These constructs were labeled 73, 74, and 75 in FIG. 11. Knockdown controls were non-template shRNA vectors, labeled NT in FIG. 11.

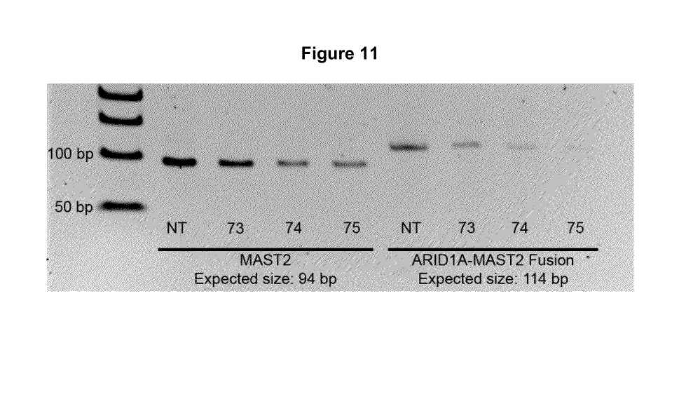

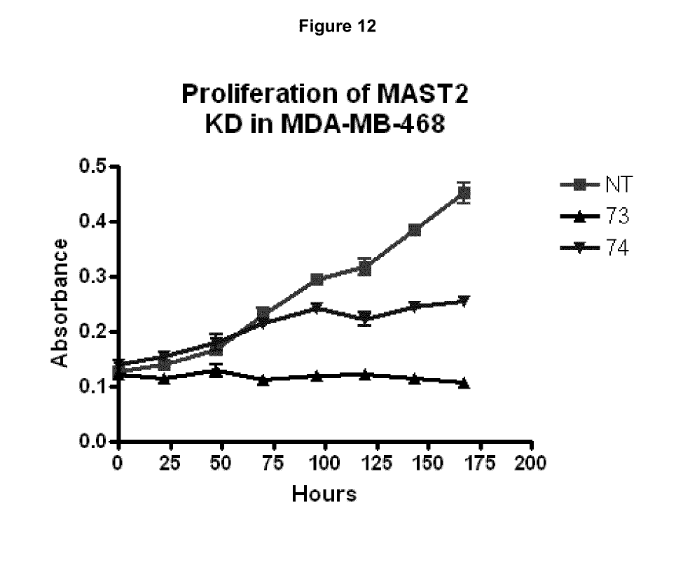

Culture growth of MDA-MB-468 cells, which express the ARID1A->MAST2 fusion product, was inhibited by transduction with shRNA knockdown constructs that attenuate expression of the fusion transcript (FIG. 12).

Taken together, the results provided herein demonstrate that fusion transcripts are recurrent in breast cancer and can serve as biomarkers or therapeutic targets. The results provided herein also demonstrate that fusion transcripts such as the ARID1A->MAST2 fusion product are “driver mutations” (i.e., mutations necessary for survival and/or growth of breast cancer cells). In addition, the results provided herein demonstrate that fusion partners such as MAST2 can be therapeutic targets in breast cancer.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A primer pair comprising first and second primers, wherein an amplification reaction comprising said first and second primers has the ability to amplify a nucleic acid having a fusion partner A sequence and a fusion partner B sequence, wherein said fusion partner A sequence is present in a first human gene set forth in Table 3, 4, 5, 6, 8, or 10 and said fusion partner B sequence is present in a second human gene set forth in Table 3, 4, 5, 6, 8, or 10 as being a fusion partner with said first human gene.

2. The primer pair of claim 1, wherein said fusion partner A sequence is at least 10 nucleotides.

3. The primer pair of claim 1, wherein said fusion partner A sequence is at least 50 nucleotides.

4. The primer pair of claim 1, wherein said fusion partner A sequence is at least 100 nucleotides.

5. The primer pair of claim 1, wherein said fusion partner B sequence is at least 10 nucleotides.

6. The primer pair of claim 1, wherein said fusion partner B sequence is at least 50 nucleotides.

7. The primer pair of claim 1, wherein said fusion partner B sequence is at least 100 nucleotides.

8. The primer pair of claim 1, wherein said first primer is between 13 and 100 nucleotides in length.

9. The primer pair of claim 1, wherein said first primer is between 15 and 50 nucleotides in length.

10. The primer pair of claim 1, wherein said second primer is between 13 and 100 nucleotides in length.

11. The primer pair of claim 1, wherein said second primer is between 15 and 50 nucleotides in length.

12. The primer pair of claim 1, wherein said fusion partner A sequence is present in a human LIMA1 nucleic acid, and said fusion partner B sequence is present in a human USP22 nucleic acid; or
wherein said fusion partner A sequence is present in a human LIMA1 nucleic acid, and said fusion partner B sequence is present in a human USP22 nucleic acid; or
wherein said fusion partner A sequence is present in a human ACACA nucleic acid, and said fusion partner B sequence is present in a human STAC2 nucleic acid; or
wherein said fusion partner A sequence is present in a human FAM102A nucleic acid, and said fusion partner B sequence is present in a human CIZ1 nucleic acid; or
wherein said fusion partner A sequence is present in a human GLB1 nucleic acid, and said fusion partner B sequence is present in a human CMTM7 nucleic acid; or
wherein said fusion partner A sequence is present in a human MED1 nucleic acid, and said fusion partner B sequence is present in a human STXBP4 nucleic acid; or
wherein said fusion partner A sequence is present in a human PIP4K2B nucleic acid, and said fusion partner B sequence is present in a human RAD51C nucleic acid; or
wherein said fusion partner A sequence is present in a human RAB22A nucleic acid, and said fusion partner B sequence is present in a human MYO9B nucleic acid; or
wherein said fusion partner A sequence is present in a human RPS6KB1 nucleic acid, and said fusion partner B sequence is present in a human SNF8 nucleic acid; or
wherein said fusion partner A sequence is present in a human STARD3 nucleic acid, and said fusion partner B sequence is present in a human DOK5 nucleic acid; or
wherein said fusion partner A sequence is present in a human TRPC4AP nucleic acid, and said fusion partner B sequence is present in a human MRPL45 nucleic acid; or
wherein said fusion partner A sequence is present in a human ZMYND8 nucleic acid, and said fusion partner B sequence is present in a human CEP250 nucleic acid; or
wherein said fusion partner A sequence is present in a human CTAGE5 nucleic acid, and said fusion partner B sequence is present in a human SIP1 nucleic acid; or
wherein said fusion partner A sequence is present in a human MLL5 nucleic acid, and said fusion partner B sequence is present in a human LHFPL3 nucleic acid; or
wherein said fusion partner A sequence is present in a human SEC22B nucleic acid, and said fusion partner B sequence is present in a human NOTCH2 nucleic acid; or
wherein said fusion partner A sequence is present in a human EIF3K nucleic acid, and said fusion partner B sequence is present in a human CYP39A1 nucleic acid; or
wherein said fusion partner A sequence is present in a human RAB7A nucleic acid, and said fusion partner B sequence is present in a human LRCH3 nucleic acid; or
wherein said fusion partner A sequence is present in a human RNF187 nucleic acid, and said fusion partner B sequence is present in a human OBSCN nucleic acid; or
wherein said fusion partner A sequence is present in a human SLC37A1 nucleic acid, and said fusion partner B sequence is present in a human ABCG1 nucleic acid; or
wherein said fusion partner A sequence is present in a human EXOC7 nucleic acid, and said fusion partner B sequence is present in a human CYTH1 nucleic acid; or
wherein said fusion partner A sequence is present in a human BRE nucleic acid, and said fusion partner B sequence is present in a human DPYSL5 nucleic acid; or
wherein said fusion partner A sequence is present in a human CD151 nucleic acid, and said fusion partner B sequence is present in a human DRD4 nucleic acid; or
wherein said fusion partner A sequence is present in a human LDLRAD3 nucleic acid, and said fusion partner B sequence is present in a human TCP11L1 nucleic acid; or
wherein said fusion partner A sequence is present in a human RFT1 nucleic acid, and said fusion partner B sequence is present in a human UQCRC2 nucleic acid; or
wherein said fusion partner A sequence is present in a human GSDMC nucleic acid, and said fusion partner B sequence is present in a human PVT1 nucleic acid; or
wherein said fusion partner A sequence is present in a human INTS1 nucleic acid, and said fusion partner B sequence is present in a human PRKAR1B nucleic acid; or
wherein said fusion partner A sequence is present in a human POLDIP2 nucleic acid, and said fusion partner B sequence is present in a human BRIP1 nucleic acid; or
wherein said fusion partner A sequence is present in a human MYH9 nucleic acid, and said fusion partner B sequence is present in a human EIF3D nucleic acid; or
wherein said fusion partner A sequence is present in a human BRIP1 nucleic acid, and said fusion partner B sequence is present in a human TMEM49 nucleic acid; or
wherein said fusion partner A sequence is present in a human SUPT4H1 nucleic acid, and said fusion partner B sequence is present in a human CCDC46 nucleic acid; or
wherein said fusion partner A sequence is present in a human TMEM104 nucleic acid, and said fusion partner B sequence is present in a human CDK12 nucleic acid; or
wherein said fusion partner A sequence is present in a human RIMS2 nucleic acid, and said fusion partner B sequence is present in a human ATP6V1C1 nucleic acid; or
wherein said fusion partner A sequence is present in a human TIAL1 nucleic acid, and said fusion partner B sequence is present in a human C10orf119 nucleic acid; or
wherein said fusion partner A sequence is present in a human MECP2 nucleic acid, and said fusion partner B sequence is present in a human TMLHE nucleic acid; or
wherein said fusion partner A sequence is present in a human ARID1A nucleic acid, and said fusion partner B sequence is present in a human MAST2 nucleic acid; or
wherein said fusion partner A sequence is present in a human UBR5 nucleic acid, and said fusion partner B sequence is present in a human SLC25A32 nucleic acid; or
wherein said fusion partner A sequence is present in a human KLHDC2 nucleic acid, and said fusion partner B sequence is present in a human SNTB1 nucleic acid; or
wherein said fusion partner A sequence is present in a human ARID1A nucleic acid, and said fusion partner B sequence is present in a human WDTC1 nucleic acid; or
wherein said fusion partner A sequence is present in a human HDGF nucleic acid, and said fusion partner B sequence is present in a human S100A10 nucleic acid; or
wherein said fusion partner A sequence is present in a human PPP1R12B nucleic acid, and said fusion partner B sequence is present in a human SNX27 nucleic acid; or
wherein said fusion partner A sequence is present in a human SRGAP2 nucleic acid, and said fusion partner B sequence is present in a human PRPF3 nucleic acid; or
wherein said fusion partner A sequence is present in a human WIPF2 nucleic acid, and said fusion partner B sequence is present in a human ERBB2 nucleic acid.

13. An isolated nucleic acid comprising a fusion partner A sequence and a fusion partner B sequence, wherein said fusion partner A sequence is present in a first human gene set forth in Table 3, 4, 5, 6, 8, or 10 and said fusion partner B sequence is present in a second human gene set forth in Table 3, 4, 5, 6, 8, or 10 as being a fusion partner with said first human gene.

14. The isolated nucleic acid of claim 13, wherein said fusion partner A sequence is at least 10 nucleotides.

15. The isolated nucleic acid of claim 13, wherein said fusion partner A sequence is at least 50 nucleotides.

16. The isolated nucleic acid of claim 13, wherein said fusion partner A sequence is at least 100 nucleotides.

17. The isolated nucleic acid of claim 13, wherein said fusion partner B sequence is at least 10 nucleotides.

18. The isolated nucleic acid of claim 13, wherein said fusion partner B sequence is at least 50 nucleotides.

19. The isolated nucleic acid of claim 13, wherein said fusion partner B sequence is at least 100 nucleotides.

20. The isolated nucleic acid of claim 13, wherein said fusion partner A sequence is present in a human LIMA1 nucleic acid, and said fusion partner B sequence is present in a human USP22 nucleic acid; or
wherein said fusion partner A sequence is present in a human LIMA1 nucleic acid, and said fusion partner B sequence is present in a human USP22 nucleic acid; or
wherein said fusion partner A sequence is present in a human ACACA nucleic acid, and said fusion partner B sequence is present in a human STAC2 nucleic acid; or
wherein said fusion partner A sequence is present in a human FAM102A nucleic acid, and said fusion partner B sequence is present in a human CIZ1 nucleic acid; or
wherein said fusion partner A sequence is present in a human GLB1 nucleic acid, and said fusion partner B sequence is present in a human CMTM7 nucleic acid; or
wherein said fusion partner A sequence is present in a human MED1 nucleic acid, and said fusion partner B sequence is present in a human STXBP4 nucleic acid; or
wherein said fusion partner A sequence is present in a human PIP4K2B nucleic acid, and said fusion partner B sequence is present in a human RAD51C nucleic acid; or
wherein said fusion partner A sequence is present in a human RAB22A nucleic acid, and said fusion partner B sequence is present in a human MYO9B nucleic acid; or
wherein said fusion partner A sequence is present in a human RPS6KB1 nucleic acid, and said fusion partner B sequence is present in a human SNF8 nucleic acid; or
wherein said fusion partner A sequence is present in a human STARD3 nucleic acid, and said fusion partner B sequence is present in a human DOK5 nucleic acid; or
wherein said fusion partner A sequence is present in a human TRPC4AP nucleic acid, and said fusion partner B sequence is present in a human MRPL45 nucleic acid; or
wherein said fusion partner A sequence is present in a human ZMYND8 nucleic acid, and said fusion partner B sequence is present in a human CEP250 nucleic acid; or
wherein said fusion partner A sequence is present in a human CTAGE5 nucleic acid, and said fusion partner B sequence is present in a human SIP1 nucleic acid; or
wherein said fusion partner A sequence is present in a human MLL5 nucleic acid, and said fusion partner B sequence is present in a human LHFPL3 nucleic acid; or
wherein said fusion partner A sequence is present in a human SEC22B nucleic acid, and said fusion partner B sequence is present in a human NOTCH2 nucleic acid; or
wherein said fusion partner A sequence is present in a human EIF3K nucleic acid, and said fusion partner B sequence is present in a human CYP39A1 nucleic acid; or
wherein said fusion partner A sequence is present in a human RAB7A nucleic acid, and said fusion partner B sequence is present in a human LRCH3 nucleic acid; or
wherein said fusion partner A sequence is present in a human RNF187 nucleic acid, and said fusion partner B sequence is present in a human OBSCN nucleic acid; or
wherein said fusion partner A sequence is present in a human SLC37A1 nucleic acid, and said fusion partner B sequence is present in a human ABCG1 nucleic acid; or
wherein said fusion partner A sequence is present in a human EXOC7 nucleic acid, and said fusion partner B sequence is present in a human CYTH1 nucleic acid; or
wherein said fusion partner A sequence is present in a human BRE nucleic acid, and said fusion partner B sequence is present in a human DPYSL5 nucleic acid; or
wherein said fusion partner A sequence is present in a human CD151 nucleic acid, and said fusion partner B sequence is present in a human DRD4 nucleic acid; or
wherein said fusion partner A sequence is present in a human LDLRAD3 nucleic acid, and said fusion partner B sequence is present in a human TCP11L1 nucleic acid; or
wherein said fusion partner A sequence is present in a human RFT1 nucleic acid, and said fusion partner B sequence is present in a human UQCRC2 nucleic acid; or
wherein said fusion partner A sequence is present in a human GSDMC nucleic acid, and said fusion partner B sequence is present in a human PVT1 nucleic acid; or
wherein said fusion partner A sequence is present in a human INTS1 nucleic acid, and said fusion partner B sequence is present in a human PRKAR1B nucleic acid; or
wherein said fusion partner A sequence is present in a human POLDIP2 nucleic acid, and said fusion partner B sequence is present in a human BRIP1 nucleic acid; or
wherein said fusion partner A sequence is present in a human MYH9 nucleic acid, and said fusion partner B sequence is present in a human EIF3D nucleic acid; or
wherein said fusion partner A sequence is present in a human BRIP1 nucleic acid, and said fusion partner B sequence is present in a human TMEM49 nucleic acid; or
wherein said fusion partner A sequence is present in a human SUPT4H1 nucleic acid, and said fusion partner B sequence is present in a human CCDC46 nucleic acid; or
wherein said fusion partner A sequence is present in a human TMEM104 nucleic acid, and said fusion partner B sequence is present in a human CDK12 nucleic acid; or
wherein said fusion partner A sequence is present in a human RIMS2 nucleic acid, and said fusion partner B sequence is present in a human ATP6V1C1 nucleic acid; or
wherein said fusion partner A sequence is present in a human TIAL1 nucleic acid, and said fusion partner B sequence is present in a human C10orf119 nucleic acid; or
wherein said fusion partner A sequence is present in a human MECP2 nucleic acid, and said fusion partner B sequence is present in a human TMLHE nucleic acid; or
wherein said fusion partner A sequence is present in a human ARID1A nucleic acid, and said fusion partner B sequence is present in a human MAST2 nucleic acid; or
wherein said fusion partner A sequence is present in a human UBR5 nucleic acid, and said fusion partner B sequence is present in a human SLC25A32 nucleic acid; or
wherein said fusion partner A sequence is present in a human KLHDC2 nucleic acid, and said fusion partner B sequence is present in a human SNTB1 nucleic acid; or
wherein said fusion partner A sequence is present in a human ARID1A nucleic acid, and said fusion partner B sequence is present in a human WDTC1 nucleic acid; or
wherein said fusion partner A sequence is present in a human HDGF nucleic acid, and said fusion partner B sequence is present in a human S100A10 nucleic acid; or
wherein said fusion partner A sequence is present in a human PPP1R12B nucleic acid, and said fusion partner B sequence is present in a human SNX27 nucleic acid; or
wherein said fusion partner A sequence is present in a human SRGAP2 nucleic acid, and said fusion partner B sequence is present in a human PRPF3 nucleic acid; or
wherein said fusion partner A sequence is present in a human WIPF2 nucleic acid, and said fusion partner B sequence is present in a human ERBB2 nucleic acid.
